(19) United States
(12) Patent Application Publication
(10) Pub. No.: US 2026/0165982 A1
(43) Pub. Date: Jun. 18, 2026

(54) COMPOSITIONS AND METHODS FOR DELIVERY OF NUCLEIC ACID THERAPEUTICS

(71) Applicant: Entrada Therapeutics, Inc., Boston, MA (US)

(72) Inventors: ZIQING QIAN, Lexington, MA (US); MAHBOUBEH KHEIRABADI, Natick, MA (US); PINAKIN SUKTHANKAR, Boston, MA (US); PATRICK DOUGHERTY, Boston, MA (US); XIANG LI, Belmont, MA (US); JOANNA CHIU, Boston, MA (US); NANCHER YEO, Boston, MA (US); JOHN BUZZO, Boston, MA (US); ANUSHREE PATHAK, Boston, MA (US)

(21) Appl. No.: 18/862,670

(22) PCT Filed: May 8, 2023
(86) PCT No.: PCT/US23/21324

§ 371 Date: Nov. 4, 2024

Related U.S. Application Data
(60) Provisional application No. 63/339,758, filed on May 9, 2022.
(60) Provisional application No. 63/340,892, filed on May 11, 2022.
(60) Provisional application No. 63/411,839, filed on Sep. 30, 2022.
(60) Provisional application No. 63/462,130, filed on Apr. 26, 2023.

(51) Int. Cl. A61K 9/51; A61K 47/62; A61K 47/69; C12N 15/88

(52) U.S. Cl. CPC A61K 9/5169; A61K 9/5146; A61K 47/62; A61K 47/6911; A61K 47/6929; C12N 15/88

(57) ABSTRACT

Provided herein are conjugated lipids and lipid-based particles, such as liposomes and lipid nanoparticles, containing the conjugated lipids. Methods of making the conjugated lipids and the lipid-based particles are described. Conjugated lipids include a lipid conjugated to a cell penetrating peptide. The lipid-based particles may include a payload. Compositions including the lipid-based particles may be administered to a subject.

### RELATED APPLICATIONS

This application claims the benefit of U.S. Provisional Patent Application No. 63/339,758, filed on May 9, 2022; U.S. Provisional Patent Application No. 63/411,839, filed on Sep. 30, 2022; U.S. Provisional Patent Application No. 63/340,892, filed on May 11,2022; and U.S. Provisional Patent Application No. 63/462,130, filed on Apr. 26, 2023, each of which is hereby incorporated herein by reference in their respective entireties.

### FIELD OF THE INVENTION

Compounds comprising delivery constructs conjugated to lipids and/or gene editing machinery are disclosed. Lipid-based particles containing such compounds are also disclosed. Methods of making the compounds and the lipid nanoparticles are disclosed herein. Also disclosed are compositions that include the lipid nanoparticles.

### BACKGROUND

Discovery of gene editing systems has revolutionized modern molecular biology. Gene editing systems employ several components that work in harmony to introduce precise edits in target locations of genomes. One gene editing system that is being heavily explored is the CRISPR-Cas system. CRISPR-Cas systems include a CRISPR associated (Cas) nuclease and a guide RNA (gRNA) that complex to form a ribonucleoprotein (RNP). The gRNA serves to guide the Cas nuclease to a target gene location for editing.

Effective delivery of the components of a gene editing system into the cytosol and nucleus of mammalian cells would open the door to a wide range of applications including treatment of many currently intractable diseases. However, effective delivery in a clinical setting is yet to be accomplished and has been hampered by lack of cell permeability. Additional strategies for enhancing the cell-permeability of the components of gene editing systems for a variety of therapeutic and research purposes are needed.

Lipid-based particles such as liposomes and lipid nanoparticles (LNP), are being explored to deliver gene editing systems and other payloads into cells. A lipid-based particle may enter cell through endocytosis. Once within the cell, the lipid-based particle may free its payload, allowing the payload to interact with an intended target. However, traditional lipid-based particles may have low payload delivery efficiencies due to poor endosomal escape. As such, new techniques and methods are needed to improve the endosomal escape efficiency of lipid-based particles to increase their effectiveness as payload delivery systems.

### SUMMARY

Provided herein, among other things, are compounds comprising a delivery construct conjugated to a lipid. Lipid-based particles may include the compounds comprising the delivery construct conjugated to a lipid. The delivery construct may enhance endosomal escape of payloads of the lipid-based particles. The payloads, which may include one or more components of a gene editing system may be conjugated to a delivery construct.

A lipid-based particle, comprising a lipid conjugate, is provided wherein the lipid conjugate comprises:
a lipid delivery construct conjugated to a PEGylated lipid,
the lipid delivery construct comprising a cCPP comprising 6 to 12 amino acids;
the PEGylated lipid comprising:

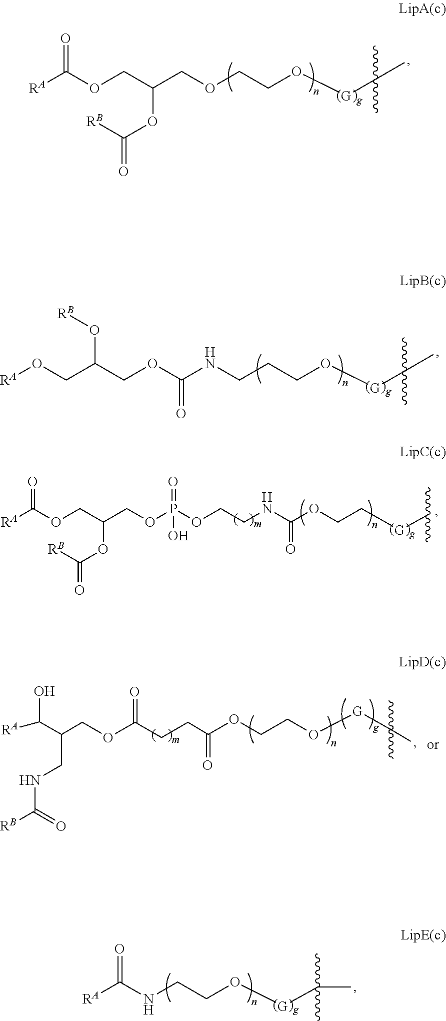

wherein:
R^{A} and R^{B} are each independently an alkyl or alkenyl of C5 to C25, wherein one or more carbons of the alkyl or alkenyl are optionally replaced with a catenated heteroatom, optionally substituted with O to form a carbonyl, or both;
n is an integer between 1 and 50;
m is an integer between 0 and 10;
g is 0 or 1; and
G is

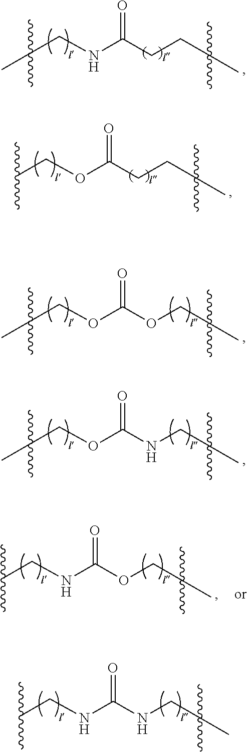

wherein l′ and l″ are each independently an integer from 0 to 10.

In embodiments, R^{A} and R^{B} are the same. In embodiments, R^{A} and R^{B} are different. In embodiments, R^{A}, R^{B}, or both are an alkyl or alkenyl of C10 to C20. In embodiments, R^{A}, R^{B}, or both are an alkyl or alkenyl of C15 to C20. In embodiments, R^{A}, R^{B}, or both are an alkyl or alkenyl of C17.

In embodiments, m is 1, 2, or 3. In embodiments, m is 1.

In embodiments, n is an integer between 30 and 50. In embodiments, n is an integer between 40 and 50.

In embodiments, g is 1.

In embodiments, l′ and l′ are 2. In embodiments, l′ is 1 and 1″ is 2.

In embodiments, the PEGylated lipid comprises:

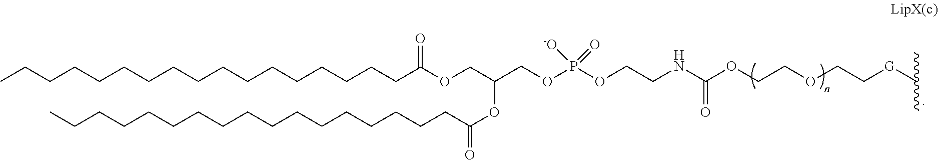

In embodiments, the PEGylated lipid comprises:

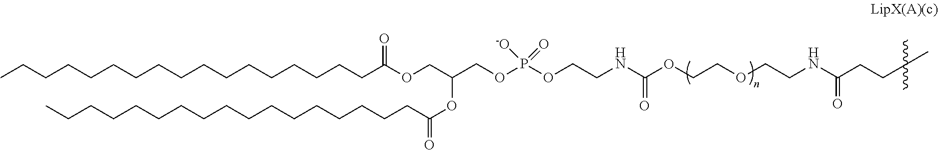

In embodiments, n is an integer between 40 and 50.

In embodiments, the lipid-based particle is a liposome. In embodiments, the lipid-based particle is a lipid nanoparticle.

A lipid conjugate is provided that comprises:
a PEGylated lipid conjugate comprising a PEGylated lipid conjugated to a lipid delivery construct,
the lipid delivery construct comprising a cyclic cell penetrating peptide (cCPP) comprising 6 to 12 amino acids;
the PEGylated lipid comprising:

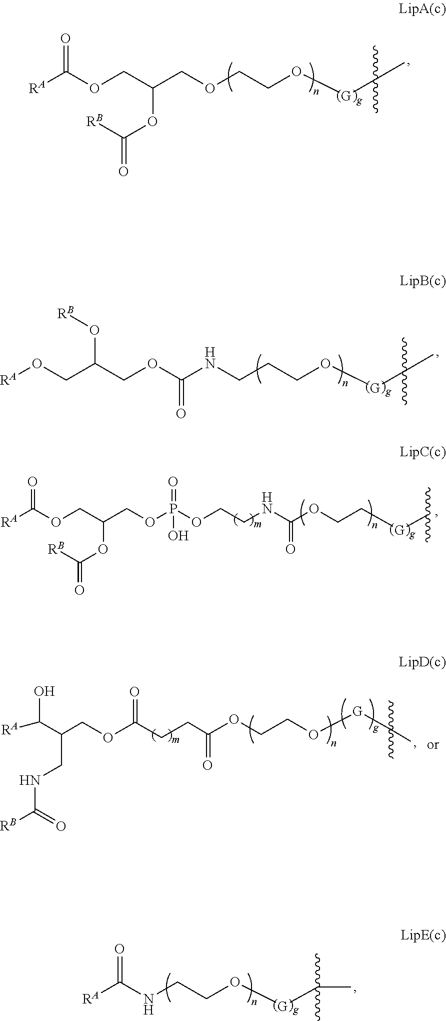

wherein:
R^{A} and R^{B} are each independently an alkyl or alkenyl of C5 to C25, wherein one or more carbons of the alkyl or alkenyl are optionally replaced with a catenated heteroatom, optionally substituted with O to form a carbonyl, or both;
n is an integer between 1 and 50;
m is an integer between 0 and 10;
g is 0 or 1; and
G is

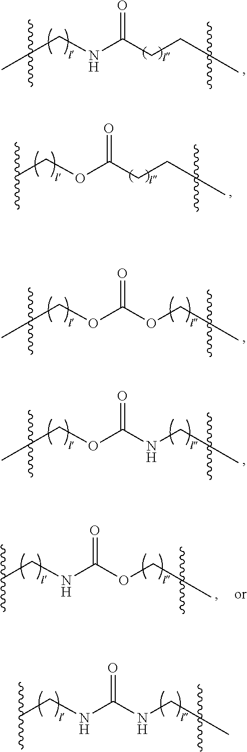

wherein l′ and l″ are each independently an integer from 0 to 10.

In embodiments, R^{A} and R^{B} are the same. In embodiments, R^{A} and R^{B} are different. In embodiments, R^{A}, R^{B}, or both are an alkyl or alkenyl of C10 to C20. In embodiments, R^{A}, R^{B}, or both are an alkyl or alkenyl of C15 to C20. In embodiments, R^{A}, R^{B}, or both are an alkyl or alkenyl of C17.

In embodiments, m is 1, 2, or 3. In embodiments, m is 1.

In embodiments, n is an integer between 30 and 50. In embodiments, n is an integer between 40 and 50.

In embodiments, g is 1.

In embodiments, l′ and l″ are 2. In embodiments, l′ is 1 and l″ is 2.

In embodiments, the PEGylated lipid comprises:

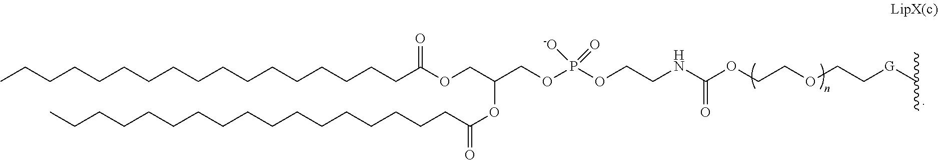

In embodiments, the PEGylated lipid comprises:

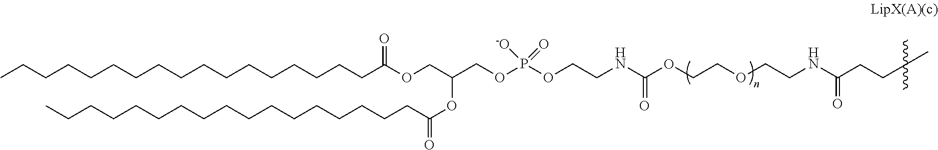

In embodiments, n is an integer between 40 and 50.

In embodiments, a lipid nanoparticle (LNP) is provided that comprises:
0.001 mol-% to 3.0 mol-% of an PEGylated lipid conjugate comprising a lipid delivery construct conjugated to a PEGylated lipid, the lipid delivery construct comprising a first cyclic cell penetrating peptide (cCPP) comprising 6 to 12 amino acids wherein at least two amino acids are charged amino acids; at least two amino acids are aromatic hydrophobic amino acids; and at least two amino acids are uncharged, and non-aromatic amino acids;
an ionizable lipid;
a helper lipid; and
a sterol.

In embodiments, the ionizable lipid is SM-102 or MC3. In embodiments, the helper lipid is DSPC. In embodiments, the LNP further comprises a non-conjugated PEGylated lipid. In embodiments, the non-conjugated PEGylated lipid is DMG-PEG2K. In embodiments, the total amount of the non-conjugated PEGylated lipid and PEGylated lipid conjugate is 3 mol-% or less. In embodiments, the total amount of the non-conjugated PEGylated lipid and PEGylated lipid conjugate is 1.5 mol-% or less. In embodiments, the LNP comprises 0.0075 mol-% to 0.2 mol-% of the PEGylated lipid conjugate. In embodiments, the LNP comprises 0.0075 mol-% to 0.08 mol-% of the PEGylated lipid conjugate. In embodiments, the LNP comprises 0.01 mol-% to 0.06 mol-% of the PEGylated lipid conjugate. In embodiments, the LNP comprises 30 mol-% to 60 mol-% of the ionizable lipid. In embodiments, the LNP comprises 40 mol-% to 60 mol-% of the ionizable lipid. In embodiments, the LNP comprises 45 mol-% to 55 mol-% of the ionizable lipid. In embodiments, the LNP comprises 5.0 mol-% to 15 mol-% of the helper lipid. In embodiments, the LNP comprises 7.5 mol-% to 15 mol-% of the helper lipid. In embodiments, the LNP comprises 7.5 mol-% to 12.5 mol-% of the helper lipid. In embodiments, the LNP comprises 20 mol-% to 60 mol-% of the sterol. In embodiments, the LNP comprises 30 mol-% to 40 mol-% of the sterol. In embodiments, the LNP comprises 35 mol-% to 40 mol-% of the sterol.

In embodiments, the LNP further comprises a payload. In embodiments, the payload comprises a ribonucleoprotein (RNP) comprising gRNA and a nuclease, or wherein the payload comprises gRNA and a nucleic acid encoding a nuclease. In embodiments, the payload is conjugated to a payload delivery construct comprising a second cCPP. In embodiments, the payload delivery construct is conjugated to the gRNA. In embodiments, the payload delivery construct is conjugated to the nuclease.

In embodiments, an EEV-ribonucleoprotein (RNP) complex conjugate is provided comprising:
a gRNA;
a nuclease; and
a payload delivery construct conjugated to the nuclease, gRNA, or both.

In embodiments, at least two amino acids of cCPP of the lipid delivery construct, payload delivery construct, or both, are, independently, charged amino acids; at least two amino acids of the cCPP are, independently, aromatic hydrophobic amino acids; and at least two amino acids of the cCPP are, independently, uncharged, and non-aromatic amino acids. In embodiments, the at least two aromatic hydrophobic amino acids of the lipid delivery construct, payload delivery construct, or both, are, independently, phenylalanine, naphthylalanine, or combinations thereof. In embodiments, the at least two uncharged, non-aromatic amino acids of the cCPP are, independently, citrulline, glycine, or combinations thereof. In embodiments, the at least two charged amino acids are, independently, arginine.

In embodiments, the lipid delivery construct, payload delivery construct, or both, independently, comprises a cCPP comprising 6-12 amino acids, wherein at least two amino acids are arginine, at least two amino acids comprise a hydrophobic side chain, and at least one amino acid is a D amino acid.

In embodiments, the lipid delivery construct, payload delivery construct, or both, independently, comprise a cCPP comprising:

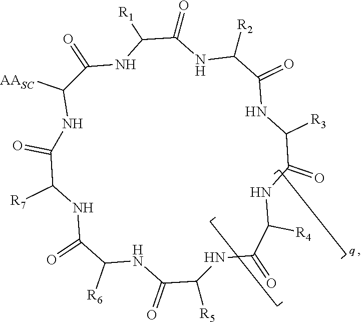

or a protonated form thereof, wherein:
R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, and R₇ are independently H or an amino acid side chain;
at least one of R₄, R₅, R₆, and R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl)alanine;
AA_{SC} is an amino acid side chain; and
q is 1, 2, 3 or 4.

In embodiments, the lipid delivery construct, payload delivery construct, or both, independently, comprise a cCPP comprising:

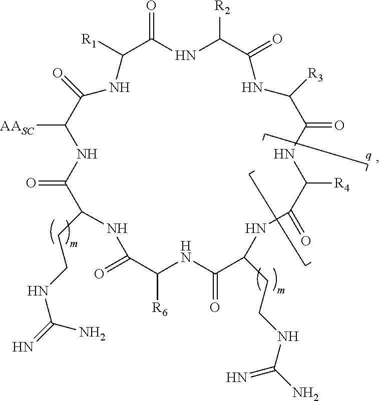

or a protonated form or salt thereof,
wherein each m is independently an integer from 0-3.

In embodiments, R₁, R₂, and R₃ are independently H or a side chain comprising an aryl group.

In embodiments, the side chain comprising an aryl group is a side chain of phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, or 3-(9-anthryl)-alanine. In embodiments, the side chain comprising an aryl group is a side chain of phenylalanine. In embodiments, two of R₁, R₂, and R₃ are a side chain of phenylalanine.

In embodiments, two of R₁, R₂, R₃, and R₄ are H.

In embodiments, the cCPP comprises:

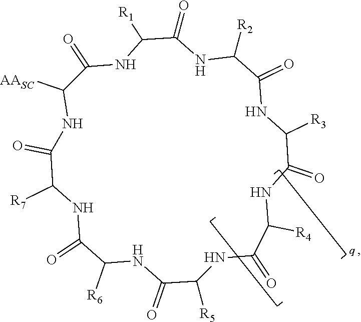

or a protonated form thereof, wherein.
at least two of R₁, R₂, R₃, R₄, R₅, R₆, and R₇ are independently the side chain of lysine; mono-methyl lysine; dimethyl lysine; trimethyl lysine; 2,4-diaminobutanoic acid; or 2,3-diaminopropionic acid;
each of R₁, R₂, R₃, R₄, R₅, R₆, and R₇ are independently H or an amino acid side chain;
AA_{SC} is an amino acid side chain; and
q is 1, 2, 3 or 4.

In embodiments, at least two of R₁, R₂, R₃, R₄, R₅, R₆, and R₇ are phenylalanine. In embodiments, at least one of R₁, R₂, R₃, R₄, R₅, R₆, and R₇ is glycine.

In embodiments, the cCPP comprises:

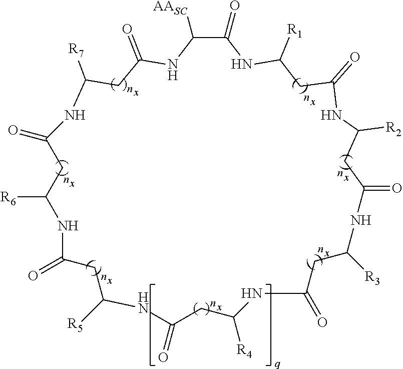

or a protonated form thereof, wherein
R₁, R₂, R₃, R₄, R₅, R₆, and R₇ are independently H or an amino acid side chain;
at least two of R₁, R₂, and R₃ are independently a side chain of phenylalanine, or naphthylalanine;
at least two of R₄, R₅, R₆, and R₇ are independently a side chain of arginine;
AA_{SC} is an amino acid side chain; and
each nₓ is 0 or 1 and at least one nₓ is 1; and
q is 1, 2, 3 or 4.

In embodiments, nₓ associated with R₁ is 1.

In embodiments, the cCPP comprises:

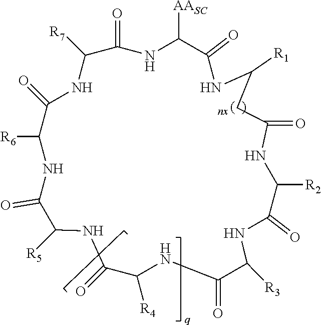

or a protonated form thereof, wherein at least one of R₁, R₂, R₃, R₄, R₅, R₆, and R₇ is the amino acid side chain of serine or histidine;

each of R₁, R₂, R₃, R₄, R₅, R₆, and R₇ are independently H or an amino acid side chain;
AA_{SC} is an amino acid side chain;
nₓ is 0 or 1; and
q is 1, 2, 3 or 4.

In embodiments, at least two of R₁, R₂, and R₃ are independently a side chain of phenylalanine, or naphthylalanine; and at least two of R₄, R₅, R₆, or R₇ are independently a side chain of arginine. In embodiments, at least two of R₄, R₅, R₆, or R₇ are independently a side chain of serine or histidine. In embodiments, R₁ and R₃ are the side chain of phenylalanine. In embodiments, R₁ is the side chain of phenylalanine and R₃ is the side chain of naphthylalanine. In embodiments, R₅ and R₇ are the side chain of arginine. In embodiments, R₄ and R₆ are the side chain of serine or histidine.

In embodiments, the lipid delivery construct, payload delivery construct, or both, independently, comprise a CPP selected from (SEQ ID NOS 16, 24, 40-41 and 132-133, respectively, in order of appearance):

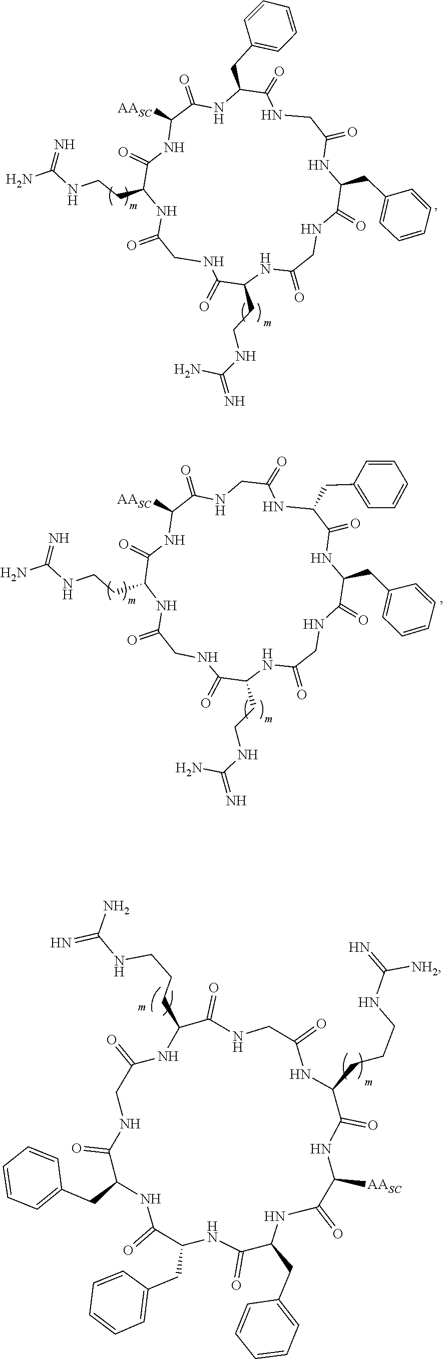

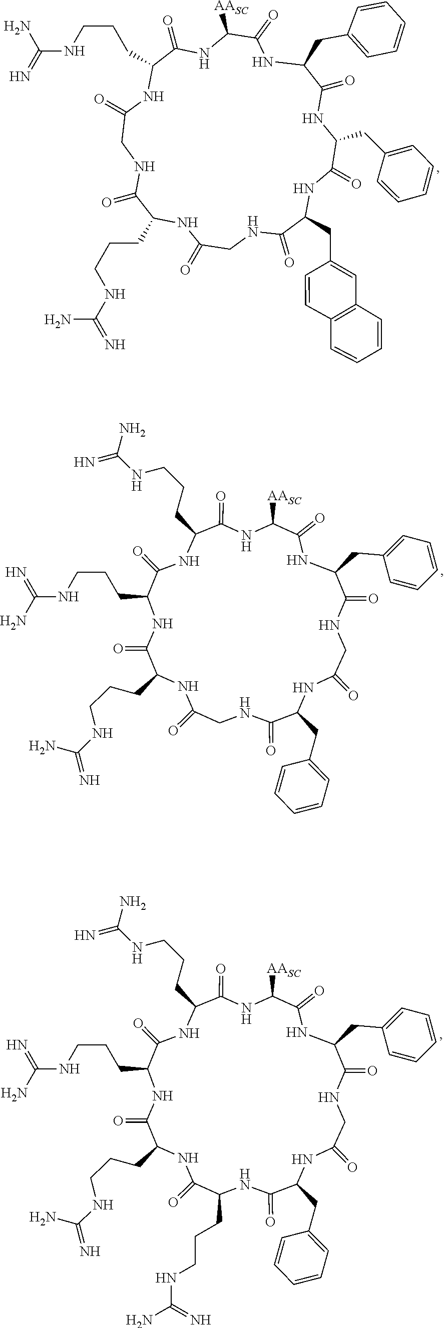

or a protonated form thereof, wherein each m independently an integer from 0-3 and AA_{SC} is an amino acid side chain.

In embodiments, AA_{SC} is a side chain of an asparagine residue, aspartic acid residue, glutamic acid residue, homoglutamic acid residue, or homoglutamate residue. In embodiments, AA_{SC} is a side chain of a glutamic acid residue. In embodiments, AA_{SC} is:

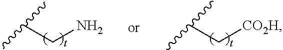

wherein t is an integer from 0 to 5.

In embodiments, the lipid delivery construct, payload delivery construct, or both, independently, comprise a cCPP selected from (SEQ ID NOS 141, 157-158, 166-168, 247, 251, 255, 257, 259, 264, 267 and 270, respectively, in order of appearance):

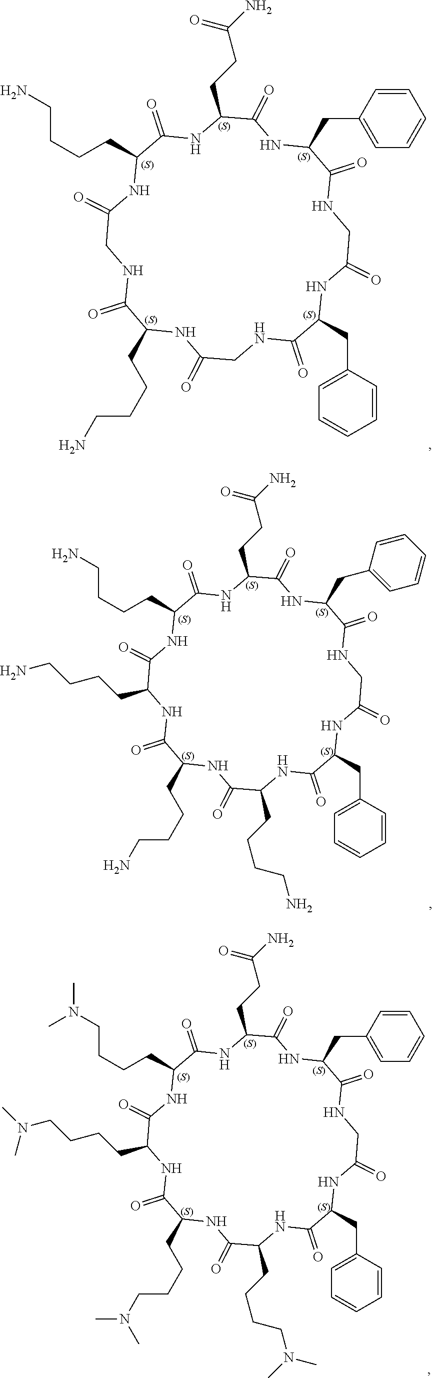

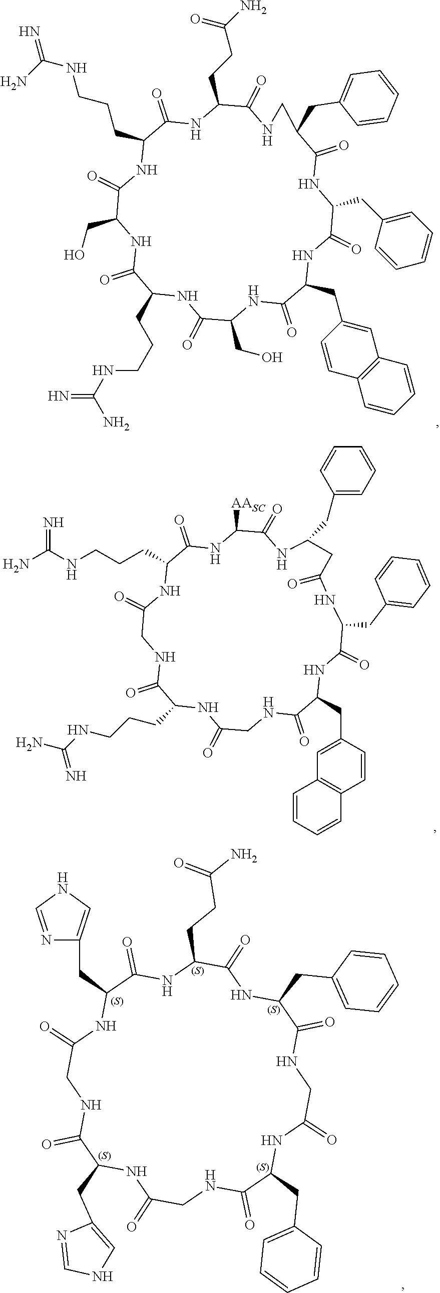

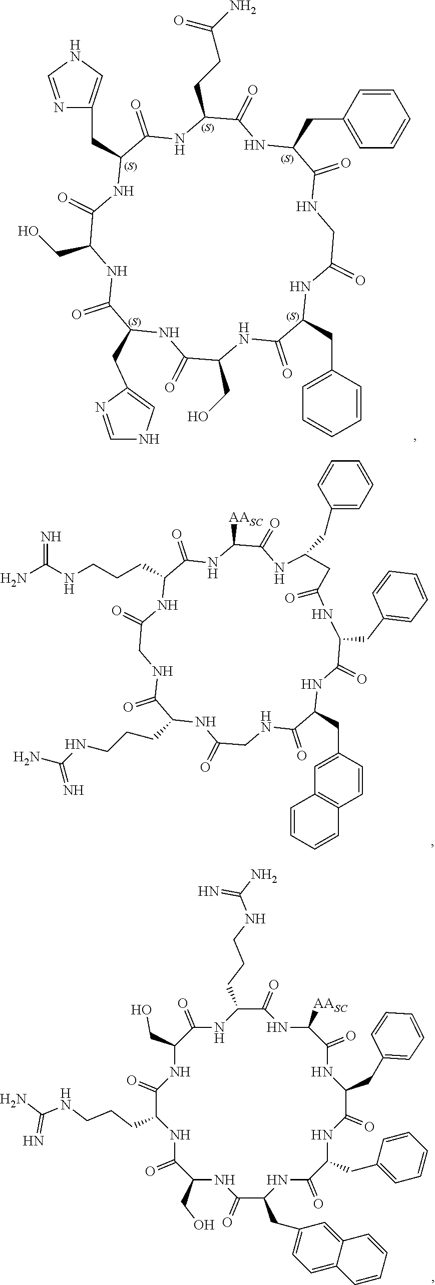

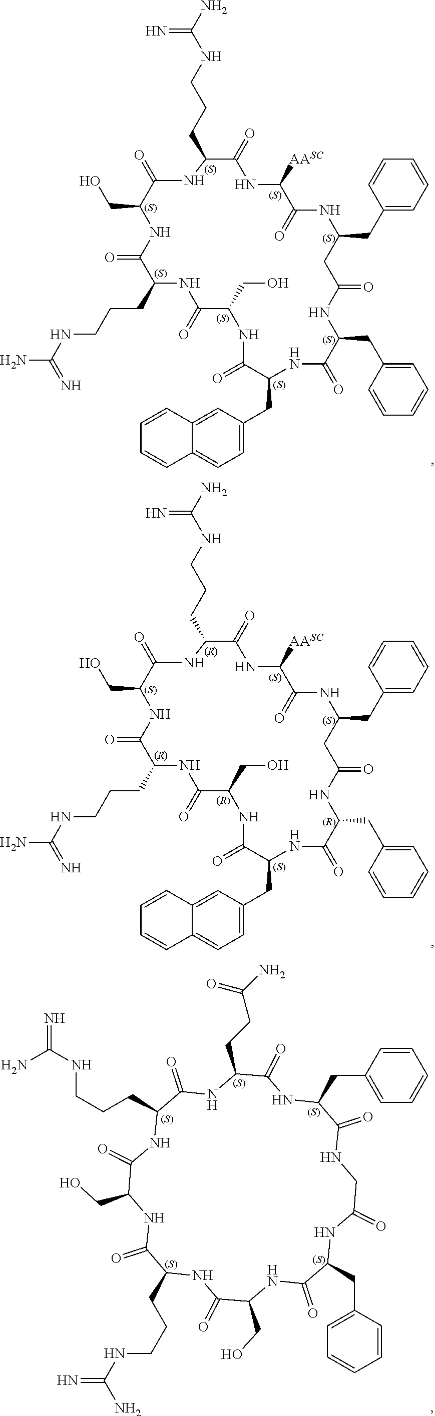

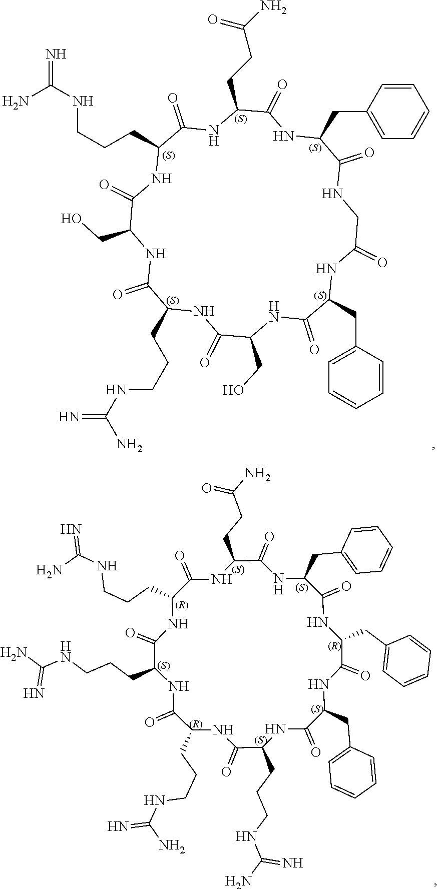

or a protonated form thereof.

In embodiments, the delivery construct comprises a cCPP and an exocyclic peptide (EP). In embodiments, the exocyclic peptide (EP) comprises from 4 to 8 amino acid residues. In embodiments, the exocyclic peptide (EP) comprises 1 or 2 amino acid residues comprising a side chain comprising a guanidine group, or a protonated form or salt thereof. In embodiments, the exocyclic peptide (EP) comprises 2, 3, or 4 lysine residues. In embodiments, the amino group on the side chain of each lysine residue is substituted with a trifluoroacetyl (—COCF3), allyloxycarbonyl (Alloc), 1-(4,4-dimethyl-2,6-dioxocyclohexylidene) ethyl (Dde), or (4,4-dimethyl-2,6-dioxocyclohex-1-ylidene-3)-methylbutyl (ivDde) group. In embodiments, the exocyclic peptide (EP) comprises at least 2 amino acid residues with a hydrophobic side chain. In embodiments, the amino acid residue with a hydrophobic side chain is selected from valine, proline, alanine, leucine, isoleucine, and methionine.

In embodiments, the exocyclic peptide (EP) comprises one of the following sequences. KK, KR, RR, HH, HK, HR, RH, KKK, KGK, KBK, KBR, KRK, KRR, RKK, RRR, KKH, KHK, HKK, HRR, HRH, HHR, HBH, HHH, HHHH (SEQ ID NO: 1), KHKK (SEQ ID NO: 2), KKHK (SEQ ID NO: 3), KKKH (SEQ ID NO: 4), KHKH (SEQ ID NO: 5), HKHK (SEQ ID NO: 6), KKKK (SEQ ID NO: 7), KKRK (SEQ ID NO: 8), KRKK (SEQ ID NO: 9), KRRK (SEQ ID NO: 10), RKKR (SEQ ID NO: 11), RRRR (SEQ ID NO: 12), KGKK (SEQ ID NO: 13), KKGK (SEQ ID NO: 14), HBHBH, HBKBH, RRRRR (SEQ ID NO: 17), KKKKK (SEQ ID NO: 18), KKKRK (SEQ ID NO: 19), RKKKK (SEQ ID NO: 20), KRKKK (SEQ ID NO: 21), KKRKK (SEQ ID NO: 22), KKKKR (SEQ ID NO: 23), KBKBK, RKKKKG (SEQ ID NO: 25), KRKKKG (SEQ ID NO: 26), KKRKKG (SEQ ID NO: 27), KKKKRG (SEQ ID NO: 28), RKKKKB (SEQ ID NO: 29), KRKKKB (SEQ ID NO: 30), KKRKKB (SEQ ID NO: 31), KKKKRB (SEQ ID NO: 32), KKKRKV (SEQ ID NO: 33), RRRRRR (SEQ ID NO: 34), HHHHHH (SEQ ID NO: 35), RHRHRH (SEQ ID NO: 36), HRHRHR (SEQ ID NO: 37), KRKRKR (SEQ ID NO: 38), RKRKRK (SEQ ID NO: 39), RBRBRB, KBKBKB, PKKKRKV (SEQ ID NO: 42), PGKKRKV (SEQ ID NO: 43), PKGKRKV (SEQ ID NO: 44), PKKGRKV (SEQ ID NO: 45), PKKKGKV (SEQ ID NO: 46), PKKKRGV (SEQ ID NO: 47) or PKKKRKG (SEQ ID NO: 48), wherein B is β-alanine.

In embodiments, the delivery construct comprises:

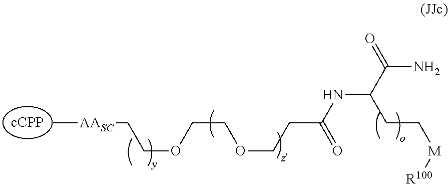

wherein:
cCPP is the cCPP of the lipid delivery construct or the payload delivery construct;
R¹⁰⁰ is the PEGylated lipid or the RNP;
y is an integer from 1 to 5;
z′ is an integer from 1-23;
AA_{SC} is any AA_{SC} as disclosed herein;
o is an integer from 1 to 5; and
M is

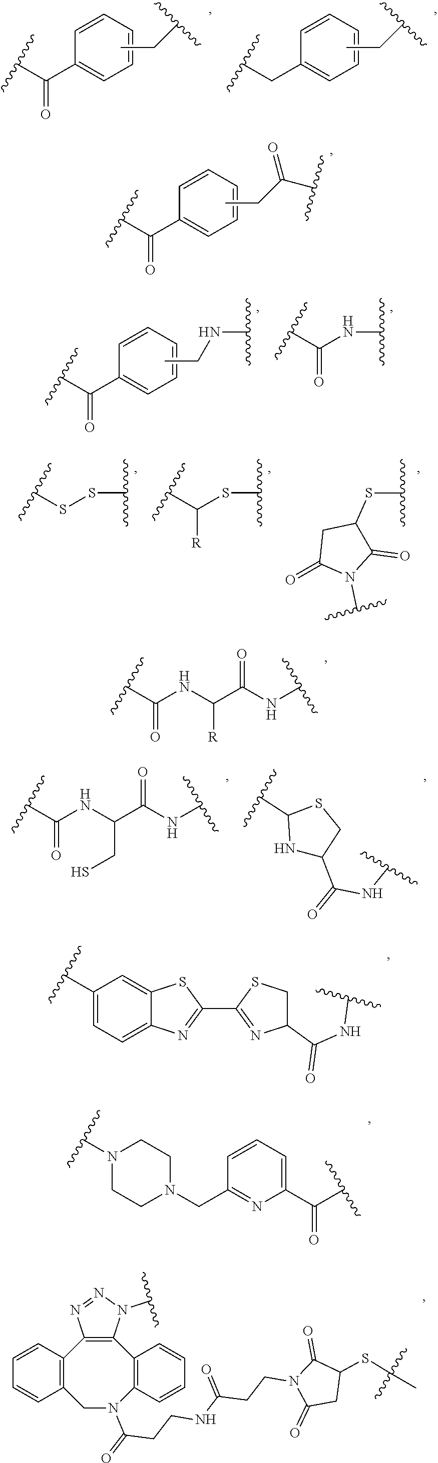

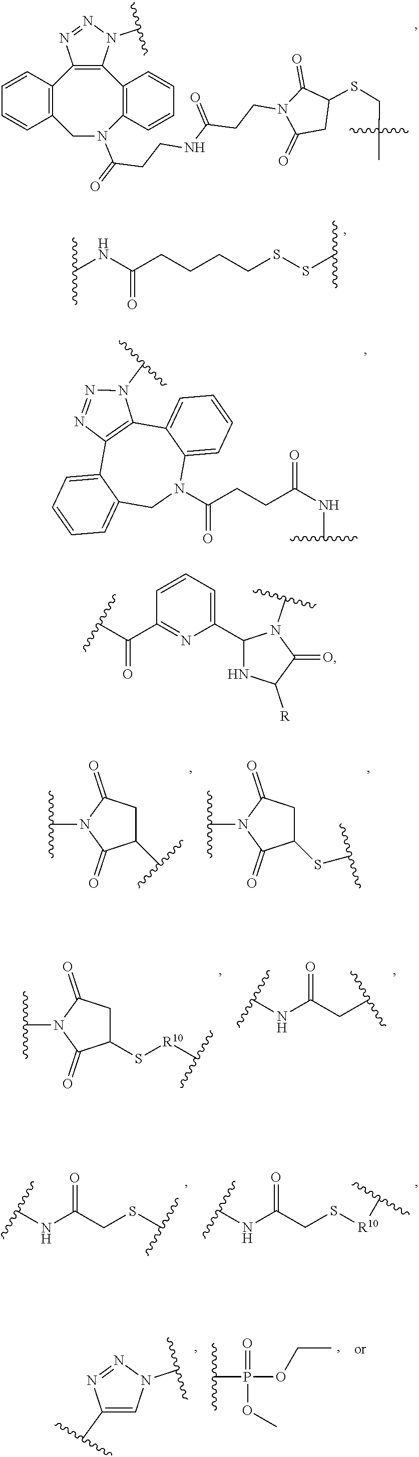

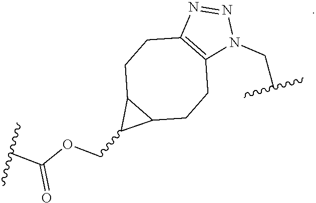

wherein R is alkyl, alkenyl, alkynyl, carbocyclyl, or heterocyclyl; and R¹⁰ is alkylene, cycloalkyl, or

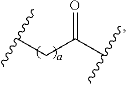

wherein a is 0 to 10.

In embodiments, the delivery construct comprises:

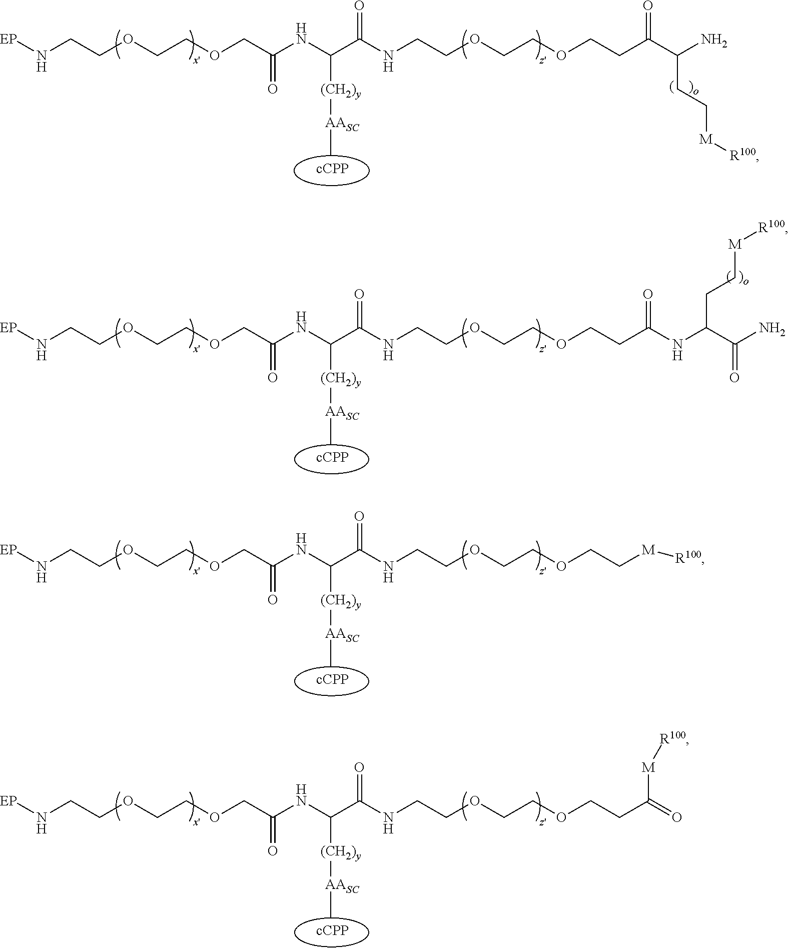

wherein:
cCPP is the cCPP of the lipid delivery construct or the payload delivery construct;
R¹⁰⁰ is the PEGylated lipid or the RNP;
EP is the exocyclic peptide;
y is an integer from 1 to 5;
x′ is an integer from 1-20;
z′ is an integer from 1-23;
AA_{SC} is an amino acid side chain of the cCPP;
o is an integer from 1 to 5; and
M is

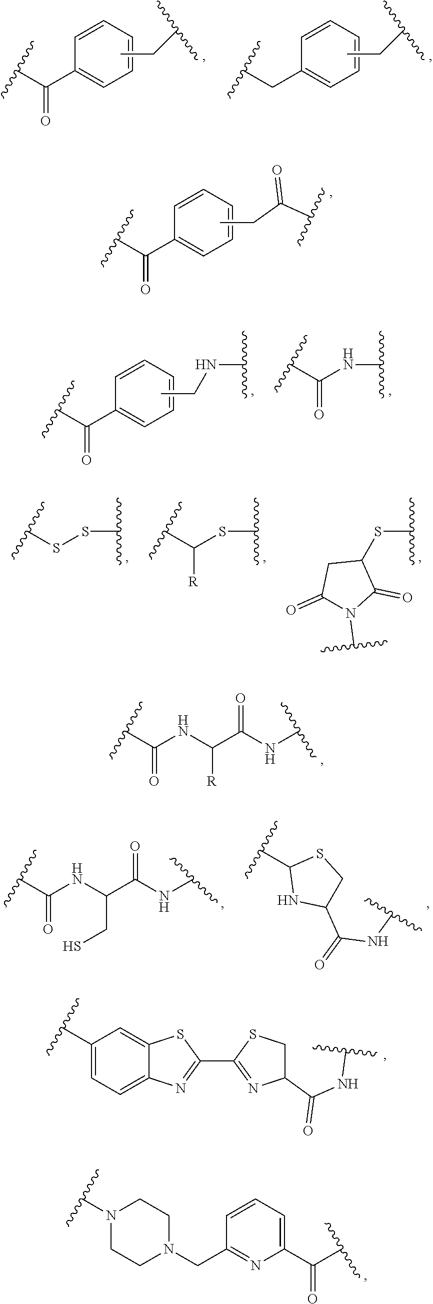

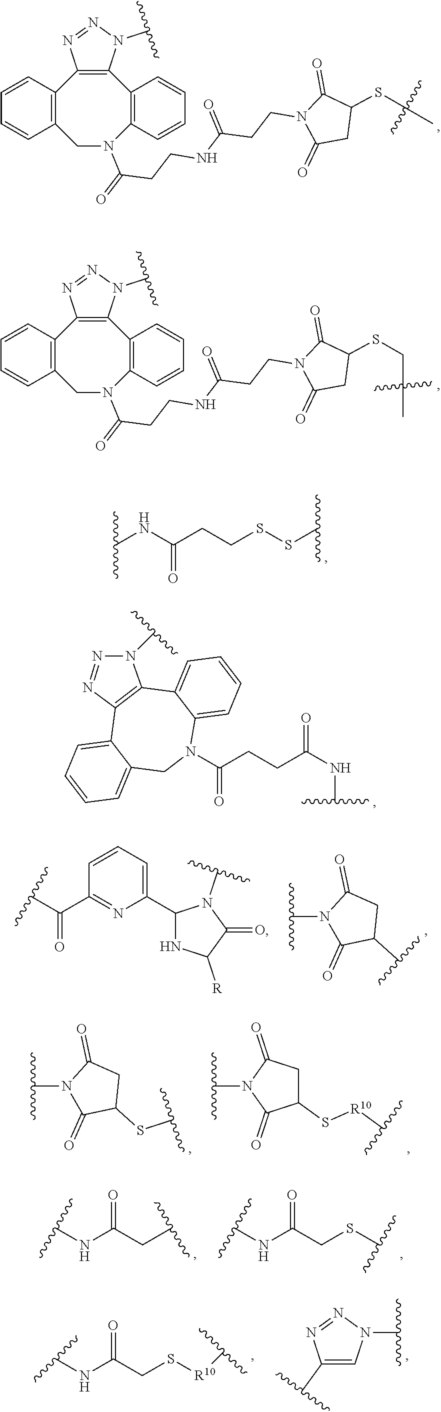

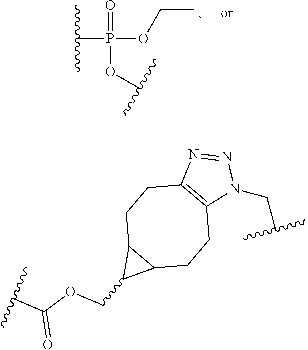

wherein R is alkyl, alkenyl, alkynyl, carbocyclyl, or heterocyclyl; and R¹⁰ is alkylene, cycloalkyl, or

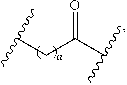

wherein a is 0 to 10.

### BRIEF DESCRIPTION OF THE DRAWINGS

The following detailed description of illustrative embodiments of the present disclosure may be best understood when read in conjunction with the following drawings.

FIGS. 1A-1B are schematic theoretical structures of a liposome (1A) and lipid nanoparticle (1B) loaded with a payload.

FIGS. 2A-C show the structures of DSPE-PEG2K-DC1 (2A), DSPE-PEG2K-DC2 (2B), and DSPE-PEG2K-DC3 (2C). Figure discloses SEQ ID NOS 483-487, respectively, in order of appearance.

FIG. 3 is a schematic theoretical structure of a lipid nanoparticle comprising a lipid conjugates.

FIG. 4 shows the structure of SM-102, DSPC, cholesterol, DSPE-PEG2K-DBCO, and D-Lin-MC3-DMA (MC3).

FIGS. 5A-SB shows a plot quantifying the mean fluorescence intensity (5A) and a fluorescence activated cell sorting (FACS) plot (5B) after HeLa cells were treated with various lipid nanoparticle formulations comprising DMG-PEG2K-DC1 lipid conjugates.

FIGS. 6A-6B are plots comparing lipid nanoparticle (LNP) size and polydispersity (6A) and polydispersity and percent encapsulation (6B) of LNPs formulated with DSPE-PEG2K-DC1 and DSPE-PEG2K-DC2 lipid conjugates.

FIG. 7 is a plot quantifying the mean fluorescence intensity for cells treated with LNPs formulated with various amounts of DSPE-PEG2K-DC1 and DSPE-PEG2K-DC2 lipid conjugates.

FIG, 8 is a plot quantifying the mean fluorescence intensity for cells treated with LNPs formulated with various total PEGylated lipid amounts.

FIG. 9 is a plot quantifying the mean fluorescence intensity for cells treated with LNPs formulated with various total PEGylated lipid amounts, various ionizable lipids, and various lipid conjugates.

FIG. 10 shows plots quantifying the mean fluorescence intensity of cells treated with various amounts of LNPs formulated with and without DSPE-PEG2K-DC2 lipid conjugates.

FIG. 11 shows the percent of GFP-negative population of cells after treatment with various concentrations of gene editing machinery formulated in LNPs comprising a lipid conjugate, LNPs lacking a lipid conjugate, and using the MESSENGERMAX reagent.

FIG. 12 is a plot showing the relationship between the size of the LNPs including a lipid conjugate and the ionic strength of the buffer.

FIG. 13A and 13B are plots showing the results of a (13A) FACS assay and a (13B) a T7 Endonuclease I assay after cells were treated with various concentrations of an DC4-RNP construct via free uptake or via lipofectamine added transfection and incubated for one day, two days, or three days. FIG. 13A shows the percent of cells displaying a GFP signal. FIG. 13B shows the amount of CRISP-induced GFP knockout.

FIG. 14A-14C are the structure of DC4 (14A), DC5 (14B), and DC6 (14C) prior to conjugation to a cargo. Figure discloses SEQ ID NOS 488-489, 15 and 490-491, respectively, in order of appearance.

FIGS. 15A-15C are plots showing the percent of GFP negative cells after exposure to a variety of LNP formulations.

### DETAILED DESCRIPTION

Disclosed herein are constructs comprising a delivery construct (DC) conjugated to a cargo such as a lipid or one or more gene editing machinery components (GEM). Such constructs can be referred to herein as cargo conjugates. As used herein, the term “delivery construct” refers to compound comprising; a cyclic cell penetrating peptide (cCPP); a compound comprising cCPP and a linker; a compound comprising cCPP and an exocyclic peptide; or a compound comprising an endosomal escape vehicle which comprises a cCPP, an exocyclic peptide, and a linker. As used herein, the term “gene editing system” refers to the combination of gene editing machinery components that can affect an edit in a target genome. As used herein, the term “gene-editing machinery” or “GEM” can be used to refer to the one or more components of a gene editing system.

Disclosed herein are compounds comprising a delivery construct conjugated to a lipid (referred to herein as a lipid conjugate or lipid delivery construct) and lipid-based particles containing the lipid conjugate. In embodiments, the delivery construct of the lipid conjugate comprises a cell penetrating peptide (CPP). In embodiments, the delivery construct of the lipid conjugate comprises a cyclic cell penetrating peptide (cCPP). In embodiments, the delivery construct of the lipid conjugate comprises a CPP or cCPP, conjugated to a linker. In embodiments, delivery construct of the lipid conjugate comprises an endosomal escape vehicle (EEV). In embodiments, the EEV comprises a cCPP, a linker, and an exocyclic peptide (EP).

In embodiments the lipid-based particles containing the lipid conjugate include a payload that includes one or more gene editing machinery components of a gene editing system. In embodiments, the one or more components of a gene editing machinery (GEM) are conjugated to delivery construct and are referred to herein as GEM conjugates. In embodiments, the lipid-based particle is a lipid nanoparticle (LNP) or a liposome.

Disclosed herein are compounds comprising a delivery construct (DC) conjugated to one or more components of a gene editing machinery (GEM), referred to herein as a GEM conjugate or a GEM construct, and lipid-based particles containing the GEM conjugate. While not wishing to be bound by theory, it is believed that conjugating the GEM to a delivery construct faciliates entry of the GEM conjugate into a cell. Conjugating the GEM to a delivery construct may facilaite endosomal escape of the GEM conjugate. In embodiments, the delivery construct of the GEM conjugate comprises a cyclic cell penetrating peptide (cCPP). In embodiments, the delivery construct of the GEM conjugate comprises a cyclic cell penetrating peptide (cCPP) and a linker. In embodiments, the delivery construct of the GEM conjugate comprises an endosomal escape vehicle (EEV). In embodiments, the EEV comprises a cCPP, a linker, and an exocyclic peptide (EP).

### Lipid-Based Particles

Liposomes and lipid nanoparticles (LNPs) are lipid-based particles that have at least one lipid layer surrounding an interior compartment. As used herein, “lipid” refers to an amphiphilic compound having a hydrophobic portion covalently attached to a hydrophilic head group or atom. The hydrophobic portion may be in the form of one or more hydrophobic tails. The hydrophobic tails may be saturated or unsaturated and may include one or more heteroatoms. Lipids include saturated fatty acids and unsaturated fatty acids; neutral glycerides and phosphoglycerides; glycolipids, and non-glyceride lipids such as sphingolipids and steroids. The lipids may be biomolecules (i.e., found in nature) or derived from biomolecules or engineered lipids. Lipids may be categorized by their chemical properties and/or their functionality within a nanoparticle. For example, LNPs may include one or more types of ionizable lipids, PEGylated lipids, and helper lipids.

An ionizable lipid is a lipid that is neutral above a particular pH and positively charged below a particular pH. In embodiments, an ionizable lipid is neutral at physiological pH (e.g., pH 7.3 to 7.5) and charged at acidic pH (e.g., pH less than 7). It is thought that ionizable lipids may function to protect the payload encapsulated within the lipid-based particle; increase encapsulation efficiency of the payload; facilitate cellular uptake; and/or to facilitate lipid-based particle cytosolic transport. For example, ionizable lipids may be neutral at physiological pH and then protonated in the endosome to enhance endosomal escape.

A PEGylated lipid or PEG-lipid is a lipid that includes at least one polyethylene glycol (PEG) unit conjugated to the head group or atom of a lipid. In embodiments, the PEGylated lipid includes a PEG chain that includes 10 or more, 30 or more, 40 or more, 45 or more, 50 or more, 60 or more, 70 or more, 80 or more, or 90 or more PEG units. In embodiments, the PEGylated lipid includes a PEG chain that includes 100 or less, 90 or less, 80 or less, 70 or less, 60 or less, 50 or less, 45 or less, 40 or less, 30 or less, or 20 or less PEG units. It is thought that PEGylated lipids improve circulation and stability of lipid-based particles in vivo. Additionally, the identity and amount of the PEGylated lipid may impact the average size and polydispersity of a population of lipid-based particles. In embodiments, the PEG portion of the PEGylated lipid may be conjugated to a delivery construct.

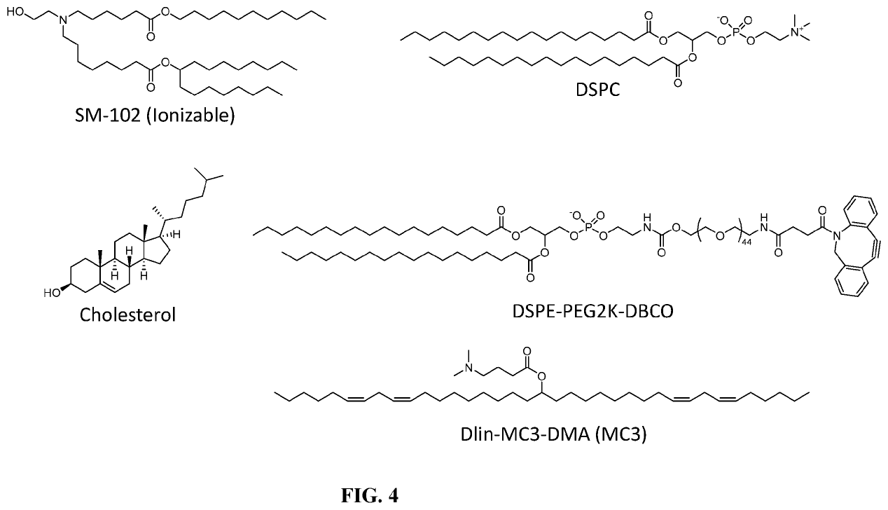

A helper lipid is any lipid included in a lipid-based particle (e.g., LNP) that is not an ionizable lipid or PEGylated lipid. Helper lipids are thought to improve stability of lipid-based particles. Specific types of helper lipids include sterols and phospholipids. Sterols are a subclass of steroids having a hydroxyl group at the 3-postion of the A-ring. Sterols may include unsaturated rings and/or carbon-containing groups appended to the fused ring structure. Examples of sterols include cholesterol (FIG. 4), phytosterol, campersterol, β-sitosterol, stigmasterol, brassicasterol, fucosterol, phytostenol, schottenol, and spinasterol. Phospholipids include a phosphate group in the hydrophilic head group. In embodiments, the helper lipid is a neutral lipid. As used herein “neutral lipid” refers to a lipid that exists in an uncharged or neutral zwitterionic form at physiological pH. In embodiments, the helper lipid is a cationic lipid. A cationic lipid is a lipid that has a formal positive charge at pH 1 to pH 10. For example, a cationic lipid may be a lipid that includes a quaternary amine.

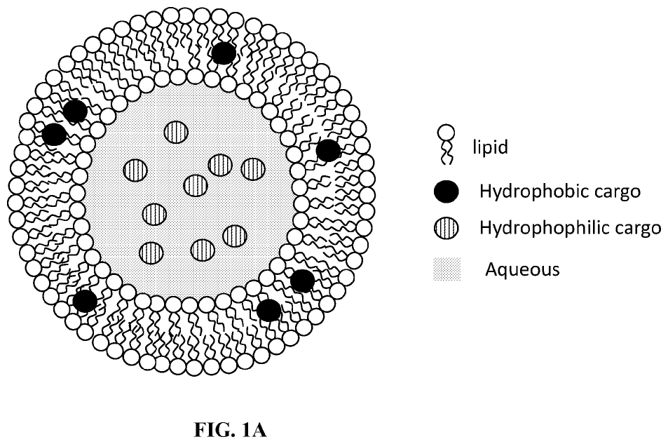

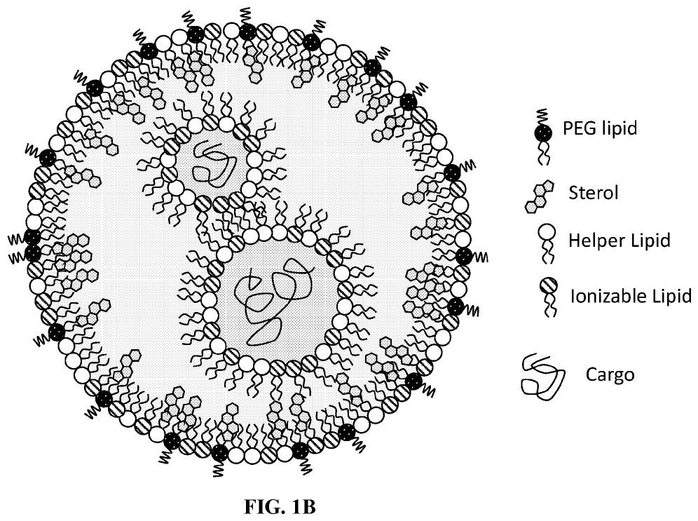

Both liposomes and LNPs may be used as drug delivery systems to delivery various payloads (e.g., drugs substances) to cells. Liposomes include a lipid bilayer that surrounds an aqueous core (FIG. 1A). Liposomes may be used to deliver hydrophilic payloads, hydrophobic payloads, or both. Hydrophilic payloads are encapsulated in the aqueous core of the liposome and hydrophobic payloads are embedded within the lipid bilayer of the liposome (FIG. 1A).

LNPs include a lipid layer defining an interior compartment that includes non-aqueous portions as well as additional lipid layers defining sub compartments having aqueous cores (FIG. 1B). Hydrophilic cargos may be encapsulated in the sub compartments of LNPs (FIG. 1B).

LNPs generally include four components: an ionizable lipid, a helper lipid, a sterol, and a PEGylated lipid. The ionizable lipid or cationic lipid, the helper lipid, the PEGylated lipid, and, sometimes, the sterol, organize into a spherical membrane that has an interior compartment, or core, and an exterior face wherein the hydrophobic tails of the various lipids are arranged within the interior compartment and the hydrophilic heads of the various lipids are arranged on the exterior face. Ionizable or cationic lipids, helper lipids, sterols, and PEGylated lipids may also be fully encapsulated within the core.

LNPs may be loaded with a payload. LNPs described herein are not limited to any particular organization or configuration of the payload and the ionizable or cationic lipids, helper lipids, sterols, and PEGylated lipids that are encapsulated within the LNP compartment. In embodiments, the LNPs described herein have a component orientation and configuration as shown in FIG. 1B. In such configuration, the hydrophilic and/or ionized or ionizable heads of the various lipids form reverse micelles (hydrophilic tails are exterior facing and hydrophilic heads are interior facing) within the compartment of the LNP further encapsulating the payload. In embodiments, the hydrophilic and/or ionized or ionizable heads of the various lipids aggregate with the payload in an unorganized fashion. In embodiments, the components of the LNPs have a unilamellar, multilamellar, bilamellar, polymorphic or facete, or polymorphic and multilamellar configuration and orientation as described in more detail in Eygeris et al., Nano Lett. (2020), 20, 4543-4549.

Liposomes and LNPs can be loaded with various types of payload. Example payload types include peptides, small molecules, and oligonucleotides. Oligonucleotide payloads may include RNA such as mRNA, siRNA, guide RNA; and/or DNA such as vectors encoding RNA (e.g., mRNA) and/or proteins. In embodiments, the payload may include both a protein and an oligonucleotide. In embodiments, the payload may be a nucleoprotein such as a ribonucleoprotein.

### Delivery Constructs

A delivery construct may be conjugated to a cargo. The cargo may be lipid, a component of gene editing machinery (GEM), or a component of a payload of a lipid-based particle, which may be a component of GEM. In embodiments, delivery construct is conjugated to a lipid to form a lipid conjugate. In embodiments, a delivery construct is conjugated to one or more components of GEM, to form a-GEM conjugate. For example, in embodiments, the delivery construct is conjugated to a ribonucleoprotein (RNP). In embodiments, GEM conjugates are used as lipid-based particle payloads. In embodiments, GEM conjugates are delivered to a cell independently of a lipid-based particle.

### Cell Penetrating Peptides (CPP)

In embodiments, the delivery construct includes a cell penetrating peptide (CPP). The CPP can be a cyclic cell penetrating peptide (cCPP). The cargo may be lipid, a component of gene editing machinery (GEM), or a payload of a lipid-based particle. In embodiments, the payload of a lipid-based particle may be a component of GEM. In embodiments, a CPP is conjugated to a lipid to form a lipid conjugate. In embodiments, a CPP is conjugated to one or more components of GEM to form a GEM conjugate. In embodiments, the CPP is conjugated to a ribonucleoprotein (RNP). In embodiments, GEM conjugate is delivered to a cell as a payload of a lipid-based particle. In embodiments, GEM conjugates are delivered to a cell independently of a lipid-based particle.

The delivery construct may include one or more linkers (L) that link the CPP or cCPP to the cargo. Two or more components that are linked are a part of a single compound. In embodiments, the delivery construct comprises a linker conjugating a CPP to a lipid cargo thereby forming a lipid conjugate. In embodiments, the delivery construct comprises a linker conjugating a CPP to a GEM cargo thereby forming a GEM conjugate.

In embodiments, the cell penetrating peptide (CPP) comprises 6 to 20 amino acid residues. The cell penetrating peptide can be a cyclic cell penetrating peptide (cCPP). The cCPP is capable of penetrating a cell membrane.

In embodiments, the cCPP can direct a payload of a lipid nanoparticle to penetrate the membrane of a cell. The cCPP can deliver the payload of a lipid nanoparticle to the cytosol of the cell. The cCPP can deliver the payload of a lipid nanoparticle to a cellular location where a target is located. To conjugate the cCPP to a cargo (e.g., lipid or payload), at least one bond or lone pair of electrons on the cCPP can be replaced.

In embodiments, the cCPP can direct a GEM conjugate to penetrate the membrane of a cell. The cCPP can deliver the GEM conjugate to the cytosol of the cell. The cCPP can deliver the GEM conjugate to a cellular location where a target is located. To conjugate the cCPP to a cargo (e.g., a component of gene editing machinery or “GEM”), at least one bond or lone pair of electrons on the cCPP can be replaced.

The total number of amino acid residues in the cCPP is in the range of from 6 to 20 amino acid residues, e.g., 6, 7, 8, 9, 10, 11, 12, 13, 14, 15, 16, 17, 18, 19, or 20 amino acid residues, inclusive of all ranges and subranges therebetween. The cCPP can comprise 6 to 13 amino acid residues. The cCPP can comprise 6 to 10 amino acids. By way of example, cCPP comprising 6-10 amino acid residues can have a structure according to any of Formula I-A to I-E:

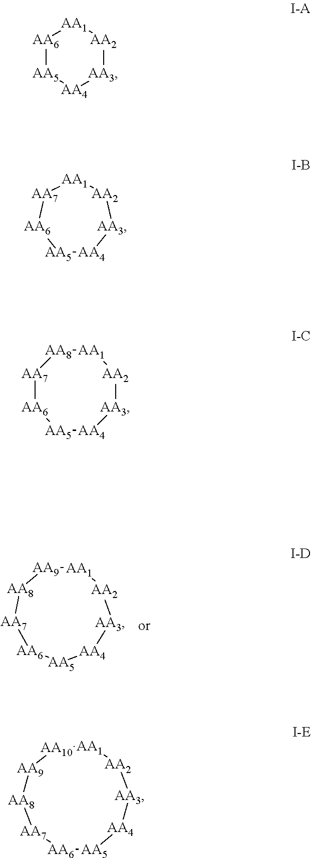

wherein AA₁, AA₂, AA₃, AA₄, AA₅, AA₆, AA₇, AA₈, AA₉, and AA₁₀ are amino acid residues.

The cCPP can comprise 6 to 8 amino acids. The cCPP can comprise 8 amino acids.

Each amino acid in the cCPP may be a natural or non-natural amino acid. Abbreviations used herein for some natural and non-natural amino acids are shown in Table 1.

As used herein, the term “amino acid” refers to compounds having an amino group and a carboxylic acid group. Most amino acids (except for glycine) also have a side chain. As used herein, “amino acid side chain” or “side chain” refers to the characterizing substituent bound to the α-carbon of the amino acid.

An “α-amino acid” is an amino acid in which the amino group is attached to the first (alpha) carbon adjacent to the carboxylic acid group, such that the carbon atom of the carbonyl is separated from the nitrogen atom of the amino group by one carbon atom. A “b-amino acid” (also called “beta-amino acid,” and “β-amino acid”) is an analog of an α-amino acid in which the amino group is attached to the second (beta) carbon, rather than the alpha-carbon, such that the carbon atom of the carbonyl is separated from the nitrogen atom of the amino group by two carbon atoms. Examples of b-amino acids include but are not limited to b-alanine and b-homophenylalanine. An “uncharged” amino acid is an amino acid that does not have a charge at a physiological pH (between 5.0 and 8.0). It is noted that histidine can exist in neutral or positively charged forms at physiological pH.

A side chain that does not comprise an aryl or heteroaryl group, can be referred to herein as a “non-aryl” side chain. In embodiments, the side chain that does not comprise an aryl or heteroaryl group can be uncharged and is referred to herein as an uncharged, non-aryl side chain. Amino acids with uncharged non-aryl amino side chains include, but are not limited to, histidine, threonine, serine, leucine, isoleucine, valine, neopentylglycine, alanine, homoalanine, homoserine, 3-(4-Thiazolyl)-alanine, 3-(4-furanyl)-alanine, 3-(4-thienyl)-alanine, and b-amino acid derivatives thereof.

TABLE 1
Amino Acid Abbreviations

| | Abbreviations* | Abbreviations* |
|---|---|---|
| Amino Acid | L-amino acid | D-amino acid |
| Alanine | Ala (A) | ala (a) |
| Allo-isoleucine | Aile | Aile |
| Arginine | Arg (R) | arg (r) |
| Asparagine | Asn (N) | asn (n) |
| Aspartic acid | Asp (D) | asp (d) |
| Cysteine | Cys (C) | cys (c) |
| Citrulline | Cit | Cit |
| Cyclohexylalanine | Cha | cha |
| 2,3-diaminopropionic acid | Dap | dap |
| 4-fluorophenylalanine | Fpa (Σ) | pfa |
| Glutamic acid | Glu (E) | glu (e) |
| Glutamine | Gln (Q) | gln (q) |
| Glycine | Gly (G) | gly (g) |
| Histidine | His (H) | his (h) |
| Homoproline (aka pipecolic acid) | Pip (Θ) | pip (⊖) |
| Isoleucine | Ile (I) | ile (i) |
| Leucine | Leu (L) | leu (l) |
| Lysine | Lys (K) | lys (k) |
| Methionine | Met (M) | met (m) |
| 3-(2-naphthyl)-alanine | Nal (Φ) | nal (φ) |
| 3-(1-naphthyl)-alanine | 1-Nal | 1-nal |
| Norleucine | Nle (Ω) | nle |
| Phenylalanine | Phe (F) | phe (f) |
| Phenylglycine | Phg (Ψ) | phg |
| 4-(phosphonodifluoromethyl)phenylalanine | F₂Pmp (Λ) | f₂pmp |
| Proline | Pro (P) | pro (p) |
| Sarcosine | Sar (Ξ) | sar |
| Selenocysteine | Sec (U) | sec (u) |
| Serine | Ser (S) | ser (s) |
| Threonine | Thr (T) | thr (y) |
| Tyrosine | Tyr (Y) | tyr (y) |
| Tryptophan | Trp (W) | trp (w) |
| Valine | Val (V) | val (v) |
| Tert-butyl-alanine | Tle | tle |
| Penicillamine | Pen | Pen |
| Homoarginine | HomoArg | homoarg |
| Nicotinyl-lysine | Lys(NIC) | lys(NIC) |
| Triflouroacetyl-lysine | Lys(TFA) | lys(TFA) |
| Methyl-leucine | MeLeu | meLeu |
| 3-(3-benzothienyl)-alanine | Bta | bta |
| N-methyl lysine (monomethyl lysine) | K(me) | k(me) |
| N,N-dimethyl lysine (dimethyl lysine) | K(me)2 | k(me)2 |
| N,N,N-trimethyl lysine (trimethyl lysine) | K(me)3 | K(me)3 |
| Beta-homophenylalanine | βhF, B-hF, b-hF | βhf |

*single letter abbreviations: capital letters indicate the L-amino acid form, lower case letter indicate the D-amino acid form. Beta amino acids are denoted by a β, B, or b followed by the amino acid abbreviation.

As used herein, “polyethylene glycol” and “PEG” are used interchangeably. “PEGm,” and “PEGₘ,” are, or are derived from, a molecule of the formula HO(CO)—(CH₂)ₙ—(OCH₂CH₂)ₘ—NH₂ where n is any integer from 1 to 5 and m is any integer from 1 to 23. In embodiments, n is 1 or 2. In embodiments, n is 1. In embodiments, n is 2. In embodiments, n is 1 and m is 2. In embodiments, n is 2 and m is 2. In embodiments, nis 1 and m is 4. In embodiments, n is 2 and m is 4. In embodiments, n is 1 and m is 12. In embodiments, n is 2 and m is 12.

As used herein, “miniPEGm” or “miniPEGₘ” are, or are derived from, a molecule of the formula HO(CO)—(CH₂)ₙ—(OCH₂CH₂)ₘ—NH₂ where n is 1 and m is any integer from 1 to 23. For example, “miniPEG2” or “miniPEG₂” is, or is derived from, (2-[2-[2-aminoethoxy]ethoxy]acetic acid), and “miniPEG4” or “miniPEG₄” is, or is derived from, HO(CO)—(CH₂)ₙ—(OCH₂CH₂)ₘ-NH₂ where n is 1 and m is 4.

In embodiments, one or two amino acids in the CPP (e.g., cCPP) can have no side chain. In embodiments, all amino acids in the CPP (e.g., cCPP) have a side chain. As used herein, when no side chain is present, the amino acid has two hydrogen atoms on the carbon atom(s) (e.g., —CH₂—) linking the amine and carboxylic acid of the amino acid residue. The amino acid having no side chain can be glycine or beta-alanine.

The cCPP (e.g., cCPP) can comprise from 6 to 20, from 6 to 10, or from 6 to 8 amino acid residues, wherein: (i) at least one amino acid can be glycine, b-alanine, serine, histidine or 4-aminobutyric acid; (ii) at least one amino acid can have a side chain comprising an aryl or heteroaryl group; and (iii) at least one amino acid has a side chain comprising a guanidine group, or a protonated form thereof.

In embodiments, one amino acid of the CPP (e.g., cCPP) can be glycine, b-alanine, serine, histidine, or 4-aminobutyric acid. In embodiments, two amino acids can be, independently, glycine, b-alanine, serine, histidine or 4-aminobutyric acid. In embodiments, three amino acids can be glycine, b-alanine, serine, histidine, or 4-aminobutyric acid.

In embodiments, one amino acid of the CPP (e.g., cCPP) can have a side chain comprising an aryl or heteroaryl group. In embodiments, two amino acids of the CPP (e.g., cCPP) can have a side chain comprising an aryl or heteroaryl group. In embodiments, three amino acids of the CPP (e.g., cCPP) can have a side chain comprising an aryl or heteroaryl group.

In embodiments, one amino acid of the CPP (e.g., cCPP) can have a side chain that does not comprise an aryl or heteroaryl group, referred to herein as a “non-aryl” side chain. In embodiments, the side chain that does not comprise an aryl or heteroaryl group can be uncharged and is referred to herein as an uncharged, non-aryl side chain. In embodiments, two amino acids of the CPP (e.g., cCPP) can have an uncharged, non-aryl side chain. In embodiments, three amino acids of the CPP (e.g., cCPP) can have an uncharged, non-aryl side chain. Amino acids with uncharged non-aryl amino side chains include, but are not limited to, histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine.

The CPP (e.g., cCPP) can comprise 6 to 20 amino acids, wherein: (i) at least one amino acid has a side chain comprising a guanidine group, or a protonated form thereof; (ii) at least one amino acid has no side chain or a side chain comprising

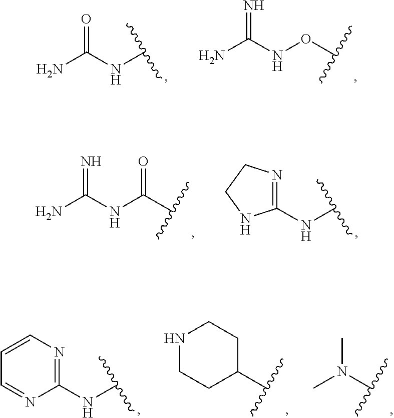

or a protonated form thereof; and (iii) at least two amino acids independently have a side chain comprising an aromatic or heteroaromatic group.

At least two amino acids can have no side chain or a side chain comprising

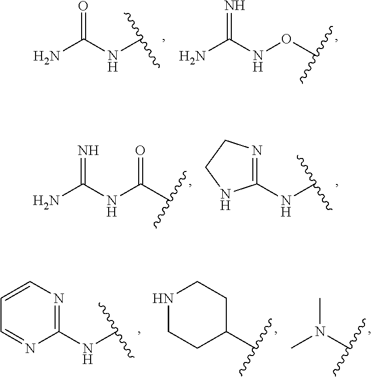

or a protonated form thereof. As used herein, when no side chain is present, the amino acid has two hydrogen atoms on the carbon atom(s) (e.g., —CH₂—) linking the amine and carboxylic acid.

The amino acid having no side chain can be glycine or beta-alanine.

The CPP (e.g., cCPP) can comprise from 6 to 20 amino acid residues which form the CPP (e.g., cCPP), wherein: (i) at least one amino acid can be glycine, b-alanine, or 4-aminobutyric acid residues; (ii) at least one amino acid can have a side chain comprising an aryl or heteroaryl group; and (iii) at least one amino acid has a side chain comprising a guanidine group,

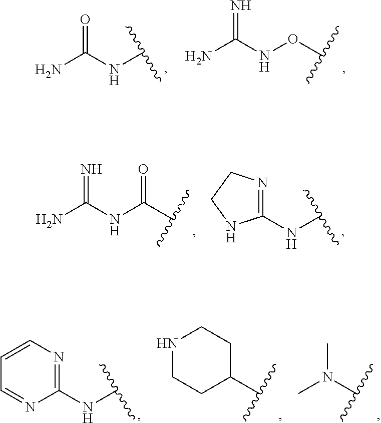

or a protonated form thereof.

The CPP (e.g., cCPP) can comprise from 6 to 20 amino acid residues which form the cCPP, wherein: (i) at least two amino acids can independently be glycine, b-alanine, or 4-aminobutyric acid residues; (ii) at least one amino acid can have a side chain comprising an aryl or heteroaryl group; and (iii) at least one amino acid has a side chain comprising a guanidine group,

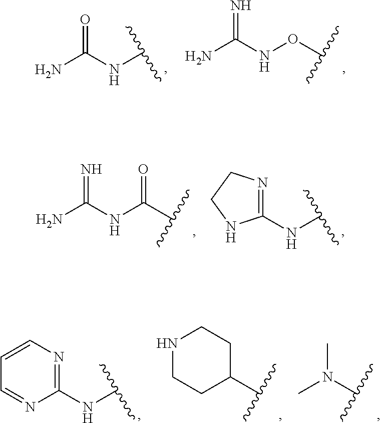

or a protonated form thereof.

The CPP (e.g., cCPP) can comprise from 6 to 20 amino acid residues which form the CPP (e.g., cCPP), wherein: (i) at least three amino acids can independently be glycine, b-alanine, or 4-aminobutyric acid residues; (ii) at least one amino acid can have a side chain comprising an aromatic or heteroaromatic group; and (iii) at least one amino acid can have a side chain comprising a guanidine group,

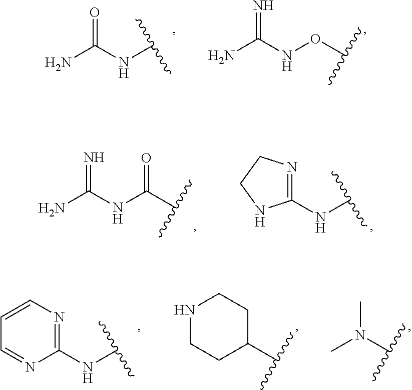

or a protonated form thereof.

The CPP (e.g., cCPP) can comprise 1 or 2 amino acid residues selected from uncharged non-aryl amino acids residues.

The CPP (e.g., cCPP) can comprise 2 contiguous amino acids with hydrophobic side chains The CPP (e.g., cCPP) can comprise 3 contiguous amino acids with hydrophobic side chains.

In embodiments, one amino acid of the CPP (e.g., cCPP) can have a side chain that does not comprise an aryl or heteroaryl group, referred to herein as a “non-aryl” side chain. In embodiments, the side chain that does not comprise an aryl or heteroaryl group can be uncharged and is referred to herein as an uncharged, non-aryl side chain. In embodiments, two amino acids of the CPP (e.g., cCPP) can have an uncharged, non-aryl side chain. In embodiments, three amino acids of the CPP (e.g., cCPP) can have an uncharged, non-aryl side chain. Amino acids with uncharged non-aryl amino side chains include, but are not limited to, histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine.

In embodiments, one amino acid of the CPP (e.g., cCPP) has a side chain comprising a guanidine group, or a protonated form thereof. In embodiments, two amino acids of the CPP (e.g., cCPP) can have a side chain comprising a guanidine group, or a protonated form thereof. In embodiments, three amino acids of the CPP (e.g., cCPP) can have a side chain comprising a guanidine group, or a protonated form thereof. In embodiments, four amino acids of the CPP (e.g., cCPP) can have a side chain comprising a guanidine group, or a protonated form thereof.

### Glycine and Related Amino Acid Residues

The cCPP can comprise (i) 1, 2, 3, 4, 5, or 6 glycine, b-alanine, 4-aminobutyric acid residues, or combinations thereof. The cCPP can comprise (i) 2 glycine, b-alanine, 4-aminobutyric acid residues, or combinations thereof. The cCPP can comprise (i) 3 glycine, b-alanine, 4-aminobutyric acid residues, or combinations thereof. The cCPP can comprise (i) 4glycine, b-alanine, 4-aminobutyric acid residues, or combinations thereof. The cCPP can comprise (i) 5 glycine, b-alanine, 4-aminobutyric acid residues, or combinations thereof. The cCPP can comprise (i) 6 glycine, b-alanine, 4-aminobutyric acid residues, or combinations thereof. The cCPP can comprise (i) 3, 4, or 5 glycine, b-alanine, 4-aminobutyric acid residues, or combinations thereof. The cCPP can comprise (i) 3 or 4 glycine, b-alanine, 4-aminobutyric acid residues, or combinations thereof.

The cCPP can comprise (i) 1, 2, 3, 4, 5, or 6 glycine residues. The cCPP can comprise (i) 2 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues. The cCPP can comprise (i) 2 or 3 glycine residues. The cCPP can comprise (i) 1 or 2 glycine residues.

The cCPP can comprise (1) 3, 4, 5, or 6 glycine, b-alanine, 4-aminobutyric acid residues, or combinations thereof. The cCPP can comprise (i) 3 glycine, b-alanine, 4-aminobutyric acid residues, or combinations thereof. The cCPP can comprise (i) 4 glycine, b-alanine, 4-aminobutyric acid residues, or combinations thereof. The cCPP can comprise (i) 5 glycine, b-alanine, 4-aminobutyric acid residues, or combinations thereof. The cCPP can comprise (i) 6 glycine, b-alanine, 4-aminobutyric acid residues, or combinations thereof. The cCPP can comprise (i) 3, 4, or 5 glycine, b-alanine, 4-aminobutyric acid residues, or combinations thereof. The cCPP can comprise (i) 3 or 4 glycine, b-alanine, 4-aminobutyric acid residues, or combinations thereof.

The cCPP can comprise at least three glycine residues. The cCPP can comprise (i) 3, 4, 5, or 6 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (1) 3, 4, or 5 glycine residues. The cCPP can comprise (i) 3 or 4 glycine residues

In embodiments, none of the glycine, b-alanine, or 4-aminobutyric acid residues in the cCPP are contiguous. Two or three glycine, b-alanine, 4-or aminobutyric acid residues can be contiguous. Two glycine, b-alanine, or 4-aminobutyric acid residues can be contiguous.

In embodiments, none of the glycine residues in the cCPP are contiguous. Each glycine residue in the cCPP can be separated by an amino acid residue that is not glycine. Two or three glycine residues can be contiguous. Two glycine residues can be contiguous

Amino Acid Side Chains with an Aromatic or Heteroaromatic Group

The cCPP can comprise (ii) 2, 3, 4, 5 or 6 amino acid residues independently having a side chain comprising an aromatic or heteroaromatic group. The cCPP can comprise (ii) 2 amino acid residues independently having a side chain comprising an aromatic or heteroaromatic group. The cCPP can comprise (ii) 3 amino acid residues independently having a side chain comprising an aromatic or heteroaromatic group. The cCPP can comprise (ii) 4 amino acid residues independently having a side chain comprising an aromatic or heteroaromatic group. The cCPP can comprise (ii) 5 amino acid residues independently having a side chain comprising an aromatic or heteroaromatic group. The cCPP can comprise (ii) 6 amino acid residues independently having a side chain comprising an aromatic or heteroaromatic group. The cCPP can comprise (ii) 2, 3, or 4 amino acid residues independently having a side chain comprising an aromatic or heteroaromatic group. The cCPP can comprise (ii) 2 or 3 amino acid residues independently having a side chain comprising an aromatic or heteroaromatic group.

The cCPP can comprise (ii) 2, 3, 4, 5 or 6 amino acid residues independently having a side chain comprising an aromatic group. The cCPP can comprise (ii) 2 amino acid residues independently having a side chain comprising an aromatic group. The cCPP can comprise (ii) 3 amino acid residues independently having a side chain comprising an aromatic group. The cCPP can comprise (ii) 4 amino acid residues independently having a side chain comprising an aromatic group. The cCPP can comprise (ii) 5 amino acid residues independently having a side chain comprising an aromatic group. The cCPP can comprise (ii) 6 amino acid residues independently having a side chain comprising an aromatic group. The cCPP can comprise (ii) 2, 3, or 4 amino acid residues independently having a side chain comprising an aromatic group. The cCPP can comprise (ii) 2 or 3 amino acid residues independently having a side chain comprising an aromatic group.

The aromatic group can be a 6- to 14-membered aryl. Aryl can be phenyl, naphthyl or anthracenyl, each of which is optionally substituted. Aryl can be phenyl or naphthyl, each of which is optionally substituted. The heteroaromatic group can be a 6- to 14-membered heteroaryl having 1, 2, or 3 heteroatoms selected from N, O, and S. Heteroaryl can be pyridyl, quinolyl, or isoquinolyl.

The amino acid residue having a side chain comprising an aromatic or heteroaromatic group can each independently be bis (homonaphthylalanine), homonaphthylalanine, naphthylalanine, phenylglycine, bis (homophenylalanine), homophenylalanine, phenylalanine, tryptophan, 3-(3-benzothienyl)-alanine, 3-(2-quinolyl)-alanine, O-benzylserine, 3-(4-(benzyloxy) phenyl)-alanine, S-(4-methylbenzyl)cysteine, N-(naphthalen-2-yl)glutamine, 3-(1,1′-biphenyl-4-yl)-alanine, 3-(3-benzothienyl)-alanine or tyrosine, each of which is optionally substituted with one or more substituents. The amino acid having a side chain comprising an aromatic or heteroaromatic group can each independently be selected from:

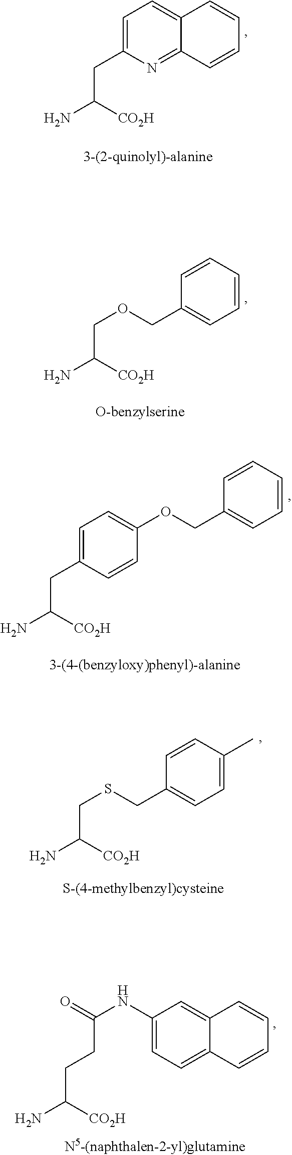 [SMILES: NC(Cc1ccc2ccccc2n1)C(=O)O ; NC(COCc1ccccc1)C(=O)O ; NC(Cc1ccc(OCc2ccccc2)cc1)C(=O)O ; Cc1ccc(CSCC(N)C(=O)O)cc1 ; NC(CCC(=O)Nc1ccc2ccccc2c1)C(=O)O]

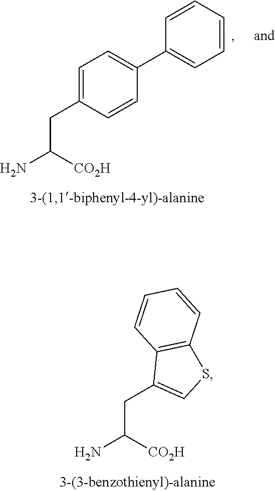 [SMILES: NC(Cc1ccc(-c2ccccc2)cc1)C(=O)O ; NC(Cc1csc2ccccc12)C(=O)O]

wherein the H on the N-terminus and/or the H on the C-terminus are replaced by a peptide bond.

The amino acid residue having a side chain comprising an aromatic or heteroaromatic group can each be independently a residue of phenylalanine, naphthylalanine, phenylglycine, homophenylalanine, homonaphthylalanine, bis (homophenylalanine), bis-(homonaphthylalanine), tryptophan, or tyrosine, each of which is optionally substituted with one or more substituents. The amino acid residue having a side chain comprising an aromatic group can each independently be a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine. The amino acid residue having a side chain comprising an aromatic group can each independently be a residue of phenylalanine, naphthylalanine, phenylglycine, homophenylalanine, or homonaphthylalanine, each of which is optionally substituted with one or more substituents. The amino acid residue having a side chain comprising an aromatic group can each be independently a residue of phenylalanine, naphthylalanine, homophenylalanine, homonaphthylalanine, bis(homonaphthylalanine), or bis(homonaphthylalanine), each of which is optionally substituted with one or more substituents. The amino acid residue having a side chain comprising an aromatic group can each be independently a residue of phenylalanine or naphthylalanine, each of which is optionally substituted with one or more substituents. At least one amino acid residue having a side chain comprising an aromatic group can be a residue of phenylalanine. At least two amino acid residues having a side chain comprising an aromatic group can be residues of phenylalanine. Each amino acid residue having a side chain comprising an aromatic group can be a residue of phenylalanine.

In embodiments, none of the amino acids having the side chain comprising the aromatic or heteroaromatic group are contiguous. Two amino acids having the side chain comprising the aromatic or heteroaromatic group can be contiguous. Two contiguous amino acids can have opposite stereochemistry. The two contiguous amino acids can have the same stereochemistry. Three amino acids having the side chain comprising the aromatic or heteroaromatic group can be contiguous. Three contiguous amino acids can have the same stereochemistry. Three contiguous amino acids can have alternating stereochemistry.

The amino acid residues comprising aromatic or heteroaromatic groups can be L-amino acids. The amino acid residues comprising aromatic or heteroaromatic groups can be D-amino acids. The amino acid residues comprising aromatic or heteroaromatic groups can be a mixture of D- and L-amino acids.

The optional substituent can be any atom or group which does not significantly reduce (e.g., by more than 50%) the cytosolic delivery efficiency of the cCPP, e.g., compared to an otherwise identical sequence which does not have the substituent. The optional substituent can be a hydrophobic substituent or a hydrophilic substituent. The optional substituent can be a hydrophobic substituent. The substituent can increase the solvent-accessible surface area (as defined herein) of the hydrophobic amino acid. The substituent can be halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. The substituent can be halogen.

The hydrophobicity of amino acid residues can be measured and/or calculated using a variety of techniques. In embodiments, the hydrophobicity of an amino acid residue can be determined by calculating its consensus value on the consensus scale of D. Eisenberg et al., using the method described in D. Eisenberg et al., “Hydrophobic Moments and Protein Structure,” Faraday Symp. Chem. Soc. 1982, 17, 109-120 (e.g., D. Eisenberg et al.). A hydrophobic amino acid is an amino acid that has a hydrophobic side chain.

Amino Acid Residues having a Side Chain Comprising a Guanidine Group, Guanidine Replacement Group, or Protonated Form Thereof

As used herein, guanidine refers to the structure:

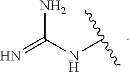 [SMILES: *NC(=N)N]

As used herein, a protonated form of guanidine refers to the structure:

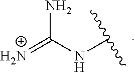 [SMILES: *NC(N)=[NH2+]]

Guanidine replacement groups refer to functional groups on the side chain of amino acids that will be positively charged at or above physiological pH or those that can recapitulate the hydrogen bond donating and accepting activity of guanidinium groups.

The guanidine replacement groups facilitate cell penetration and delivery of therapeutic agents while reducing toxicity associated with guanidine groups or protonated forms thereof. The cCPP can comprise at least one amino acid having a side chain comprising a guanidine or guanidinium replacement group. The cCPP can comprise at least two amino acids having a side chain comprising a guanidine or guanidinium replacement group. The cCPP can comprise at least three amino acids having a side chain comprising a guanidine or guanidinium replacement group

The guanidine or guanidinium group can be an isostere of guanidine or guanidinium. The guanidine or guanidinium replacement group can be less basic than guanidine.

As used herein, a guanidine replacement group refers to

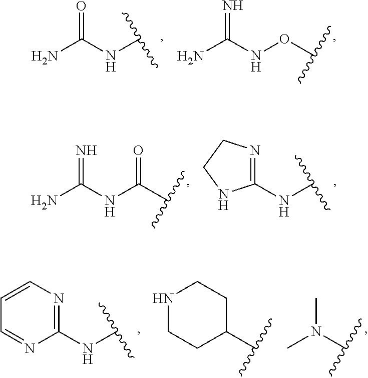

or a protonated form thereof.

The disclosure relates to a cCPP comprising from 6 to 20 amino acids residues, wherein: (i) at least one amino acid has a side chain comprising a guanidine group, or a protonated form thereof; (ii) at least one amino acid residue has no side chain or a side chain comprising

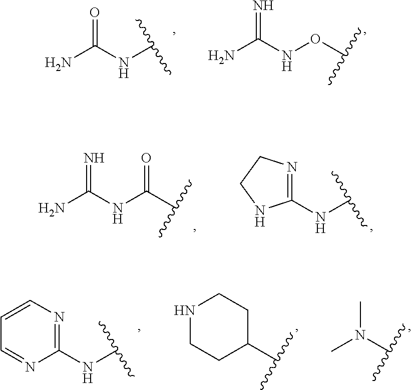

or a protonated form thereof, and (iii) at least two amino acids residues independently have a side chain comprising an aromatic or heteroaromatic group.

At least two amino acids residues can have no side chain or a side chain comprising

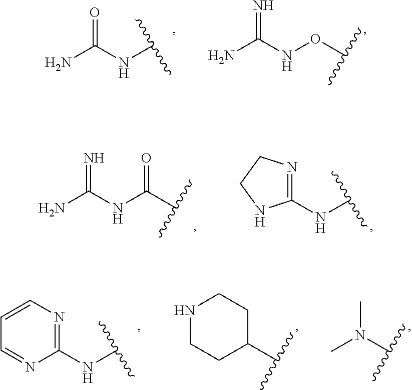

or a protonated form thereof. As used herein, when no side chain is present, the amino acid residue has two hydrogen atoms on the carbon atom(s) (e.g., —CH₂—) linking the amine and carboxylic acid.

The cCPP can comprise at least one amino acid having a side chain comprising one of the following moieties:

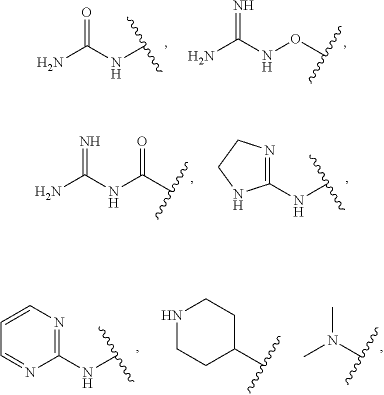

or a protonated form thereof.

The cCPP can comprise at least two amino acids each independently having one of the following moieties

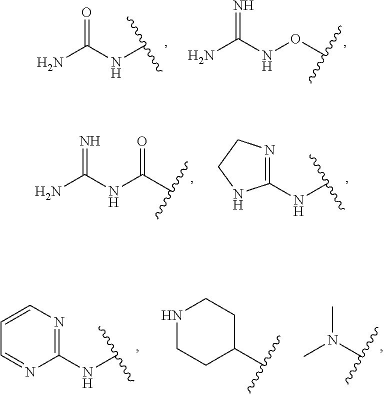

or a protonated form thereof. At least two amino acids can have a side chain comprising the same moiety selected from:

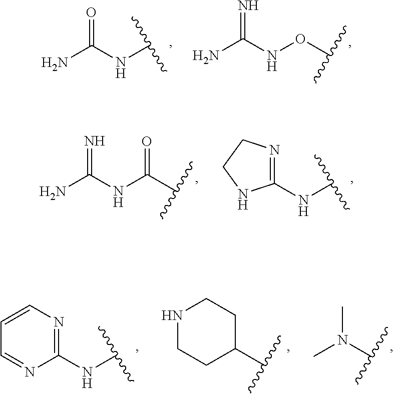

or a protonated form thereof. At least one amino acid can have a side chain comprising

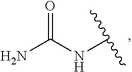 [SMILES: *NC(N)=O]

or a protonated form thereof. At least two amino acids can have a side chain comprising

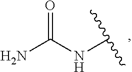 [SMILES: *NC(N)=O]

or a protonated form thereof. One, two, three, or four amino acids can have a side chain comprising

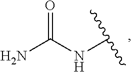 [SMILES: *NC(N)=O]

or a protonated form thereof. One amino acid can have a side chain comprising

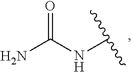 [SMILES: *NC(N)=O]

or a protonated form thereof. Two amino acids can have a side chain comprising

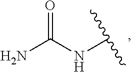 [SMILES: *NC(N)=O]

or a protonated form thereof.

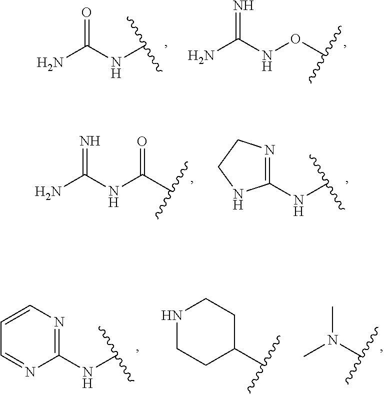

or a protonated form thereof, can be attached to the terminus of the amino acid side chain

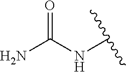 [SMILES: *NC(N)=O]

can be attached to the terminus of the amino acid side chain.

The cCPP can comprise (iii) 2, 3, 4, 5 or 6 amino acid residues independently having a side chain comprising a guanidine group, guanidine replacement group, or a protonated form thereof. The cCPP can comprise (iii) 2 amino acid residues independently having a side chain comprising a guanidine group, guanidine replacement group, or a protonated form thereof. The cCPP can comprise (iii) 3 amino acid residues independently having a side chain comprising a guanidine group, guanidine replacement group, or a protonated form thereof. The cCPP can comprise (iii) 4 amino acid residues independently having a side chain comprising a guanidine group, guanidine replacement group, or a protonated form thereof. The cCPP can comprise (iii) 5 amino acid residues independently having a side chain comprising a guanidine group, guanidine replacement group, or a protonated form thereof. The cCPP can comprise (iii) 6 amino acid residues independently having a side chain comprising a guanidine group, guanidine replacement group, or a protonated form thereof. The cCPP can comprise (iii) 2, 3, 4, or 5 amino acid residues independently having a side chain comprising a guanidine group, guanidine replacement group, or a protonated form thereof. The cCPP can comprise (iii) 2, 3, or 4 amino acid residues independently having a side chain comprising a guanidine group, guanidine replacement group, or a protonated form thereof. The cCPP can comprise (iii) 2 or 3 amino acid residues independently having a side chain comprising a guanidine group, guanidine replacement group, or a protonated form thereof. The cCPP can comprise (iii) at least one amino acid residue having a side chain comprising a guanidine group or protonated form thereof. The cCPP can comprise (iii) two amino acid residues having a side chain comprising a guanidine group or protonated form thereof. The cCPP can comprise (iii) three amino acid residues having a side chain comprising a guanidine group or protonated form thereof.

The amino acid residues can independently have the side chain comprising the guanidine group, guanidine replacement group, or the protonated form thereof that are not contiguous. Two amino acid residues can independently have the side chain comprising the guanidine group, guanidine replacement group, or the protonated form thereof can be contiguous. Three amino acid residues can independently have the side chain comprising the guanidine group, guanidine replacement group, or the protonated form thereof can be contiguous. Four amino acid residues can independently have the side chain comprising the guanidine group, guanidine replacement group, or the protonated form thereof can be contiguous. The contiguous amino acid residues can have the same stereochemistry. The contiguous amino acids can have alternating stereochemistry.

The amino acid residues independently having the side chain comprising the guanidine group, guanidine replacement group, or the protonated form thereof, can be L-amino acids. The amino acid residues independently having the side chain comprising the guanidine group, guanidine replacement group, or the protonated form thereof, can be D-amino acids. The amino acid residues independently having the side chain comprising the guanidine group, guanidine replacement group, or the protonated form thereof, can be a mixture of L- or D-amino acids.

Each amino acid residue having the side chain comprising the guanidine group, or the protonated form thereof, can independently be a residue of arginine, homoarginine, 2-amino-3-propionic acid, 2-amino-4-guanidinobutyric acid or a protonated form thereof. Each amino acid residue having the side chain comprising the guanidine group, or the protonated form thereof, can independently be a residue of arginine or a protonated form thereof.

Each amino acid having the side chain comprising a guanidine replacement group, or protonated form thereof, can independently be

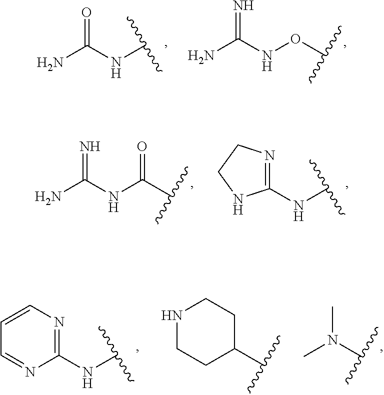

or a protonated form thereof.

Without being bound by theory, it is hypothesized that guanidine replacement groups have reduced basicity, relative to arginine and in some cases are uncharged at physiological pH (e.g., a —N(H)C(O)), and are capable of maintaining the bidentate hydrogen bonding interactions with phospholipids on the plasma membrane that is believed to facilitate effective membrane association and subsequent internalization. The removal of positive charge is also believed to reduce toxicity of the cCPP and/or EEV.

Those skilled in the art will appreciate that the N-and/or C-termini of the above non-natural aromatic hydrophobic amino acids, upon incorporation into the peptides disclosed herein, form amide bonds.

The cCPP can comprise a first amino acid having a side chain comprising an aromatic or heteroaromatic group and a second amino acid having a side chain comprising an aromatic or heteroaromatic group, wherein an N-terminus of a first glycine forms a peptide bond with the first amino acid having the side chain comprising the aromatic or heteroaromatic group, and a C-terminus of the first glycine forms a peptide bond with the second amino acid having the side chain comprising the aromatic or heteroaromatic group. Although by convention, the term “first amino acid” often refers to the N-terminal amino acid of a peptide sequence, as used herein “first amino acid” is used to distinguish the referent amino acid from another amino acid (e.g., a “second amino acid”) in the cCPP such that the term “first amino acid” may or may refer to an amino acid located at the N-terminus of the peptide sequence.

The cCPP can comprise an N-terminus of a second glycine forms a peptide bond with an amino acid having a side chain comprising an aromatic or heteroaromatic group, and a C-terminus of the second glycine forms a peptide bond with an amino acid having a side chain comprising a guanidine group, or a protonated form thereof.

The cCPP can comprise a first amino acid having a side chain comprising a guanidine group, or a protonated form thereof, and a second amino acid having a side chain comprising a guanidine group, or a protonated form thereof, wherein an N-terminus of a third glycine forms a peptide bond with a first amino acid having a side chain comprising a guanidine group, or a protonated form thereof, and a C-terminus of the third glycine forms a peptide bond with a second amino acid having a side chain comprising a guanidine group, or a protonated form thereof.

The cCPP can comprise a residue of asparagine, aspartic acid, glutamine, glutamic acid, or homoglutamine. The cCPP can comprise a residue of asparagine. The cCPP can comprise a residue of glutamine.

The cCPP can comprise a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

While not wishing to be bound by theory, it is believed that the chirality of the amino acids in the cCPPs may impact cytosolic uptake efficiency. The cCPP can comprise at least one D amino acid. The cCPP can comprise one to fifteen D amino acids. The cCPP can comprise one to ten D amino acids. The cCPP can comprise 1, 2, 3, or 4 D amino acids. The cCPP can comprise 2, 3, 4, 5, 6, 7, or 8 contiguous amino acids having alternating D and L chirality. The cCPP can comprise three contiguous amino acids having the same chirality. The cCPP can comprise two contiguous amino acids having the same chirality. At least two of the amino acids can have the opposite chirality. The at least two amino acids having the opposite chirality can be adjacent to each other. At least three amino acids can have alternating stereochemistry relative to each other. The at least three amino acids having the alternating chirality relative to each other can be adjacent to each other. At least four amino acids have alternating stereochemistry relative to each other. The at least four amino acids having the alternating chirality relative to each other can be adjacent to each other. At least two of the amino acids can have the same chirality. At least two amino acids having the same chirality can be adjacent to each other. At least two amino acids have the same chirality and at least two amino acids have the opposite chirality. The at least two amino acids having the opposite chirality can be adjacent to the at least two amino acids having the same chirality. Accordingly, adjacent amino acids in the cCPP can have any of the following sequences: D-L; L-D; D-L-L-D; L-D-D-L; L-D-L-L-D; D-L-D-D-L; D-L-L-D-L; or L-D-D-L-D. The amino acid residues that form the cCPP can all be L-amino acids. The amino acid residues that form the cCPP can all be D-amino acids.

At least two of the amino acids can have a different chirality. At least two amino acids having a different chirality can be adjacent to each other. At least three amino acids can have different chirality relative to an adjacent amino acid. At least four amino acids can have different chirality relative to an adjacent amino acid. At least two amino acids have the same chirality and at least two amino acids have a different chirality. One or more amino acid residues that form the cCPP can be achiral. The cCPP can comprise a motif of 3, 4, or 5 amino acids, wherein two amino acids having the same chirality can be separated by an achiral amino acid. The cCPPs can comprise the following sequences: D/L-X-D/L; D/L-X-D/L-X; D/L-X-D/L-X-D/L; D-X-D; D-X-D-X; D-X-D-X-D; L-X-L; L-X-L-X; or L-X-L-X-L, wherein D/L indicates that the amino acid can be a D or an L amino acid and X is an achiral amino acid. The achiral amino acid can be glycine.

An amino acid having a side chain comprising:

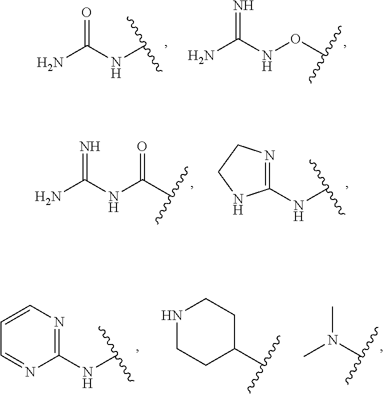

or a protonated form thereof, can be adjacent to an amino acid having a side chain comprising an aromatic or heteroaromatic group. An amino acid having a side chain comprising:

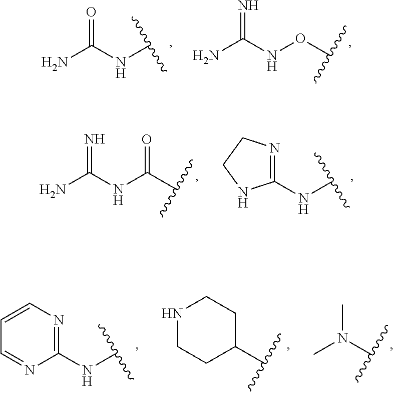

or a protonated form thereof, can be adjacent to at least one amino acid having a side chain comprising a guanidine or protonated form thereof. An amino acid having a side chain comprising a guanidine or protonated form thereof can be adjacent to an amino acid having a side chain comprising an aromatic or heteroaromatic group. Two amino acids having a side chain comprising:

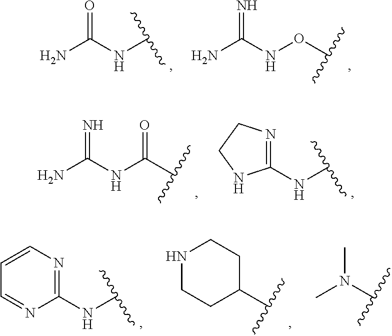

or protonated forms thereof, can be adjacent to each other. Two amino acids having a side chain comprising a guanidine or protonated form thereof are adjacent to each other. The cCPPs can comprise at least two contiguous amino acids having a side chain can comprise an aromatic or heteroaromatic group and at least two non-adjacent amino acids having a side chain comprising:

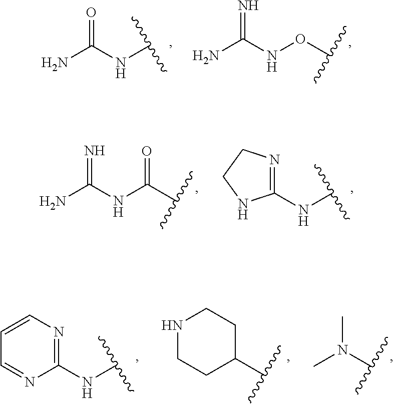

or a protonated form thereof. The cCPPs can comprise at least two contiguous amino acids having a side chain comprising an aromatic or heteroaromatic group and at least two non-adjacent amino acids having a side chain comprising

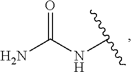 [SMILES: *NC(N)=O]

or a protonated form thereof. The adjacent amino acids can have the same chirality. The adjacent amino acids can have the opposite chirality. Other combinations of amino acids can have any arrangement of D and L amino acids, e.g., any of the sequences described in the preceding paragraph.

At least two amino acids having a side chain comprising:

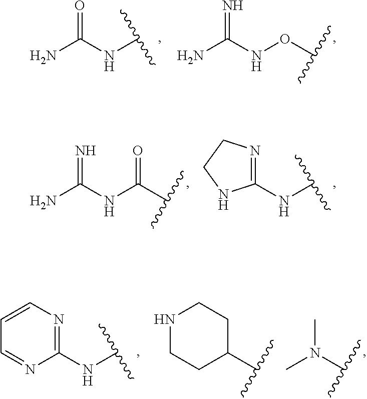

or a protonated form thereof, are alternating with at least two amino acids having a side chain comprising a guanidine group or protonated form thereof.

In embodiments, the cCPP can comprise the general Formula (IA):

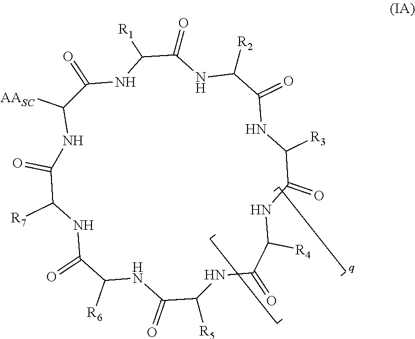

or a protonated form thereof,
wherein:
R₁, R₂, R₃, R₄, R₅, R₆, and R₇ are independently H or an amino acid side chain;
AA_{SC} is an amino acid side chain; and
q is 1, 2, 3 or 4.

The cCPP of the general Formula (IA) can have any configuration and/or amino acid side chain as described in the published PCT application NO. US2020/066459 (WO2021127650A1) or U.S. Pat. No. 11,225,506.

In embodiments, the cCPP are of the general Formula (IA) or a protonated form thereof,
wherein:
R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, and R₇ are independently H or an amino acid side chain;
at least one of R₄, R₅, R₆, and R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl)alanine;
AA_{SC} is an amino acid side chain; and
q is 1, 2, 3 or 4.

In embodiments, the cCPP are of Formula (IA) where at least one of R₄, R₅, R₆, and R₇ are independently an uncharged, non-aromatic side chain of an amino acid. In embodiments, at least one of R₄, R₅, R₆, and R₇ are independently H or a side chain of citrulline.

In embodiments, compounds are provided that include a cyclic peptide having 6 to 12 amino acids, wherein at least two amino acids of the cyclic peptide are charged amino acids, at least two amino acids of the cyclic peptide are aromatic hydrophobic amino acids and at least two amino acids of the cyclic peptide are uncharged, non-aromatic amino acids. In embodiments, at least two charged amino acids of the cyclic peptide are arginine. In embodiments, at least two aromatic, hydrophobic amino acids of the cyclic peptide are phenylalanine, naphtha alanine (3-Naphth-2-yl-alanine) or a combination thereof. In embodiments, at least two uncharged, non-aromatic amino acids of the cyclic peptide are citrulline, glycine or a combination thereof. In embodiments, the compound is a cyclic peptide having 6 to 12 amino acids wherein two amino acids of the cyclic peptide are arginine, at least two amino acids are aromatic, hydrophobic amino acids selected from phenylalanine, naphtha alanine and combinations thereof, and at least two amino acids are uncharged, non-aromatic amino acids selected from citrulline, glycine and combinations thereof.

The cCPP of general Formula (IA) can comprise the general Formula (I):

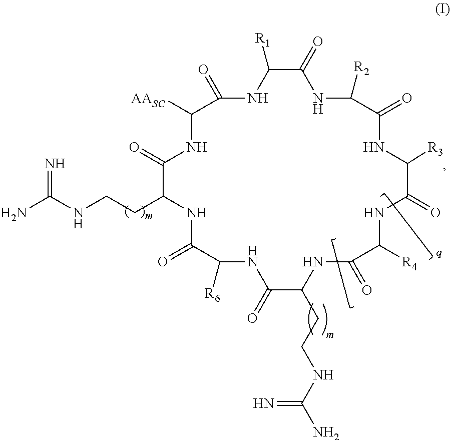

or a protonated form thereof,
wherein:
R₁, R₂, and R₃ can each independently be H or an aromatic or heteroaromatic side chain of an amino acid;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4; and
each m is independently 0 or an integer of 1, 2, or 3.

In embodiments, the cCPP are of Formula (IA) or (I) where R₁, R₂, and R₃ can each independently be H, -alkylene-aryl, or -alkylene-heteroaryl. R₁, R₂, and R₃ can each independently be H, -C₁₋₃alkylene-aryl, or -C₁₋₃alkylene-heteroaryl. R₁, R₂, and R₃ can each independently be H or -alkylene-aryl. R₁, R₂, and R₃ can each independently be H or -C₁₋₃alkylene-aryl. C₁₋₃alkylene can be methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can be phenyl. Heteroaryl can be pyridyl, quinolyl, and isoquinolyl. R₁, R₂, and R₃ can each independently be H, -C₁₋₃alkylene-Ph or -C₁₋₃alkylene-Naphthyl. R₁, R₂, and R₃ can each independently be H, -CH₂Ph, or -CH₂Naphthyl. R₁, R₂, and R₃ can each independently be H or -CH₂Ph.

In embodiments, the cCPP are of Formula (I) or (IA) where R₁, R₂, and R₃ can each independently be the side chain of phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

In embodiments, the cCPP are of Formula (I) or (IA) where R₁ can be the side chain of phenylalanine. R₁ can be the side chain of 1-naphthylalanine. R₁ can be the side chain of 2-naphthylalanine. R₁ can be the side chain of tryptophan. R₁ can be the side chain of 3-benzothienylalanine. R₁ can be the side chain of 4-phenylphenylalanine. R₁ can be the side chain of 3,4-difluorophenylalanine. R₁ can be the side chain of 4-trifluoromethylphenylalanine. R₁ can be the side chain of 2,3,4,5,6-pentafluorophenylalanine. R₁ can be the side chain of homophenylalanine. R₁ can be the side chain of β-homophenylalanine. R₁ can be the side chain of 4-tert-butyl-phenylalanine. R₁ can be the side chain of 4-pyridinylalanine. R₁ can be the side chain of 3-pyridinylalanine. R₁ can be the side chain of 4-methylphenylalanine. R₁ can be the side chain of 4-fluorophenylalanine. R₁ can be the side chain of 4-chlorophenylalanine. R₁ can be the side chain of 3-(9-anthryl)-alanine.

In embodiments, the cCPP are of Formula (I) or (IA) where R₂ can be the side chain of phenylalanine. R₂ can be the side chain of 1-naphthylalanine. R₁ can be the side chain of 2-naphthylalanine. R₂ can be the side chain of tryptophan. R₂ can be the side chain of 3-benzothienylalanine. R₂ can be the side chain of 4-phenylphenylalanine. R₂ can be the side chain of 3,4-difluorophenylalanine. R₂ can be the side chain of 4-trifluoromethylphenylalanine. R₂ can be the side chain of 2,3,4,5,6-pentafluorophenylalanine. R₂ can be the side chain of homophenylalanine. R₂ can be the side chain of β-homophenylalanine. R₂ can be the side chain of 4-tert-butyl-phenylalanine. R₂ can be the side chain of 4-pyridinylalanine. R₂ can be the side chain of 3-pyridinylalanine. R₂ can be the side chain of 4-methylphenylalanine. R₂ can be the side chain of 4-fluorophenylalanine. R₂ can be the side chain of 4-chlorophenylalanine. R₂ can be the side chain of 3-(9-anthryl)-alanine.

In embodiments, the cCPP are of Formula (I) or (IA) where R₃ can be the side chain of phenylalanine. R₃ can be the side chain of 1-naphthylalanine. R₃ can be the side chain of 2-naphthylalanine. R₃ can be the side chain of tryptophan. R₃ can be the side chain of 3-benzothienylalanine. R₃ can be the side chain of 4-phenylphenylalanine. R₃ can be the side chain of 3,4-difluorophenylalanine. Rs can be the side chain of 4-trifluoromethylphenylalanine. R₃ can be the side chain of 2,3,4,5,6-pentafluorophenylalanine. R₃ can be the side chain of homophenylalanine. R₃ can be the side chain of β-homophenylalanine. R₃ can be the side chain of 4-tert-butyl-phenylalanine. R₃ can be the side chain of 4-pyridinylalanine. R₃ can be the side chain of 3-pyridinylalanine. R₃ can be the side chain of 4-methylphenylalanine. R₃ can be the side chain of 4-fluorophenylalanine. R₃ can be the side chain of 4-chlorophenylalanine. R₃ can be the side chain of 3-(9-anthryl)-alanine.

In embodiments, the cCPP are of Formula (I) or (IA) where R₄ can be H, -alkylene-aryl, -alkylene-heteroaryl. R₄ can be H, -C₁₋₃alkylene-aryl, or -C₁₋₃alkylene-heteroaryl. R₄ can be H or -alkylene-aryl. R₄ can be H or -C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₄ can be H, -C₁₋₃alkylene-Ph or -C₁₋₃alkylene-Naphthyl. R₄ can be H or the side chain of an amino acid in Table 1. R₄ can be H or an amino acid residue having a side chain comprising an aromatic group. R₄ can be H, -CH₂Ph, or -CH₂Naphthyl. R₄ can be H or -CH₂Ph.

In embodiments, the cCPP are of Formula (IA) where R₅ can be H, -alkylene-aryl, -alkylene-heteroaryl. R₅ can be H, -C₁₋₃alkylene-aryl, or -C₁₋₃alkylene-heteroaryl. R₅ can be H or -alkylene-aryl. R₅ can be H or -C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₅ can be H, -C₁₋₃alkylene-Ph or -C₁₋₃alkylene-Naphthyl. R₅ can be H or the side chain of an amino acid in Table 1. R₄ can be H or an amino acid residue having a side chain comprising an aromatic group. R₅ can be H, -CH₂Ph, or -CH₂Naphthyl. R₅ can be H or -CH₂Ph.

In embodiments, the cCPP are of Formula (I) or (IA) where R₆ can be H, -alkylene-aryl, -alkylene-heteroaryl. R₆ can be H, -C₁₋₃alkylene-aryl, or -C₁₋₃alkylene-heteroaryl. R₆ can be H or -alkylene-aryl. R₆ can be H or -C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₆ can be H, -C₁₋₃alkylene-Ph or -C₁₋₃alkylene-Naphthyl. R₆ can be H or the side chain of an amino acid in Table 1 or. R₆ can be H or an amino acid residue having a side chain comprising an aromatic group. Re can be H, -CH₂Ph, or -CH₂Naphthyl. R₆ can be H or -CH₂Ph.

In embodiments, the cCPP are of Formula (IA) where R₇ can be H, -alkylene-aryl, -alkylene-heteroaryl. R₇ can be H, -C₁₋₃alkylene-aryl, or -C₁₋₃alkylene-heteroaryl. R₇ can be H or -alkylene-aryl. R₇ can be H or -C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₇ can be H, -C₁₋₃alkylene-Ph or -C₁₋₃alkylene-Naphthyl. R₇ can be H or the side chain of an amino acid in Table 1 or. R₇ can be H or an amino acid residue having a side chain comprising an aromatic group. R₇ can be H, -CH₂Ph, or -CH₂Naphthyl. R₇ can be H or -CH₂Ph.

In embodiments, the cCPP re of Formula (I) or (IA) where one, two or three of R₁, R₂, R₃, R₄, R₅, R₆, and R₇ can be -CH₂Ph. One of R₁, R₂, R₃, R₄, R₅, R₆, and R₇ can be -CH₂Ph. Two of R₁, R₂, R₃, R₄, R₅, R₆, and R₇ can be -CH₂Ph. Three of R₁, R₂, R₃, R₄, R₅, R₆, and R₇ can be -CH₂Ph. At least one of R₁, R₂, R₃, R₄, R₅, R₆, and R₇ can be -CH₂Ph. No more than four of R₁, R₂, R₃, R₄, R₅, R₆, and R₇ can be -CH₂Ph.

In embodiments, the cCPP re of Formula (I) or (IA) where one, two or three of R₁, R₂, R₃, and R₄ are -CH₂Ph. One of R₁, R₂, R₃, and R₄ is -CH₂Ph. Two of R₁, R₂, R₃, and R₄ are -CH₂Ph. Three of R₁, R₂, R₃, and R₄ are -CH₂Ph. At least one of R₁, R₂, R₃, and R₄ is -CH₂Ph.

In embodiments, the cCPP are of Formula (I) where one, two or three of R₁, R₂, R₃, R₄, R₅, R₆, and R₇ can be H. One of R₁, R₂, R₃, R₄, R₅, R₆, and R₇ can be H. Two of R₁, R₂, R₃, R₄, R₅, R₆, and R₇ are H. Three of R₁, R₂, R₃, R₅, R₆, and R₇ can be H. At least one of R₁, R₂, R₃, R₄, R₅, R₆, and R₇ can be H. No more than three of R₁, R₂, R₃, R₄, R₅, R₆, and R₇ can be -CH₂Ph.

In embodiments, the cCPP are of Formula (I) or (IA) where one, two or three of R₁, R₂, R₃, and R₄ are H. One of R₁, R₂, R₃, and R₄ is H. Two of R₁, R₂, R₃, and R₄ are H. Three of R₁, R₂, R₃, and R₄ are H. At least one of R₁, R₂, R₃, and R₄ is H.

In embodiments, the cCPP are of Formula (I) or (IA) where at least one of R₄, R₅, R₆, and R₇ can be side chain of 3-guanidino-2-aminopropionic acid. At least one of R₄, R₅, R₆, and R₇ can be side chain of 4-guanidino-2-aminobutanoic acid. At least one of R₄, R₅, R₆, and R₇ can be side chain of arginine. At least one of R₄, R₅, R₆, and R₇ can be side chain of homoarginine. At least one of R₄, R₅, R₆, and R₇ can be side chain of N-methylarginine. At least one of R₄, R₅, R₆, and R₇ can be side chain of N,N-dimethylarginine. At least one of R₄, R₅, R₆, and R₇ can be side chain of 2,3-diaminopropionic acid. At least one of R₄, R₅, R₆, and R₇ can be side chain of 2,4-diaminobutanoic acid, lysine. At least one of R₄, R₅, R₆, and R₇ can be side chain of N-methyllysine. At least one of R₄, R₅, R₆, and R₇ can be side chain of N,N-dimethyllysine. At least one of R₄, R₅, R₆, and R₇ can be side chain of N-ethyllysine. At least one of R₄, R₅, R₆, and R₇ can be side chain of N,N,N-trimethyllysine, 4-guanidinophenylalanine. At least one of R₄, R₅, R₆, and R₇ can be side chain of citrulline. At least one of R₄, R₅, R₆, and R₇ can be side chain of N,N-dimethyllysine, β-homoarginine. At least one of R₄, R₅, R₆, and R₇ can be side chain of 3-(1-piperidinyl)alanine.

In embodiments, the cCPP are of Formula (I) where at least two of R₄, R₅, R₆, and R₇ can be side chain of 3-guanidino-2-aminopropionic acid. At least two of R₄, R₅, R₆, and R₇ can be side chain of 4-guanidino-2-aminobutanoic acid. At least two of R₄, R₅, R₆, and R₇ can be side chain of arginine. At least two of R₄, R₅, R₆, and R₇ can be side chain of homoarginine. At least two of R₄, R₅, R₆, and R₇ can be side chain of N-methylarginine. At least two of R₄, R₅, R₆, and R₇ can be side chain of N,N-dimethylarginine. At least two of R₄, R₅, R₆, and R₇ can be side chain of 2,3-diaminopropionic acid. At least two of R₄, R₅, R₆, and R₇ can be side chain of 2,4-diaminobutanoic acid, lysine. At least two of R₄, R₅, R₆, and R₇ can be side chain of N-methyllysine. At least two of R₄, R₅, R₆, and R₇ can be side chain of N,N-dimethyllysine. At least two of R₄, R₅, R₆, and R₇ can be side chain of N-ethyllysine. At least two of R₄, R₅, R₆, and R₇ can be side chain of N,N,N-trimethyllysine, 4-guanidinophenylalanine. At least two of R₄, R₅, R₆, and R₇ can be side chain of citrulline. At least two of R₄, R₅, R₆, and R₇ can be side chain of N,N-dimethyllysine, β-homoarginine. At least two of R₄, R₅, R₆, and R₇ can be side chain of 3-(1-piperidinyl)alanine.

In embodiments, the cCPP re of Formula (I) where at least three of R₄, R₅, R₆, and R₇ can be side chain of 3-guanidino-2-aminopropionic acid. At least three of R₄, R₅, R₆, and R₇ can be side chain of 4-guanidino-2-aminobutanoic acid. At least three of R₄, R₅, R₆, and R₇ can be side chain of arginine. At least three of R₄, R₅, R₆, and R₇ can be side chain of homoarginine. At least three of R₄, R₅, R₆, and R₇ can be side chain of N-methylarginine. At least three of R₄, R₅, R₆, and R₇ can be side chain of N,N-dimethylarginine. At least three of R₄, R₅, R₆, and R₇ can be side chain of 2,3-diaminopropionic acid. At least three of R₄, R₅, R₆, and R₇ can be side chain of 2,4-diaminobutanoic acid, lysine. At least three of R₄, R₅, R₆, and R₇ can be side chain of N-methyllysine. At least three of R₄, R₅, R₆, and R₇ can be side chain of N,N-dimethyllysine. At least three of R₄, R₅, R₆, and R₇ can be side chain of N-ethyllysine. At least three of R₄, R₅, R₆, and R₇ can be side chain of N,N,N-trimethyllysine, 4-guanidinophenylalanine. At least three of R₄, R₅, R₆, and R₇ can be side chain of citrulline. At least three of R₄, R₅, R₆, and R₇ can be side chain of N,N-dimethyllysine, β-homoarginine. At least three of R₄, R₅, R₆, and R₇ can be side chain of 3-(1-piperidinyl)alanine.

AA_{SC} of general Formula (IA) and (I) can be a side chain of a residue of asparagine, glutamine, or homoglutamine. AA_{SC} can be a side chain of a residue of glutamine. The cCPP can further comprise a linker conjugated the AA_{SC}, e.g., the residue of asparagine, glutamine, or homoglutamine. Hence, the cCPP can further comprise a linker conjugated to the asparagine, glutamine, or homoglutamine residue. The cCPP can further comprise a linker conjugated to the glutamine residue.

In embodiments, the cCPP are of Formula (I) where q can be 1, 2, or 3. q can be 1 or 2. q can be 1. q can be 2. q can be 3. q can be 4.

In embodiments, the cCPP re of Formula (I) where m can be 1, 2, or3. m can be 1 or 2. m can be 0. m can be 1. m can be 2. m can be 3.

The cCPP of Formula (IA) or (I) can comprise Formula (I-a) or Formula (I-b):

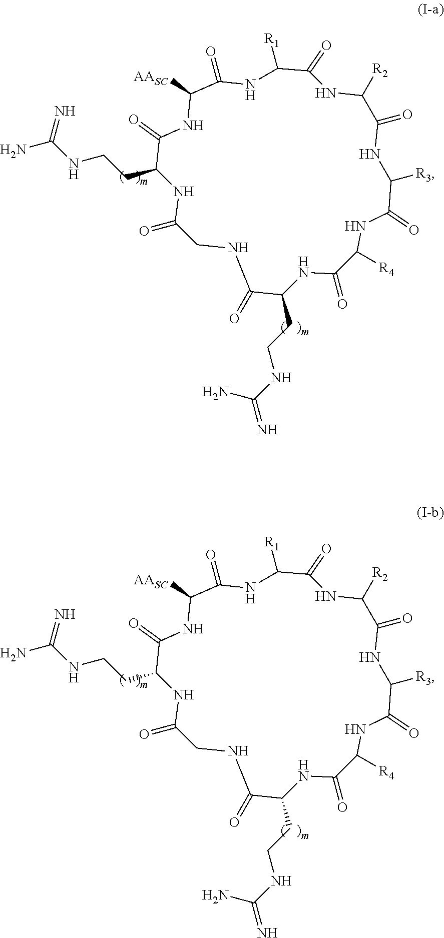

or protonated form thereof, wherein AA_{SC}, R₁, R₂, R₃, R₄, and m are as defined herein relative to Formula (IA) and/or Formula (I).

The cCPP of Formula (IA) or (I) can comprise the structures of (I-1), (I-2), (I-3), (I-4), (I-5), (I-6) or (I-7) (SEQ ID NOS 16, 24, 40-41, 132-133 and 270, respectively, in order of appearance):

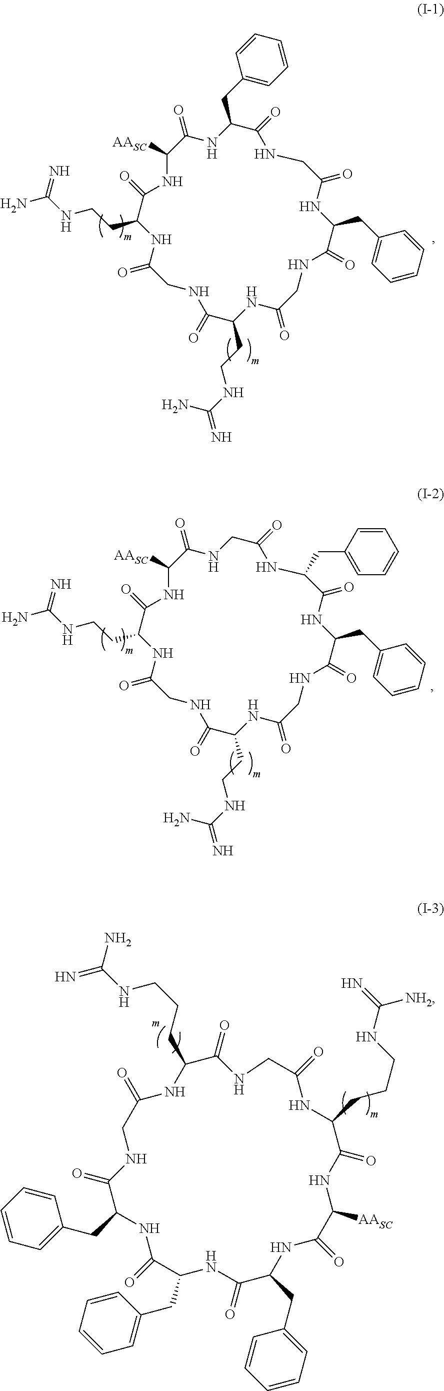

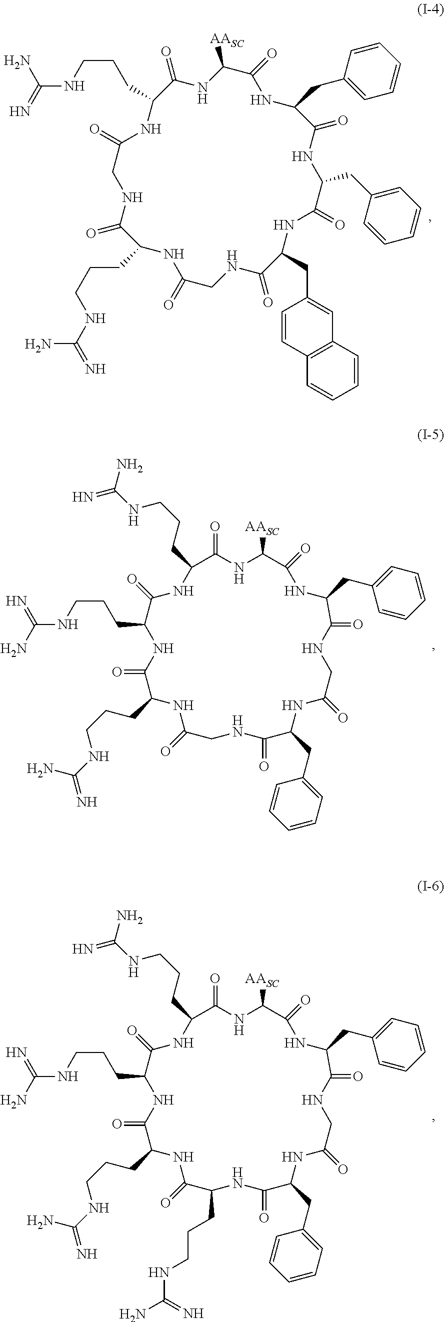

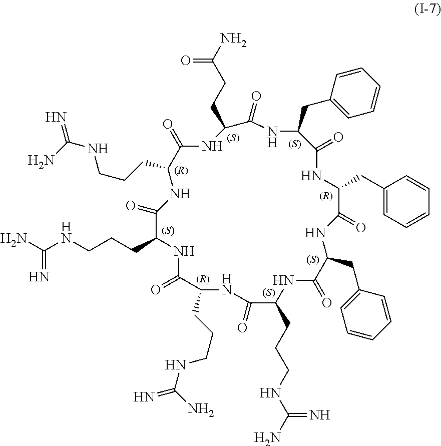

or a protonated form thereof, wherein AA_{SC} and m are as defined herein relative to Formula (IA) and/or Formula (I).

In embodiments, the cCPP of the general Formula (IA) is of general Formula (IX):

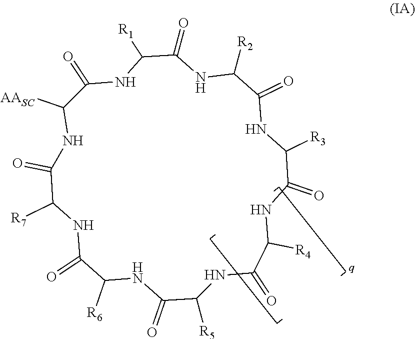

wherein:
at least two of R₁, R₂, R₃, R₄, R₅, R₆, or R₇ are independently the side chain of lysine, mono-methyl lysine, dimethyl lysine, trimethyl lysine, 2,4-diaminobutanoic acid, or 2,3-diaminopropionic acid;
   R₁, R₂, R₃, R₄, R₅, R₆, R₇ are independently H or an amino acid side chain;
   AA_{SC} is an amino acid side chain; and
   q is 1, 2, 3 or 4.

In embodiments, the CPP is of the general Formula (IX), wherein at least two of R₄, R₅, R₆, or R₇ are independently the amino acid side chain of lysine, mono-methyl lysine, dimethyl lysine, trimethyl lysine, 2,4-diaminobutanoic acid, or 2,3-diaminopropionic acid.

In embodiments, the CPP is of the general Formula (IX), wherein at least three of R₄, R₅, R₆, or R₇ are independently the amino acid side chain of lysine, mono-methyl lysine, dimethyl lysine, or trimethyl lysine.

In embodiments, the CPP is of the general Formula (IX), wherein R₄, R₅, R₆, R₇ are independently the amino acid side chain of lysine, mono-methyl lysine, dimethyl lysine, trimethyl lysine, 2,4-diaminobutanoic acid, or 2,3-diaminopropionic acid.

In embodiments, the CPP is of the general Formula (IX), wherein at least one of R₁, R₂, R₃, R₄, R₅, R₆, or R₇ is H.

In embodiments, the CPP is of the general Formula (IX), wherein at least one of R₁, R₂, or R₃ is H. In embodiments, the CPP is of the general Formula (IX), wherein at least one of R₄, R₅, R₆, or R₇ is H. In embodiments, the CPP is of the general Formula (IX), wherein at least two of R₁, R₂, R₃, R₄, R₅, R₆, or R₇ are independently H. In embodiments, the CPP is of the general Formula (IX), wherein at least one of R₁, R₂, or R₃ is H; and at least one of R₄, R₄, R₆, or R₇ is H.

In embodiments, the CPP is of the general Formula (IX), wherein at least one of R₁, R₂, R₃, R₄, R₅, R₆, or R₇ is an aromatic or heteroaromatic side chain of an amino acid. In embodiments, the CPP is of the general Formula (IX), wherein at least one of R₁, R₂, R₃, is an aromatic or heteroaromatic side chain of an amino acid. In embodiments, the CPP is of the general Formula (IX), wherein at least two of R₁, R₂, R₃, are independently an aromatic or heteroaromatic side chain of an amino acid.

In embodiments, the CPP of the general Formula (IX) is of the general formula IX(1),

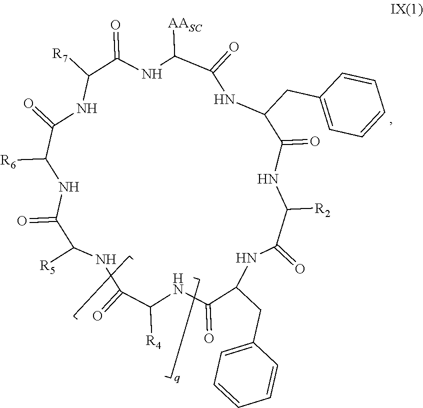

or a protonated form thereof, wherein:
R₁, R₂, R₃, R₄, R₅, R₆, and R₇ are independently H or the side chain of an amino acid;
at least two of R₄, R₅, R₆, or R₇ are independently the side chain of lysine, mono-methyl lysine, dimethyl lysine, trimethyl lysine, 2,4-diaminobutanoic acid, or 2,3-diaminopropionic acid;
R₂ is H or an amino acid side chain;
AA_{SC} is an amino acid side chain; and
q is 1, 2, 3 or 4.

In embodiments, the CPP is of the general Formula IX(1), wherein, R₁, R₃, or both have S stereochemistry.

In embodiments, the CPP is of the general Formula IX(1), wherein R₂ is H.

In embodiments, the CPP is of the general Formula IX(1), wherein at least two of R₄, R₅, R₆, or R₇ are independently the amino acid side chain of lysine, mono-methyl lysine, dimethyl lysine, trimethyl lysine, 2,4-diaminobutanoic acid, or 2,3-diaminopropionic acid.

In embodiments, the CPP is of the general Formula IX(1), wherein at least three of R₄, R₅, R₆, or R₇ are independently the amino acid side chain of lysine, mono-methyl lysine, dimethyl lysine, trimethyl lysine, 2,4-diaminobutanoic acid, or 2,3-diaminopropionic acid.

In embodiments, the CPP is of the general Formula IX(1), wherein at least R₅ and R₇ are independently the side chain of lysine, mono-methyl lysine, dimethyl lysine, trimethyl lysine, 2,4-diaminobutanoic acid, or 2,3-diaminopropionic acid.

In embodiments, the CPP is of the general Formula IX(1), wherein; R₂ is H; q is one; and at least R₅ and R₇ are independently the side chain of lysine, mono-methyl lysine, dimethyl lysine, trimethyl lysine, 2,4-diaminobutanoic acid, or 2,3-diaminopropionic acid.

The AA_{sc} of Formula IX or IX(1) may be any AA_{sc} as described relative to Formula IA. AA_{SC} can be conjugated to a linker.

In embodiments, the cCPP of Formula (IA), (IX), (IX(1)), has the structure of IX(a), IX(b), IX(c) (SEQ ID NOS 271-272 and 158, respectively, in order of appearance), or a protonated form thereof:

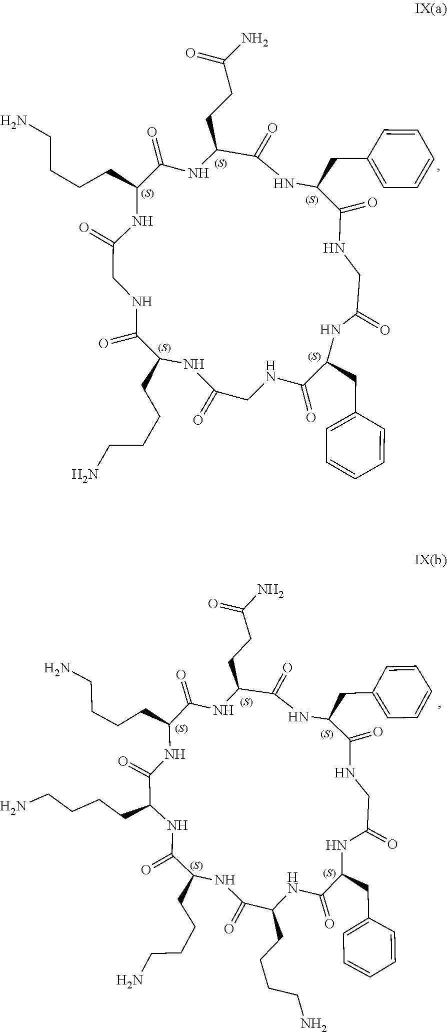

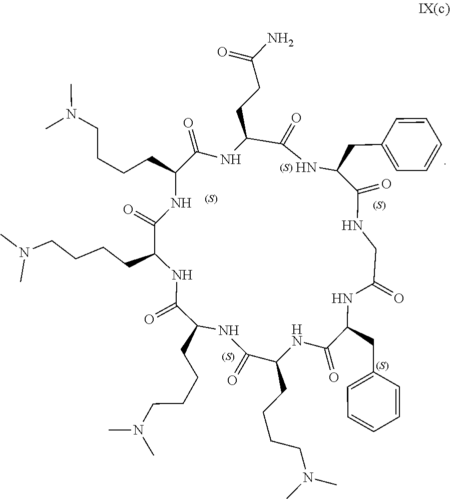

In embodiments, the CPP of general Formula (IA), (IX), or IX(1) may comprise one of the sequences: FGFGKGK (SEQ ID NO: 49); FGFKKKK (SEQ ID NO: 50); FGFK(me2)K(me2)K(me2)K(me2) (SEQ ID NO: 51); FGFGKGKQ (SEQ ID NO: 52); FGFKKKKQ (SEQ ID NO: 53); or FGFK(me2)K(me2)K(me2)K(me2)Q (SEQ ID NO: 54) (Kme2 is dimethyl lysine).

The cCPP can comprise one of the following sequences: FGFGRGR (SEQ ID NO: 55); GfFGrGr (SEQ ID NO: 56), FfΦGRGR (SEQ ID NO: 57); FfFGRGR (SEQ ID NO: 58); or FfΦGrGr (SEQ ID NO: 59). The cCPP can have one of the following sequences: FGFGRGRQ (SEQ ID NO: 60); GfFGrGrQ (SEQ ID NO: 61), FfΦGRGRQ (SEQ ID NO: 62), FfFGRGRQ (SEQ ID NO: 63); FfΦGrGrQ (SEQ ID NO: 64); or FfFRrRrQ (SEQ ID NO: 65).

The disclosure also relates to a cCPP having the general Formula (II)

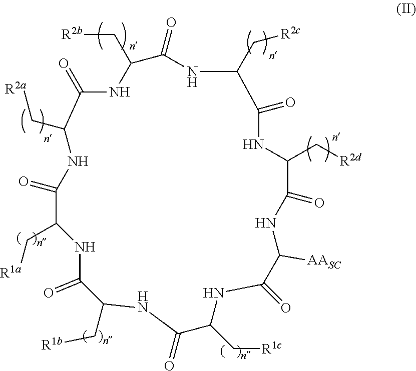

wherein:
AA_{SC} is an amino acid side chain;
R^{1a}, R^{1b}, and R^{1c} are each independently a 6- to 14-membered aryl or a 6- to 14-membered heteroaryl;
R^{2a}, R^{2b}, R^{2c} and R^{2d} are independently an amino acid side chain; at least one of R^{2a}, R^{2b}, R^{2c} and R^{2d} is

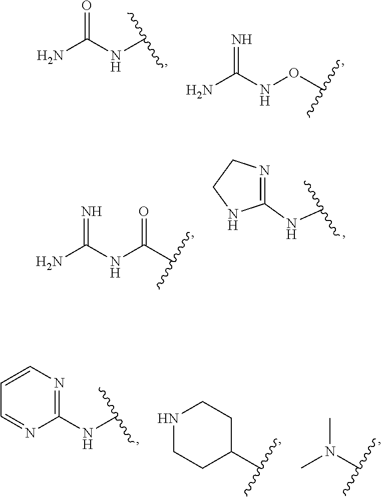

or a protonated form thereof;
at least one of R^{2a}, R^{2b}, R^{2c} and R^{2d} is guanidine or a protonated form thereof;
each n″ is independently an integer 0, 1, 2, 3, 4, or 5;
each n′ is independently an integer from 0, 1, 2, or 3; and
if n′ is 0 then R^{2a}, R^{2b}, R^{2b} or R^{2d} is absent.

In embodiments, the cCPP is of Formula (II) where at least two of R^{2a}, R^{2b}, R^{2c} and R^{2d} can be

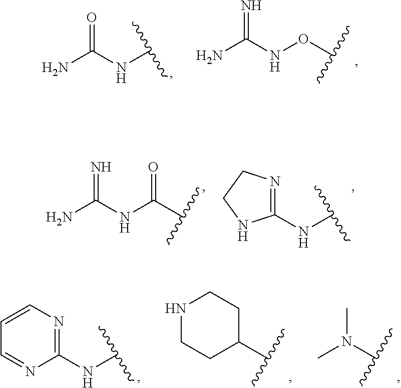

or a protonated form thereof. Two or three of R^{2a}, R^{2b}, R^{2c} and R^{2d} can be

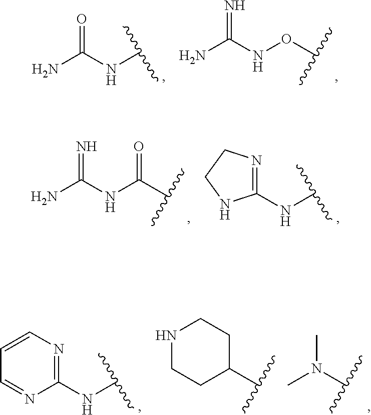

or a protonated form thereof. One of R^{2a}, R^{2b}, R^{2c} and R^{2d} can be

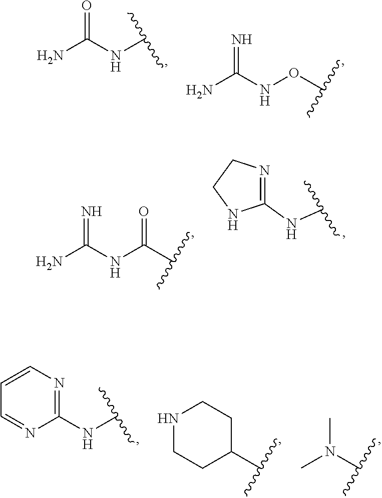

or a protonated form thereof. At least one of R^{2a}, R^{2b}, R^{2c} and R^{2d} can be

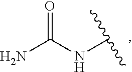 [SMILES: *NC(N)=O]

or a protonated form thereof, and the remaining of R^{2a}, R^{2b}, R^{2c} and R^{2d} can be guanidine or a protonated form thereof. At least two of R^{2a}, R^{2b}, R^{2c} and R^{2d} can be

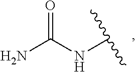 [SMILES: *NC(N)=O]

or a protonated form thereof, and the remaining of R^{2a}, R^{2b}, R^{2c} and R^{2d} can be guanidine, or a protonated form thereof.

In embodiments, the cCPP is of Formula (II) where all of R^{2a}, R^{2b}, R^{2c} and R^{2d} can be

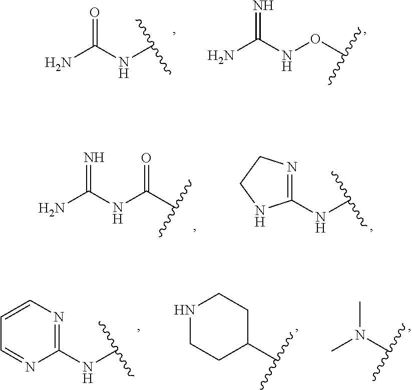

or a protonated form thereof. At least of R^{2a}, R^{2b}, R^{2c} and R^{2d} can be

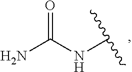 [SMILES: *NC(N)=O]

or a protonated form thereof, and the remaining of R^{2a}, R^{2b}, R^{2c} and R^{2d} can be guanidine or a protonated form thereof. At least two R^{2a}, R^{2b}, R^{2c} and R^{2d} groups can be

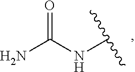 [SMILES: *NC(N)=O]

or a protonated form thereof, and the remaining of R^{2a}, R^{2b}, R^{2c} and R^{2d} are guanidine, or a protonated form thereof.

In embodiments, the cCPP is of Formula (II) where each of R^{2a}, R^{2b}, R^{2c} and R^{2d} can independently be 2,3-diaminopropionic acid, 2,4-diaminobutyric acid, the side chains of ornithine, lysine, methyllysine, dimethyllysine, trimethyllysine, homo-lysine, serine, homo-serine, threonine, allo-threonine, histidine, 1-methylhistidine, 2-aminobutanedioic acid, aspartic acid, glutamic acid, or homo-glutamic acid.

In embodiments, the cCPP is of Formula (II) where AA_{SC} can be

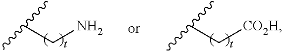

wherein t can be an integer from 0 to 5. AA_{SC} can be

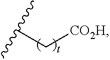

wherein t can be an integer from 0 to 5. t can be 1 to 5. tis 2 or 3. t can be 2. t can be 3.

In embodiments, the cCPP is of Formula (II) where R^{1a}, R^{1b}, and R^{1c} can each independently be 6- to 14-membered aryl. R^{1a}, R^{1b}, and R^{1c} can be each independently a 6- to 14-membered heteroaryl having one or more heteroatoms selected from N, O, or S. R^{1a}, R^{1b}, and R^{1c} can each be independently selected from phenyl, naphthyl, anthracenyl, pyridyl, quinolyl, or isoquinolyl. R^{1a}, R^{1b}, and R^{1c} can each be independently selected from phenyl, naphthyl, or anthracenyl. R^{1a}, R^{1b}, and R^{1c} can each be independently phenyl or naphthyl. R^{1a}, R^{1b}, and R^{1c} can each be independently selected pyridyl, quinolyl, or isoquinolyl.

In embodiments, the cCPP is of Formula (II) where each n′ can independently be 1 or 2. Each n′ can be 1. Each n′ can be 2. At least one n′ can be 0. At least one n′ can be 1. At least one n′ can be 2. At least one n′ can be 3. At least one n′ can be 4. At least one n′ can be 5.

In embodiments, the cCPP is of Formula (II) where each n″ can independently be an integer from 1 to 3. Each n″ can independently be 2 or 3. Each n″ can be 2. Each n″ can be 3. At least one n″ can be 0. At least one n″ can be 1. At least one n″ can be 2. At least one n″ can be 3.

In embodiments, the cCPP is of Formula (II) where each n″ can independently be 1 or 2 and each n′ can independently be 2 or 3. Each n″ can be 1 and each n′ can independently be 2 or 3. Each n″ can be 1 and each n′ can be 2. Each n″ is 1 and each n′is 3.

The cCPP of Formula (II) can be of Formula (II-1):

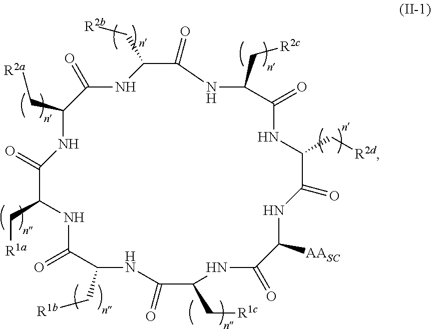

wherein R^{1a}, R^{1b}, R^{1c}, R^{2a}, R^{2b}, R^{2c}, R^{2d}, AA_{SC}, n′ and n″ are as defined herein.

The cCPP of Formula (II) can be of Formula (IIa):

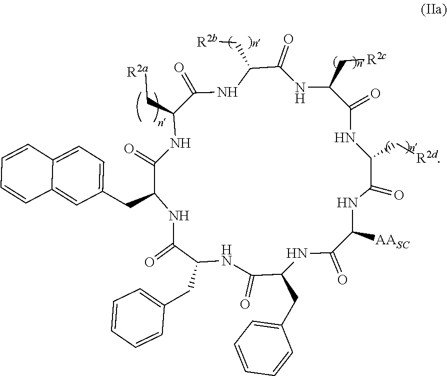

wherein R^{1a}, R^{1b}, R^{1c}, R^{2a}, R^{2b}, R^{2c}, R^{2d}, AA_{SC} and n′ are as defined herein.

The cCPP of formula (II) can be of Formula (IIb):

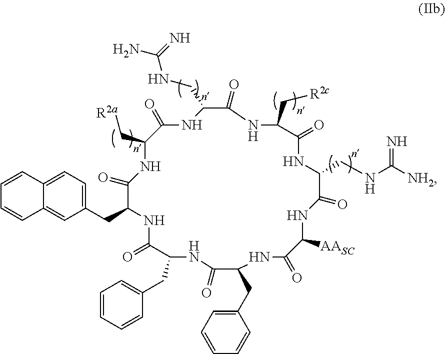

wherein R^{2a}, R^{2b}, AA_{SC}, and n′ are as defined herein.

The cCPP can be of Formula (II) can be of Formula (IIc):

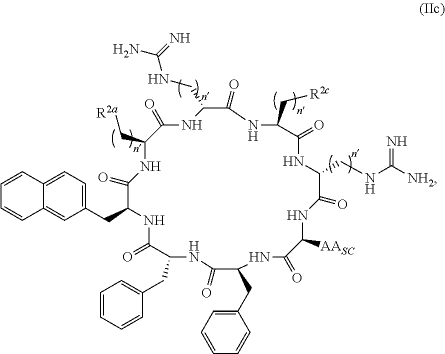

or a protonated form thereof,
wherein:
AA_{SC} and n′ are as defined herein.

The cCPP can be of Formula (III):

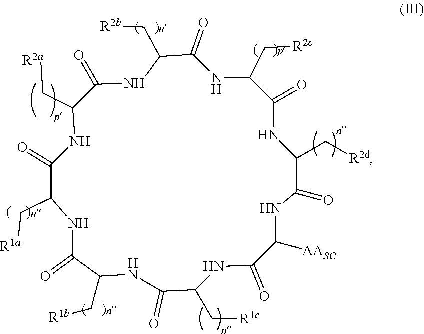

wherein:
AA_{SC} is an amino acid side chain;
R^{1a}, R^{1b}, and R^{1c} are each independently a 6- to 14-membered aryl or a 6- to 14-membered heteroaryl;
R^{2a} and R^{2c} are each independently H,

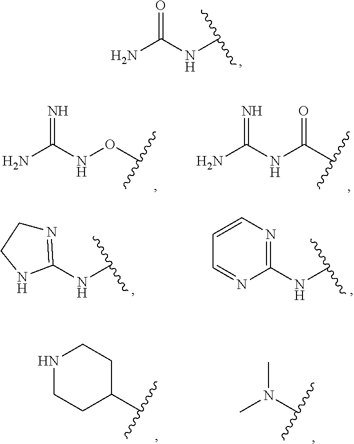

or a protonated form thereof;
R^{2b} and R^{2d} are each independently guanidine or a protonated form thereof,
each n″ is independently an integer from 1 to 3;
each n′ is independently an integer from 1 to 5; and
each p′ is independently an integer from 0 to 5.

The cCPP of Formula (III) can be of Formula (III-1):

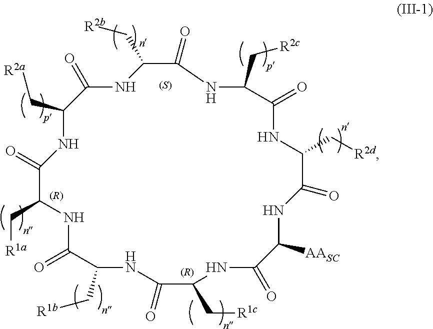

wherein:
AA_{SC}, R^{1a}, R^{1b}, R^{1c}, R^{2a}, R^{2c}, R^{2b}, R^{2d} n′, n″, and p′ are as defined herein.

The cCPP of Formula (III) can be of Formula (IIIa):

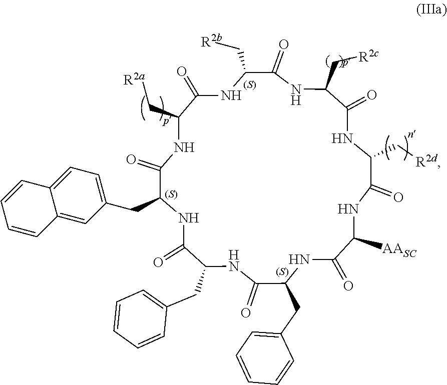

wherein:
AA_{SC}, R^{2a}, R^{2c}, R^{2b}, R^{2d} n′, n″, and p′ are as defined herein.

In Formulas (III), (III-1), and (IIIa), R^{a} and R^{c} can be H. R^{a} and R^{c} can be H and R^{b} and R^{d} can each independently be guanidine or protonated form thereof. R^{a} can be H. R^{b} can be H. p′ can be 0. R^{a} and R^{c} can be H and each p′ can be 0.

In Formulas (III), (III-1), and (IIIa), R^{a} and R^{c} can be H, R^{b} and R^{d} can each independently be guanidine or protonated form thereof, n″ can be 2 or 3, and each p′ can be 0.

p′ can 0. p′ can 1. p′ can 2. p′ can 3. p′ can 4. p′ can be 5.

The cCPP can have the structure (SEQ ID NO: 283):

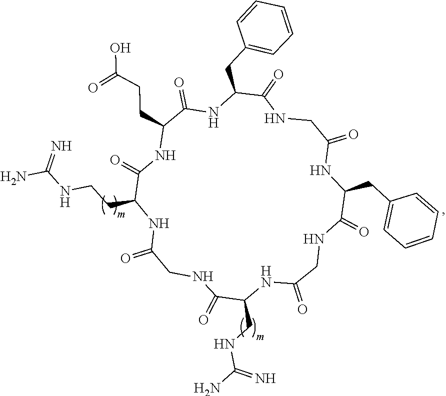

or a protonated from thereof wherein m is defined herein.

The cCPP of Formula (IA) can be selected from:

CPP Sequence
(SEQ ID NO: 66)
(FfΦRrRrQ)
(SEQ ID NO: 67)
(FfΦCit-r-Cit-rQ)
(SEQ ID NO: 64)
(FfΦGrGrQ)
(SEQ ID NO: 63)
(FfFGRGRQ)
(SEQ ID NO: 60)
(FGFGRGRQ)
(SEQ ID NO: 61)
(GfFGrGrQ)
(SEQ ID NO: 68)
(FGFGRRRQ)
(SEQ ID NO: 69)
(FGFRRRRQ)

The cCPP of Formula (IA) can be selected from:

CPP Sequence

FΦRRRRQ (SEQ ID NO: 70)
fΦRrRrQ (SEQ ID NO: 71)
FfΦRrRrQ (SEQ ID NO: 66)
FfΦCit-r-Cit-rQ (SEQ ID NO: 67)
FfΦGrGrQ (SEQ ID NO: 64)
FfΦRGRGQ (SEQ ID NO: 72)
FfFGRGRQ (SEQ ID NO: 63)
FGFGRGRQ (SEQ ID NO: 60)
GfFGrGrQ (SEQ ID NO: 61)
FGFGRRRQ (SEQ ID NO: 68)
FGFRRRRQ (SEQ ID NO: 69)

In embodiments, the cCPP is selected from:

CPP Sequence

| | | |
|---|---|---|
| FΦRRRQ (SEQ ID NO: 73) | RRFRΦRQ (SEQ ID NO: 74) | FΦRRRRQK (SEQ ID NO: 75) |
| FΦRRRC (SEQ ID NO: 76) | FRRRRΦQ (SEQ ID NO: 77) | FΦRRRRQC (SEQ ID NO: 78) |
| FΦRRRU (SEQ ID NO: 79) | rRFRΦRQ (SEQ ID NO: 80) | fΦRrRrRQ (SEQ ID NO: 81) |
| RRRΦFQ (SEQ ID NO: 82) | RRΦFRRQ (SEQ ID NO: 83) | FΦRRRRRQ (SEQ ID NO: 84) |
| RRRRΦF (SEQ ID NO: 85) | CRRRRFWQ (SEQ ID NO: 86) | RRRRΦFDΩC (SEQ ID NO: 87) |
| FΦRRRR (SEQ ID NO: 88) | FfΦRrRrQ (SEQ ID NO: 66) | FΦRRR (SEQ ID NO: 89) |
| FφrRrRq (SEQ ID NO: 90) | FFΦRRRRQ (SEQ ID NO: 91) | FWRRR (SEQ ID NO: 92) |
| FφrRrRQ (SEQ ID NO: 93) | RFRFRΦRQ (SEQ ID NO: 94) | RRRΦF (SEQ ID NO: 95) |
| FΦRRRRQ (SEQ ID NO: 70) | URRRRFWQ (SEQ ID NO: 96) | RRRWF (SEQ ID NO: 97) |
| fΦRrRrQ (SEQ ID NO: 71) | CRRRRFWQ (SEQ ID NO: 86) | |

Where Φ=L-naphthylalanine; φ=D-naphthylalanine; Ω=L-norleucine

The cCPP can comprise Formula (D)

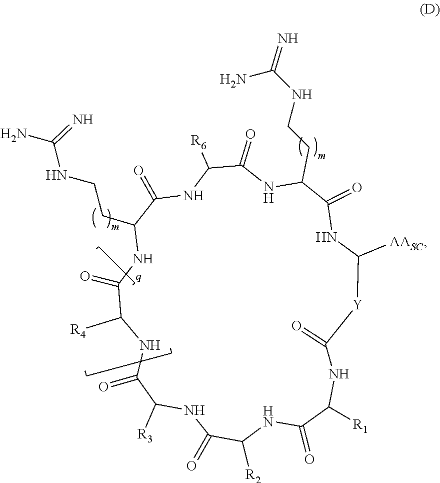

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
Y is

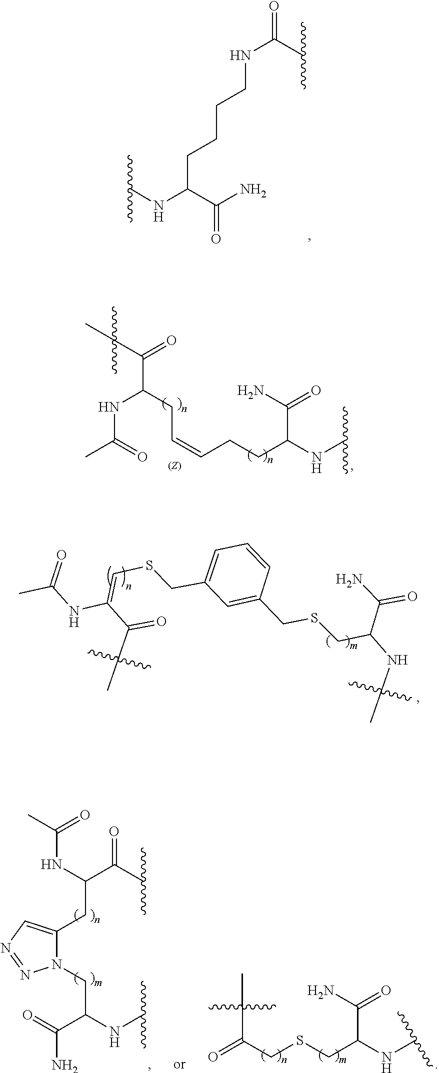

q is 1, 2, 3 or 4;
each m is independently an integer 0, 1, 2, or 3, and
each n is independently an integer 0, 1, 2, or 3.

The cCPP can comprise Formula (AV):

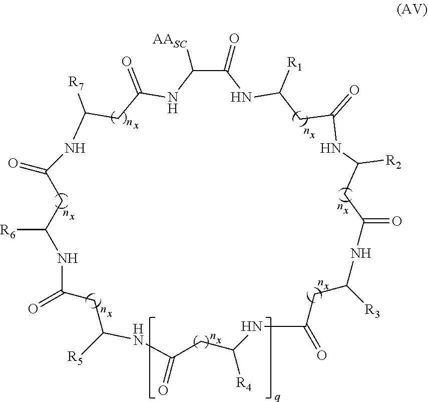

wherein;
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
AA_{SC} is an amino acid side chain; and
nₓ is 0 or 1 and at least one nₓ is 1; and
q is 1, 2, 3 or 4.

In embodiments, the cCPP is of Formula (AV), wherein only one nₓ is 1. In embodiments, the cCPP is of Formula (AV), wherein the nₓ associated with R₁ is 1; that is, the amino acid residue of R₁ is a beta amino acid.

In embodiments, the cCPP is of Formula (AV), wherein R₁, R₂, R₃, R₄, R₅, R₆, R₇ are independently H or an amino acid side chain;
at least two of R₁, R₂, and R₃ are independently a side chain of phenylalanine, naphthylalanine;
at least two of R₄, R₅, R₆, or R₇ are independently a side chain of arginine;
AA_{SC} is an amino acid side chain; and
each nₓ is 1 or 0;
residue R1 is a beta-amino acid (i.e., nₓ associated with R₁ is 1) and
q is 1, 2, 3 or 4.

In embodiments, the cCPP is of Formula (AV), wherein at least one of R₁, R₂, R₃, R₄, or R₇ are a B-amino acid (i.e., at least one nₓ is 1). In embodiments, at least one of R₁, R₂, R₃ is a side chain of B-hF. In embodiments, at least one of R₁, R₂, R₃ is a side chain of b-alanine. In embodiments, at least one of R₄, or R₇ is a side chain of B-alanine. In embodiments, at least one of R₄, or R₇ is a side chain of B-hF.

In embodiments, the cCPP can be of the Formula (Y1):

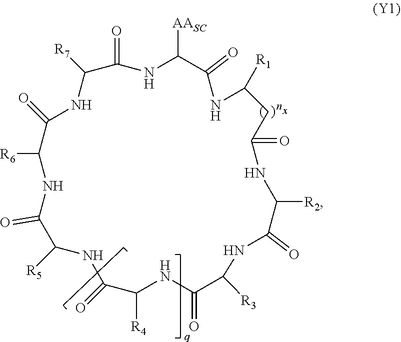

or a protonated form thereof,
wherein:
at least one of R₁, R₂, R₃, R₄, R₅, R₆, or R₇ is the amino acid side chain of serine or histidine;
R₁, R₂, R₃, R₄, R₅, R₆, R₇ are independently H or an amino acid side chain;
AA_{SC} is an amino acid side chain;
nₓ is 0 or 1; and
q is 1, 2, 3 or 4.

In embodiments the cCPP is of Formula (Y1) where at least two of R₁, R₂, R₃, R₄, R₅, R₆, or R₇ is the amino acid side chain of serine or histidine. In embodiments the cCPP is of Formula (Y1) where at least three of R₁, R₂, R₃, R₄, R₅, R₆, or R₇ is the amino acid side chain of serine or histidine. In embodiments the cCPP is of Formula (Y1) where at least four of R₁, R₂, R₃, R₄, R₅, R₆, or R₇ is the amino acid side chain of serine or histidine.

The cCPP of Formula Y1 can comprise the general Formula (Y1′):

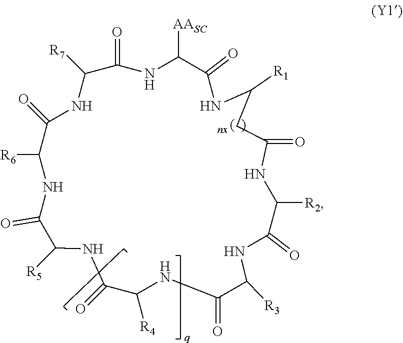

or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
at least two of R₄, R₅, R₆, or R₇ are independently a side chain of serine or histidine;
AA_{SC} is an amino acid side chain;
nₓ is 0 or 1; and
q is 1, 2, 3 or 4.

In embodiments, the cCPP is of Formula (Y1′), where three of R₄, R₅, R₆, or R₇ are independently a side chain of serine or histidine.

In embodiments, the cCPP is of formula (Y1′), wherein q is 1.

In embodiments, the cCPP be of the Formula (Y2):

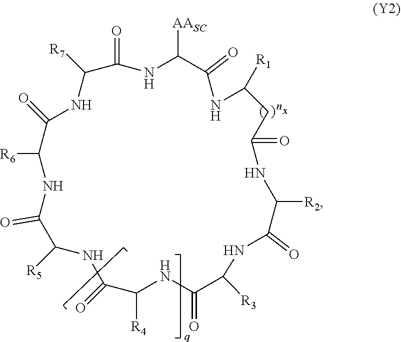

or a protonated form thereof,
wherein:
R₁, R₂, R₃, R₄, R₅, R₆, R₇ are independently H or an amino acid side chain;
AA_{SC} is an amino acid side chain;
nₓ is 1; and
q is 1, 2, 3 or 4.

The cCPP of Formula Y can be of the general Formula (Y240 ):

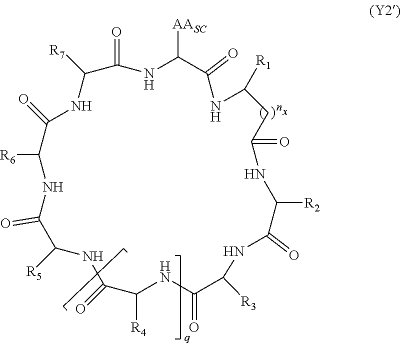

or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine or naphthylalanine;
at least two of R₄, R₅, R₆, or R₇ are independently a side chain of arginine;
AA_{SC} is an amino acid side chain; and
nₓ is 1; and
q is 1, 2, 3 or 4.

In embodiments, the CPP is of Formula (Y2) or (Y2′) wherein:
R₁, R₂, R₃, R₄, R₅, R₆, R₇ are independently H or an amino acid side chain;
at least two of R₁, R₂, and R₃ are independently a side chain of phenylalanine or naphthylalanine;
at least two of R₄, R₅, R₆, or R₇ are independently a side chain of arginine;
at least two of R₄, R₅, R₆, or R₇ are independently a side chain of an uncharged non-aryl amino acid selected from histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine;
AA_{SC} is an amino acid side chain;
nₓ is 0 or 1; and
q is 1, 2, 3 or 4.

In embodiments, the CPP is of Formula (Y2) or (Y2′) wherein:
R₁, R₂, R₃, R₄, R₅, R₆, R₇ are independently H or an amino acid side chain;
at least two of R₁, R₂, and R₃ are independently a side chain of phenylalanine or naphthylalanine;
at least two of R₄, R₅, R₆, or R₇ are independently a side chain of arginine;
at least two of R₄, R₅, R₆, or R₇ are independently a side chain of serine or histidine;
AA_{SC} is an amino acid side chain;
nₓ is 0 or 1; and
q is 1.

In embodiments, the CPP is of Formula (Y2) or (Y2′) wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine.
at least two of R₄, R₅, R₆, or R₇ are independently a side chain of histidine or serine;
AA_{SC} is an amino acid side chain;
nₓ is 0 or 1; and
q is 1.

In embodiments, the CPP is of the general Formula (AV), (Y1), (Y1′), (Y2), or (Y2′) wherein at least one of R₁, R₂, or R₃ is H. In embodiments, the CPP is of the general Formula (AV), (Y1), (Y1′), (Y2), or (Y2′), wherein at least one of R₁, R₂, or R₃ is a side chain of phenylalanine. In embodiments, the CPP is of the general Formula (AV), (Y1), (Y1′), (Y2), or (Y2′), wherein at least two of R₁, R₂, or R₃ are a side chain of naphthylalanine.

In embodiments, the CPP is of the general Formula (AV), (Y1), (Y1′), (Y2), or (Y2′), wherein q is 1. In embodiments, the CPP is of the general Formula (AV), (Y1), (Y1′), (Y2), or (Y2′), wherein q is 1 and nₓ is 1 (at least one nₓ of Formula Y is 1). In embodiments, the CPP is of the general Formula (AV), (Y1), (Y1′), (Y2), or (Y2′), wherein q is 1 and nₓ is 0 (all nₓ of Formula Y is 1).

In embodiments, the CPP is of the general Formula (AV), (Y1), (Y1′), (Y2), or (Y2′), wherein at least two of R₄, R₅, R₆, or R₇ are independently a side chain of serine or histidine.

In embodiments, the CPP is of the general Formula (AV), (Y1), (Y1′), (Y2), or (Y2′), at least two of R₄, R₅, R₆, or R₇ are independently a side chain of arginine.

In embodiments, the CPP is of the general Formula (AV), (Y1), (Y2), or (Y2′), wherein at least one of R₄, R₅, R₆, R₇ are independently an uncharged, non-aryl side chain of an amino acid. In embodiments, at least two of R₄, R₅, R₆, or R₇ are independently side chains of an uncharged non-aryl amino acid (e.g., histidine, threonine, serine, leucine, isoleucine, valine, neopentylglycine, alanine, homoalanine, homoserine, 3-(4-Thiazolyl)-alanine, 3-(4-furanyl)-alanine, and 3-(4-thienyl)-alanine). In embodiments, the CPP is of the general Formula (AV), (Y1), (Y2), or (Y2′), wherein at least two of R₄, R₅, R₆, or R₇ are independently side chains of an uncharged non-aryl amino acid selected from histidine, threonine, serine, leucine, isoleucine, valine, neopentylglycine, alanine, homoalanine, homoserine, 3-(4-Thiazolyl)-alanine, 3-(4-furanyl)-alanine, and 3-(4-thienyl)-alanine.

In embodiments, the CPP is of the general Formula (AV), (Y1), (Y2), or (Y2′), wherein at least one of R₄, R₅, R₆, R₇ are independently H.

In embodiments, compounds are provided that include a cyclic peptide having 6 to 12 amino acids, wherein at least two amino acids of the cyclic peptide are charged amino acids, at least two amino acids of the cyclic peptide are aromatic hydrophobic amino acids and at least two amino acids of the cyclic peptide are uncharged, non-aryl amino acids. In embodiments, at least two charged amino acids of the cyclic peptide are arginine. In embodiments, at least two aromatic, hydrophobic amino acids of the cyclic peptide are phenylalanine, naphthylalanine (3-naphth-2-yl-alanine) or a combination thereof. In embodiments, at least two uncharged, non-aryl amino acids of the cyclic peptide are glycine. In embodiments, two of the uncharged amino acids are serine, histidine or a combination thereof.

Is embodiments, the CPP is of the general Formula (AV), (Y1), (Y1′), (Y2), or (Y2′), wherein at least one of R₄, R₅, R₆, or R₇ is the amino acid side chain of serine or histidine. In embodiments, the CPP is of the general Formula (AV), (Y1), (Y1′), (Y2), or (Y2′), wherein at least two of R₁, R₂, R₃, R₄, R₅, R₆, or R₇ are independently the amino acid side chain of serine or histidine. In embodiments, the CPP is of the general Formula (AV), (Y1), (Y1′), (Y2), or (Y2′), wherein at least two of R₄, R₅, R₆, or R₇ are independently the amino acid side chain of serine or histidine.

Is embodiments, the CPP is of the general Formula (AV), (Y1), (Y2), or (Y2′), wherein at least one of R₁, R₂, R₃, R₄, R₅, R₆, or R₇ is the amino acid side chain of serine or histidine; and at least one of R₁, R₂, R₃, R₄, R₅, R₆, or R₇ is H. In embodiments, the CPP is of the general Formula (AV), (Y1), (Y2), or (Y2′), wherein at least one of R₁, R₂, R₃, R₄, R₅, R₆, or R₇ is the amino acid side chain of serine or histidine; and at least two of R₁, R₂, R₃, R₄, R₅, R₆, or R₇ are independently H. In embodiments, the CPP is of the general Formula (AV), (Y1), (Y2), or (Y2′), wherein at least one of R₁, R₂, R₃, R₄, R₅, R₆, or R₇ is the amino acid side chain of serine or histidine; and at least two of R₂, R₄, and R₆ are independently H.

In embodiments, the CPP is of the general Formula (AV), (Y1), (Y2), or (Y2′), wherein at least one of R₁, R₂, R₃, R₄, R₅, R₆, or R₇ is the amino acid side chain of serine or histidine; and nₓ is 1. In embodiments, the CPP is of the general Formula (AV), (Y1), (Y2), or (Y2′), wherein at least two of R₁, R₂, R₃, R₄, R₅, R₆, or R₇ are independently the amino acid side chain of serine or histidine; and nₓ is 1. In embodiments, the CPP is of the general Formula (AV), (Y1), (Y2), or (Y2′), wherein at least two of R₄, R₅, R₆, or R₇ are independently the amino acid side chain of serine or histidine; and nₓ is 1.

In embodiments, the CPP is of the general Formula (AV), (Y1), (Y2), or (Y2′), wherein at least one of R₁, R₂, R₃, R₄, R₅, R₆, or R₇ is the amino acid side chain of serine or histidine; and R₁, R₂, and R₃ are independently H or an aromatic or heteroaromatic side chain of an amino acid. In embodiments, the CPP is of the general Formula (AV), (Y1), (Y2), or (Y2′), wherein at least one of R₁, R₂, R₃, R₄, R₅, R₆, or R₇ is the amino acid side chain of serine or histidine; R₁, R₂, and R₃ are independently H or an aromatic or heteroaromatic side chain of an amino acid; and at least one of R₁, R₂, and R₃ is an aromatic or heteroaromatic side chain of an amino acid. In embodiments, the CPP is of the general Formula (AV), (Y1), (Y2), or (Y2′), wherein at least one of R₁, R₂, R₃, R₄, R₅, R₆, or R₇ is the amino acid side chain of serine or histidine; and R₁, R₂, and R₃ are independently aromatic or heteroaromatic side chain of an amino acid. In embodiments, the CPP is of the general Formula (AV), (Y1), (Y2), or (Y2′), wherein at least one of R₁, R₂, R₃, R₄, R₅, R₆, or R₇ is the amino acid side chain of serine or histidine; two of R₁, R₂, and R₃ are independently an aromatic or heteroaromatic side chain of an amino acid; and one of R₁, R₂, and R₃ is H. In embodiments, the CPP is of the general Formula (AV), (Y1), (Y2), or (Y2′), wherein at least one of R₁, R₂, R₃, R₄, R₅, R₆, or R₇ is the amino acid side chain of serine or histidine; and R₁, R₂, and R₃ are independently an aromatic or heteroaromatic side chain of an amino acid.

In embodiments, the CPP is of the general Formula (AV), (Y1), (Y2), or (Y2′), wherein at least one of R₁, R₂, R₃, R₄, R₅, R₆, or R₇ is the amino acid side chain of serine or histidine; and at least one of R₁, R₂, R₃, R₄, R₅, R₆, or R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl) alanine.

In embodiments, the CPP is of the general Formula (AV), (Y-1), (Y-2), or (Y-2′) wherein at least one of R₄, R₅, R₆, R₇ are independently H.

In embodiments, the CPP is of the general Formula (AV), (Y1), (Y2), or (Y2′), wherein at least one of R₄, R₅, R₆, R₇ are independently an uncharged, non-aryl side chain of an amino acid. In embodiments, at least two of R₄, R₅, R₆, or R₇ are independently side chains of an uncharged non-aryl amino acid (e.g., histidine, threonine, serine, leucine, isoleucine, valine, neopentylglycine, alanine, homoalanine, homoserine, 3-(4-Thiazolyl)-alanine, 3-(4-furanyl)-alanine, and 3-(4-thienyl)-alanine). In embodiments, the CPP is of the general Formula (AV), (Y1), (Y2), or (Y2′), wherein at least two of R₄, R₅, R₆, or R₇ are independently side chains of an uncharged non-aryl amino acid selected from histidine, threonine, serine, leucine, isoleucine, valine, neopentylglycine, alanine, homoalanine, homoserine, 3-(4-Thiazolyl)-alanine, 3-(4-furanyl)-alanine, and 3-(4-thienyl)-alanine.

In embodiments, the CPP is of the general Formula (AV), (Y1), (Y2), or (Y2′), wherein at least one of R₁, R₂, R₃, R₄, R₅, R₆, or R₇ is the amino acid side chain of serine or histidine; and at least one of R₄, R₅, R₆, or R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl) alanine. In embodiments, the CPP is of the general Formula (AV), (Y1), (Y2), or (Y2′), wherein at least one of R₄, R₅, R₆, or R₇ is the amino acid side chain of serine; and at least one of R₄, R₅, R₆, or R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl)alanine.

In embodiments, the CPP is of the general Formula (AV), (Y1), (Y2), or (Y2′) wherein at least one of R₄, R₅, R₆, R₇ are independently an uncharged, non-aryl side chain of an amino acid. In embodiments the CPP is of the general Formula (AV), (Y1), (Y2), or (Y2′), wherein at least two of R₄, R₅, R₆, or R₇ are independently side chains of an uncharged non-aryl amino acid. In embodiments the CPP is of the general Formula (AV), (Y1), (Y2), or (Y2′), wherein at least two of R₄, R₅, R₆, or R₇ are independently side chains of an uncharged non-aryl amino acid selected from histidine, threonine, serine, leucine, isoleucine, valine, neopentylglycine, alanine, homoalanine, homoserine, 3-(4-Thiazolyl)-alanine, 3-(4-furanyl)-alanine, and 3-(4-thienyl)-alanine. In embodiments the CPP is of the general Formula (AV), (Y1), (Y2), or (Y2′), at least two of R₄, R₅, R₆, or R₇ are independently side chains of an uncharged non-aryl amino acid selected from serine or histidine.

The cCPP can comprise Formula (Y2), (Y2′), or a protonated form thereof, wherein:
R₁, R₂, R₃, R₄, R₅, R₆, R₇ are independently H or an amino acid side chain,
at least two of R₁, R₂, and R₃ are independently a side chain of phenylalanine or naphthylalanine;
at least two of R₄, R₅, R₆, or R₇ are independently a side chain of arginine;
AA_{SC} is an amino acid side chain; and
nₓ is 1; and
q is 1, 2, 3 or 4.

The cCPP may be Formula (Y-2) or a protonated form thereof,
wherein:
R₁, R₂, R₃, R₄, R₅, R₆, R₇ are independently H or an amino acid side chain;
at least two of R₁, R₂, or R₃ are independently a side chain of an aromatic hydrophobic amino acid,
at least two of R₄, R₅, R₆, or R₇ are independently a side chain of an amino acid comprising a guanidium group;
at least two of R₄, R₅, R₆, or R₇ are independently an uncharged non-aryl amino acid side chain;
AA_{SC} is an amino acid side chain;
nₓ is 1; and
q is 1, 2, 3 or 4.

In embodiments, the CPP is of the general Formula (Y2) or (Y2′), wherein: R₁, R₂, R₃, R₄, R₅, R₆, R₇ are independently H or an amino acid side chain; at least two of R₁, R₂, and R₃ are independently a side chain of phenylalanine, or naphthylalanine; at least two of R₄, R₅, R₆, or R₇ are independently a side chain of arginine; at least two of R₄, R₅, R₆, or R₇ are independently a side chain of an uncharged non-aryl amino acid selected from histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine; AA_{SC} is an amino acid side chain; nₓ is 1; and q is 1, 2, 3 or 4. In embodiments, q is 1.

In embodiments, the CPP is of the structure (AA(a) or (AA(b)) (SEQ ID NOS 168 and 409, respectively, in order of appearance)

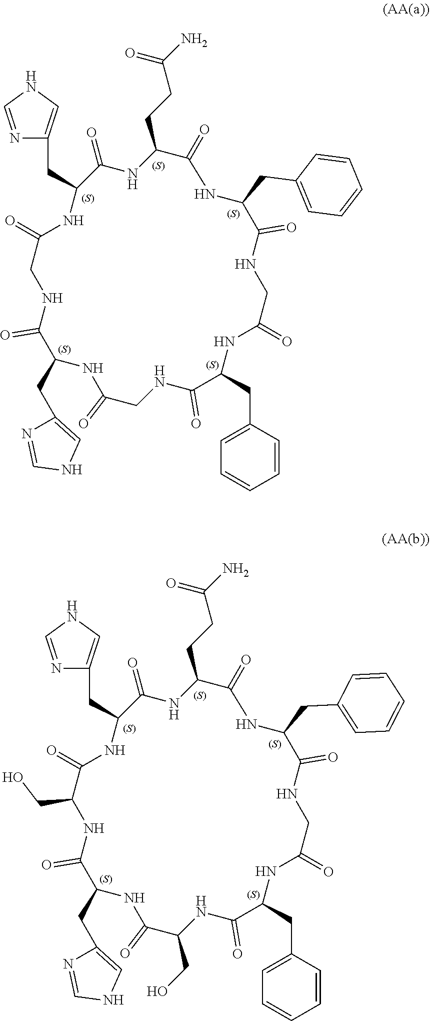

In embodiments, the CPP of general Formula (AV) may comprise one of the following sequences: FGFGHGH (SEQ ID NO: 98); FGFSHSH (SEQ ID NO: 99); FGFGHGHQ (SEQ ID NO: 100); or FGFSHSHQ (SEQ ID NO: 101).

The cCPP of Formula Y1 or Y2 can comprise Formula (Y-a):

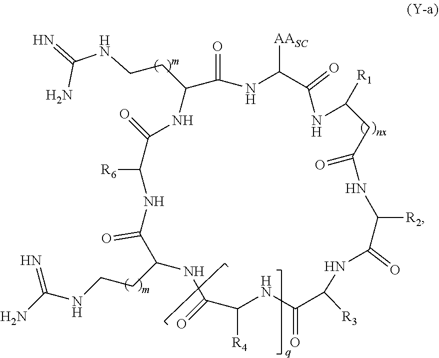

or a protonated form thereof,
wherein:
R₁, R₂, and R₃ can each independently be H or an amino acid residue having a side chain comprising an aromatic or heteroaromatic group;
at least two of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
nₓ is 0 or 1 (according to Formula Y1) or nₓ is 1 (according to Formula Y2); and
each m is independently an integer of 0, 1, 2, or 3.

In embodiments the CPP is of the general Formula (Y-a), wherein R₄ and R₆ are independently H or the side chain of serine or histidine. In embodiments the CPP is of the general Formula (Y-a), wherein R₄ and R₆ are independently H or the side chain of serine or histidine and nₓ is 1. In embodiments the CPP is of the general Formula (Y-a), wherein R₄ and R₆ are independently H or the side chain of serine or histidine; nₓ is 1; and q is 0 (according to Formula Y1 or Y2). In embodiments the CPP is of the general Formula (Y-a) wherein, R₄ and R₆ are independently H or the side chain of serine or histidine and nₓ is 0 (according to Formula Y1). In embodiments the CPP is of the general Formula (Y-a) wherein, R₄ and R₆ are independently H or the side chain of serine or histidine; nₓ is 0; and q is 1 (according to Formula Y1).

In embodiments, the CPP is of the general Formula (Y-a), wherein R₁, R₂, and R₃ can each independently be H, -alkylene-aryl, or -alkylene-heteroaryl. R₁, R₂, and R₃ can each independently be H, -C₁₋₃alkylene-aryl, or -C₁₋₃alkylene-heteroaryl. R₁, R₂, and R₃ can each independently be H or -alkylene-aryl. R₁, R₂, and R₃ can each independently be H or -C₁₋₃alkylene-aryl. C₁₋₃alkylene can be methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can be phenyl. Heteroaryl can be pyridyl, quinolyl, and isoquinolyl. R₁, R₂, and R₃ can each independently be H, -C₁₋₃alkylene-Ph or -C₁₋₃alkylene-Naphthyl. R₁, R₂, and R₃ can each independently be H, -CH₂Ph, or -CH₂-naphthyl. R₁, R₂, and R₃ can each independently be H or -CH₂Ph.

In embodiments, the CPP is of the general Formula (Y-a), wherein R₁, R₂, and R₃ can each independently be the side chain of phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

In embodiments, the CPP is of the general Formula (Y-a), wherein R₁ and R₂ can be side chains of phenylalanine and R₃ can be a side chain of 2-naphthylalanine.

In embodiments, the CPP is of the general Formula (Y-a) wherein R₄ can be H. R₄ can be H or the side chain of an amino acid in Table 1. R₄ can be a residue of an uncharged non-aryl amino acid. In embodiments, R₄ is a side chain of an uncharged non-aryl amino acid selected from histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine. R₄ can be a side chain of serine. R₄ can be a side chain of histidine.

In embodiments, the CPP is of the general Formula (Y-a) wherein R₆ can be H or the side chain of an amino acid in Table 1. R₆ can be a residue of an uncharged non-aryl amino acid. In embodiments, R₆ is a side chain of an uncharged non-aryl amino acid selected from histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine. R₆ can be a side chain of serine. R₆ can be a side chain of histidine.

In embodiments, the CPP is of the general Formula (Y-a) wherein one, two or three of R₁, R₂, R₃, R₄, R₅, and R₆ can be -CH₂Ph. One of R₁, R₂, R₃, R₄, R₅, R₆ , and R₇ can be -CH₂Ph. Two of R₁, R₂, R₃, R₄, R₅, and R₆ can be-CH₂Ph. Three of R₁, R₂, R₃, R₄, R₅, R₆ , and R₇ can be -CH₂Ph. At least one of R₁, R₂, R₃, R₄, R₅, and R₆ can be -CH₂Ph. No more than four of R₁, R₂, R₃, R₄, R₅, R₆, and R₇ can be -CH₂Ph.

In embodiments, the CPP is of the general Formula (Y-a) wherein one, two or three of R₁, R₂, R₃, and R₄ are -CH₂Ph. One of R₁, R₂, R₃, and R₄ is -CH₂Ph. Two of R₁, R₂, R₃, and R₄ are -CH₂Ph. Three of R₁, R₂, R₃, and R₄ are -CH₂Ph. At least one of R₁, R₂, R₃, and R₄ is -CH₂Ph.

In embodiments, the CPP is of the general Formula (Y-a) wherein one, two or three of R₁, R₂, R₃, R₄, R₅, and R₆ can be H. One of R₁, R₂, R₃, R₄, R₅, and R₆ , can be H. Two of R₁, R₂, R₃, R₄, R₅, and R₆ are H. Three of R₁, R₂, R₃, R₅, and R₆ can be H. At least one of R₁, R₂, R₃, R₄, R₅, and R₆ can be H. No more than three of R₁, R₂, R₃, R₄, R₅, and R₆ can be -CH₂Ph.

In embodiments, the CPP is of the general Formula (Y-a) wherein one, two or three of R₁, R₂, R₃, and R₄ are H. One of R₁, R₂, R₃, and R₄ is H. Two of R₁, R₂, R₃, and R₄ are H. Three of R₁, R₂, R₃, and R₄ are H. At least one of R₁, R₂, R₃, and R₄ is H.

In embodiments, the CPP is of the general Formula (Y-a), wherein AA_{SC} can be a side chain of a residue of asparagine, glutamine, or homoglutamine. AA_{SC} can be a side chain of a residue of glutamine. The cCPP can further comprise a linker conjugated the AA_{SC}, e.g., the residue of asparagine, glutamine, or homoglutamine. Hence, the cCPP can further comprise a linker conjugated to the asparagine, glutamine, or homoglutamine residue. The cCPP can further comprise a linker conjugated to the glutamine residue.

In embodiments, the CPP is of the general Formula (Y-a) wherein q can be 1, 2, or 3. q can 1 or 2. q can be 1. q can be 2. q can be 3. q can be 4.

In embodiments, the CPP is of the general Formula (Y-a) wherein m can be 1-3. m can be 1 or 2. m can be 0. m can be 1. m can be 2. m can be 3.

In embodiments, the CPP is of the general Formula (Y-a) wherein nₓ can be 0. nₓ can be 1.

In embodiments, the CPP is of Formula (Y-a), wherein:
R₁, R₂, and R₃ can each independently be H or an amino acid residue having a side chain comprising an aromatic or heteroaromatic group;
at least two of R₁, R₂, and R₃ is an aromatic or heteroaromatic side chain of an amino acid;
R₄ and R₆ are independently H or side chain of serine or histidine;
AA_{SC} is an amino acid side chain;
q is 1, 2, 3 or 4;
nₓ is 1; and
each m is independently an integer 0, 1, 2, or 3.

In embodiments, the CPP is of Formula (Y-a), wherein
R₁, R₂, and Rs can each independently be H or an amino acid residue having a side chain comprising an aromatic or heteroaromatic group;
at least two of R₁, R₂, and R₃ is an aromatic or heteroaromatic side chain of an amino acid;
R₄ and R₆ are independently a side chain of serine or histidine;
AA_{SC} is an amino acid side chain;
q is 1, 2, 3 or 4;
nₓ is 1; and
each m is independently an integer 0, 1, 2, or 3.

The cCPP of Formula (Y-a) can comprise the structure of Formula (Y-aa) or Formula (Y-ab):

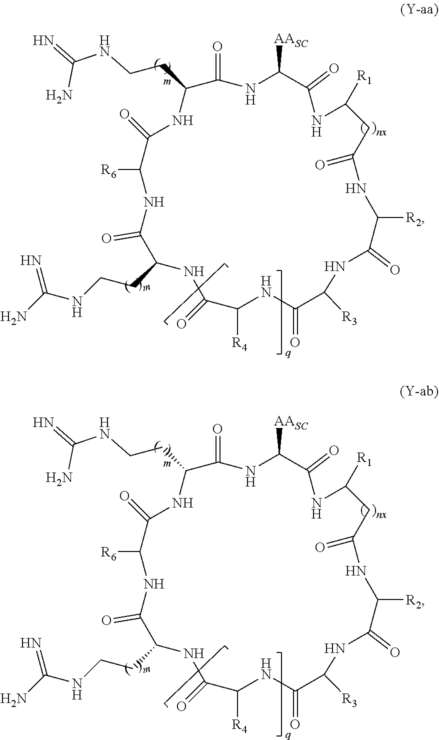

or protonated form thereof, wherein AA_{SC}, R₁, R₂, R₃, R₄, R₇, m and nₓ are as defined herein.

The cCPP can comprise the structure of Formula (Ym), (Yn), (Yo), or (Yp) (SEQ ID NOS 167, 255, 257 and 259, respectively, in order of appearance),:

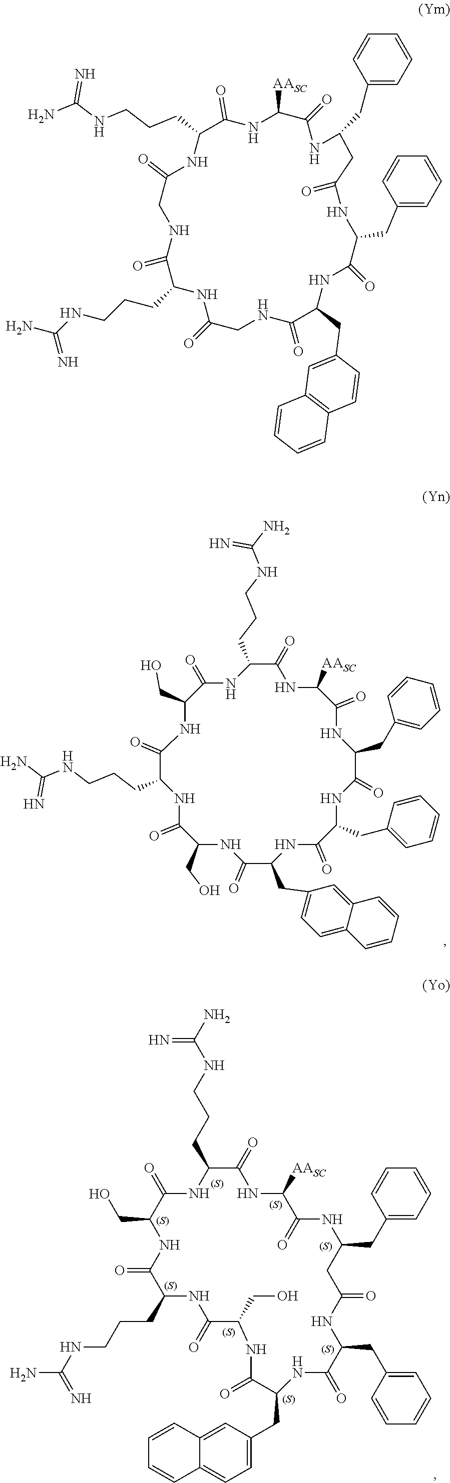

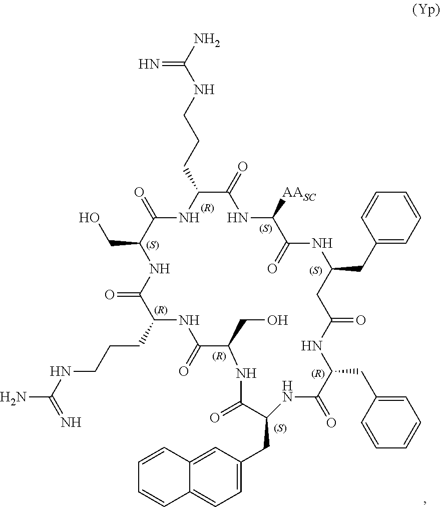

or a protonated form thereof,
wherein AA_{SC} is as defined herein.

The cCPP can comprise one of the following sequences: hFfΦGrGr (SEQ ID NO: 102); bhFfΦSRSR (SEQ ID NO: 103); or FfΦSrSr (SEQ ID NO: 104). The cCPP can comprise one of the following sequences: bhFfΦGrGrQ (SEQ ID NO: 105); bhFfΦSRSRQ (SEQ ID NO: 106); or FfΦSrSrQ (SEQ ID NO: 107).

The cCPP can comprise the structure of Formula AA(c), AA(d), or AA(e) (SEQ ID NOS 259, 427 and 264, respectively, in order of appearance).

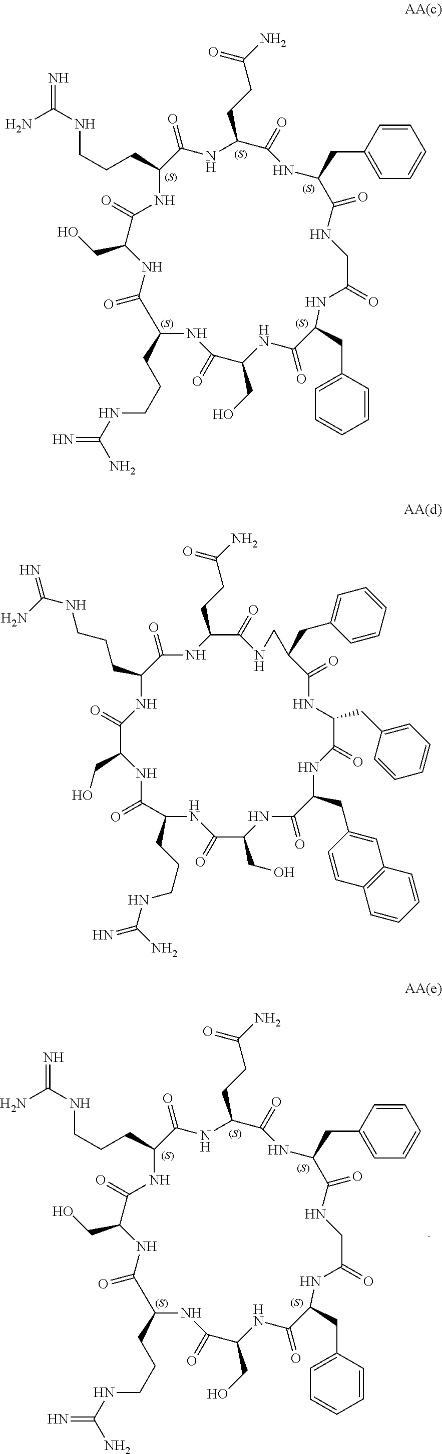

In embodiments, the cCPP can comprise one of the following sequences: FfFSRSR (SEQ ID NO: 108); FGFSRSR (SEQ ID NO: 109); βhFf-Nal-SRSR (SEQ ID NO: 103); FfFSRSRQ (SEQ ID NO: 110); FGFSRSRQ (SEQ ID NO: 111); or βhFf-Nal-SRSRQ (SEQ ID NO: 106).

The disclosure also relates to a cCPP having the structure of Formula (A-II):

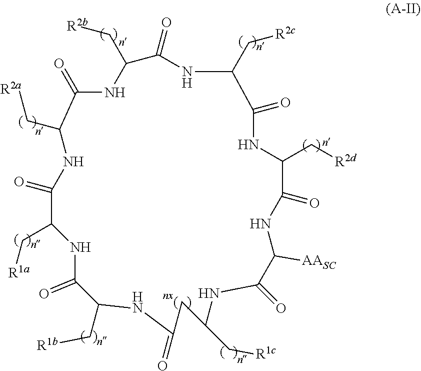

wherein:
AA_{SC} is an amino acid side chain;
R^{1a}, R^{1b}, and R^{1c} are independently a 6- to 14-membered aryl or a 6- to 14-membered heteroaryl;
R^{2a}, R^{2b}, R^{2c} and R^{2d} are independently an amino acid side chain;
at least one of R^{2a}, R^{2b}, R^{2c} and R^{2d} is guanidine or a protonated form thereof;
each n″ is independently an integer 0, 1, 2, 3, 4, or 5;
each n′ is independently an integer from 0, 1, 2, or 3;
nₓ is 0 or 1; and
if n′ is 0 then R^{2a}, R^{2b}, R^{2b} or R^{2d} is absent.

In embodiments where the cCPP is of Formula (A-II), ne or two of R^{2a}, R^{2b}, R^{2c} or R^{2d} are guanidine, or a protonated form thereof, and the remaining of R^{2a}, R^{2b}, R^{2c} or R^{2d} are uncharged non-aryl amino acid side chains. Amino acids with uncharged non-aryl side chains include, but are not limited to, histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine.

In embodiments where the cCPP is of Formula (A-II), each of R^{2a}, R^{2b}, R^{2c} and R^{2d} can independently be serine, homo-serine, threonine, allo-threonine, histidine, or 1-methylhistidine.

AA_{SC} can be

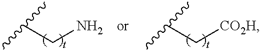

wherein t can be an integer from 0 to 5.

AA_{SC} can be

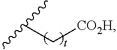

wherein t can be 0 or an integer from 1 to 5. t can be 1 to 5. t is 2 or 3. t can be 2. t can be 3.

In embodiments where the cCPP is of Formula (A-II), R^{1a}, R^{1b}, and R^{1c} can each independently be 6- to 14-membered aryl. R^{1a}, R^{1b}, and R^{1c} can be each independently a 6- to 14-membered heteroaryl having one or more heteroatoms selected from N, O, or S. R^{1a}, R^{1b}, and R^{1c} can each be independently selected from phenyl, naphthyl, anthracenyl, pyridyl, quinolyl, or isoquinolyl. R^{1a}, R^{1b}, and R^{1c} can each be independently selected from phenyl, naphthyl, or anthracenyl. R^{1a}, R^{1b}, and R^{1c} can each be independently phenyl or naphthyl. R^{1a}, R^{1b}, and R^{1c} can each be independently selected pyridyl, quinolyl, or isoquinolyl.

In embodiments where the cCPP is of Formula (A-II), each n′ can independently be 1 or 2. Each n′ can be 1. Each n′ can be 2. At least one n′ can be 0. At least one n′ can be 1. At least one n′ can be 2. At least one n′ can be 3. At least one n′ can be 4. At least one n′ can be 5.

In embodiments where the cCPP is of Formula (A-II), each n″ can independently be an integer from 1 to 3. Each n″ can independently be 2 or 3. Each n″ can be 2. Each n″ can be 3. At least one n″ can be 0. At least one n″ can be 1. At least one n″ can be 2. At least one n″ can be 3.

In embodiments where the cCPP is of Formula (A-II), each n″ can independently be 1 or 2 and each n′ can independently be 2 or 3. Each n″ can be 1 and each n′ can independently be 2 or 3. Each n″ can be 1 and each n′ can be 2. Each n″ is 1 and each n′ is 3.

In embodiments where the cCPP is of Formula (A-II), each nₓ can independently be 0 or 1. nₓ can be 0. nₓ can be 1.

The cCPP of Formula (A-II) can have the structure of Formula (A-II-1):

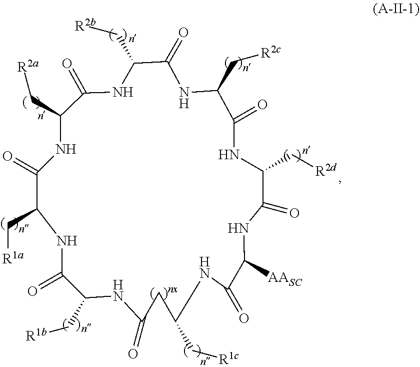

wherein R₁ₐ, R_{1b}, R_{1c}, R₂ₐ, R_{2b}, R_{2c}, R_{2d}, AA_{SC}. n′,n″, and nₓ are as defined herein.

The cCPP of Formula (A-II) or (A-II-1) can have the structure of Formula (A-IIa):

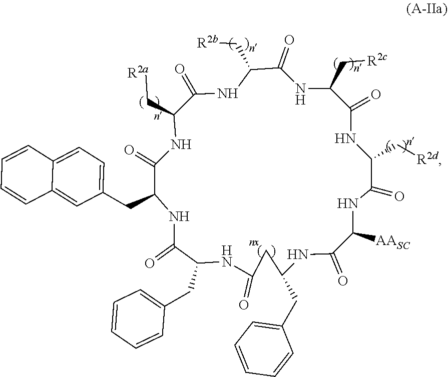

wherein R₁ₐ, R_{1b}, R_{1c}, R₂ₐ, R_{2b}, R_{2c}, R_{2d}, AA_{SC}, n′, and nₓ are as defined herein.

The cCPP of formula (A-II) or (A-II-1) can have the structure of Formula (A-IIb):

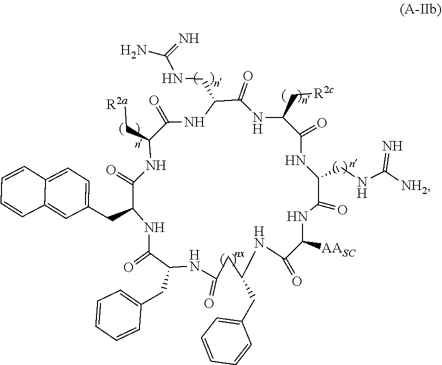

wherein R^{2a}, R^{2b}, AA_{SC}, n′, and nₓ are as defined herein.

The cCPP can have the structure of Formula (A-III):

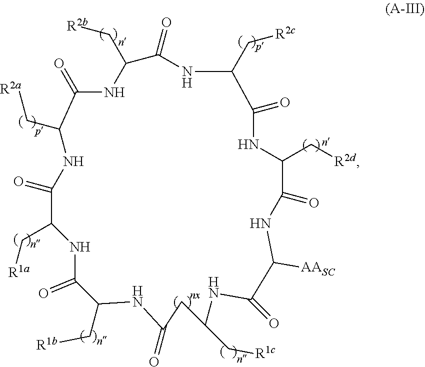

or a protonated form thereof, wherein:
AA_{SC} is an amino acid side chain;
R^{1a}, R^{1b}, and R^{1c} are independently a 6- to 14-membered aryl or a 6- to 14-membered heteroaryl;
R^{2a} and R^{2c} are independently H, or uncharged non-aryl amino acid side chain;
R^{2b} and R^{2d} are independently guanidine or a protonated form thereof;
each n″ is independently an integer from 1 to 3;
each n′ is independently an integer from 1 to 5;
each nₓ is 0 or 1; and
each p′ is independently 0 or 1.

The cCPP of Formula (A-III) can have the structure of Formula (A-III-1):

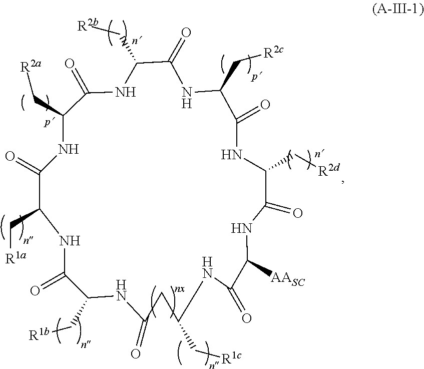

or a protonated form thereof, wherein:
AA_{SC}, R^{1a}, R^{1b}, R^{1c}, R^{2a}, R^{2c}, R^{2b}, R^{2d} n′, n″, nₓ, and p′ are as defined herein.

The cCPP of Formula (A-III) can have the structure of Formula (A-IIIa):

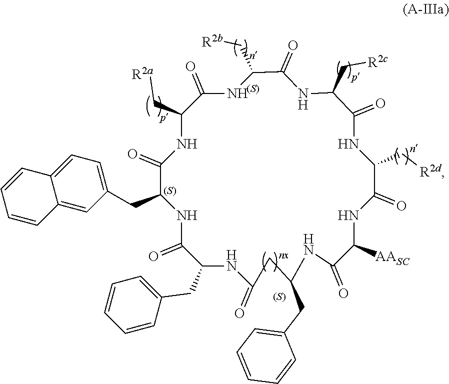

wherein:
AA_{SC}, R^{2a}, R^{2c}, R^{2b}, R^{2d} n′, n″, nₓ, and p′ are as defined herein.

In Formulas (A-III), (A-III-1), and (A-IIIa), R^{2a} and R^{2c} can be H. R^{2a} and R^{2c} can be H and R^{2b} and R^{2d} can each independently be guanidine or protonated form thereof. R^{2a} can be H. R^{2b} can be H. p′ can be 0. R^{2a} and R^{2c} can be H or uncharged non-aryl amino acid side chain and each p′ can be 0, or 1.

In Formulas (A-III), (A-III-1), and (A-IIIa), R^{2a} and R^{2c} can be H or uncharged non-aryl amino acid side chain, R^{2b} and R^{2d} can each independently be guanidine or protonated form thereof, n″ can be 2 or 3, and each p′ can be 0, or 1.

In Formulas (A-III), (A-III-1), and (A-IIIa) p′ can 0. p′ can 1.

In Formulas (A-III), (A-III-1), and (A-IIIa) nₓ can be 0. nₓ can be 1

The cCPP can have the structure (SEQ ID NOS 411, 413 and 415, respectively, in order of appearance):

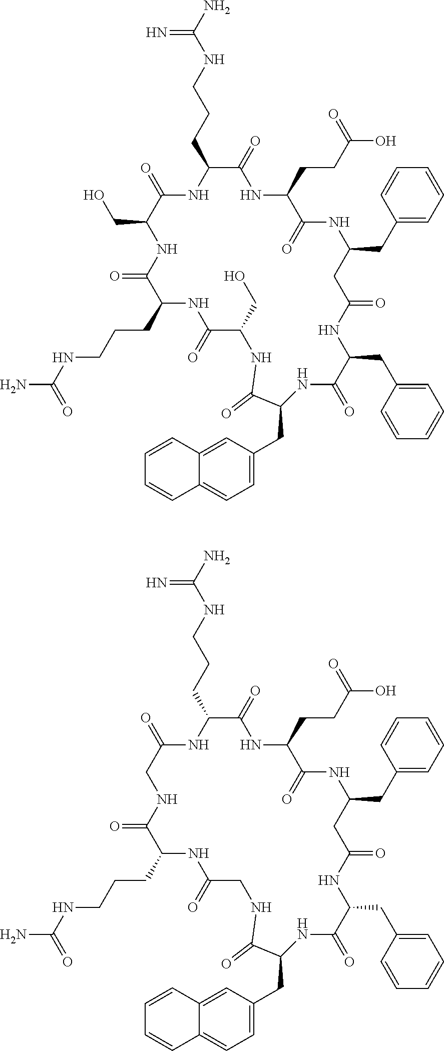 [SMILES: N=C(N)NCCC[C@@H]1NC(=O)[C@H](CO)NC(=O)[C@H](CCCNC(N)=O)NC(=O)[C@H](CO)NC(=O)[C@H](Cc2ccc3ccccc3c2)NC(=O)[C@H](Cc2ccccc2)NC(=O)C[C@H](Cc2ccccc2)NC(=O)[C@H](CCC(=O)O)NC1=O ; N=C(N)NCCC[C@H]1NC(=O)CNC(=O)[C@@H](CCCNC(N)=O)NC(=O)CNC(=O)[C@H](Cc2ccc3ccccc3c2)NC(=O)[C@@H](Cc2ccccc2)NC(=O)C[C@H](Cc2ccccc2)NC(=O)[C@H](CCC(=O)O)NC1=O]

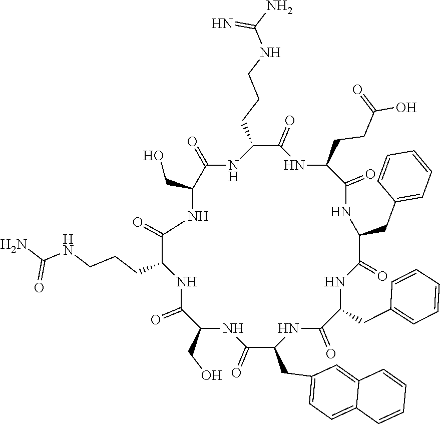 [SMILES: N=C(N)NCCC[C@H]1NC(=O)[C@H](CO)NC(=O)[C@@H](CCCNC(N)=O)NC(=O)[C@H](CO)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

The cCPP of Formula (Y) can be selected from:

CPP Sequence

(bhFfΦGrGrQ) (SEQ ID NO: 105)
(bhFfΦSrSrQ) (SEQ ID NO: 112
(bhFfΦHrHrQ) (SEQ ID NO: 113)
(bhFFΦSRSRQ) (SEQ ID NO: 114
(bhFFΦGRGRQ) (SEQ ID NO: 115)
(bhFFΦHRHRQ) (SEQ ID NO: 116)
(FfΦSrSrQ) (SEQ ID NO: 107)
(FfΦHrHrQ) (SEQ ID NO: 117)

The cCPP can comprise the structure of Formula (A-D)

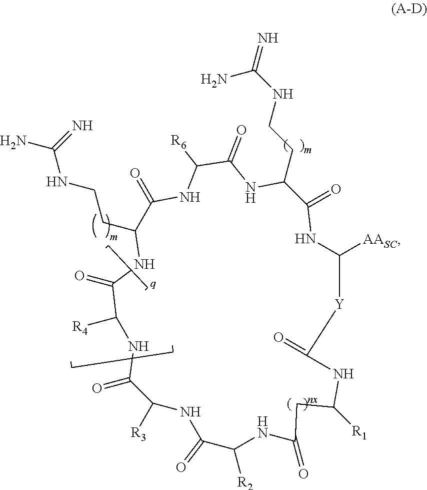

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an uncharged non-aryl amino acid side chain;
AA_{SC} is an amino acid side chain;
Y is

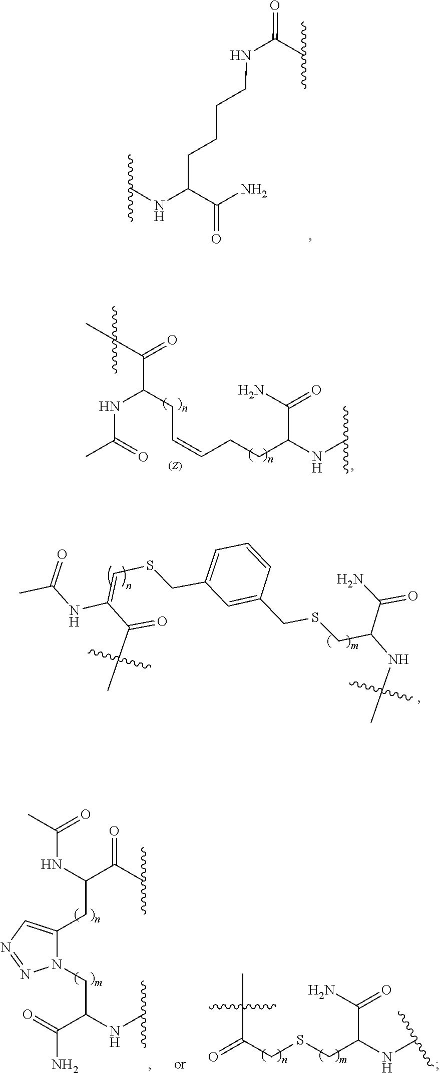

q is 1, 2, 3 or 4;
each m is independently an integer 0, 1, 2, or 3,
each n is independently an integer 0, 1, 2, or 3, and
nₓ is 0 or 1.

In embodiments, the cCPP is of Formula (A-D), wherein Y is

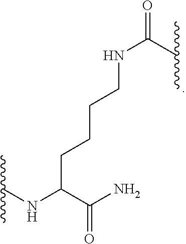 [SMILES: *NC(CCCCNC(*)=O)C(N)=O]

In embodiments, the cCPP is of Formula (A-D), wherein Y is

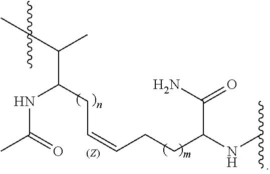

and each of m and n are independently 0, 1, 2, or 3.

In embodiments, the cCPP is of Formula (A-D), wherein Y is

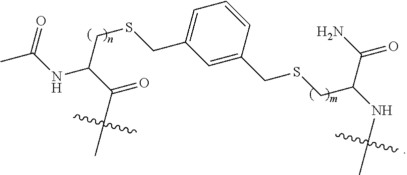

and each of m and n are independently 0, 1, 2, or 3.

In embodiments, the cCPP is of Formula (A-D), wherein Y is

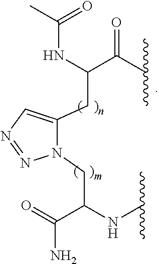

and each of. m and n are independently 0, 1, 2, or 3.

AA_{SC} can be conjugated to a linker.

### Endosomal Escape Vehicles (EEVs)

In embodiments, the delivery construct includes an endosomal escape vehicle (EEV). An EEV comprises a cell penetrating peptide (CPP), for example, a cyclic cell penetrating peptide (cCPP). In embodiments, the EEV comprises a cCPP and an exocyclic peptide (EP). In embodiments, the EEV comprises a cCPP, and EP and a linker (L). The linker may include a reactive handle that allows for conjugation to a cargo. The linker may conjugate the cCPP and the EP. The linker may include a reactive handle that can react with a reactive handle on a cargo to form a cargo conjugate.

The cargo may be lipid, a component of gene editing machinery (GEM), or a payload of a lipid-based particle. The payload of a lipid-based particle may be a component of GEM. In embodiments, an EEV is conjugated to a lipid to form a lipid conjugate. In embodiments, an EEV is conjugated to one or more components of GEM to form a GEM conjugate. In embodiments, the EEV is conjugated to a ribonucleoprotein (RNP). In embodiments, GEM conjugate is delivered to a cell as a payload of a lipid-based particle. In embodiments, GEM conjugates are delivered to a cell independently of a lipid-based particle.

The lipid conjugate can include a lipid coupled to an endosomal escape vehicle (EEV). The inclusion of a lipid conjugate in a lipid-based particle may enhance the transport of the payload across a cellular membrane as compared to a lipid-based particle that does not include a lipid conjugate. For example, the inclusion of a lipid conjugate in a lipid-based particle may enhance the transport of the payload to the cytosol or nucleus of a cell as compared to a lipid-based particle that does not include a lipid conjugate.

The GEM conjugates include an EEV coupled to one or more components of a gene editing machinery (GEM). The EEV may enhance the transport of the GEM across a cellular membrane as compared to a GEM the is not conjugated to an EEV.

### Exocyclic Peptide (EP)

In embodiments, an EEV includes an exocyclic peptide (EP). The EP can comprise from 2 to 10 amino acid residues e.g., 2, 3, 4, 5, 6, 7, 8, 9, or 10 amino acid residues, inclusive of all ranges and values therebetween. In embodiments, the EP comprises 6 to 9 amino acid residues. In embodiments, the EP comprises from 4 to 8 amino acid residues.

Each amino acid in the EP may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in Table 1 along with their abbreviations used herein. For example, the amino acids can be A, G, P, K, R, V, F, H, Nal, or citrulline.

The EP can comprise at least one positively charged amino acid residue, e.g., at least one lysine residue and/or at least one amine acid residue comprising a side chain comprising a guanidine group, or a protonated form thereof. The EP can comprise 1 or 2 amino acid residues comprising a side chain comprising a guanidine group, or a protonated form thereof. The amino acid residue comprising a side chain comprising a guanidine group can be an arginine residue. Protonated forms can mean salt thereof throughout the disclosure.

The EP can comprise at least two, at least three or at least four lysine residues. The EP can comprise 2 lysine residues. The EP can comprise 3 lysine residues. The EP can comprise 4 lysine residues. The amino group on the side chain of each lysine residue can be substituted with a protecting group, including, for example, trifluoroacetyl (—COCF₃), allyloxycarbonyl (Alloc), 1-(4,4-dimethyl-2,6-dioxocyclohexylidene)ethyl (Dde), or (4,4-dimethyl-2,6-dioxocyclohex-1-ylidene-3)-methylbutyl (ivDde) group. The amino group on the side chain of each lysine residue can be substituted with a trifluoroacetyl (—COCF₃) group. The protecting group can be included to enable amide conjugation. The protecting group can be removed after the EP is conjugated to a cCPP. The protecting group can be removed after the EEV is conjugated to a cargo.

The EP can comprise at least 2 amino acid residues with a hydrophobic side chain. The amino acid residue with a hydrophobic side chain can be selected from valine, proline, alanine, leucine, isoleucine, methionine, or combinations thereof. The amino acid residue with a hydrophobic side chain can be valine or proline.

The EP can comprise at least one positively charged amino acid residue, e.g., at least one lysine residue and/or at least one arginine residue. The EP can comprise at least two, at least three, or at least four lysine residues and/or arginine residues.

The EP can comprise KK, KR, RR, HH, HK, HR, RH, KKK, KGK, KBK, KBR, KRK, KRR, RKK, RRR, KKH, KHK, HKK, HRR, HRH, HHR, HBH, HHH, HHHH (SEQ ID NO: 118), KHKK (SEQ ID NO: 119), KKHK (SEQ ID NO: 120), KKKH (SEQ ID NO: 121), KHKH (SEQ ID NO: 122), HKHK (SEQ ID NO: 123), KKKK (SEQ ID NO: 124), KKRK (SEQ ID NO: 125), KRKK (SEQ ID NO: 126), KRRK (SEQ ID NO: 127), RKKR (SEQ ID NO: 128), RRRR (SEQ ID NO: 129), KGKK (SEQ ID NO: 130), KKGK (SEQ ID NO: 131), HBHBH, HBKBH, RRRRR (SEQ ID NO: 134), KKKKK (SEQ ID NO: 135), KKKRK (SEQ ID NO: 136), RKKKK (SEQ ID NO: 137), KRKKK (SEQ ID NO: 138), KKRKK (SEQ ID NO: 139), KKKKR (SEQ ID NO: 140), KBKBK, RKKKKG (SEQ ID NO: 142), KRKKKG (SEQ ID NO: 143), KKRKKG (SEQ ID NO: 144), KKKKRG (SEQ ID NO: 145), RKKKKB (SEQ ID NO: 146), KRKKKB (SEQ ID NO: 147), KKRKKB (SEQ ID NO: 148), KKKKRB (SEQ ID NO: 149), KKKRKV (SEQ ID NO: 150), RRRRRR (SEQ ID NO: 151), HHHHHH (SEQ ID NO: 152), RHRHRH (SEQ ID NO: 153), HRHRHR (SEQ ID NO: 154), KRKRKR (SEQ ID NO: 155), RKRKRK (SEQ ID NO: 156), RBRBRB, KBKBKB, PKKKRKV (SEQ ID NO: 159), PGKKRKV (SEQ ID NO: 160), PKGKRKV (SEQ ID NO: 161), PKKGRKV (SEQ ID NO: 162), PKKKGKV (SEQ ID NO: 163), PKKKRGV (SEQ ID NO: 164), or PKKKRKG (SEQ ID NO: 165), wherein B is β-alanine. The amino acids in the EP can have D or L stereochemistry.

The EP can comprise KK, KR, RR, KKK, KGK, KBK, KBR, KRK, KRR, RKK, RRR, KKKK (SEQ ID NO: 124), KKRK (SEQ ID NO: 125), KRKK (SEQ ID NO: 126), KRRK (SEQ ID NO: 127), RKKR (SEQ ID NO: 128), RRRR (SEQ ID NO: 129), KGKK (SEQ ID NO: 130), KKGK (SEQ ID NO: 131), KKKKK (SEQ ID NO: 135), KKKRK (SEQ ID NO: 136), KBKBK, KKKRKV (SEQ ID NO: 150), PKKKRKV (SEQ ID NO: 159), PGKKRKV (SEQ ID NO: 160), PKGKRKV (SEQ ID NO: 161), PKKGRKV (SEQ ID NO: 162), PKKKGKV (SEQ ID NO: 163), PKKKRGV (SEQ ID NO: 164), or PKKKRKG (SEQ ID NO: 165). The EP can comprise PKKKRKV (SEQ ID NO: 159), RR, RRR, RHR, RBR, RBRBR, RBHBR, or HBRBH, wherein B is β-alanine. The amino acids in the EP can have D or L stereochemistry.

The EP can consist of KK, KR, RR, KKK, KGK, KBK, KBR, KRK, KRR, RKK, RRR, KKKK (SEQ ID NO: 124), KKRK (SEQ ID NO: 125), KRKK (SEQ ID NO: 126), KRRK (SEQ ID NO: 127), RKKR (SEQ ID NO: 128), RRRR (SEQ ID NO: 129), KGKK (SEQ ID NO: 130), KKGK (SEQ ID NO: 131), KKKKK (SEQ ID NO: 135), KKKRK (SEQ ID NO: 136), KBKBK, KKKRKV (SEQ ID NO: 150), PKKKRKV (SEQ ID NO: 159), PGKKRKV (SEQ ID NO: 160), PKGKRKV (SEQ ID NO: 161), PKKGRKV (SEQ ID NO: 162), PKKKGKV (SEQ ID NO: 163), PKKKRGV (SEQ ID NO: 164), or PKKKRKG (SEQ ID NO: 165). The EP can consist of PKKKRKV (SEQ ID NO: 159), RR, RRR, RHR, RBR, RBRBR, RBHBR, or HBRBH, wherein B is β-alanine. The amino acids in the EP can have D or L stereochemistry.

The EP can comprise an amino acid sequence identified in the art as a nuclear localization sequence (NLS). The EP can comprise an NLS comprising the amino acid sequence PKKKRKV (SEQ ID NO: 42). The EP can consist of an NLS comprising the amino acid sequence PKKKRKV (SEQ ID NO: 42). The EP can comprise an NLS comprising or consisting of an amino acid sequence selected from NLSKRPAAIKKAGQAKKKK (SEQ ID NO: 169), PAAKRVKLD (SEQ ID NO: 170), RQRRNELKRSF (SEQ ID NO: 171), RMRKFKNKGKDTAELRRRRVEVSVELR (SEQ ID NO: 172), KAKKDEQILKRRNV (SEQ ID NO: 173), VSRKRPRP (SEQ ID NO: 174), PPKKARED (SEQ ID NO: 175), PQPKKKPL (SEQ ID NO: 176), SALIKKKKKMAP (SEQ ID NO: 177), DRLRR (SEQ ID NO: 178), PKQKKRK (SEQ ID NO: 179), RKLKKKIKKL (SEQ ID NO: 180), REKKKFLKRR (SEQ ID NO: 181), KRKGDEVDGVDEVAKKKSKK (SEQ ID NO: 182), and RKCLQAGMNLEARKTKK (SEQ ID NO: 183).

All exocyclic sequences can also contain an N-terminal acetyl group (Ac). Hence, for example, the EP can have the structure: Ac-PKKKRKV (SEQ ID NO: 184).

The EP can be coupled to the cargo (e.g., a lipid or a GEM component). The EP can be coupled to the cCPP. The EP can be coupled to the cargo and the cCPP. Coupling between the EP, cargo, cCPP, or combinations thereof, may be non-covalent or covalent. The EP can be attached through a peptide bond to the N-terminus of the cCPP. The EP can be attached through a peptide bond to the C-terminus of the cCPP. The EP can be attached to the cCPP through a side chain of an amino acid in the cCPP. The EP can be attached to the cCPP through a side chain of a lysine which can be conjugated to the side chain of a glutamine in the cCPP. The EP can be coupled to a linker. The EP can be conjugated to an amino group of the linker. The EP can be coupled to a linker via the C-terminus of an EP and a cCPP through a side chain on the cCPP and/or EP. For example, an EP may comprise a terminal lysine which can then be coupled to a cCPP containing a glutamine through an amide bond. When the EP contains a terminal lysine, and the side chain of the lysine can be used to attach the cCPP, the C-or N-terminus may be attached to a linker on the cargo.

### Linker

One or more linkers (L) may be used to link the components of a delivery construct and/or the cargo conjugates.

Linkers may link individual components of a compound together through one or more conjugation reactions to form conjugates. As used herein, a “conjugate” is a compound formed by the joining of two or more compounds. Conjugates may or may not include a linker between the two or more components. The linker can be any appropriate moiety that can link a cCPP, EP, EEV, or cargo to one or more additional moieties. A linker can be conjugated to first component to form a first component-linker conjugate, and the first component-linker conjugate can be conjugated to a second component to form a first component-linker-second component conjugate, which may also be referred to herein as a first component-second component conjugate. The first component-second component conjugate can be further conjugated to a third component to form a first component-second component-third component conjugate. For example, a linker may be conjugated to a cCPP to form a linker-cCPP conjugate. A linker may be conjugated to an EP to form a linker-EP conjugate. A linker-cCPP conjugate or a linker-EP conjugate may be conjugated to an EP or cCPP, respectively, to form an EEV that includes a cCPP, an EP, and a linker (e.g., an EP-linker-cCPP conjugate). A delivery construct may be conjugated to a lipid cargo to form a lipid conjugate. A delivery construct may be conjugated to a GEM cargo to form a GEM conjugate.

Prior to conjugation, the linker and the component to be coupled may include a pair of cooperative reactive handles. Following conjugation, the conjugate may include the reaction product of the pair of cooperative reactive handles.

Cooperative handles are two or more reactive handles (Rh) that, when exposed to each other under favorable reaction conditions, undergo a conjugation reaction to form a reaction product between the reactive handles. Any pair of cooperative reactive handles may be used to form the conjugates. Examples of cooperative handles include an activated ester and an amine; an amine and an NHS-ester; a hydroxyl and an NHS-ester; a hydroxyl and an epoxide; an acyl chloride and an amine; an acyl chloride and an alcohol; an amine and an epoxide; a thiol and an epoxide; a thiol and a maleimide; a disulfide and a thiol; an azide and an alkyne (azide and a linear alkyne in the presence of Cu(I); an azide and a cyclic alkyne such as cyclooctyne, difluorinated cyclooctyne, dibenxocyclooctyne, TMTH-SulfoxImine, biarylazacyclooctynone, aryl-less cyclooctyne, or bicyclo[6.1.0] nonyne); an amine and an isocyanate; an amine and an isothiocyanate, a amine and a benzoyl fluoride; a thiol and a iodoacetamide; a thiol and a bromoacetamide; a disulfide and 2-thiopyridine; a thiol and 3-arylpropiolonitirle; a phenol and a diazonium salt; a phenol and 4-phenyl-1,2,4-triazoline-3,5-dione; a phenol and aldehyde, and a aniline; a hydroxyl and sodium periodate; a thiol and an iodoacetamide; an amine and a pyridoxal phosphate; an azide and a functionalized triphenyl phosphine; a tetrazine and a strained alkene; and the like.

Examples of individual reactive handles that may be used to form the conjugates include Rh^{A} (hydroxyl), Rh^{B} (thiol), Rh^{C} (amine), Rh^{D} (activated ester), Rh^{E} (azide), Rh^{F} (alkyne), Rh^{G}(NHS-ester), Rh^{H} (maleimide), Rh^{I} (2-haloacetamides, where a halo group is a -chloro, -bromo, or -jodo leaving group attached to carbon that can undergo nucleophilic substitution; e.g., a bromoacetamide or iodoacetamide), Rh^{J} (azadibenzocyclooctyne (ADIBO or DBCO or DIBAC)), Rh^{K} (isocyanate), Rh^{L} (isothiocyanate), Rh^{M} (alkylhalides, where halide is a -chloro, -bromo, or -iodo leaving group attached to carbon that can undergo nucleophilic substitution), Rh^{N} (an epoxide), Rh^{O} (an acyl chloride), Rh^{P} (an aldehyde) and isomers thereof. Chemical structures of Rh^{A}-Rh^{P} are depicted below.

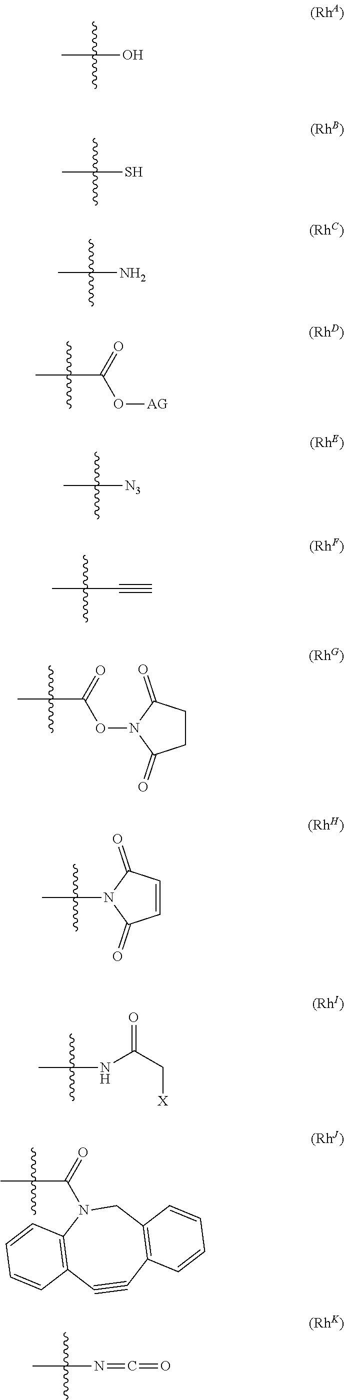

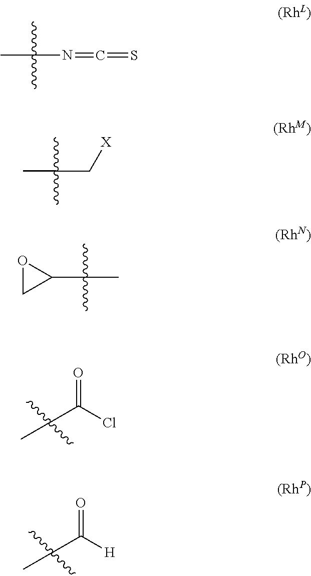

X in Rh^{M} and Rh^{I} may be -choro, bromo, or -iodo.

Rh^{D} is an activated ester where AG is an activating group. An activated ester is an ester that is reactive with an activated ester cooperative reaction handle (e.g., an amine) in a conjugation reaction. Activated esters may be denoted as the type of activated ester or by the activating group. Examples of activating groups include O-acylisoureas, benzotriazoles (with a bond between the ester oxygen and one nitrogen of the triazole), and pentafluorophenyl or tetrafluorophenyl. In embodiments, Rh^{D} may be an activated ester of a carboxylic acid. The activated ester can be formed through reaction of a carboxylic acid with one or more reagents that install the activating group. For example, a carboxylic acid may be converted into an activated ester having a O-acylisoureas activating group by treating the carboxylic acid with various carbodiimide reagents (e.g., N,N′-dicyclohexylcarbodiimide (DCC), 1-ethyl-3-(3-dimethylaminopropyl)carbodiimide (EDC), or diisopropylcarbodiimide (DIC)) under favorable reaction conditions. A carboxylic acid may be converted into an activated ester having a benzotriazole activating group by treating the carboxylic acid with various carbodiimide reagents followed by treatment with hydroxybenzotriazole (HOBT) or by treating the carboxylic acid with various benzotriazole containing compounds (e.g., O-(benzotriazol-1-yl)-N,N,N′,N′-tetramethyluronium hexafluorophosphate or 2-(1H-benzotriazol-1-yl)-1,1,3,3-tetramethyluronium hexafluorophosphate (HBTU); O-(benzotriazol-1-yl)-N,N,N′,N′-tetramethyluronium tetrafluoroborate (TBTU); benzotriazol-1-yloxy)tris(dimethylamino)phosphonium hexafluorophosphate (BOP); (benzotriazol-1-yloxy)tripyrrolidinophosphonium hexafluorophosphate (PyBOP); and O-(7-azabenzotriazol-1-yl)-N,N,N′,N′-tetramethyluronium tetrafluoroborate (TATU)) under favorable reaction conditions. Other reagents are available for making activated esters from carboxylic acids including bromotripyrrolidinophosphonium hexafluorophosphate (PyBrOP); O-(N-Succinimidyl)-1,1,3,3-tetramethyl-uronium tetrafluoroborate (TSTU); O-(5-Norbornene-2,3-dicarboximido)-N,N,N′,N′-tetramethyluronium tetrafluoroborate (TNTU); O-(1,2-Dihydro-2-oxo-1-pyridyl-N,N,N′,N′-tetramethyluronium tetrafluoroborate (TPTU); and 3-(diethylphosphoryloxy)-1,2,3-benzotriazin-4(3H)-one (DEPBT); carbonyldiimidazole (CDI). In embodiments, the activated ester may be created in situ from a carboxylic acid and not isolated prior to a conjugation reaction.

Reactive handles Rh^{A}, Rh^{B}, Rh^{C}, Rh^{D}, Rh^{E}, Rh^{F}, Rh^{G}, Rh^{H}, Rh^{I}, Rh^{J}, Rh^{K}, Rh^{L}, Rh^{M}, Rh^{N}, Rh^{O}, and Rh^{P} include various pairs of cooperative handles that can form the reaction products of Rp^{A}, Rp^{B}, Rp^{C}, Rp^{D}, Rp^{E}, Rp^{F}, Rp^{G}, Rp^{H}, Rp^{I}, Rp^{J}, and Rp^{K} (shown below). Such reaction products may also be referred to as bonding groups (M, as disclosed herein). In embodiments, the conjugates include one or more of the reaction products Rp^{A}, Rp^{B}, Rp^{C}, Rp^{D}, Rp^{E}, Rp^{F}, Rp^{G}, Rp^{H}, Rp^{I}, Rp^{J}, and Rp^{K}.

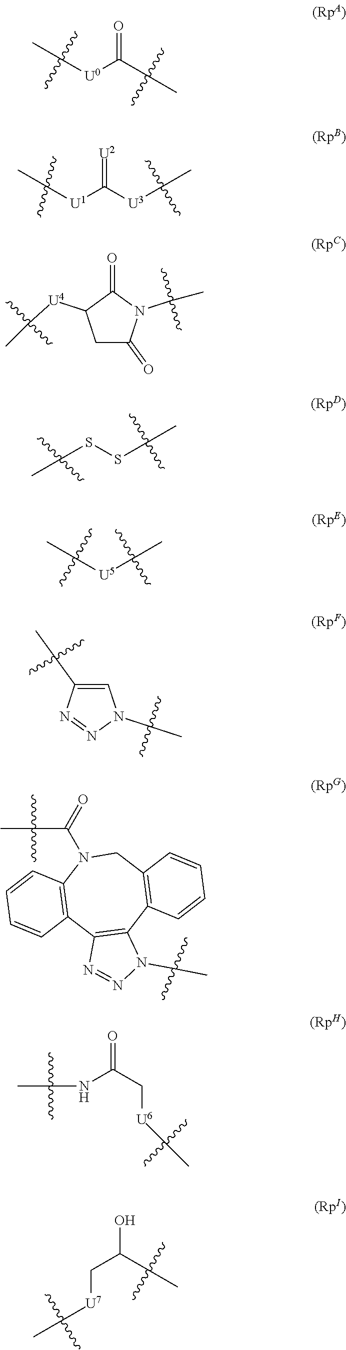

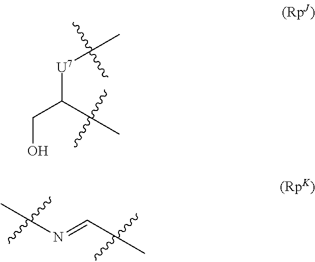

For example, under favorable reaction conditions, a conjugation reaction between Rh^{A} and Rh^{D} forms Rp^{A} where U⁰ is O. Under favorable reaction conditions, a conjugation reaction between Rh^{D} and Rh^{C} forms Rp⁴ where U⁰ is NH. Under favorable reaction conditions, a conjugation reaction between Rh^{C} and Rh^{G} forms Rp^{A} where U⁰ is NH. Under favorable reaction conditions, a conjugation reaction between Rh^{B} and Rh^{H} forms Rp^{C} where U⁴ is S. Under favorable reaction conditions, a conjugation reaction between two Rh^{B} forms Rp^{D}. Under favorable reaction conditions, a conjugation reaction between Rh^{C} and Rh^{I} forms Rp^{H} where U⁶ is NH. Under favorable reaction conditions, a conjugation reaction between Rh^{B} and Rh^{I} forms Rp^{H} where U⁶ is S. Under favorable reaction conditions, a conjugation reaction between Rh^{M} and Rh^{B} forms Rp^{E} where U⁵ is S. Under favorable reaction conditions, a conjugation reaction between Rh^{M} and Rh^{C} forms Rp^{E} where U⁵ is NH. Under favorable reaction conditions, a conjugation reaction between Rh^{K} and Rh^{C} forms Rp^{B} where U¹ and U³ are NH and U² is O. Under favorable reaction conditions, a conjugation reaction between Rh^{L} and Rh^{C} forms Rp^{B} where U¹ and U³ are N^{H} and U² is S. Under favorable reaction conditions, a conjugation reaction between Rh^{F} and Rh^{E} forms Rp^{F}. Under favorable reaction conditions, a conjugation reaction between Rh^{J} and Rh^{E} forms Rp^{G}. Under favorable reaction conditions, a conjugation reaction between Rh^{N} and Rh^{A} forms Rp⁴ or Rp^{J} where U⁷ is O. Under favorable reaction conditions, a conjugation reaction between Rh^{N} and Rh^{B} forms Rp^{I} or Rp^{J} where U⁷ is S. Under favorable reaction conditions, a conjugation reaction between Rh^{N} and Rh^{C} forms Rp^{I} or Rp^{J}where U⁷ is N. Under favorable reaction conditions, a conjugation reaction between Rh^{O} and Rh^{A} forms Rp^{A} where U⁰ is O. Under favorable reaction conditions, a conjugation reaction between Rh^{O}and Rh^{B} forms Rp^{A} where U⁰ is NH. Under favorable reaction conditions, a conjugation reaction between Rh^{P} and Rh^{C} forms Rp^{K}.

In embodiments, the linker includes one or more of the reactive handles disclosed herein capable of forming one or more of the reaction products disclosed herein. In embodiments, the cCPP, EP, cargo, or any combination thereof includes one or more reactive handles disclosed herein capable of reaction with the reactive handle of a linker disclosed herein to form a conjugate comprising one or more of the reaction products disclosed herein. In embodiments, the conjugate includes one or more of the reaction products described herein. In embodiments, the linker includes one or more of the reaction products described herein.

In embodiments, a delivery construct is conjugated to cargo through a direct conjugation reaction. A direct conjugation reaction is a reaction in which the two components that are being covalently linked have the proper cooperative functional handles without the need for an intermediary bifunctional bioconjugation compound. Direct bioconjugation reactions can be accomplished using any suitable cooperative reaction handles, such as any of the cooperative functional handles disclosed herein, to result in the reaction products disclosed herein.

In embodiments, a delivery construct is conjugated to a cargo using a bifunctional conjugation compound in an indirect bioconjugation reaction. An indirect bioconjugation reaction is the conjugation of two components through an intermediary bifunctional conjugation compound. A bifunctional conjugation compound includes a first reactive handle and a second reactive handle that are configured to react with cooperative functional handles on the components to be conjugated. Examples of pairs of reactive handles on a bifunctional bioconjugation compound include NHS-ester and an alkyne, a maleimide and an NHS-ester, an NHS ester and a disulfide, a dibenzocyclooctyne (DBCO) and an NHS ester, DBCO and a tetrafluophenyl ester, and the like. Indirect conjugation reactions often include two consecutive conjugation reactions; a first conjugation reaction to attach a first component to the bifunctional conjugation compound and a second conjugation reaction to attach the second component to the bifunctional conjugation compound. Generally, the two conjugation reactions are orthogonal. The first component has a reactive handle that is cooperative with a first reactive handle on the bifunctional conjugation compound, and the second component has a reactive handle that is cooperative with a second reactive handle on the bifunctional conjugation compound. Generally, the two pairs of cooperative functional handles allow for orthogonal conjugation reactions. Any conjugation chemistry and any two pairs of cooperative functional handles, such as cooperative reaction handles described herein, may be used. In embodiments, where a delivery construct is conjugated to a cargo through a bifunctional conjugation compound, the bonding group (M) connecting the two components includes the reaction products of the two conjugation reactions and any chemical group of the bifunctional bioconjugation compound that separates the two reactive handles of the bifunctional bioconjugation compound.

A delivery construct or component of a delivery construct (e.g., a linker) can be linked to a cargo through a bonding group (“M”). In embodiments where the delivery construct or component of the delivery construct (e.g., a linker) is conjugated to the cargo through a direct conjugation reaction the bonding group may include or be any reaction product as disclosed herein. In embodiments, where a delivery construct is conjugated to a cargo through a bifunctional conjugation compound, the bonding group (M) connecting the two components includes the reaction products of the two conjugation reactions and any chemical group of the bifunctional bioconjugation compound that separates the two reactive handles of the bifunctional bioconjugation compound. The two reaction products of an indirect conjugation reaction may be any indirect conjugation reaction products, such as those as disclosed herein.

The cCPPs of a delivery construct can be conjugated to a linker. The linker can be attached to the side chain of an amino acid of the cCPP. A cargo can be attached at a suitable position on linker. In embodiments, the linker is attached to the AA_{sc} of the cCPP. The location of attachment of a cCPP and/or cargo to a linker may comprise a reaction product between a pair of cooperative reactive handles. The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

In embodiments, the linker can be bivalent and link the cCPP to a cargo. In embodiments, the linker can be bivalent and link the cCPP to an exocyclic peptide (EP). In embodiments, the linker can be a bivalent linker and link a delivery construct such as an EEV (comprising a cCPP and an exocyclic peptide) to a cargo.

In embodiments, the linker can be trivalent and link a cCPP, an EP, and a cargo in a single compound (e.g., a lipid conjugate or GEM conjugate).

The linker can comprise hydrocarbon linker.

The linker can comprise a cleavage site. The cleavage site can be a disulfide that can be reduced under appropriate conditions, or caspase-cleavage site (e.g, Val-Cit-PABC).

The linker can comprise: (i) one or more D or L amino acids, each of which is optionally substituted; (ii) optionally substituted alkylene; (iii) optionally substituted alkenylene; (iv) optionally substituted alkynylene; (v) optionally substituted carbocyclyl; (vi) optionally substituted heterocyclyl; (vii) one or more —(R¹—J—R²)z″— subunits, wherein each of R¹ and R², at each instance, are independently selected from alkylene, alkenylene, alkynylene, carbocyclyl, and heterocyclyl, each J is independently C, NR³, —NR³C(O)—, S, and O, wherein R³ is independently selected from H, alkyl, alkenyl, alkynyl, carbocyclyl, and heterocyclyl, each of which is optionally substituted, and z″ is an integer from 1 to 50; (viii) —(R¹-J)z″— or —(J—R¹)z″—, wherein each of R¹, at each instance, is independently alkylene, alkenylene, alkynylene, carbocyclyl, or heterocyclyl, each J is independently C, NR₃, —NR³C(O)—, S, or O, wherein R₃ is H, alkyl, alkenyl, alkynyl, carbocyclyl, or heterocyclyl, each of which is optionally substituted, and z″ is an integer from 1 to 50; or (ix) the linker can comprise one or more of (i) through (x).

The linker can comprise one or more D or L amino acids and/or —(R¹—J—R²)z″—, wherein each of R¹ and R², at each instance, are independently alkylene, each J is independently C, NR³, —NR³C(O)—, S, and O, wherein R⁴ is independently selected from H and alkyl, and z″ is an integer from 1 to 50; or combinations thereof.

The linker can comprise a —(OCH₂CH₂)_{z}′—(e.g., as a spacer), wherein z′ is an integer from 1 to 23, e.g., 2, 3, 4, 5, 6, 7, 8, 9, 10, 11, 12, 13, 14, 15, 16, 17, 18, 19, 20, 21, 22, or 23. “—(OCH₂CH₂) z′ can also be referred to as polyethylene glycol (PEG).

The linker can comprise one or more amino acids. The linker can comprise a peptide. The linker can comprise a —(OCH₂CH₂)_{z}′—, wherein z′ is an integer from 1 to 23, and a peptide. The peptide can comprise from 2 to 10 amino acids.

The linker can comprise (i) a β alanine residue and lysine residue; (ii) —(J—R¹)z″; or (iii) a combination thereof. Each R¹ can independently be alkylene, alkenylene, alkynylene, carbocyclyl, or heterocyclyl, each J is independently C, NR₃, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O.

The linker can comprise (i) residues of β-alanine, glycine, lysine, 4-aminobutyric acid, 5-aminopentanoic acid, 6-aminohexanoic acid or combinations thereof; and (ii) —(R₁ J) z″— or —(J—R¹)z″. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R′ can be alkylene and each J can be O. The linker can comprise glycine, β-alanine, 4-aminobutyric acid, 5-aminopentanoic acid, 6-aminohexanoic acid, or a combination thereof.

The linker can be a trivalent linker. The linker can have the structure:

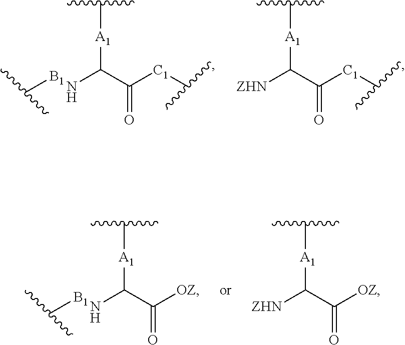

wherein A₁, B₁, and C₁, can independently be a hydrocarbon linker (e.g., NRH—(CH₂)ₙ—COOH), a PEG linker (e.g., NRH—(CH₂O)ₙ—COOH, wherein R is H, methyl or ethyl) or one or more amino acid residue, and Z is independently a protecting group. The linker can also incorporate a cleavage site, including a disulfide [NH₂-(CH₂O)ₙ—S—S—(CH₂O)ₙ—COOH], or caspase-cleavage site (Val-Cit-PABC).

The hydrocarbon can be a residue of glycine or β-alanine.

In embodiments, the cargo conjugates may include two to more cCPPs (e.g, 2, 3, 4, 5, 6, 7, 8, 9, or 10). As such, the linker can be multivalent and link two or more cCPPs to a cargo and/or EP, thereby forming an EEV comprising two or more cCPPs. In embodiments, the compound may include two or more linkers that allow for two or more cCPPs, one or more EPs, and one or more cargos to be linked in a single compound. For example, a delivery construct may comprise (cCPP¹)-linker¹-K(cCPP²)-linker²-Rh where linker¹ and linker² may be distinct linkers or a single linker, Rh is a reactive group that is a part of a linker, cCPP¹ and cCPP² are two cCPPs that may be the same or different. An EEV may comprise EP-linker¹-K/k(cCPP¹)-linker²-K/k(cCPP²)-linker³-Rh where the linkers may be distinct linkers, or two or more linkers may be a part of the same linker, K/k indicates that lysine can be either isomer; cCPP¹ and cCPP² are two cCPPs that may be the same or different; and Rh is a reactive handle that is a part of a linker. The Rh may be used to conjugate the delivery construct comprising two or more cCPPs to a cargo.

In embodiments, a delivery construct may be of Formula (M.cCPP):

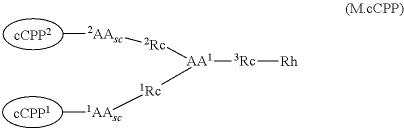

In Formula (M.cCPP), cCPP¹ and cCPP² are cyclic cell penetrating peptides. cCPP¹ and cCPP² may be the same or different. ¹AA_{sc} is the amino acid side chain of cCPP¹ and AA_{sc}² is the amino acid side chain of cCPP². ¹AA_{sc} and ²AA_{sc} may be any AAsc. ¹Rc, ²Rc, and ³Rc are each independently a connecting group comprising a hydrocarbon group or a hydrocarbon group with (i) one or more catenated heteroatoms and/or (ii) one or more catenated carbonyls. In embodiments, Rc comprises one or more polyethylene repeat units. Rh is a reactive handle that may be used to conjugate the cargo to the delivery construct. AA¹ is a trivalent amino acid residue comprising a side chain, a N-terminus, and a C-terminus. The N-terminus of AA¹ is covalently coupled to ¹Rc, ²Rc, or ³Rc. The C-terminus of AA¹ is covalently coupled to ¹Rc, ²Rc, or ³Rc. The side chain of AA¹ is covalently coupled to ¹Rc, ²Rc, or ³Rc. Additionally cCPPs may be added to Formula (M.cCPP) by including, for example, additional trivalent amino acids and/or connecting groups at any location in Formula (M.cCPP). For example, a third cCPP may be added to Formula (M.cCPP) by adding three additional Rc groups (⁴Rc, ⁵Rc, and ⁶Rc) and a second amino acid residue (AA²) between AA¹ and ³Rc (e.g., -AA¹-⁴Rc-AA²(-⁵Rc-³AAsc-cCPP³)-⁶Rc-³Rc-).

In embodiments, the delivery construct may be of formula (M.cCPP.i):

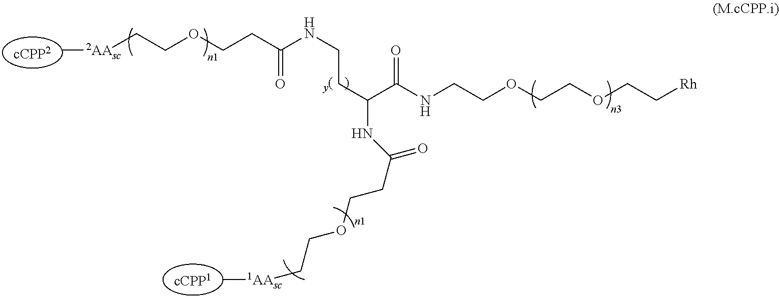

wherein cCPP¹, cCPP², ¹AAsc, ²AAsc, and Rh are described herein; each of n1, n2, and n3 are independently an integer from 0 to 20; and y is an integer from 1 to 6.

The linker can be a bivalent or trivalent C₁-C₅₀ alkylene, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, S(O)₂—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)—, —C(O)N(C₁-C₄ alkyl), —C(O)N(cycloalkyl), aryl, heterocyclyl, heteroaryl, cycloalkyl, or cycloalkenyl. The linker can be a bivalent or trivalent C₁-C₅₀ alkylene, wherein 1-25 methylene groups are optionally and independently replaced by —N(H)—, —O—, —C(O)N(H)—, or a combination thereof.

The linker can have the structure of L1:

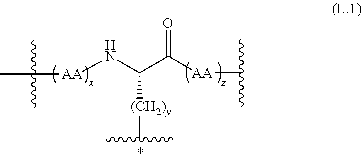

wherein: each AA is independently an amino acid residue; * is the point of attachment to the AA_{SC}, and AA_{SC} is side chain of an amino acid residue of the cCPP; x is an integer from 1-10; y is an integer from 1-5; and z is an integer from 1-10. x can be an integer from 1-5. x can be an integer from 1-3. x can be 1. y can be an integer from 2-4. y can be 4. z can be an integer from 1-5. z can be an integer from 1-3. z can be 1. Each AA can independently be selected from glycine, b-alanine, 4-aminobutyric acid, 5-aminopentanoic acid, and 6-aminohexanoic acid.

The linker can have the structure of L2:

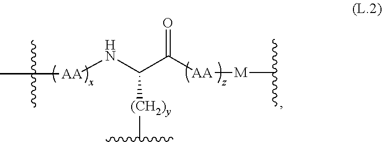

wherein: x is an integer from 1-10; y is an integer from 1-5; z is an integer from 1-10; each AA is independently an amino acid residue; * is the point of attachment to the AA_{SC}, and AA_{SC} is side chain of an amino acid residue of the cCPP; and M is a bonding group defined herein.

The linker can have the structure of L3:

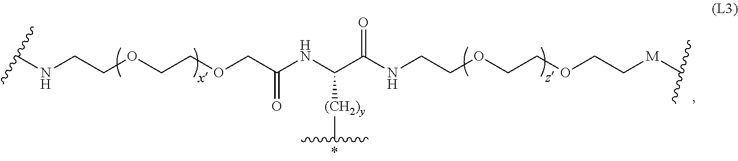

wherein: x′ is an integer from 1-23; y is an integer from 1-5; z′ is an integer from 1-23; * is the point of attachment to the AA_{SC}, and AA_{SC} is a side chain of an amino acid residue of the cCPP; and M is a bonding group defined herein.

The linker can have the structure of (LA):

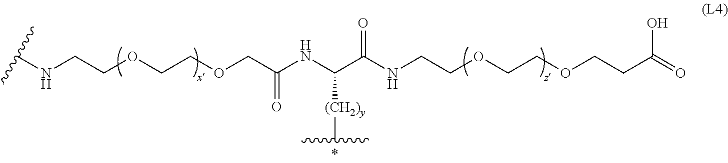

wherein: x′ is an integer from 1-23; y is an integer from 1-5; and z′ is an integer from 1-23; * is the point of attachment to the AA_{SC}, and AA_{SC} is a side chain of an amino acid residue of the cCPP.

The linker can have the structure of L5a or L6a:

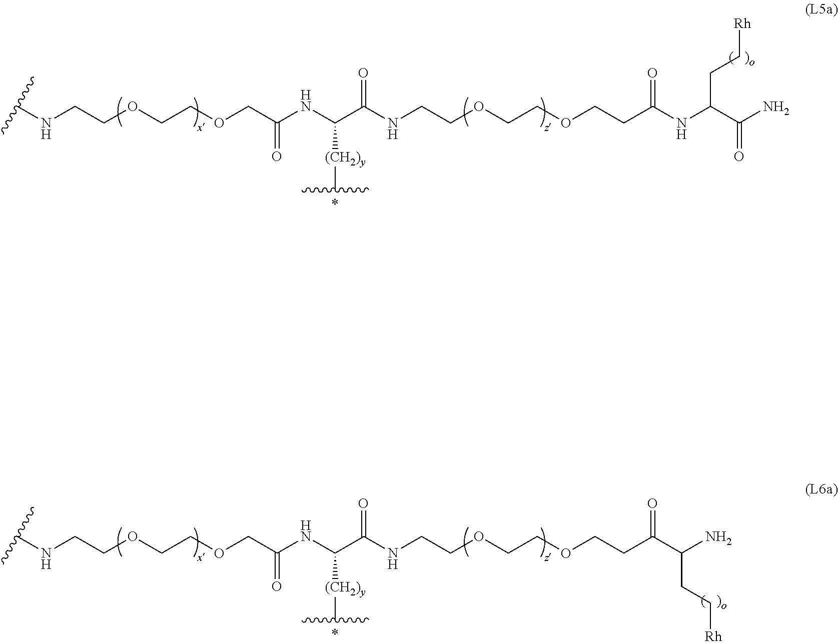

where Rh is reactive handle that is cooperative with a reactive handle on a cargo or exocyclic peptide; x′ is an integer from 1-23; y is an integer from 1-5; and z′ is an integer from 1-23; * is the point of attachment to the AA_{SC}, and AA_{SC} is a side chain of an amino acid residue of the cCPP. In embodiments, Rh is an azide. In embodiments, Rh is OH. In embodiments, Rh is SH. In embodiments, Rh is NH₂.

Following a conjugation reaction where the FG of linker L-A or L-B reacts with a cooperative reactive handle on a cargo or exocyclic peptide, the linker may can have the structure of L5 or L6:

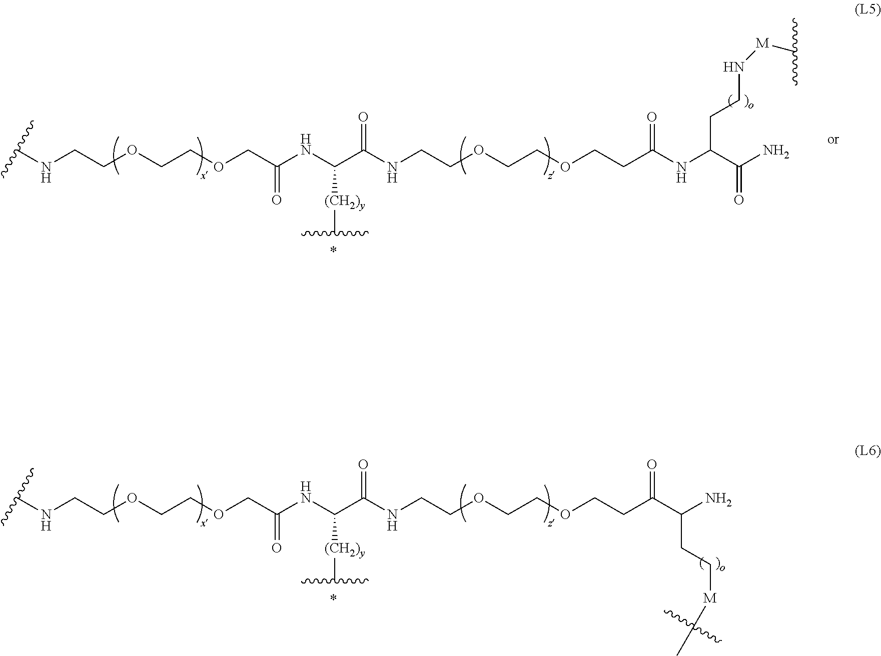

wherein: x′ is an integer from 1-23; y is an integer from 1-5; and z′ is an integer from 1-23; * is the point of attachment to the AA_{SC}; AA_{SC} is a side chain of an amino acid residue of the cCPP; and M is any bonding group as described herein.

In L1 and L2, y can be an integer from 1-5, e.g., 1, 2, 3, 4, or 5, inclusive of all ranges and subranges therebetween. y can be an integer from 2-5. y can be an integer from 3-5. y can be 3 or 4. y can be 4 or 5. y can be 3. y can be 4. y can be 5.

In L1 and L2, x can be an integer from 1-10, e.g., 1, 2, 3, 4, 5, 6, 7, 8, 9, or 10, inclusive of all ranges and subranges therebetween.

In L1 and L2, z can be an integer from 1-10, e.g., 1, 2, 3, 4, 5, 6, 7, 8, 9, or 10, inclusive of all ranges and subranges therebetween.

In L3, L4, L5a, L5, L6a, and L6, x′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. x′ can be an integer from 5-15. x′ can be an integer from 9-13. x′ can be an integer from 1-5. x′ can be 1.

In L3, L4, L5a, L5, L6a, and L6, z′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. z′ can be an integer from 5-15. z′ can be an integer from 9-13. z′ can be 11.

In L3, L4, L5a, L5, L6a, and L6, as discussed above, the linker or M (wherein M is part of the linker) can be covalently bound to a cargo at any suitable location on the cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

The linker can have a structure of L7:

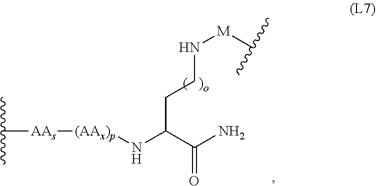

wherein
M is a group that conjugates linker to a cargo (bonding group);
AAₛ is a side chain or terminus of an amino acid on the cCPP;
each AAₓ is independently an amino acid residue;
o is an integer from 0 to 10; and
p is an integer from 0 to 5.

The linker can have a structure of L8:

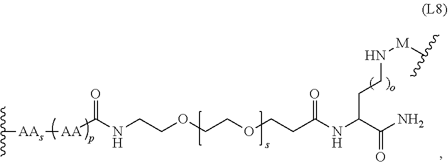

wherein
M is a group that conjugates L to a cargo;
AAₛ is a side chain or terminus of an amino acid on the cCPP;
each AAₓ is independently an amino acid residue;
s is an integer from 0 to 15 (e.g., 1, 2, 11, or 12);
o is an integer from 0 to 10; and
p is an integer from 0 to 5.

In embodiments, the bonding group M is or includes any reaction product of a direct conjugation reaction as disclosed herein or any may include the two reaction products of an indirect conjugation reaction as well as the intervening moiety of a bifunctional conjugation compound separating the two reaction products. In embodiments, the bonding group M comprises an alkylene, alkenylene, alkynylene, carbocyclyl, or heterocyclyl, each of which is optionally substituted. In embodiments, M can be selected from:

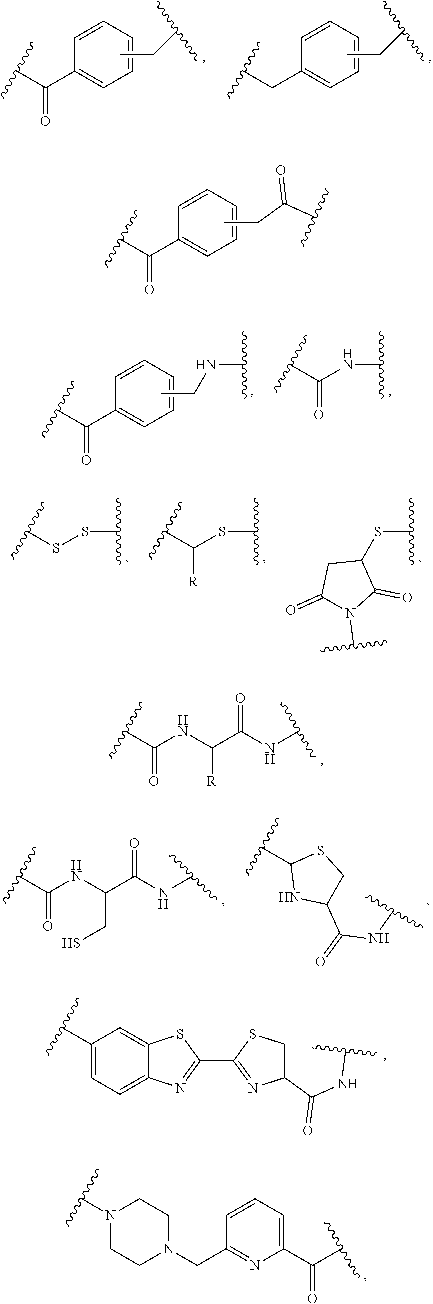

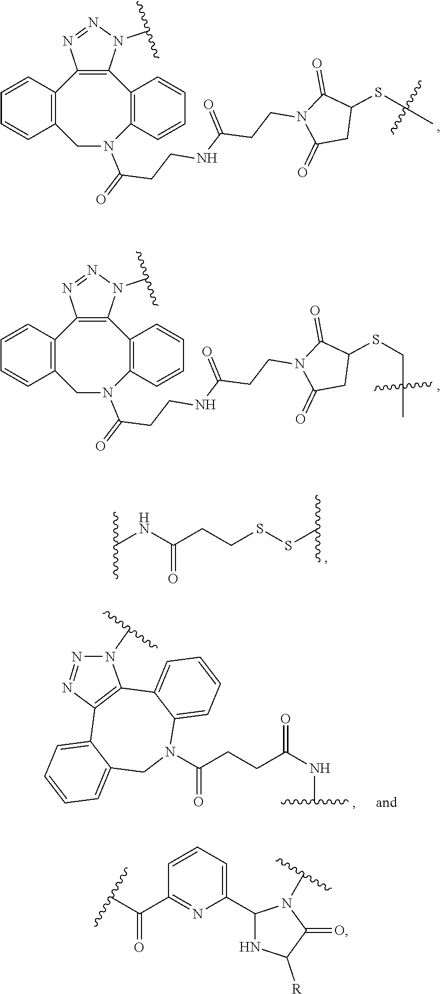

wherein R is alkyl, alkenyl, alkynyl, carbocyclyl, or heterocyclyl.

In embodiments, M is selected from:

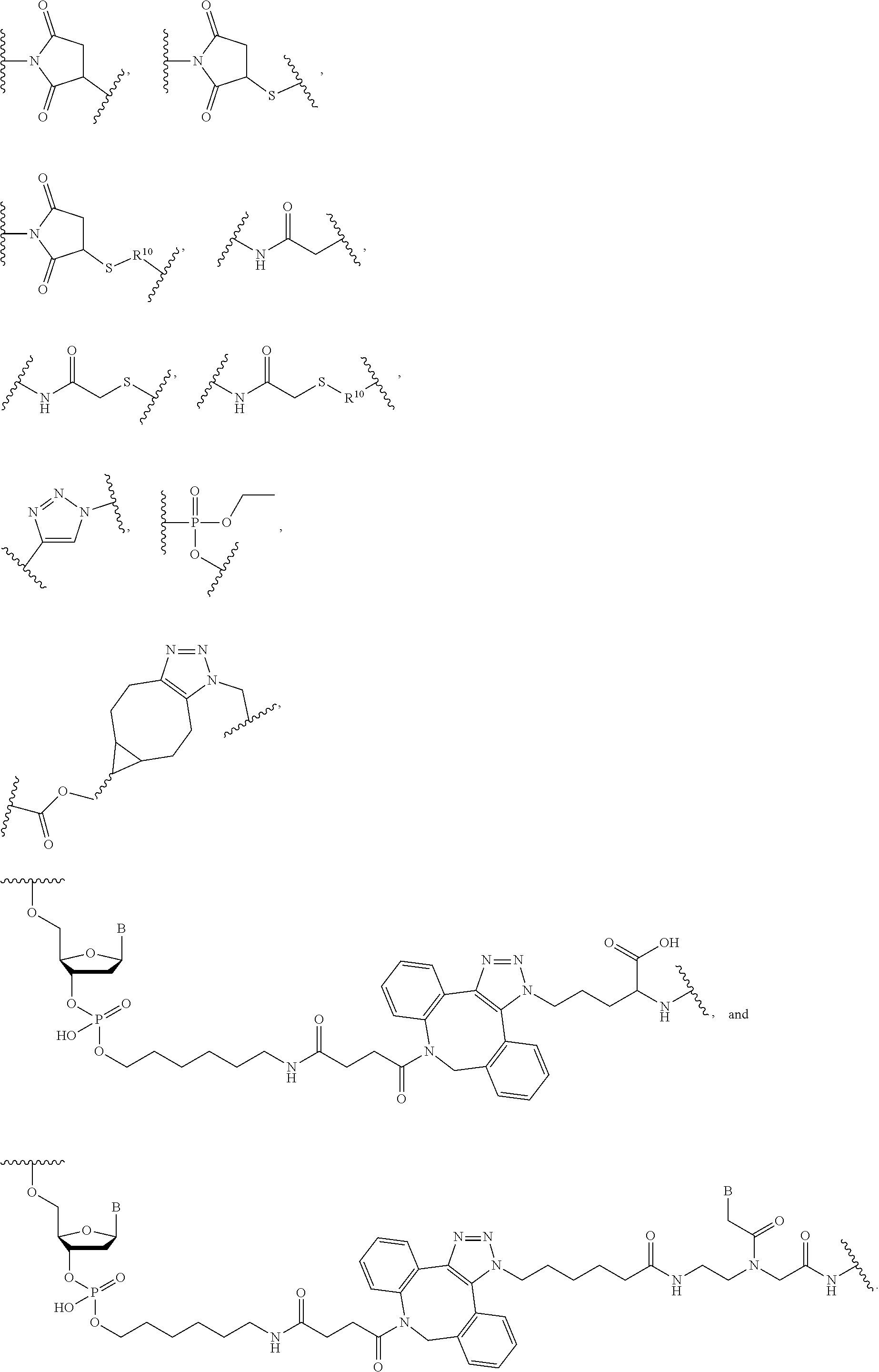

wherein: R¹⁰ is alkylene, cycloalkyl, or

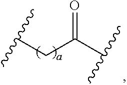

wherein a is 0 to 10.

In embodiments, M is

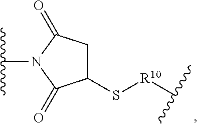

R¹⁰ can be

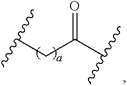

and a is 0 to 10.

In embodiments, M is

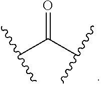 [SMILES: *C(*)=O]

In embodiments, M is a heterobifunctional crosslinker, e.g.,

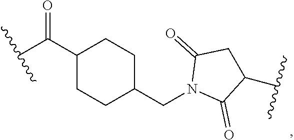 [SMILES: *C(=O)C1CCC(CN2C(=O)CC(*)C2=O)CC1]

which is disclosed in Williams et al. Curr. Protoc Nucleic Acid Chem. 2010, 42, 4.41.1-4.41.20, incorporated herein by reference its entirety.

In embodiments, M is —C(O)—.

AAₛ can be a side chain or terminus of an amino acid on the cCPP. Non-limiting examples of AAₛ include aspartic acid, glutamic acid, glutamine, asparagine, or lysine, or a modified side chain of glutamine or asparagine (e.g., a reduced side chain having an amino group). AAₛ can be any AA_{SC} as defined herein.

Each AAₓ is independently a natural or non-natural amino acid. One or more AAₓ can be a natural amino acid. One or more AAₓ can be a non-natural amino acid. One or more AAₓ can be a beta-amino acid. The beta-amino acid can be beta-alanine.

o can be an integer from 0 to 10, e.g., 0, 1, 2, 3, 4, 5, 6, 7, 8, 9, and 10. o can be 0, 1, 2, or 3. o can be 0. o can be 1. o can be 2. o can be 3.

p can be 0 to 5, e.g., 0, 1, 2, 3, 4, or 5. p can be 0. p can be 1. p can be 2. p can be 3. p can be 4. p can be 5.

The linker can have the structure:

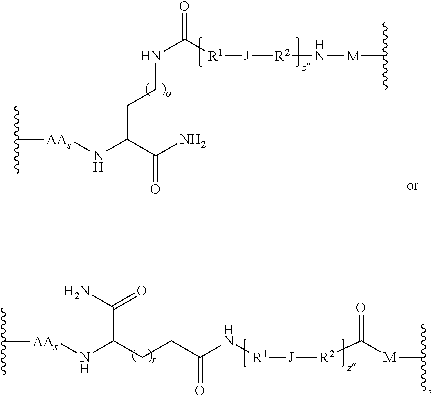

wherein M, AAₛ, each —(R¹—J—R²)z″—, o and z″ are defined herein; r can be 0 or 1.

The linker can have the structure:

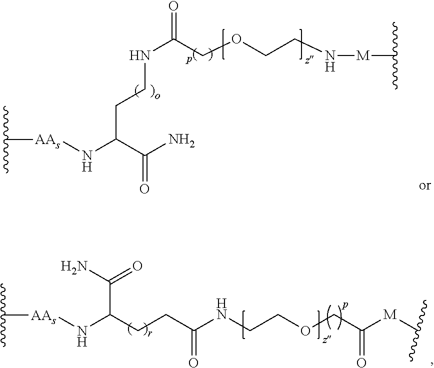

wherein each of M, AAₛ, o, p, q, r and z″ can be as defined herein. z″ can be an integer from 1 to 50, e.g., 1, 2, 3, 4, 5, 6, 7, 8, 9, 10, 11, 12, 13, 14, 15, 16, 17, 18, 19, 20, 21, 22, 23, 24, 2 5, 26, 27, 28, 29, 30, 31, 32, 33, 34, 35, 36, 37, 38, 39, 40, 41, 42, 43, 44, 45, 46, 47, 48, 49, and 50, inclusive of all ranges and values therebetween. z″ can be an integer from 5-20. z″ can be an integer from 10-15.

The linker can have the structure:

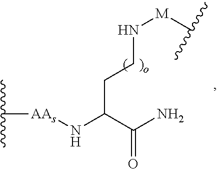

wherein M, AAₛ and o are as defined herein.

Other non-limiting examples of suitable linkers include:

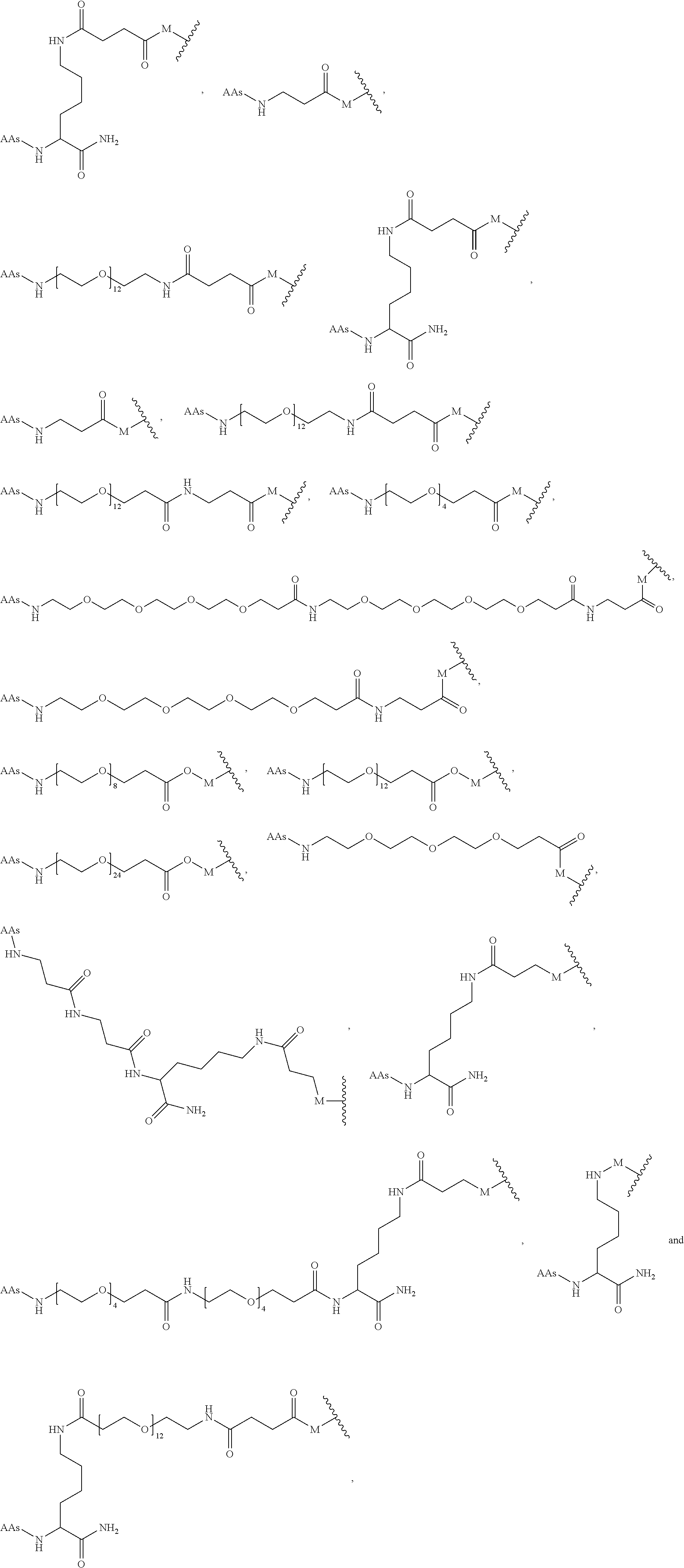

wherein M and AA_{sc} are as defined herein.

Provided herein is cargo conjugate comprising a cCPP and a cargo further comprising L, wherein the linker is conjugated to the cargo through a bonding group (M), wherein M is

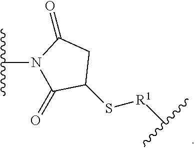

Provided herein is a cargo conjugate comprising a cCPP and a cargo that further comprises L, wherein the linker is conjugated to the cargo through a bonding group (M), wherein M is selected from:

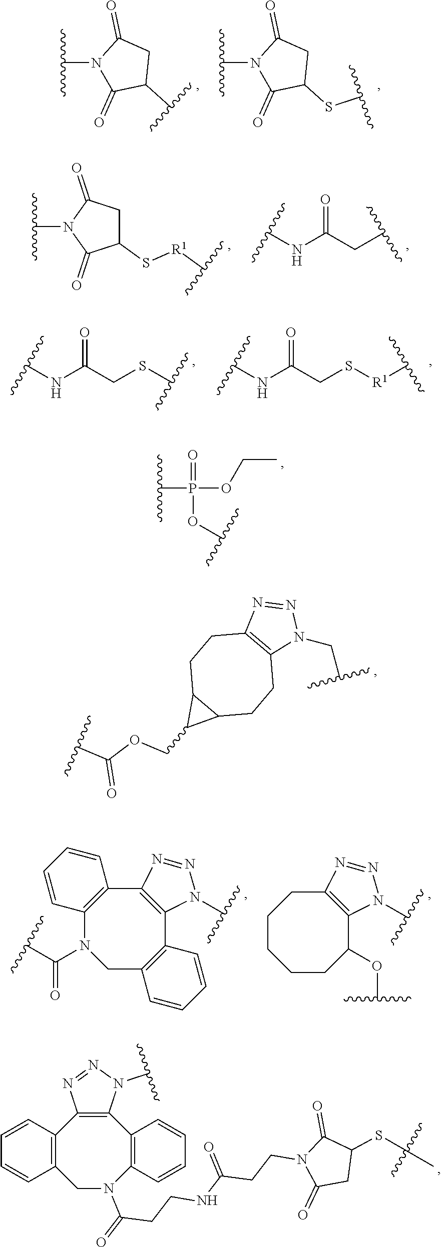

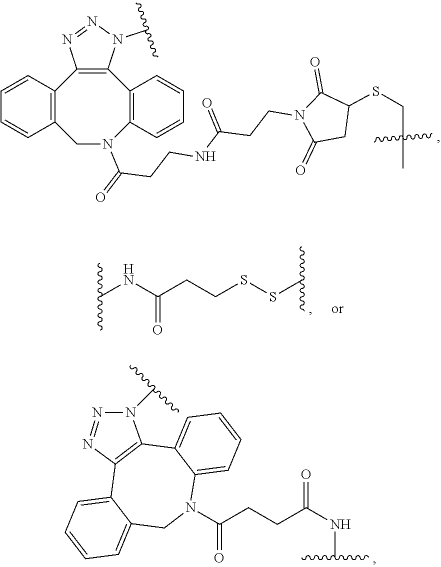

wherein: R¹ is alkylene, cycloalkyl, or

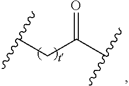

wherein t′ is 0 to 10 wherein each R is independently an alkyl, alkenyl, alkynyl, carbocyclyl, or heterocyclyl, wherein R¹ is

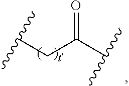

and t′ is 2.

In embodiments, the linker has the structure:

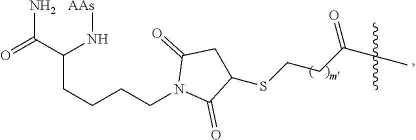

wherein AAₛ is as defined herein, and m′ is 0-10.

In embodiments, the linker can have the structure:

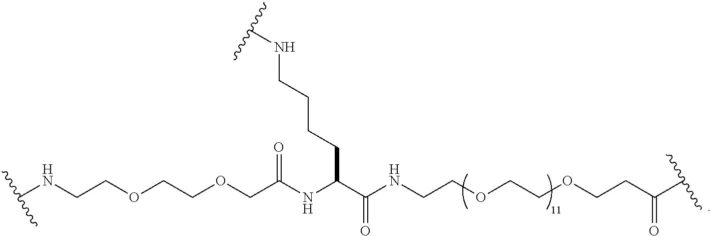

### Delivery Constructs

The linker can be conjugated to an AA_{SC} of the cCPP as defined herein. The linker can comprise a —(OCH₂CH₂)_{z}′— subunit (e.g., as a spacer), wherein z′ is an integer from 1 to 23, e.g., 1, 2, 3, 4, 5, 6, 7, 8, 9, 10, 11, 12, 13, 14, 15, 16, 17, 18, 19, 20, 21, 22 or 23. “—(OCH₂CH₂)_{z}′ is also referred to as PEG. The delivery conjugate can have a structure selected from Table 2:

TABLE 2
Delivery constructs

cyclo(FfΦ-4gp-r-4gp-rQ)-PEG₄-K-NH₂ (SEQ ID NO: 185)
cyclo(FfΦ-Cit-r-Cit-rQ)-PEG₄-K-NH₂ (SEQ ID NO: 186)
cyclo(FfΦ-Pia-r-Pia-rQ)-PEG₄-K-NH₂ (SEQ ID NO: 187)
cyclo(FfΦ-Dml-r-Dml-rQ)-PEG₄-K-NH₂ (SEQ ID NO: 188)
cyclo(FfΦ-Cit-r-Cit-rQ)-PEG₁₂-OH (SEQ ID NO: 189)
cyclo(fΦR-Cit-R-Cit-Q)-PEG₁₂-OH (SEQ ID NO: 190)
cyclo(BhFfΦGrGrQ) (SEQ ID NO: 191)
cyclo(BhFfΦGrGrQ)-PEG₄-K-NH₂ (SEQ ID NO: 192)
cyclo(BhFfΦGrGrQ)-PEG₁₂-OH (SEQ ID NO: 193)
cyclo(BhFfΦSrSrQ) (SEQ ID NO: 194)
cyclo(BhFfΦSrSrQ)-PEG₄-K-NH₂ (SEQ ID NO: 195)
cyclo(BhFfΦSrSrQ)-PEG₁₂-OH (SEQ ID NO: 196)
cyclo(BhFfΦHrHrQ) (SEQ ID NO: 197)
cyclo(BhFfΦHrHrQ)-PEG₄-K-NH₂ (SEQ ID NO: 198)
cyclo(BhFfΦHrHrQ)-PEG₁₂-OH (SEQ ID NO: 199)
cyclo(BhFFΦSRSRQ) (SEQ ID NO: 200)
cyclo(BhFFΦSRSRQ)-PEG₄₋K-NH₂ (SEQ ID NO: 201)
cyclo(BhFFΦSRSRQ)-PEG₁₂-OH (SEQ ID NO: 202)
cyclo(BhFFΦGRGRQ) (SEQ ID NO: 203)
cyclo(BhFFΦGRGRQ)-PEG₄-K-NH₂ (SEQ ID NO: 204)
cyclo(BhFFΦGRGRQ)-PEG₁₂-OH (SEQ ID NO: 205)
cyclo(BhFFΦHRHRQ)(SEQ ID NO: 206)
cyclo(BhFFΦHRHRQ)-PEG₄-K-NH₂ (SEQ ID NO: 207)
cyclo(BbFFΦHRHRQ)-PEG₁₂-OH (SEQ ID NO: 208)

In embodiments, the delivery construct comprises an endosomal escape vehicle (EEV). EEVs comprising a cyclic cell penetrating peptide (cCPP), linker, and exocyclic peptide (EP) are provided. In embodiments, an EP and a cCPP of an EEV can be conjugated to a bivalent or trivalent linker. In embodiments, an EP and a cCPP of an EEV can be conjugated to two or more linkers. In embodiments, the linker linking an EP and a cCPP in an EP-cCPP conjugate (EP-cCPP-linker conjugate) can comprise a —(OCH₂CH₂)_{z}′— subunit, wherein z′ is an integer from 1 to 23, and a peptide subunit. The peptide subunit can comprise from 2 to 10 amino acids. The cCPP-linker conjugate can have a structure selected from Table 3:

TABLE 3
Endosomal Escape Vehicles (EEVs)

Ac-PKKKRKV-Lys(cyclo[FfΦ-R-r-Cit-rQ])-PEG₁₂-K(N₃)-NH₂
(SEQ ID NO: 209 and 441, respectively, in order of appearance)
Ac-PKKKRKV-Lys(cyclo[FfΦ-Cit-r-R-rQ])-PEG₁₂-K(N₃)-NH₂
(SEQ ID NO: 210 and 443, respectively, in order of appearance)
Ac-PKKKRKV-K(cyclo(FfΦR-cit-R-cit-Q))-PEG₁₂-K(N₃)-NH₂
(SEQ ID NO: 211 and 445, respectively, in order of appearance)
Ac-PKKKRKV-PEG2-Lys(cyclo[FfΦ-Cit-r-Cit-rQ])-B-k(N₃)-NH₂
(SEQ ID NOS 212 and 213, respectively, in order of appearance)
Ac-PKKKRKV-PEG2-Lys(cyclo[FfΦ-Cit-r-Cit-rQ])-PEG2-k(N₃)-NH₂
(SEQ ID NOS 214 and 215, respectively, in order of appearance)
Ac-PKKKRKV-PEG2-Lys(cyclo[FfΦ-Cit-r-Cit-rQ])-PEG4-k(N₃)-NH₂
(SEQ ID NOS 216 and 217, respectively, in order of appearance)
Ac-PKKKRKV-Lys(cyclo[FfΦ-Cit-r-Cit-rQ])-PEG12-k(N₃)-NH₂
(SEQ ID NO: 218 and 472, respectively, in order of appearance)
Ac-pkkkrkv-PEG2-Lys(cyclo[FfΦ-Cit-r-Cit-rQ])-PEG12-k(N₃)-NH₂
(SEQ ID NOS 219 and 220, respectively, in order of appearance)
Ac-rrv-PEG2-Lys(cyclo[FfΦ-Cit-r-Cit-rQ])-PEG12-OH (SEQ ID NO: 221)
Ac-PKKKRKV-PEG2-Lys(cyclo[FfΦ-Cit-r-Cit-r-Q])-PEG12-k(N3)-NH₂
(SEQ ID NOS 222 and 223, respectively, in order of appearance)
Ac-PKKK-Cit-KV-PEG2-Lys(cyclo[FfΦ-Cit-r-Cit-r-Q])-PEG12-k(N₃)-NH₂
(SEQ ID NOS 224 and 225, respectively, in order of appearance)
Ac-PKKKRKV-PEG2-Lys(cyclo[FfΦ-Cit-r-Cit-r-Q]-PEG12-K(N₃)-NH₂
(SEQ ID NOS 226 and 227, respectively, in order of appearance)

An EEV can comprise the structure of Formula (X):

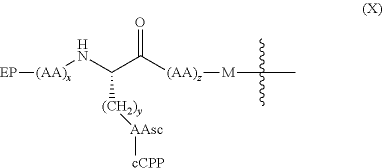

wherein EP, AA, x, z, y, and M are defined elsewhere herein; AA_{SC} is an amino acid side chain a residue in the cCPP; and the cCPP may be any cCPP having any combination of amino acid residues as described herein.

EEVs comprising a cyclic cell penetrating peptide, a linker, and an EP are provided having the general formula EP-linker(a)-cCPP-linker(b), wherein linker(a) and linker(b) are a part of the same trivalent linker. The linker can be conjugated to the cCPP through the AA_{SC} of the CCP. The linker may be conjugated to the EP through a conjugation reaction between a functional group on the EP and a functional group on the linker. In embodiments, the linker is conjugated to the EP through a reaction with a functional group on the EP that is at or is near (e.g., the size chain of the C-terminal amino acid) the C-terminus of the EP. Linker(b) may have a functional group that can react in a conjugation reaction (e.g., a bioconjugation reaction) with a functional group on a cargo to form a compound of the general formula EP-linker(a)-cCPP-linker(b)-cargo.

EEVs comprising a cyclic cell penetrating peptide (cCPP), linker and exocyclic peptide (EP) are provided. An EEV can comprise the structure of Formula (J1), (J2), or (J3):

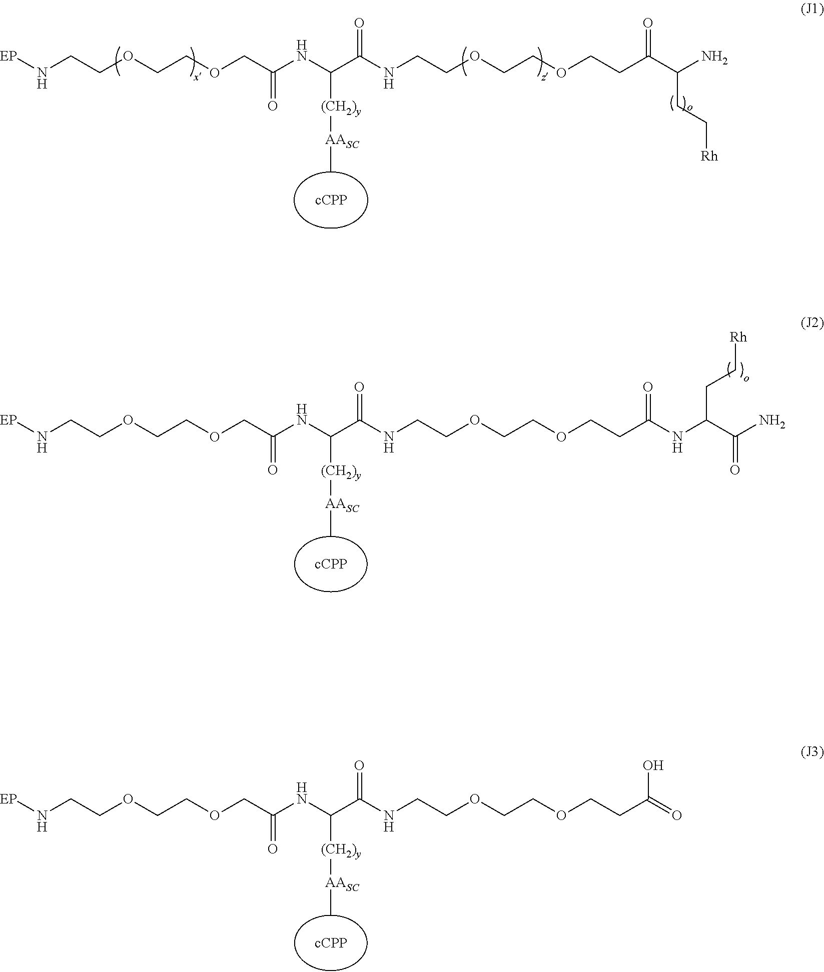

wherein EP is any exocyclic peptide disclosed herein; y is an integer from 1 to 5; x′ is an integer from 1-20; z′ is an integer from 1-23; cCPP is any cCPP disclosed herein; AA_{sc} is any AA_{sc} as disclosed herein; o is an integer from 1 to 5; and Rh is a reactive handle configured to react with a reactive handle on a cargo to form any bonding group (M) disclosed herein. The stereochemistry of the stereocenters may be S or R.

Delivery constructs comprising a cyclic cell penetrating peptide (cCPP) and linker are provided. A delivery construct can comprise the structure of Formula (JJ1):

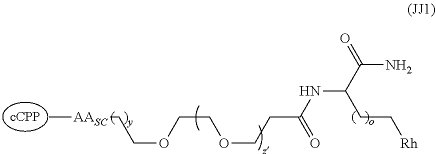

wherein: AA_{sc} is any AA_{sc} as disclosed herein; y is an integer from 1 to 5; o is an integer from 1 to 5; z′ is an integer from 1-23; cCPP is any cCPP disclosed herein; and Rh is a reactive handle configured to react with a reactive handle on a cargo to form any bonding group (M) disclosed herein. Rh may be any reactive handle disclosed herein.

In embodiments, the delivery construct is of Formula (J1), (J2), or (J3) wherein x′ is 1 or 2. In embodiments, the delivery construct is of Formula (J1), (J2), (J3), or (JJ1) wherein z′ is 1, 2, 11, or 121. In embodiments, the cCPP is of Formula (IA), (I), (I-a), (I-b), (I-2), (I-3), (I-4), (I-5), (I-6), (I-7), (IX), (IX1), (IX(a)), (IX(b)), (IX (c)), (II), (II-1), (IIa), (IIc), (III), (III-1), (IIIa), (D), (AV), (Y1), (Y1′), (Y2), (Y2′), (AA(a), (AA(b)), (Y-a), (Y-aa), (Y-ab), (Ym), (Yn), (Yo), (Yp), (AA(c)), (AA(d)), (AA(e)), (A-II), (A-II-1), (A-IIa), (A-IIb), (A-III), (A-III-1), (A-IIIa), or derivatives having the specified characteristics described herein.

In embodiments, the delivery construct is an EEV. EEVs comprising a cyclic cell penetrating peptide (cCPP), linker and exocyclic peptide (EP) are provided. An EEV can comprise the structure of Formula (B):

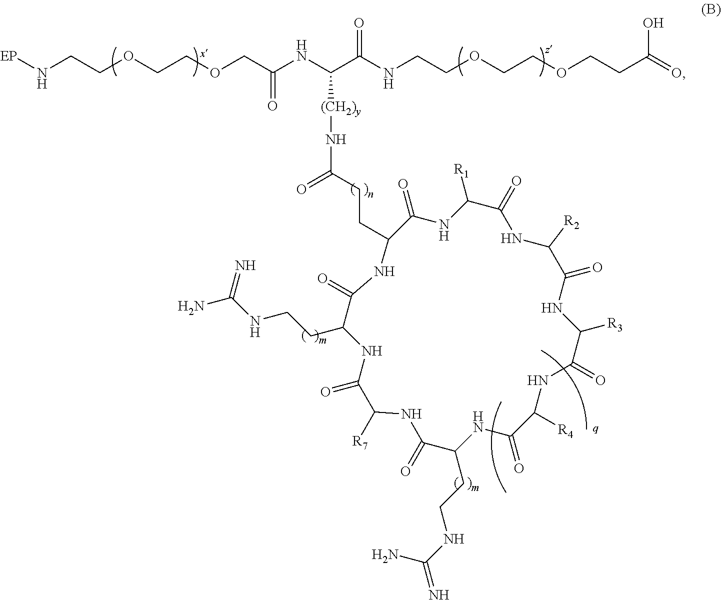

or a protonated form thereof,
wherein:
R₁, R₂, and R₃ are each independently H or an aromatic or heteroaromatic side chain of an amino acid;
R₄ and R₇ are independently H or an amino acid side chain;
EP is an exocyclic peptide as defined herein;
each m is independently an integer from 0-3;
n is an integer from 0-2;
x′ is an integer from 1-20;
y is an integer from 1-5;
q is 1-4; and
z′ is an integer from 1-23.
R₁, R₂, R₃, R₄, R₇, EP, m, q, y, x′, z′ are as described herein.
n can be 0. n can be 1. n can be 2.

The EEV can comprise the structure of Formula (B-a) or (B-b):

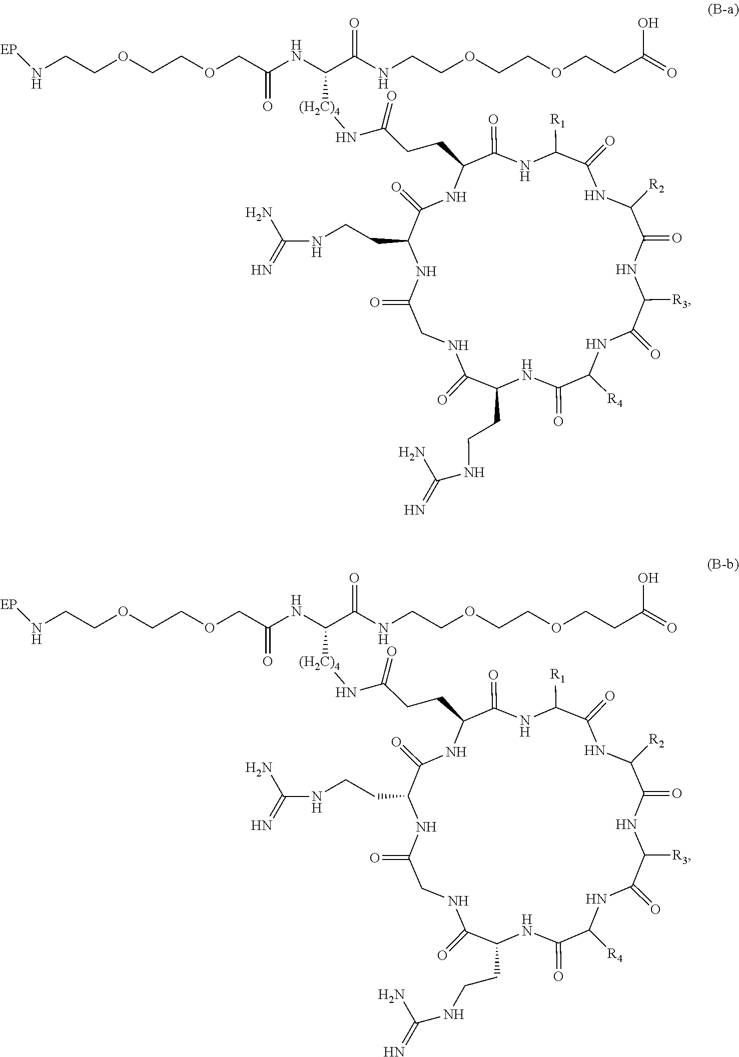

or a protonated form thereof, wherein EP, R¹, R², R³, R⁴, m and z′ are as defined above in Formula (B).

The EEV can comprise the structure of Formula (B-c):

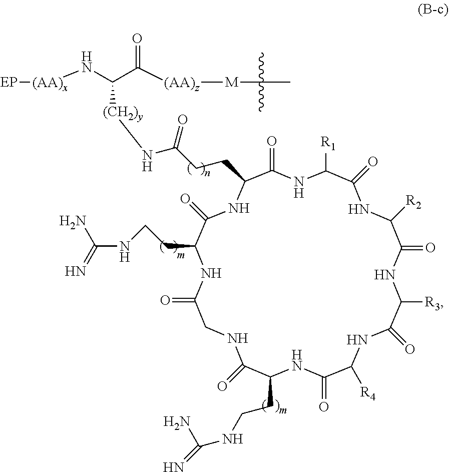

or a protonated form thereof, wherein EP, R¹, R², R³, R⁴, and m are as defined above in Formula (B); AA is an amino acid as defined herein, M is as defined herein; n is an integer from 0-2, x is an integer from 1-10; y is an integer from 1-5; and z is an integer from 1-10.

The EEV can have the structure of Formula (B-1), (B-2), (B-3), or (B-4) (SEQ ID NOS 417, 419, 421 and 423, respectively, in order of appearance):

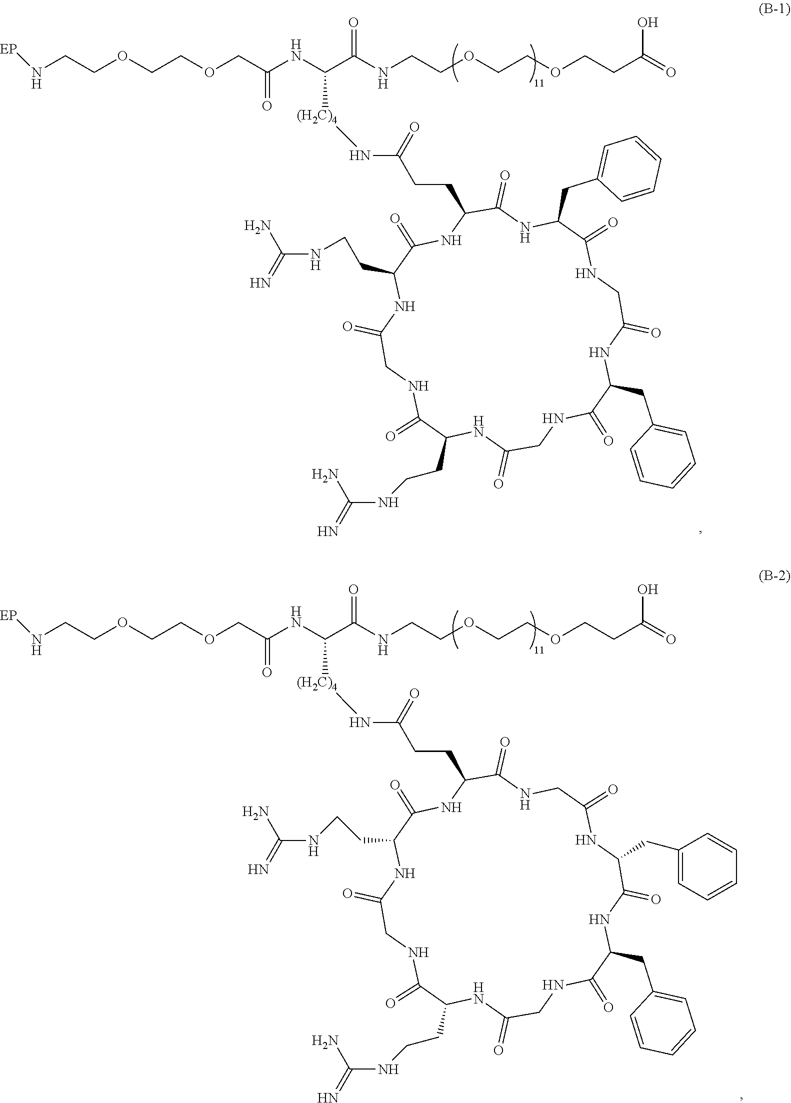

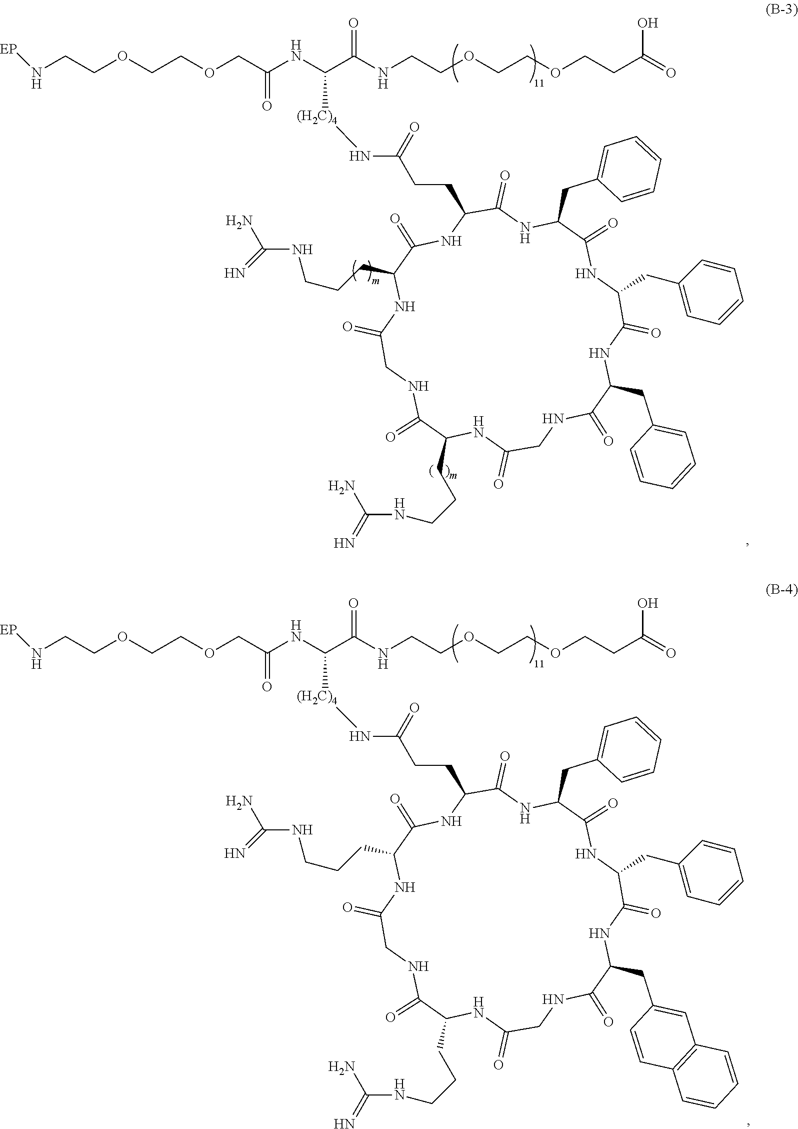

or a protonated form thereof, wherein EP is as defined above in Formula (B).

The EEV can comprise Formula (B) and can have the structure: Ac-PKKKRKV-AEEA-K (cyclo[FGFGRGRQ])-PEG₁₂-OH (SEQ ID NOS 228 and 229, respectively, in order of appearance) or Ac-PKKKRKV-AEEA-K (cyclo[GfFGrGrQ])-PEG₁₂-OH (SEQ ID NOS 230 and 231, respectively, in order of appearance).

EEVs comprising a cyclic cell penetrating peptide (cCPP), linker and exocyclic peptide (EP) are provided. An EEV can comprise the structure of Formula (C):

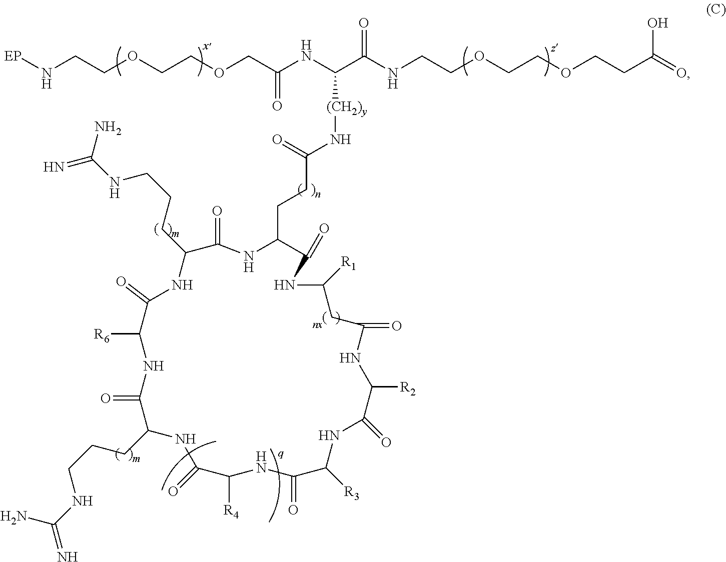

or a protonated form thereof,
wherein:
R₁, R₂, R₃, R₄, R⁵, and R₆, are independently H or an amino acid side chain;
at least two of R₁, R₂, and R₃ are independently an aromatic or heteroaromatic side chain of an amino acid; R₄ and R₆ are independently an uncharged, non-aryl amino acid side chain selected from histidine, threonine, serine, leucine, isoleucine, valine, neopentylglycine, alanine, homoalanine, homoserine, 3-(4-Thiazolyl)-alanine, 3-(4-furanyl)-alanine, and 3-(4-thienyl)-alanine; nₓ is 0 or 1;q is 1, 2, 3 or 4; EP is an exocyclic peptide as defined herein; each m is independently an integer from 0-3; n is an integer from 0-2; x′ is an integer from 1-20; y is an integer from 1-5; and z′ is an integer from 1-23.

In embodiments of an EEV of Formula (c), at least two of R₁, R₂, and R₃ are independently a side chain of phenylalanine or naphthylalanine.

In embodiments of an EEV of Formula (c), R₄ and R₆ are independently serine or histidine.

In embodiments of an EEV of Formula (c), at least two of R₁, R₂, and R₃ are independently a side chain of phenylalanine or naphthylalanine and R₄ and R₆ are independently serine or histidine.

An BEV can comprise the structure of Formula (C), or a protonated form thereof,
wherein: R₁, R₂, R₃, R₄, R₅, and R₆, are independently H or an amino acid side chain; at least two of R₁, R₂, and Rs are independently a side chain of phenylalanine, or naphthylalanine; R₄ and Re are independently a side chain of serine or histidine; nₓ is 0 or 1; q is 1, 2, 3 or 4; EP is an exocyclic peptide as defined herein; each m is independently an integer from 0-3; n is an integer from 0-2; x′ is an integer from 1-20; y is an integer from 1-5; and z′ is an integer from 1-23.

EEVs comprising a cyclic cell penetrating peptide (cCPP), linker and exocyclic peptide (EP) are provided. An EEV can comprise the structure of Formula (C), or a protonated form thereof, wherein: R₁, R₂, R₃, R₄, and R₆, are independently H or an amino acid side chain; at least two of R₁, R₂, and R₃ are independently a side chain of phenylalanine, or naphthylalanine;

nₓ is 1; q is 1, 2, 3 or 4; EP is an exocyclic peptide as defined herein; each m is independently an integer from 0-3; n is an integer from 0-2; x′ is an integer from 1-20; y is an integer from 1-5; and z′ is an integer from 1-23. R₁, R₂, R₃, R₄, R₆, EP, m, q, y, x′, z′ are as described herein. n can be 0. n can be 1. n can be 2. nₓ can be 0. nₓ can be 1. In embodiments, R₄ and R₆ can be a side chain of serine or histidine.

The EEV can comprise the structure of Formula (C-a) or (C-b):

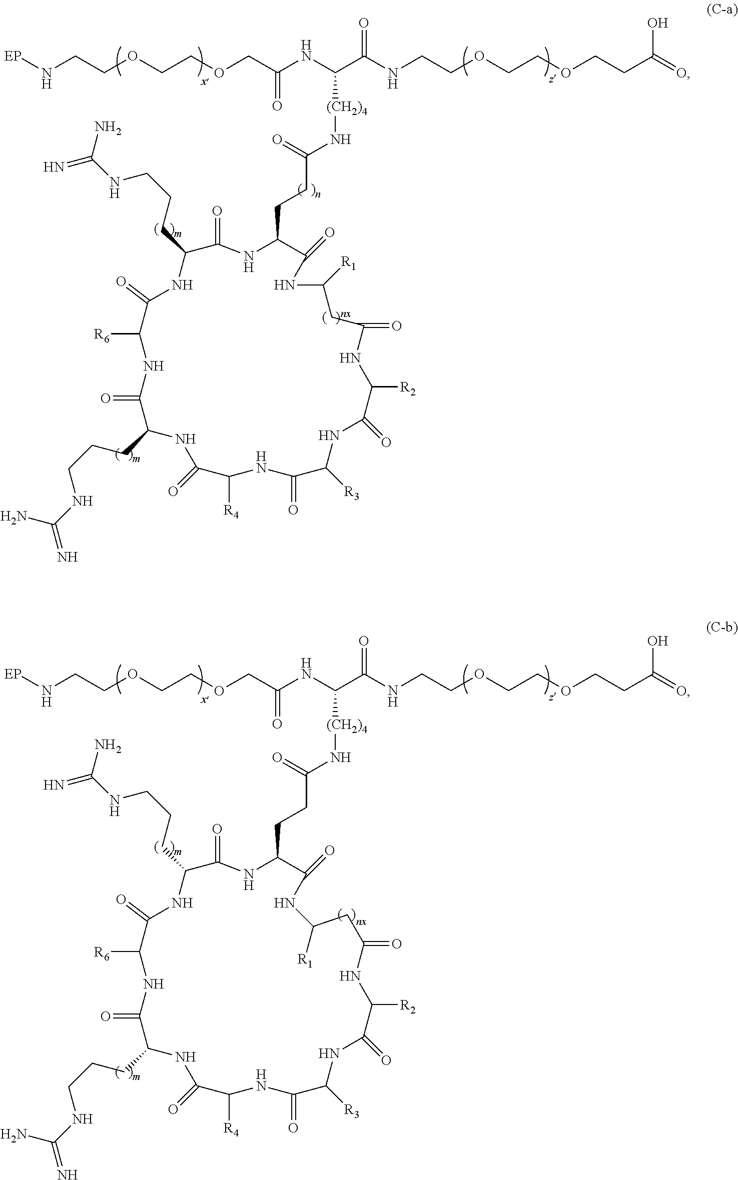

or a protonated form thereof, wherein EP, R₁, R₂, R₃, R₄, R₆, m, nₓ, and z′ are as defined in Formula (C).

The EEV can comprises the structure of Formula (C-B-c):

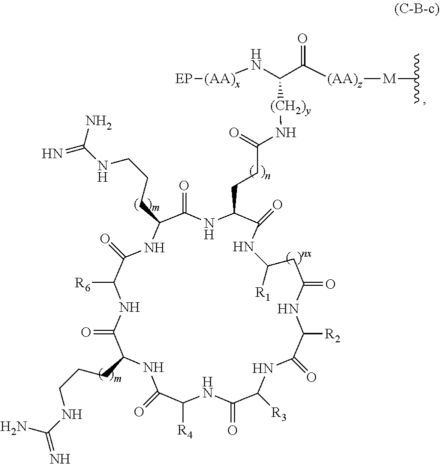

or a protonated form thereof, wherein EP, R₁, R₂, R₃, R₄, R₆, nₓ, and m are as defined above in Formula (B); AA is an amino acid as defined herein; M is as defined herein; n is an integer from 0-2; x is an integer from 1-10; y is an integer from 1-5; and z is an integer from 1-10.

The EEV can have the structure of Formula (SEQ ID NOS 425, 427 and 429, respectively, in order of appearance):

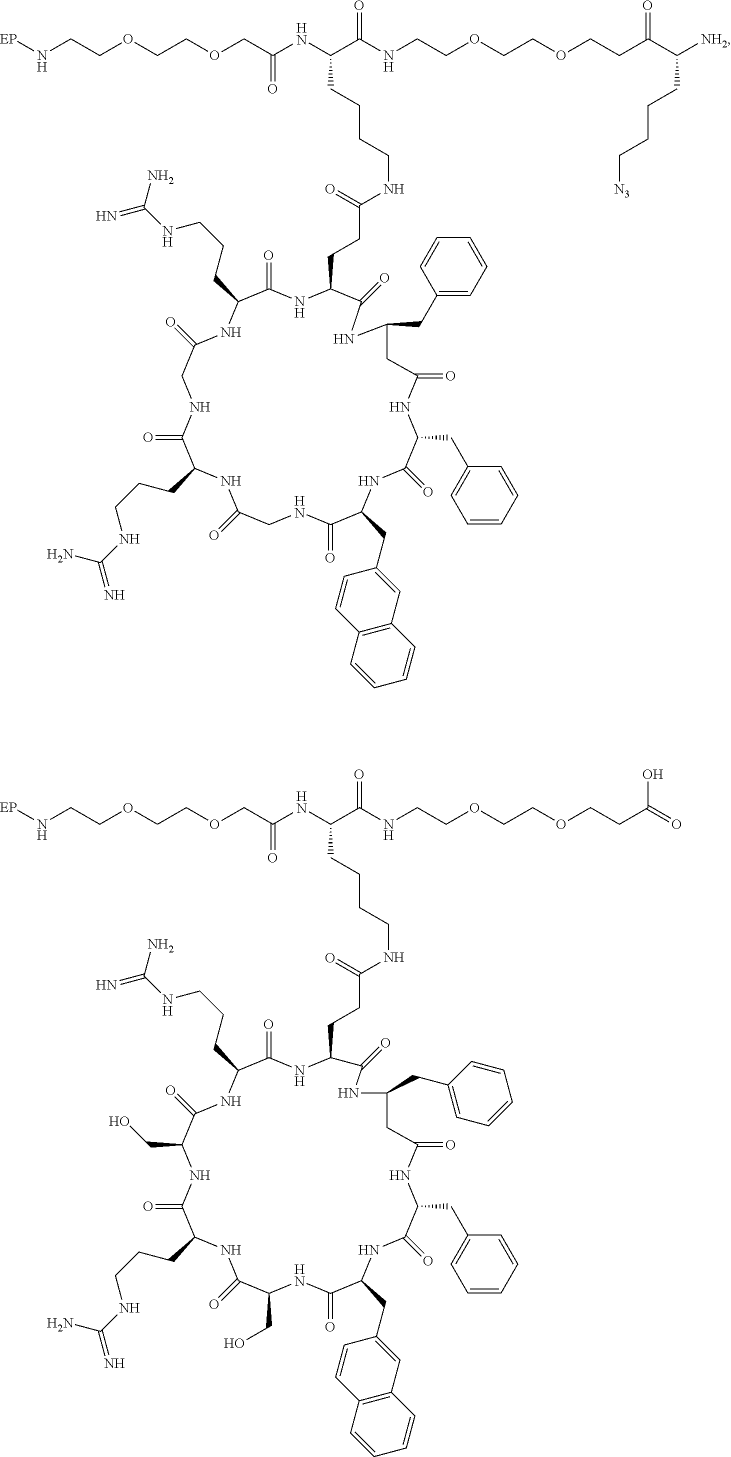

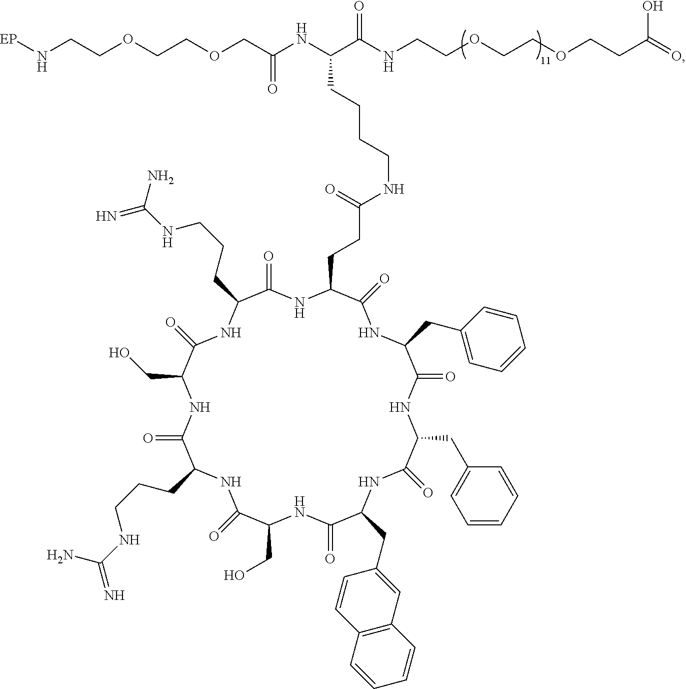

or a protonated form thereof,
wherein EP is as defined above in Formula (C).

The EEV can comprise Formula (C) and can have the structure: Ac-PKKRKV-AEEA-K (cyclo[bhFfFGrGrQ])-AEEA-K(N₃)-NH₂ (SEQ ID NOS 232 and 233, respectively, in order of appearance); Ac-PKKKRKV-AEEA-K(cyclo[FfFSrSrQ])-AEEA-K(N₃)-NH₂ (SEQ ID NOS 234and 235, respectively, in order of appearance), or Ac-PKKKRKV-AEEA-K(cyclo[bhFFFSRSRQ])-PEG₁₂-OH (SEQ ID NOS 236 and 237, respectively, in order of appearance).

The BEV can comprise two or more cCPP conjugated to the cargo. In embodiments, the EEV can be (cCPP)-linker-k(cCPP)-linker-OH.

The EEV can comprise a cCPP of formula (SEQ ID NO: 431):

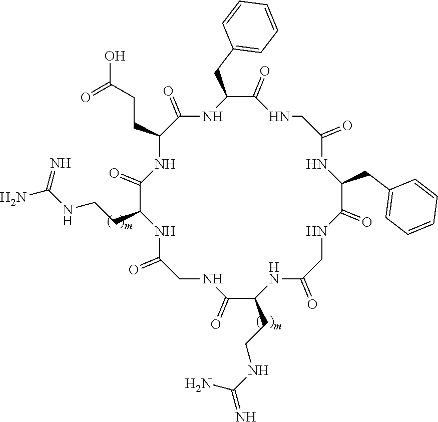 [SMILES: N=C(N)NCC[C@@H]1NC(=O)CNC(=O)[C@H](Cc2ccccc2)NC(=O)CNC(=O)[C@H](Cc2ccccc2)NC(=O)[C@H](CCC(=O)O)NC(=O)[C@H](CCNC(=N)N)NC(=O)CNC1=O]

The EEV can comprise formula: Ac-PKKKRKV-miniPEG₂-Lys(cyclo(FfFGRGRQ)-PEG₂-K(N₃)) (SEQ ID NOS 238 and 239, respectively, in order of appearance).

The EEV can be Ac-P-K(Tfa)-K(Tfa)-K(Tfa)-R-K(Tfa)-V-AEEA-K-(cyclo [FGFGRGRQ])-PEG12-OH (SEQ ID NOS 240 and 241, respectively, in order of appearance). The EEV can be (SEQ ID NOS 240-241, respectively, in order of appearance):

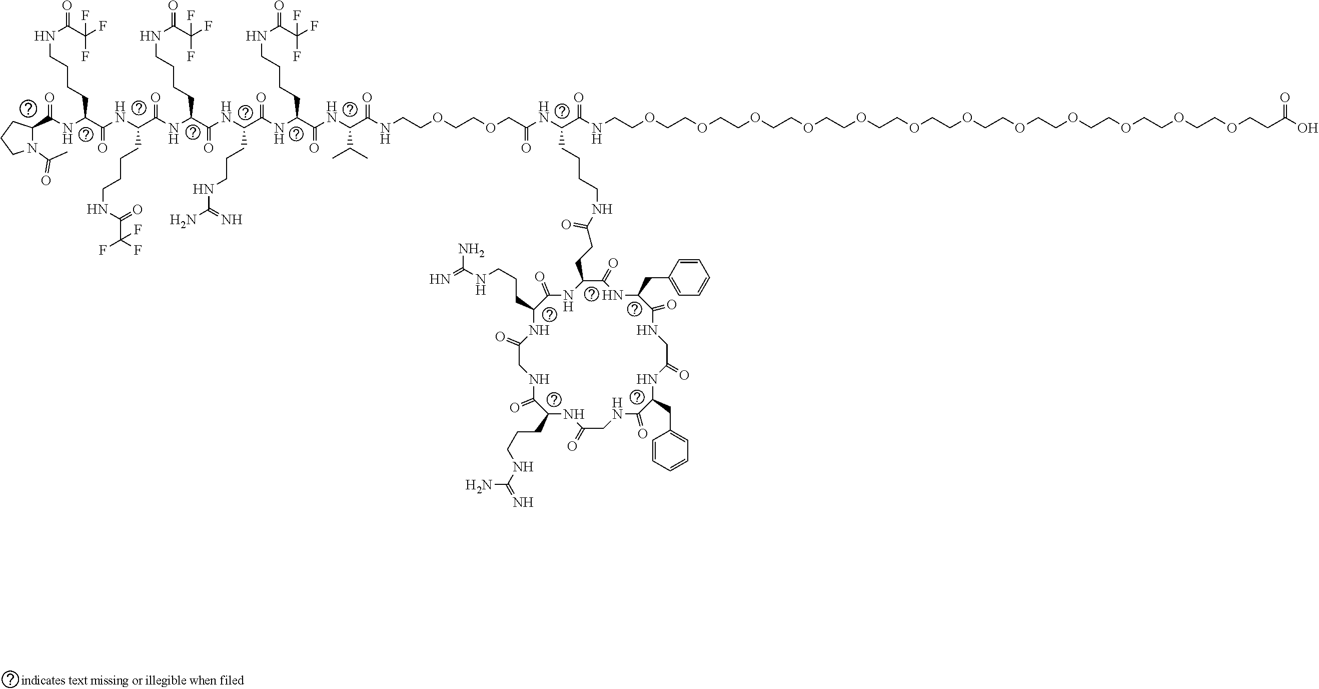 [SMILES: CC(=O)N1CCC[C@H]1C(=O)N[C@@H](CCCCNC(=O)C(F)(F)F)C(=O)N[C@@H](CCCCNC(=O)C(F)(F)F)C(=O)N[C@@H](CCCCNC(=O)C(F)(F)F)C(=O)N[C@@H](CCCNC(=N)N)C(=O)N[C@@H](CCCCNC(=O)C(F)(F)F)C(=O)N[C@H](C(=O)NCCOCCOCC(=O)N[C@@H](CCCCNC(=O)CC[C@@H]1NC(=O)[C@H](CCCNC(=N)N)NC(=O)CNC(=O)[C@H](CCCNC(=N)N)NC(=O)CNC(=O)[C@H](Cc2ccccc2)NC(=O)CNC(=O)[C@H](Cc2ccccc2)NC1=O)C(=O)NCCOCCOCCOCCOCCOCCOCCOCCOCCOCCOCCOCCOCCC(=O)O)C(C)C]

The EEV can be Ac-PKKKRKV-AEEA-Lys-(cyclo[FGFGRGRQ])-PEG12-OH (SEQ ID NOS 242 and 243, respectively, in order of appearance). The EEV can be (SEQ ID NOS 242-243, respectively, in order of appearance):

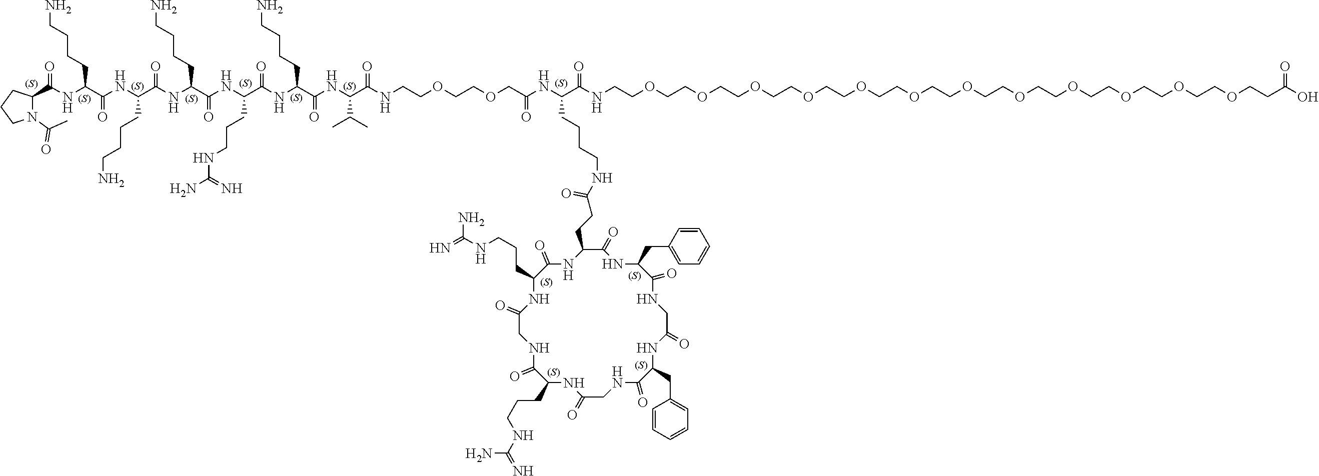

The EEV can be selected from

```
  (SEQ ID NO: 244)
Ac-rr-miniPEG₂-Dap(cyclo[FfΦ-Cit-r-Cit-rQ])-PEG₁₂-OH
  (SEQ ID NO: 245)
Ac-frr-PEG₂-Dap(cyclo[FfΦ-Cit-r-Cit-rQ])-PEG₁₂-OH
  (SEQ ID NO: 246)
Ac-rfr-PEG₂-Dap(cyclo[FfΦ-Cit-r-Cit-rQ])-PEG₁₂-OH
  (SEQ ID NO: 248)
Ac-rbfbr-PEG₂-Dap(cyclo[FfΦ-Cit-r-Cit-rQ])-PEG₁₂-OH
  (SEQ ID NO: 249)
Ac-rrr-PEG₂-Dap(cyclo[FfΦ-Cit-r-Cit-rQ])-PEG₁₂-OH
  (SEQ ID NO: 250)
Ac-rbr-PEG₂-Dap(cyclo[FfΦ-Cit-r-Cit-rQ])-PEG₁₂-OH
  (SEQ ID NO: 252)
Ac-rbrbr-PEG₂-Dap(cyclo[FfΦ-Cit-r-Cit-rQ])-PEG₁₂-OH
  (SEQ ID NO: 253)
Ac-hh-PEG₂-Dap(cyclo[FfΦ-Cit-r-Cit-rQ])-PEG₁₂-OH
  (SEQ ID NO: 254)
Ac-hbh-PEG₂-Dap(cyclo[FfΦ-Cit-r-Cit-rQ])-PEG₁₂-OH
  (SEQ ID NO: 256)
Ac-hbhbh-PEG₂-Dap(cyclo[FfΦ-Cit-r-Cit-rQ])-PEG₁₂-OH
  (SEQ ID NO: 258)
Ac-rbhbh-PEG₂-Dap(cyclo[FfΦ-Cit-r-Cit-rQ])-PEG₁₂-OH
  (SEQ ID NO: 260)
Ac-hbrbh-PEG₂-Dap(cyclo[FfΦ-Cit-r-Cit-rQ1)-PEG₁₂-OH
  (SEQ ID NO: 261)
Ac-rr-Dap(cyclo[FfΦ-Cit-r-Cit-rQ])-b-OH
  (SEQ ID NO: 262)
Ac-frr-Dap(cyclo[FfΦ-Cit-r-Cit-rQ])-b-OH
  (SEQ ID NO: 263)
Ac-rfr-Dap(cyclo[FfΦ-Cit-r-Cit-rQ1)-b-OH
(SEQ ID NO: 447)
Ac-rbfbr-Dap(cyclo[FfΦ-Cit-r-Cit-rQ])-b-OH
  (SEQ ID NO: 265)
Ac-rrr-Dap(cyclo[FfΦ-Cit-r-Cit-rQ])-b-OH
  (SEQ ID NO: 266)
Ac-rbr-Dap(cyclo[FfΦ-Cit-r-Cit-rQ])-b-OH
  (SEQ ID NO: 449)
Ac-rbrbr-Dap(cyclo[FfΦ-Cit-r-Cit-rQ])-b-OH
  (SEQ ID NO: 268)
Ac-hh-Dap(cyclo[FfΦ-Cit-r-Cit-rQ])-b-OH
  (SEQ ID NO: 269)
Ac-hbh-Dap(cyclo[FfΦ-Cit-r-Cit-rQ])-b-OH
  (SEQ ID NO: 460)
Ac-hbhbh-Dap(cyclo[FfΦ-Cit-r-Cit-rQ])-b-OH
  (SEQ ID NO: 462)
Ac-rbhbh-Dap(cyclo[FfΦ-Cit-r-Cit-rQ])-b-OH
  (SEQ ID NO: 464)
Ac-hbrbh-Dap(cyclo[FfΦ-Cit-r-Cit-rQ])-b-OH
  (SEQ ID NOS 273 and 274, respectively, in order of appearance)
Ac-KKKK-miniPEG₂-Lys(cyclo[FfΦGrGrQ])-miniPEG₂-K(N₃)-NH₂
  (SEQ ID NOS 275 and 276, respectively, in order of appearance)
Ac-KGKK-miniPEG₂-Lys(cyclo[FfΦGrGrQ])-miniPEG₂-K(N₃)-NH₂
  (SEQ ID NOS 277 and 278, respectively, in order of appearance)
Ac-KKGK-miniPEG₂-Lys(cyclo[FfΦGrGrQ])-miniPEG₂-K(N₃)-NH₂
  (SEQ ID NO: 279)
Ac-KKK-miniPEG₂-Lys(cyclo[FfΦGrGrQ])-miniPEG₂-K(N₃)-NH₂
  (SEQ ID NO: 280)
Ac-KK-miniPEG₂-Lys(cyclo[FfΦGrGrQ])-miniPEG₂-K(N₃)-NH₂
  (SEQ ID NO: 281)
Ac-KGK-miniPEG₂-Lys(cyclo[FfΦGrGrQ])-miniPEG₂-K(N₃)-NH₂
  (SEQ ID NO: 282)
Ac-KBK-miniPEG₂-Lys(cyclo[FfΦGrGrQ])-miniPEG₂-K(N₃)-NH₂
  (SEQ ID NO: 284)
Ac-KBKBK-PEG₂-Lys(cyclo[FfΦGrGrQ])-PEG₂-K(N₃)-NH₂
  (SEQ ID NO: 285)
Ac-KR-miniPEG₂-Lys(cyclo[FfΦGrGrQ])-miniPEG₂-K(N3N₃NH₂
  (SEQ ID NO: 286)
Ac-KBR-miniPEG₂-Lys(cyclo[FfΦGrGrQ])-miniPEG₂-K(N₃)-NH₂
  (SEQ ID NOS 287 and 288, respectively, in order of appearance)
Ac-PKKKRKV-miniPEG₂-Lys(cyclo[FfΦGrGrQ])-miniPEG₂-K(N₃)-NH₂
  (SEQ ID NOS 287 and 288, respectively, in order of appearance)
Ac-PKKKRKV-miniPEG₂-Lys(cyclo[FfΦGrGrQ])-miniPEG₂-K(N₃)-NH₂
  (SEQ ID NOS 289 and 290, respectively, in order of appearance)
Ac-PGKKRKV-miniPEG₂-Lys(cyclo[FfΦGrGrQ])-miniPEG₂-K(N₃)-NH₂
  (SEQ ID NOS 291 and 292, respectively, in order of appearance)
Ac-PKGKRKV-miniPEG₂-Lys(cyclo[FfΦGrGrQ])-miniPEG₂-K(N₃)-NH₂
  (SEQ ID NOS 293 and 294, respectively, in order of appearance)
Ac-PKKGRKV-miniPEG₂-Lys(cyclo[FfΦGrGrQ])-miniPEG₂-K(N₃)-NH₂
  (SEQ ID NOS 295 and 296, respectively, in order of appearance)
Ac-PKKKGKV-miniPEG₂-Lys(cyclo[FfΦGrGrQ])-miniPEG₂-K(N₃)-NH₂
  (SEQ ID NOS 297 and 298, respectively, in order of appearance)
Ac-PKKKRGV-miniPEG₂-Lys(cyclo[FfΦGrGrQ])-miniPEG₂-K(N₃)-NH₂
  (SEQ ID NOS 299 and 300, respectively, in order of appearance)
Ac-PKKKRKG-miniPEG₂-Lys(cyclo[FfΦGrGrQ])-miniPEG₂-K(N₃)-NH₂
  (SEQ ID NOS 301 and 302, respectively, in order of appearance)
Ac-KKKRK-miniPEG₂-Lys(cyclo[FfΦGrGrQ])-miniPEG₂-K(N₃)-NH₂
(SEQ ID NOS 303 and 304, respectively, in order of appearance)
Ac-KKRK-miniPEG₂-Lys(cyclo[FfΦGrGrQ])-miniPEG₂-K(N₃)-NH₂
and
  (SEQ ID NO: 305)
Ac-KRK-miniPEG2-Lys(cyclo[FfΦGrGrQ])-miniPEG₂-K(N₃)-NH₂.
```

The EEV can be selected from:

```
  (SEQ ID NO: 306 and 466, respectively, in order of appearance)
Ac-PKKKRKV-Lys(cyclo[FfΦGrGrQ])-PEG₁₂-K(N₃)-NH₂
  (SEQ ID NOS 307 and 308, respectively, in order of appearance)
Ac-PKKKRKV-miniPEG₂-Lys(cyclo[FfΦGrGrQ])-PEG₂-K(N₃)-NH₂
(SEQ ID NOS 309 and 310, respectively, in order of appearance)
Ac-PKKKRKV-miniPEG₂-Lys(cyclo[FGFGRGRQ])-miniPEG₂-K(N₃)-NH₂
  (SEQ ID NOS 311 and 312, respectively, in order of appearance)
Ac-PKKKRKV-miniPEG2-Lys(cyclo[GfFGrGrQ])-miniPEG2-K(N₃)-NH₂
  (SEQ ID NOS 313 and 314, respectively, in order of appearance
Ac-PKKKRKV-miniPEG2-Lys(cyclo[FfFGrGrQ])-miniPEG2-K(N₃)-NH₂
  (SEQ ID NOS 315 and 316, respectively, in order of appearance)
Ac-PKKKRKV-miniPEG2-Lys(cyclo[FGFRRRRQ])-miniPEG2-K(N₃)-NH₂
(SEQ ID NO: 317 and 468, respectively, in order of appearance)
Ac-PKKKRKV-Lys(cyclo(Ff-Nal-RrRrQ)
(SEQ ID NO: 318)
Ac-KR-PEG2-K(cyclo[FGFGRGRQ])-PEG₂-K(N₃)-NH₂
  (SEQ ID NOS 319 and 320, respectively, in order of appearance)
Ac-PKKKGKV-PEG₂-K(cyclo[FGFGRGRQ])-PEG2-K(N₃)-NH₂
  (SEQ ID NOS 321 and 322, respectively, in order of appearance)
Ac-PKKKRKG-PEG2-K(cyclo[FGFGRGRQ])-PEG₂-K(N₃)-NH₂
  (SEQ ID NOS 323 and 324, respectively, in order of appearance)
Ac-KKKRK-PEG₂-K(cyclo[FGFGRGRQ])-PEG₂-K(N₃)-NH₂
  (SEQ ID NOS 325 and 326, respectively, in order of appearance)
Ac-PKKKRKV-miniPEG₂-Lys(cyclo[FFΦGRGRQ])-miniPEG₂-K(N₃)-NH₂
  (SEQ ID NOS 327 and 328, respectively, in order of appearance)
Ac-PKKKRKV-miniPEG₂-Lys(cyclo[BhFfΦGrGrQ])-miniPEG₂-K(N₃)-NH₂
  (SEQ ID NOS 329 and 330, respectively, in order of appearance)
Ac-PKKKRKV-miniPEG₂-Lys(cyclo[FfΦSrSrQ])-miniPEG₂-K(N₃)-NH₂.
```

The EEV can be selected from:

```
  (SEQ ID NOS 331 and 332, respectively, in order of appearance)
Ac-PKKKRKV-miniPEG₂-Lys(cyclo(GfFGrGrQ])-PEG₁₂-OH
  (SEQ ID NOS 333 and 334, respectively, in order of appearance)
Ac-PKKKRKV-miniPEG₂-Lys(cyclo[FGFKRKRQ])-PEG₁₂-OH
  (SEQ ID NOS 335 and 336, respectively, in order of appearance)
Ac-PKKKRKV-miniPEG₂-Lys(cyclo[FGFRGRGQ])-PEG₁₂-OH
(SEQ ID NOS 337 and 338, respectively, in order of appearance)
Ac-PKKKRKV-miniPEG₂-Lys(cyclo[FGFGRGRGRQ])-PEG₁₂-OH
  (SEQ ID NOS 339 and 340, respectively, in order of appearance)
Ac-PKKKRKV-miniPEG₂-Lys(cyclo[FGFGRrRQ])-PEGPEG₁₂2-OH
(SEQ ID NOS 341 and 342, respectively, in order of appearance)
Ac-PKKKRKV-miniPEG₂-Lys(cyclo[FGFGRRRQ])-PEG₁₂-OH
and
(SEQ ID NOS 343 and 344, respectively, in order of appearance)
Ac-PKKKRKV-miniPEG₂-Lys(cyclo[FGFRRRRQ])-PEG₁₂-OH.
```

The EEV can be selected from:

```
  (SEQ ID NOS 345 and 346, respectively, in order of appearance)
Ac-KKKRKG-miniPEG₂-K(cyclo[FGFGRGRQ])-PEG₁₂-OH
  (SEQ ID NOS 347 and 348, respectively, in order of appearance)
Ac-KKKRK-miniPEG₂-K(cyclo[FGFGRGRQ])-PEG₁₂-OH
(SEQ ID NOS 349 and 350, respectively, in order of appearance)
Ac-KKRKK-PEG₄-K(cyclo[FGFGRGRQ])-PEG₁₂-OH
  (SEQ ID NOS 351 and 352, respectively, in order of appearance)
Ac-KRKKK-PEG₄-K(cyclo[FGFGRGRQ])-PEG₁₂-OH
  (SEQ ID NOS 353 and 354, respectively, in order of appearance)
Ac-KKKKR-PEG₄-K(cyclo[FGFGRGRQ])-PEG₁₂-OH
  (SEQ ID NOS 355 and 356, respectively, in order of appearance)
Ac-RKKKK-PEG₄-K(cyclo[FGFGRGRQ])-PEG₁₂-OH
and
  (SEQ ID NOS 357 and 358, respectively, in order of appearance)
Ac-KKKRK-PEG₄-K(cyclo[FGFGRGRQ])-PEG₁₂-OH.
```

The BEV can be selected from:

```
  (SEQ ID NOS 359 and 360, respectively, in order of appearance)
Ac-PKKKRKV-PEG₂-K(cyclo[FGFGRGRQ])-PEG₂-K(N₃)-NH₂
  (SEQ ID NOS 361 and 362, respectively, in order of appearance)
Ac-PKKKRKV-PEG₂-K(cyclo[FGFGRGRQ])-PEG₁₂-OH
  (SEQ ID NOS 363 and 364, respectively, in order of appearance)
Ac-PKKKRKV-PEG₂-K(cyclo[GfFGrGrQ])-PEG₂-K(N₃)-NH₂
and
  (SEQ ID NOS 365 and 366, respectively, in order of appearance)
Ac-PKKKRKV-PEG₂-K(cyclo[GIFGrGrQ])-PEG₁₂-OH.
```

The cargo can be a protein and the EEV can be selected from:

```
  (SEQ ID NOS 367 and 368, respectively, in order of appearance)
Ac-PKKKRKV-PEG₂-K(cyclo[FfΦGrGrQ])-PEG₁₂-OH
  (SEQ ID NOS 369 and 370, respectively, in order of appearance)
Ac-PKKKRKV-PEG₂-K(cyclo[FfΦCit-r-Cit-rQ])-PEG₁₂-OH
  (SEQ ID NOS 371 and 372, respectively, in order of appearance)
Ac-PKKKRKV-PEG₂-K(cyclo[FfFGRGRQ])-PEG₁₂-OH
  (SEQ ID NOS 373 and 374, respectively, in order of appearance)
Ac-PKKKRKV-PEG₂-K(cyclo[FGFGRGRQ])-PEG₁₂-OH
  (SEQ ID NOS 375 and 376, respectively, in order of appearance)
Ac-PKKKRKV-PEG₂-K(cyclo[GfFGrGrQ])-PEG₁₂-OH
  (SEQ ID NOS 377 and 378, respectively, in order of appearance)
Ac-PKKKRKV-PEG₂-K(cyclo[FGFGRRRQ])-PEG₁₂-OH
  (SEQ ID NOS 379 and 380, respectively, in order of appearance)
Ac-PKKKRKV-PEG₂-K(cyclo[FGFRRRRQ])-PEG₁₂-OH
  (SEQ ID NO: 381)
Ac-rr-PEG₂-K(cyclo[FfΦGrGrQ])-PEG₁₂-OH
  (SEQ ID NO: 382)
Ac-rr-PEG₂-K(cyclo[FfΦCit-r-Cit-rQ])-PEG₁₂-OH
  (SEQ ID NO: 383)
Ac-rr-PEG₂-K(cyclo[FfF-GRGRQ])-PEG₁₂-OH
  (SEQ ID NO: 384)
Ac-rr-PEG₂-K(cyclo[FGFGRGRQ])-PEG₁₂-OH
  (SEQ ID NO: 385)
Ac-rr-PEG₂-K(cyclo[GfFGrGrQ])-PEG₁₂-OH
  (SEQ ID NO: 386)
Ac-rr-PEG₂-K(cyclo[FGFGRRRQ])-PEG₁₂-OH
  (SEQ ID NO: 387)
Ac-rr-PEG₂-K(cyclo[FGFRRRRQ])-PEG₁₂-OH
  (SEQ ID NO: 388)
Ac-rrr-PEG₂-K(cyclo[FfΦGrGrQ])-PEG₁₂-OH
  (SEQ ID NO: 389)
Ac-rrr-PEG₂-K(cyclo[FfΦCit-r-Cit-rQ])-PEG₁₂-OH
  (SEQ ID NO: 390)
Ac-rrr-PEG₂-K(cyclo[FfFGRGRQ])-PEG₁₂-OH
  (SEQ ID NO: 391)
Ac-rrr-PEG₂-K(cyclo[FGFGRGRQ])-PEG₁₂-OH
(SEQ ID NO: 392)
Ac-rrr-PEG₂-K(cyclo[GfFGrGrQ])-PEG₁₂-OH
  (SEQ ID NO: 393)
Ac-rrr-PEG₂-K(cyclo[FGFGRRRQ])-PEG₁₂-OH
  (SEQ ID NO: 394)
Ac-rrr-PEG₂-K(cyclo[FGFRRRRQ])-PEG₁₂-OH
  (SEQ ID NO: 395)
Ac-rhr-PEG₂-K(cyclo[FfΦGrGrQ])-PEG₁₂-OH
  (SEQ ID NO: 396)
Ac-rhr-PEG₂-K(cyclo[FfΦCit-r-Cit-rQ])-PEG₁₂-OH
  (SEQ ID NO: 397)
Ac-rhr-PEG₂-K(cyclo[FfFGRGRQ])-PEG₁₂-OH
  (SEQ ID NO: 398)
Ac-rhr-PEG₂-K(cyclo[FGFGRGRQ])-PEG₁₂-OH
  (SEQ ID NO: 399)
Ac-rhr-PEG₂-K(cyclo[GfFGrGrQ])-PEG₁₂-OH
  (SEQ ID NO: 400)
Ac-rhr-PEG₂-K(cyclo[FGFGRRRQ])-PEG₁₂-OH
(SEQ ID NO: 401)
Ac-rhr-PEG₂-K(cyclo[FGFRRRRQ])-PEG₁₂-OH
  (SEQ ID NO: 402)
Ac-rbr-PEG₂-K(cyclo[FfΦGrGrQ])-PEG₁₂-OH
  (SEQ ID NO: 403)
Ac-rbr-PEG₂-K(cyclo[FfΦCit-r-Cit-rQ])-PEG₁₂-OH
(SEQ ID NO: 404)
Ac-rbr-PEG₂-K(cyclo[FfFGRGRQ])-PEG₁₂-OH
  (SEQ ID NO: 405)
Ac-rbr-PEG₂-K(cyclo[FGFGRGRQ])-PEG₁₂-OH
  (SEQ ID NO: 406)
Ac-rbr-PEG₂-K(cyclo[GfFGrGrQ])-PEG₁₂-OH
  (SEQ ID NO: 407)
Ac-rbr-PEG₂-K(cyclo[FGFGRRRQ])-PEG₁₂-OH
  (SEQ ID NO: 408)
Ac-rbr-PEG₂-K(cyclo[FGFRRRRQ])-PEG₁₂-OH
  (SEQ ID NO: 410)
Ac-rbrbr-PEG₂-K(cyclo[FfΦGrGrQ])-PEG₁₂-OH
  (SEQ ID NO: 412)
Ac-rbrbr-PEG₂-K(cyclo[FfΦCit-r-Cit-rQ])-PEG₁₂-OH
  (SEQ ID NO: 414)
Ac-rbrbr-PEG₂-K(cyclo[FfFGRGRQ])-PEG₁₂-OH
  (SEQ ID NO: 416)
Ac-rbrbr-PEG₂-K(cyclo[FGFGRGRQ])-PEG₁₂-OH
  (SEQ ID NO: 418)
Ac-rbrbr-PEG₂-K(cyclo[GfFGrGrQ])-PEG₁₂-OH
  (SEQ ID NO: 420)
Ac-rbrbr-PEG₂-K(cyclo[FGFGRRRQ])-PEG₁₂-OH
  (SEQ ID NO: 422)
Ac-rbrbr-PEG₂-K(cyclo[FGFRRRRQ])-PEG₁₂-OH
  (SEQ ID NO: 424)
Ac-rbhbr-PEG₂-K(cyclo[FfΦGrGrQ])-PEG₁₂-OH
  (SEQ ID NO: 426)
Ac-rbhbr-PEG₂-K(cyclo[FfΦCit-r-Cit-rQ])-PEG₁₂-OH
  (SEQ ID NO: 428)
Ac-rbhbr-PEG₂-K(cyclo[FfFGRGRQ])-PEG₁₂-OH
  (SEQ ID NO: 430)
Ac-rbhbr-PEG₂-K(cyclo[FGFGRGRQ])-PEG₁₂-OH
  (SEQ ID NO: 432)
Ac-rbhbr-PEG₂-K(cyclo[GfFGrGrQ])-PEG₁₂-OH
(SEQ ID NO: 434)
Ac-rbhbr-PEG₂-K(cyclo[FGFGRRRQ])-PEG₁₂-OH
  (SEQ ID NO: 436)
Ac-rbhbr-PEG₂-K(cyclo[FGFRRRRQ])-PEG₁₂-OH
  (SEQ ID NO: 438)
Ac-hbrbh-PEG₂-K(cyclo[FfΦGrGrQ])-PEG₁₂-OH
  (SEQ ID NO: 440)
Ac-hbrbh-PEG₂-K(cyclo[FfΦCit-r-Cit-rQ])-PEG₁₂-OH
  (SEQ ID NO: 442)
Ac-hbrbh-PEG₂-K(cyclo[FfFGRGRQ])-PEG₁₂-OH
  (SEQ ID NO: 444)
Ac-hbrbh-PEG₂-K(cyclo[FGFGRGRQ])-PEG₁₂-OH
  (SEQ ID NO: 446)
Ac-hbrbh-PEG₂-K(cyclo[GfFGrGrQ])-PEG₁₂-OH
(SEQ ID NO: 448)
Ac-hbrbh-PEG₂-K(cyclo[FGFGRRRQ])-PEG₁₂-OH
  (SEQ ID NO: 450)
Ac-hbrbh-PEG₂-K(cyclo[FGFRRRRQ])-PEG₁₂-OH,
wherein b is β-alanine, and the exocyclic sequence can be D or L stereochemistry
```

The EEV can be selected from:

```
  (SEQ ID NO: 451)
cyclo(FGFGHGHQ)-PEG₁₂-K(N₃)-NH₂
  (SEQ ID NO: 452)
cyclo(FGFSHSHQ)-PEG₁₂-K(N₃)-NH₂
  (SEQ ID NO: 453)
cyclo(FfFGRGRQ)-PEG₁₂-K(N₃)-NH₂
  (SEQ ID NO: 454)
cyclo(FfFSRSRQ)-PEG₁₂-K(N₃)-NH₂
  (SEQ ID NO: 455)
cyclo(FGFSRSRQ)-PEG₁₂-K(N₃)-NH₂
  (SEQ ID NO: 456)
cyclo(FGFGRGRQ)-PEG12-K(N₃)-NH₂
  (SEQ ID NO: 457)
cyclo(FGFGKGKQ)-PEG12-K(N₃)-NH₂
  (SEQ ID NO: 457)
cyclo(FGFGKGKQ)-PEG₁₂-K(N₃)-NH₂
  (SEQ ID NO: 458)
cyclo(FGFKKKK)-PEG₁₂-K(N₃)-NH₂
  (SEQ ID NO: 459)
cyclo(FGFK(me2)K(me2)K(me2)K(me2)Q)-PEG₁₂-K(N₃)-N₂
  (SEQ ID NO: 461)
Ac-RBRBR-PEG₂-K(cyclo[Ff-Nal-GrGrQ]-PEG₁₂-K(N₃)-NH₂
  (SEQ ID NO: 463)
Ac-RBRBR-PEG₂-K(cyclo[BhFf-Nal-SR.SRQ]-PEG₁₂-K(N₃)-NH₂
  (SEQ ID NO: 465)
Ac-RBRBR-PEG₂-K(cyclo[FGFSRSRQ]-PEG₁₂-K(N₃)-NH₂
  (SEQ ID NO: 467)
Ac-RBRBR-PEG₂-K(cyclo[FGFGRGRQ]-PEG₁₂-K(N₃)-NH₂
  (SEQ ID NO: 469)
Ac-RBRBR-PEG₁₂-K[cyclo(FGFSHSHQ)]-PEG1₂-K(N₃)-NH:
  (SEQ ID NOS 470 and 471, respectively, in order of appearance)
Ac-KKKK-miniPEG2-Lys(cyclo(FGFGRGRQ))-miniPEG₂-K(N₃)-NH₂
  (SEQ ID NO: 473)
Ac-KBKBK-miniPEG2-Lys(cyclo(FGFGRGRQ))-miniPEG₂-K(N₃)-NH₂
  (SEQ ID NO: 474)
Ac-KBK-miniPEG2-Lys(cyclo(FGFGRGRQ))-miniPEG₂-K(N₃)-NH₂
```

### Delivery Constructs Conjugated to a Cargo

A delivery construct can be linked to a cargo to from a cargo conjugate. The cargo can be linked to the delivery construct through a linker such as the linkers disclosed herein. The cargo can be conjugated to the linker using any conjugation reaction disclosed herein to from any bonding group (M) disclosed herein. In embodiments, the cargo can have a reactive handle able to react with a terminal carbonyl group of a linker to form a bonding group.

In embodiments, a cargo is directly conjugated to the cCPP of a delivery construct to form a cargo conjugate. In embodiments, at least one atom of the cCPP can be replaced by a cargo or at least one lone pair can form a bond to a cargo. In embodiments, at least one atom of an amino acid side chain of the cCPP is replaced by a cargo or at least one lone pair of the atom forms a bond to a cargo. In embodiments, a hydroxyl group on an amino acid side chain of the cCPP can be replaced by a bond to the cargo. In embodiments, a hydroxyl group on a glutamine side chain of the cCPP can be replaced by a bond to the cargo.

In embodiments, a cargo is linked to a delivery construct through a linker to form a cargo conjugate. In embodiments, the delivery construct comprises a cCPP and a linker. In embodiments, the AA_{sc} of a cCPP is conjugated to a linker and the cargo is conjugated to the linker thereby forming a cargo conjugate. In embodiments where the delivery construct comprises an EEV, a component of the EEV, such as the cargo, the EP, and/or the AA_{sc} of the cCPP, are conjugated to the linker thereby forming a cargo conjugate.

In embodiments, the amino acid side chain of the cCPP comprises a reactive handle to which the linker or cargo is conjugated to through a conjugation reaction. The reactive handle may comprise any reactive handle described herein. In embodiments, the reactive handle comprises an amine group, a carboxylic acid, an amide, a hydroxyl group, a sulfhydryl group, a guanidinyl group, a phenolic group, a thioether group, an imidazolyl group, or an indolyl group. In embodiments, the amino acid (i.e., the AA_{SC}) of the cCPP to which the cargo is conjugated comprises lysine, arginine, aspartic acid, glutamic acid, asparagine, glutamine, homoglutamine, serine, threonine, tyrosine, cysteine, arginine, methionine, histidine or tryptophan.

In embodiments, a cargo can be conjugated to the linker at the terminal carbonyl group of the delivery construct to provide the following structure:

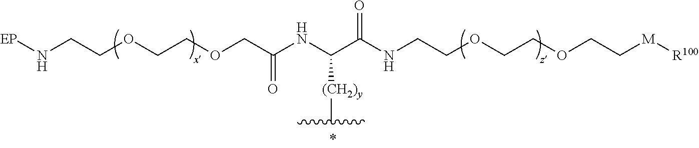

wherein:
EP is an exocyclic peptide; R¹⁰⁰ is a cargo; and M, AAsc, x′, y, and z′ are as defined above, * is the point of attachment to the AA_{SC} of any cCPP disclosed herein. x′ can be 1. y can be 4. z′ can be 11. —(OCH₂CH₂)_{z}′— and/or —(OCH₂CH₂)_{z}′— can be independently replaced with one or more amino acids, including, for example, glycine, β-alanine, 4-aminobutyric acid, 5-aminopentanoic acid, 6-aminohexanoic acid, or combinations thereof. In embodiments, the cargo is a lipid. In embodiments, the cargo is a component of a gene editing machinery (“GEM”).

A cargo conjugate may be of the Formula (J1c), (J2c), (J3c), (J4c), (J5c);

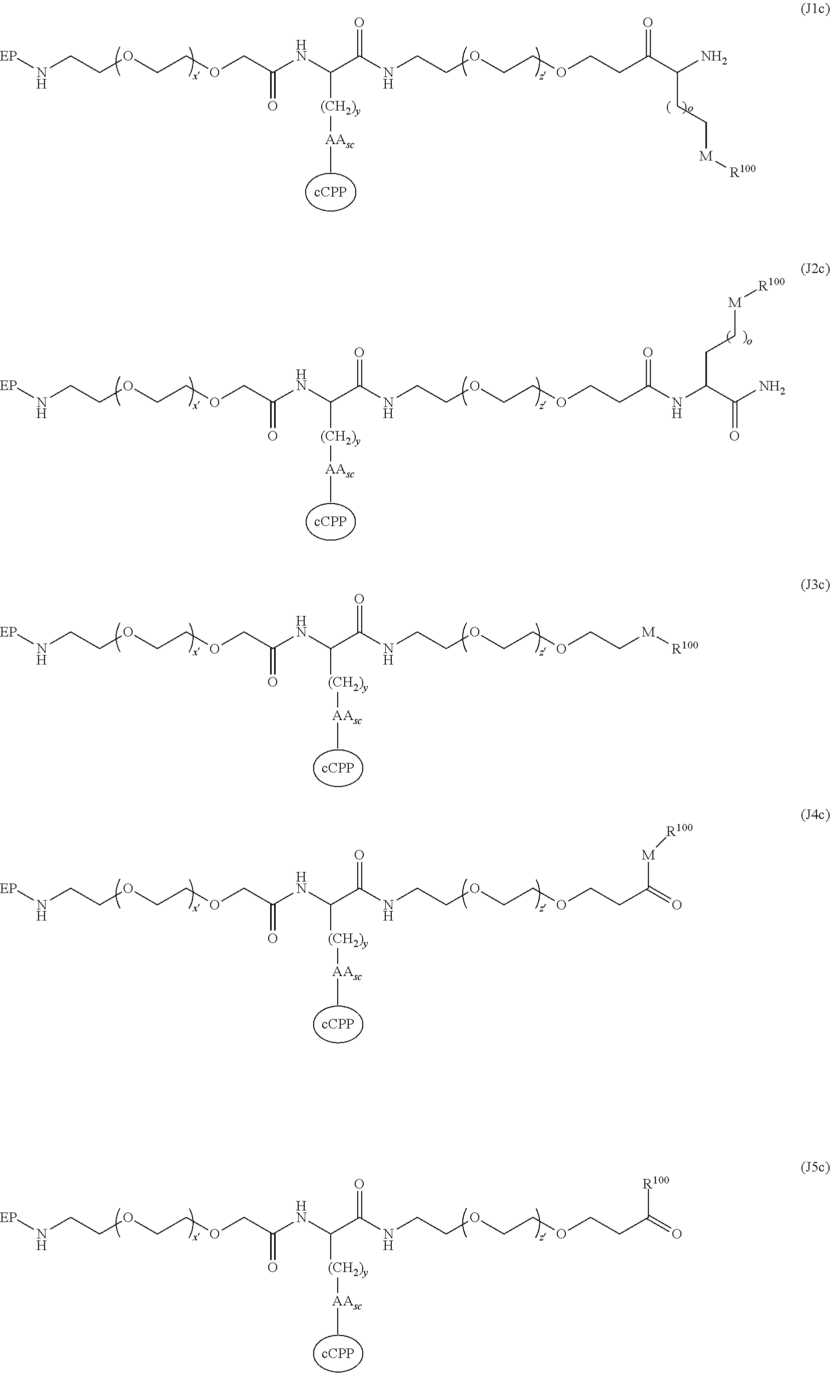

wherein R¹⁰⁰ is a cargo; EP is any exocyclic peptide disclosed herein; y is an integer from 1 to 5; x′ is an integer from 1-20; z′ is an integer from 1-23; cCPP is any cCPP disclosed herein; AA_{sc} is any AA_{sc} as disclosed herein; o is an integer from 1 to 5; and M is any bonding group disclosed herein. The stereochemistry of each of the stereocenters may be S or R.

A cargo conjugate may be of the Formula (JJ1c):

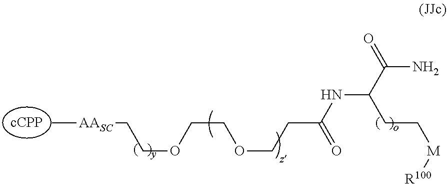

wherein R¹⁰⁰ is a cargo; y is an integer from 1 to 5; z′ is an integer from 1-23; cCPP is any cCPP disclosed herein; AA_{sc} is any AA_{sc} as disclosed herein; o is an integer from 1 to 5; and M is any bonding group disclosed herein. The stereochemistry of each of the stereocenters may be S or R.

In embodiments, the compound is of Formula (J1c), (J2c), (J3c), (J4c), (J5c), or (JJ1c) wherein x′ is 1 or 2. In embodiments, the delivery construct is of Formula (J1c), (J2c), or (J3c), wherein z′ is 1, 2, 11, or 12. In embodiments, the cCPP is of Formula (IA), (I), (I-a), (I-b), (I-2), (I-3), (I-4), (I-5), (I-6), (I-7), (IX), (IX1), (IX(a)), (IX(b)), (IX(c)), (II), (II-1), (IIa), (IIc), (III), (III-1), (IIIa), (D), (AV), (Y1), (Y1′), (Y2), (Y2′), (AA(a)), (AA(b)), (Y-a), (Y-aa), (Y-ab), (Ym), (Yn), (Yo), (Yp), (AA(c)), (AA(d)), (AA(e)), (A-II), (A-II-1), (A-IIa), (A-IIb), (A-III), (A-III-1), (A-IIIa), or derivatives having the specified characteristics described herein.

An endosomal escape vehicle (EEV) can comprise a cyclic cell penetrating peptide (cCPP), an exocyclic peptide (EP) and linker, and can be conjugated to a cargo to form a cargo conjugate comprising the structure of Formula (E):

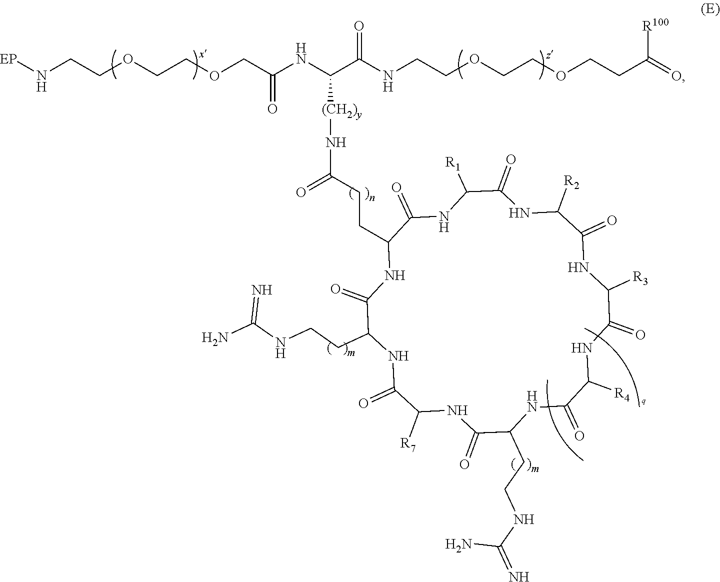

or a protonated form thereof,
wherein:
R¹⁰⁰ is a cargo;

R₁, R₂, and R₃ can each independently be H or an amino acid residue having a side chain comprising an aromatic group;
R₄ is H or an amino acid side chain;
EP is an exocyclic peptide as defined herein;
Cargo is a moiety as defined herein;
each m is independently an integer from 0-3;
n is an integer from 0-2;
x′ is an integer from 2-20;
y is an integer from 1-5;
q is an integer from 1-4; and
z′ is an integer from 2-20.

In embodiments, the cargo is a lipid. In embodiments, the cargo is a component of a gene editing machinery (“GEM”).

The EEV can be conjugated to a cargo and the cargo conjugate can comprise the structure of Formula (E-a) or (E-b):

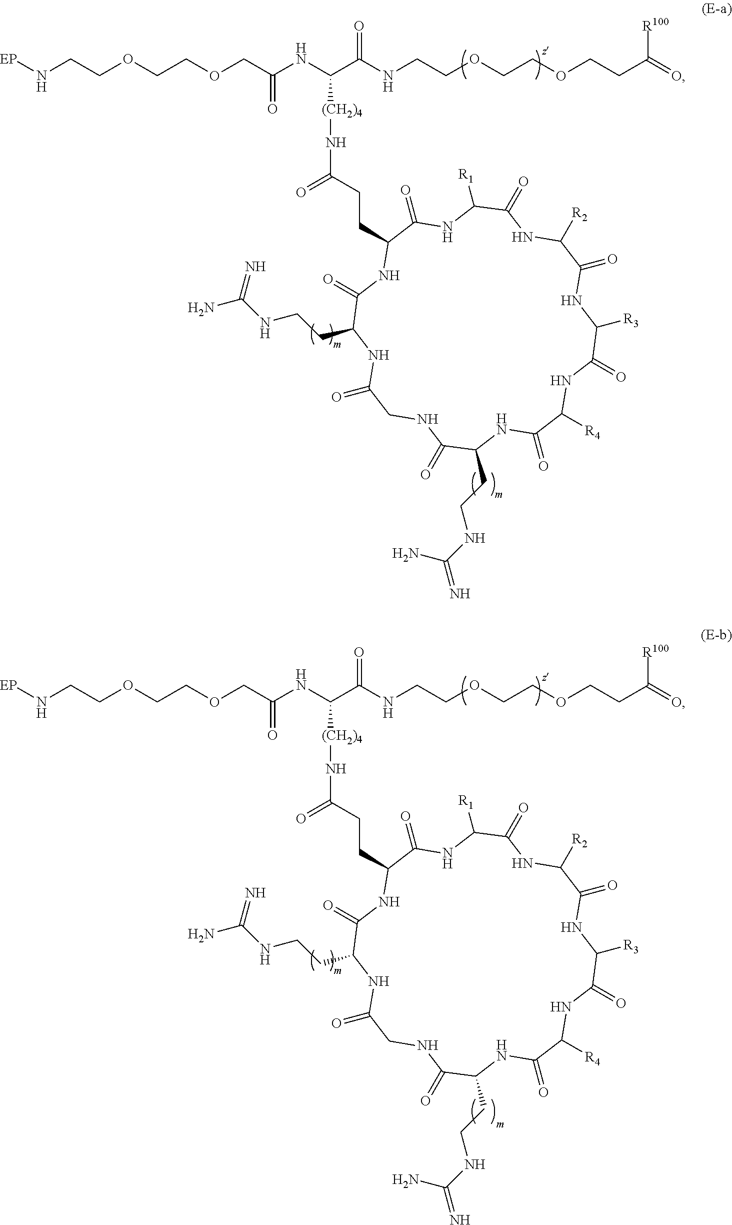

or a protonated form thereof, wherein R¹⁰⁰ is a cargo, and EP, m and z are as defined above in Formula (E).

The EEV can be conjugated to a cargo and the cargo conjugate can comprise the structure of Formula (E-c):

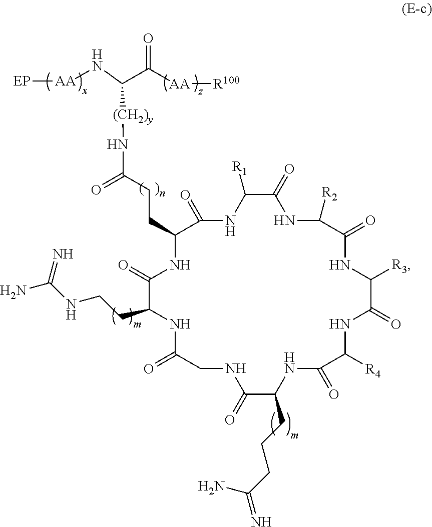

or a protonated form thereof, wherein R¹⁰⁰ is a cargo; and EP, R₁, R₂, R₃, R₄, and m are as defined above in Formula (III); AA can be an amino acid as defined herein; n can be an integer from 0-2; x can be an integer from 1-10; y can be an integer from 1-5; and z can be an integer from 1-10.

An endosomal escape vehicle (EEV) can comprise a cyclic cell penetrating peptide (cCPP), an exocyclic peptide (EP) and linker, and can be conjugated to a cargo to form a cargo conjugate comprising the structure of Formula (A-C):

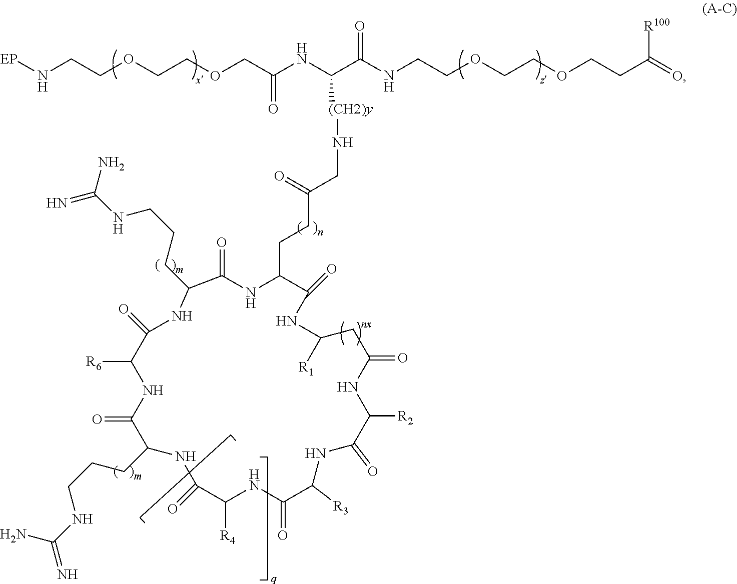

or a protonated form thereof,
wherein:
R¹⁰⁰ is a cargo;
R₁, R₂, and Rs can each independently be H or an amino acid residue having a side chain comprising an aromatic or heteroaromatic group;
R₄ or R₆ is independently H or an amino acid side chain;
EP is an exocyclic peptide as defined herein,
Cargo is a moiety as defined herein;
each m is independently an integer from 0-3;
n is an integer from 0-2;
nₓ is 1;
x′ is an integer from 2-20;
y is an integer from 1-5;
q is an integer from 1-4; and
z′ is an integer from 2-20.

In embodiments, the cargo is a lipid. In embodiments, the cargo is a protein. In embodiments, the cargo is a nucleic acid. In embodiments, the cargo is a component of a gene editing machinery (“GEM”).

The EEV can be conjugated to a cargo and the cargo conjugate can comprise the structure of Formula (A-C-a) or (A-C-b):

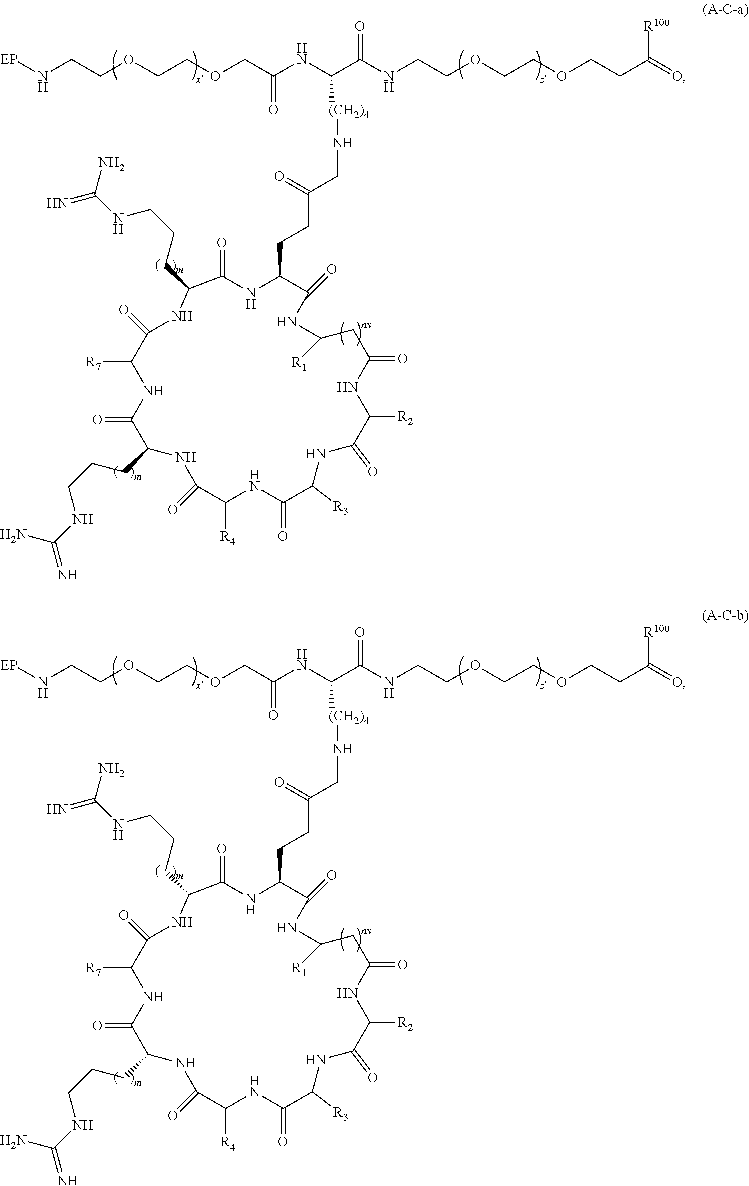

or a protonated form thereof, wherein EP, m and z are as defined above in Formula (A-C).

The EEV can be conjugated to a cargo and the cargo conjugate can comprise the structure of Formula (A-C-c):

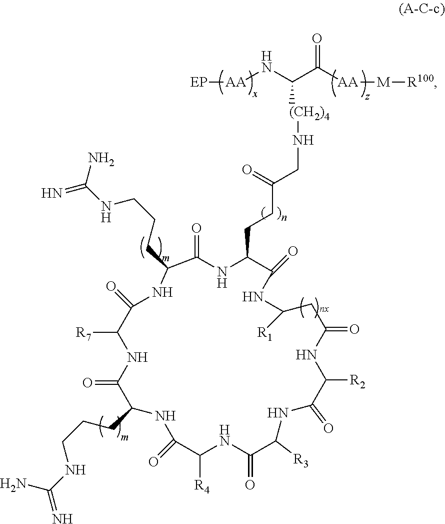

or a protonated form thereof, wherein EP, R¹, R², R³, R⁴, R¹⁰⁰, and m are as defined above in Formula (III); AA can be an amino acid as defined herein; n can be an integer from 0-2; x can be an integer from 1-10; y can be an integer from 1-5; and z can be an integer from 1-10.

The EEV can be conjugated to a cargo and the cargo conjugate can comprise a structure of Formula (SEQ ID NOS 433, 435 and 437, respectively, in order of appearance):

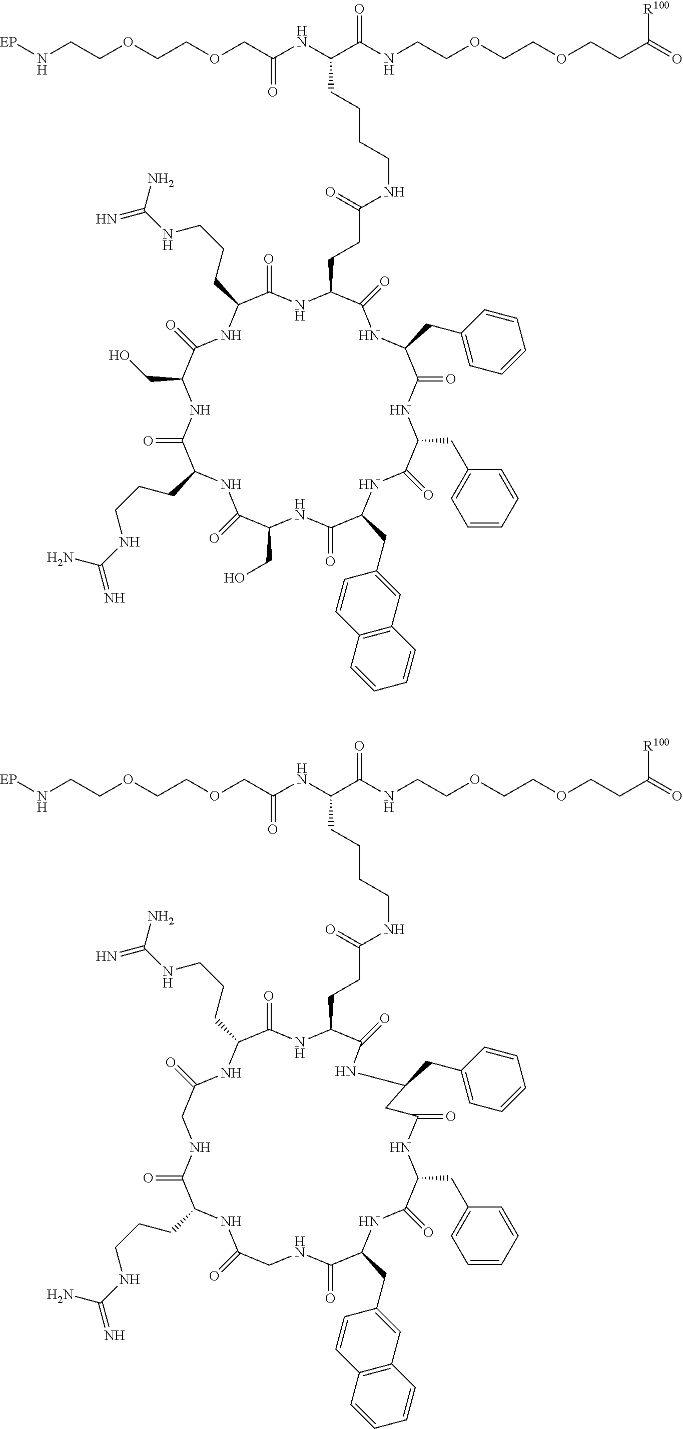

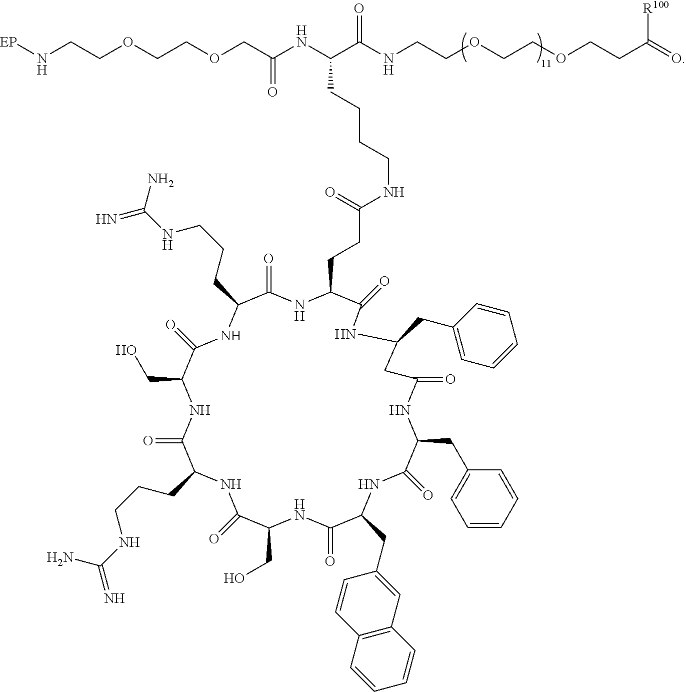

### Cytosolic Delivery Efficiency

Modifications to a cyclic cell penetrating peptide (cCPP) may improve cytosolic delivery efficiency. Improved cytosolic uptake efficiency can be measured by comparing the cytosolic delivery efficiency of a cCPP having a modified sequence to a control sequence. The control sequence does not include a particular replacement amino acid residue in the modified sequence (including, but not limited to arginine, phenylalanine, and/or glycine), but is otherwise identical.

As used herein cytosolic delivery efficiency refers to the ability of a cCPP to traverse a cell membrane and enter the cytosol of a cell. Cytosolic delivery efficiency of the cCPP is not necessarily dependent on a receptor or a cell type. Cytosolic delivery efficiency can refer to absolute cytosolic delivery efficiency or relative cytosolic delivery efficiency.

Absolute cytosolic delivery efficiency is the ratio of cytosolic concentration of a cCPP (or a cCPP-cargo conjugate) over the concentration of the cCPP (or the cCPP-cargo conjugate) in the growth medium. Relative cytosolic delivery efficiency refers to the concentration of a cCPP in the cytosol compared to the concentration of a control cCPP in the cytosol. Quantification can be achieved by fluorescently labeling the cCPP (e.g., with a FITC dye) and measuring the fluorescence intensity using techniques well-known in the art.

Relative cytosolic delivery efficiency is determined by comparing (i) the amount of a cCPP of the invention internalized by a cell type (e.g., HeLa cells) to (ii) the amount of a control cCPP internalized by the same cell type. To measure relative cytosolic delivery efficiency, the cell type may be incubated in the presence of a cCPP for a specified period of time (e.g., 30 minutes, 1 hour, 2 hours, etc.) after which the amount of the cCPP internalized by the cell is quantified using methods known in the art, e.g., fluorescence microscopy. Separately, the same concentration of the control cCPP is incubated in the presence of the cell type over the same period of time, and the amount of the control cCPP internalized by the cell is quantified.

Relative cytosolic delivery efficiency can be determined by measuring the IC₅₀ of a cCPP having a modified sequence for an intracellular target and comparing the IC₅₀ of the cCPP having the modified sequence to a control sequence (as described herein).

### Lipid Conjugates

In embodiments, the present disclosure describes lipid conjugates. As used herein, an “lipid conjugate” is a compound comprising lipid conjugated to a delivery construct. In embodiments, the delivery construct comprises a CPP. In embodiments, the CPP is conjugated to a lipid to form a lipid conjugate. CPPs are described herein. In embodiments, the CPP is a cCPP. In embodiments, the delivery construct comprises an EEV. In embodiments, the EEV is conjugated to a lipid to form a lipid conjugate. EEVs are described herein.

In embodiments, the lipid conjugates may include one or more linkers linking the components of the lipid conjugates. The linkers may be any suitable linkers, such as those described herein. In embodiments, the lipid conjugate comprises an EP, a cCPP, and a lipid all linked together through a trivalent linker. In embodiments, the lipid conjugate comprises a cCPP and a lipid linked through a bivalent linker.

One or more of the delivery constructs can be conjugated to one or more lipids using the conjugation reactions disclosed herein. As such, prior to conjugation, the delivery construct may comprise a reactive handle that is cooperative with a reactive handle on the lipid. Such reactive handles react to form a reaction product, or bonding group (M), thereby forming the lipid conjugate. The reactive handles may be any pair of reactive handles disclosed herein that react to form any reaction product or bonding group disclosed herein. Examples of conjugation chemistries useful for conjugating a delivery construct to a lipid are discussed elsewhere herein.

As used herein in the context of lipids, the statements “derived therefrom” and “derived from” refer to a compound comprising a lipid structure (e.g., a known lipid) functionalized with a reactive handle and/or a linker; and a compound comprising the reaction product between a lipid structure functionalized with a reactive handle and/or a linker with a delivery construct. Such compounds are said to be derived from the lipid structure that was functionalized and/or conjugated to the delivery construct.

In embodiments, the lipid conjugate comprises a helper lipid. In embodiments, the lipid conjugate comprises a cationic lipid. In embodiments, the lipid conjugate comprises an ionizable lipid. In embodiments, the lipid conjugate comprises a PEGylated lipid.

In embodiments, the conjugated lipid includes a ionizable lipid. The ionizable lipid may be, or derived from, any suitable ionizable lipid. Examples of suitable ionizable lipids include, but are not limtied to, D-Lin-MC3-DMA (also called just MC3; CAS No. 1224606-06-7; FIG. 4); ALC-0315 (CAS No. 2036272-55-4); and SM-102 (also called Lipid H; CAS No. 2089251-47-6; FIG. 4). Other suitable ionizable lipids that may be used include A2-Iso5-2DC18 (CAS No. 2412492-07-8); BAME-016 (CAS No. 2490668-30-7); C12-200 (CAS No. 1220890-25-4); cKK-E12 (CAS No. 1432494-65-9); OF-Deg-Lin (CAS No. 1853202-95-5); TT3 (CAS No. 1821214-50-9); 9A1P9 (CAS No. 2760467-57-8); FTT5 (CAS No. 2328129-27-5); 1,2-dilinoleyoxy-3-(dimethylamino)acetoxypropane (DLin DAC); lipid 5 (CAS No. 2089251-33-0); DLin-DMA (CAS No. 871258-12-7); D-Lin-MC3-DMA (CAS No. 1224606-06-7); DLin-KC2-DMA (CAS No. 1190197-97-7); YSK05 (CAS No. 1318793-78-0); AA3-DLin (CAS No. 2832061-33-1); SSPalmM (CAS No. 1436860-60-4); SSPalmO-Phe (CAS No. 2377474-67-2); L319 (CAS No. 1351586-50-9); Lipid A9 (CAS No. 2036272-50-9); Lipid A (CAS No. 2036272-50-9); CL4H6 (CAS No. 2256087-35-9); DODMA (also known as MBN305A; CAS No. 104162-47-2); CLI (CAS No. 1450888-71-7); ATX-001 (CAS No. 1777792-33-2); ATX-100 (CAS No. 2230647-37-5); 80-O16B (CAS No. 1624618-02-5); 93-O17S (CAS No. 2227008-67-3); 93-O170 (CAS No. 2227214-78-8); NT1-O14B (CAS No. 2739805-64-0); 306-O12B-3; 306-12B (CAS No. 2566523-06-4); 113-O16B (CAS No. 2566523-07-5); 306Oi10 (CAS No. 322290-93-5); cKK-E12 (CAS No. 1432494-65-9); OF-02 (CAS No. 1883431-67-1); C12-200 (CAS No. 1220890-25-4); 113-012B (CAS No. 2803699-72-9); LP01 (also known as BP-Lipid 215; CAS No. 1799316-64-5); TCL053 (CAS No. 2361162-70-9); Lipid C24 (CAS No. 2767561-52-2); Lipid 29 (CAS No. 2244716-55-8); 9A1P9 (CAS No. 2760467-57-8); C13-112-tri-tail (CAS No. 1381861-96-6); C13-113-tri-tail (CAS No. 1381861-86-4); C13-112-tetra-tail (CAS No. 1381861-92-2); C13-113-tetra-tail (CAS No. 1381861-97-7); DODAP (CAS No. 127512-29-2); 1,2 dilinoleyoxy-3-morpholinopropane (DLin-MA); and Dlin-KC2-DMA (CAS No. 1190197-97-7).

In embodiments, the lipid conjugate includes a helper lipid. The helper lipid may be, or derivied from, any suitable helper lipid. Any suitable helper lipid may be used. In embodiments, the conjugated lipid includes a helper lipid that is a cationic lipid. Cation lipids have a head group that has at least one postive formal charge. The cationic lipid may be, or derived from, any suitable cationic lipid. Examples of suitable cationic lipids include, but are not limtied to 14:0 TAP (CAS No. 197974-74-6); 16:0 TAP (CAS No. 139984-36-4); 18:0 TAP (CAS No. 220609-41-6); 18:1 TAP (also known as DOTAP; 144189-73-1); DC-6-14 (CAS No. 107086-76-0); 12:0 EPC (CAS No. 474945-22-7); 14:0 EPC (CAS No. 186492-53-5); 16:0 EPC (CAS No. 328250-18-6); 18:0 EPC salt (CAS No. 328268-13-9); 14:1 EPC (CAS No. 1246304-44-8); 18:1 EPC (CAS No. 474945-24-9); 16:0-18:1 EPC (CAS No. 328250-19-7); 18:0 DDAB (CAS No. 3700-67-2); DOTAP (CAS No. 132172-61-3); DOTMA (CAS No. 104162-48-3); DODAC (CAS No. 7212-69-3); DORI (CAS No. 153312-59-5); and DOSPA (CAS No. 2847775-87-3).

In embodiments, the helper lipid is a phospholipid. In embodiments, the phospholipid is zwitter-ionic at physiological pH. In embodiments, the phospholipid is an anion at physiological pH. In embodiments, the phospholipid is neutral at physiological pH. In embodiments, the phospholipid is positively charged a physiological pH. Examples of suitable helper lipids that are phospholipids include distearoylphosphatidylcholine (DSPC; CAS No. 816-94-4); dioleoylphosphatidyl choline (DOPC; CAS No. 4235-95-4); dipalmitoylphosphatidylcholine (DPPC; CAS No. 63-89-8); dioleoylphosphatidylglycerol (DOPG; CAS No. 67254-28-8); dipalmitoylphosphatidylglycerol (DPPG; CAS No. 200880-41-7); dioleoyl-phosphatidylethanolamine (DOPE; CAS NO. 4004 May 1); palmitoyloleoylphosphatidylcholine (POPC; CAS No. 26853-31-6); palmitoyloleoyl-phosphatidylethanolamine (POPE; 26662-94-2); dipalmitoyl phosphatidyl ethanolamine (DPPE; CAS No. 923-61-5); dimyristoylphosphoethanolamine (DMPE; CAS No. 998-07-2); distearoyl-phosphatidylethanolamine (DSPE; CAS No. 1069-79-0); 16-O-monomethyl PE (CAS No. 3930-13-0); 16-O-dimethyl PE (CAS No. 3922-61-0); 18-1-trans PE (CAS No. 19805-18-6); 1-stearioyl-2-oleoyl-phosphatidyethanol amine (SOPE; CAS No. 6418-95-7); 1,2-dielaidoyl-sn-glycero-3 phophoethanolamine (transDOPE); other various phosphatidylglycerols (i.e., a lipid having a two acyl chains esterified to glycerol where the glycerol is bonded to phosphate head group that has no compensating charges) such as cardiolipin, diacylphosphatidylserine, diacylphosphatidic acid, N-Succinylphosphatidylethanolamines, N-dodecanoylphosphatidylethanolamines, N-glutarylphosphatidylethanolamines, and palmitoyloleyolphosphatidylglycerol (POPG); lysylphosphatidylglycerols; 12:0 EPC (CAS No. 474945-22-7); 14:0 EPC (CAS No. 186492-53-5); 16:0 EPC (CAS No. 328250-18-6); or 18:0 EPC salt (CAS No. 328268-13-9).

### PEGylated Lipid Conjugates

In embodiments, the lipid conjugate includes a PEGylated lipid. As used herein, “PEGylated lipid conjugate” refers to a PEGylated lipid conjugated to a delivery construct.

PEGylated lipids are derived from lipids. PEGylated lipids may be derived from any suitable lipid. Examples of suitable lipids include saturated free fatty acids such as stearic acid, caprylic acid, capric acid, lauric acid, myristic acid, palmitic acid, arachidic acid, behenic acid, lignoceric acid, and/or cerotic acid; unsaturated free fatty acids such as oleic acid, palmitoleic acid, elaidic acid, linoleic acid, linoelaidic acid, arachidonic acid, and/or erucic acid; monoglycerides derived from fatty acids, diglycerides derived from fatty acids; phospholipids derived from fatty acids; phosphatidylglycerols derived from fatty acids; and combinations thereof.

As used herein, “PEGylated” lipid refers to any lipid that includes one to 50 polyethylene glycol (PEG) repeats. In embodiments, the number of PEG repeats is 1 or greater, 5 or greater, 10 or greater, 15 or greater, 20 or greater, 30 or greater, 40 or greater, 45 or greater, 50 or greater, or 60 or greater, 70 or greater, 80 or greater, or 90 or greater. In embodiments, the number of PEG repeats 100 or less, 90 or less, 80 or less, 70 or less, 60 or less, 50 or less, 45 or less, 40 or less, 30 or less, 20 or less, 15 or less, 10 or less, or 5 or less. In embodiments, the number of PEG repeats is 1 to 80, 1 to 60, 1 to 50, 1 to 45, 1 to 40, 1 to 30, 1 to 20, 1 to 15, 1 to 10, or 1 to 5. In embodiments, the number of PEG repeats is 5 to 80, 5 to 70, 5 to 60, 5 to 50, 5 to 45, 5 to 40, 5 to 30, 5 to 20, 5 to 15, or 5 to 10. In embodiments, the number of PEG repeats is 10 to 80, 10 to 70, 10 to 60, 10 to 50, 10 to 45, 10 to 40, 10 to 30, 10 to 20, or 10 to 15. In embodiments, the number of PEG repeats is 15 to 80, 15 to 70, 15 to 60, 15 to 50, 15 to 45, 15 to 40, 15 to 30, or 15 to 20. In embodiments, the number of PEG repeats is 20 to 80, 20 to 70, 20 to 60, 20 to 50, 20 to 45, 20 to 40, or 20 to 30. In embodiments, the number of PEG repeats is 40 to 80, 40 to 70, 40 to 60, 40 to 50, or 45 to 50. In embodiments, the number of PEG repeats is 40, 41, 42, 43, 44, 45, 46, 47, 48, 49, or 50.

In embodiments, the number of PEG units in a PEGylated lipid is described by the average molecular weight of the lipid. In embodiments, the PEGylated lipid has an average molecular weight of 10000 grams per mol (g/mol) or less, 7500 g/mol or less, 5000 g/mol or less, 2500 g/mol or less, 2000 g/mol or less, 1900 g/mol or less, 1800 g/mol or less, 1700 g/mol or less, 1600 g/mol or less, 1500 g/mol or less, 1400 g/mol or less, 1300 g/mol or less, 1200 g/mol or less, 1100 g/mol or less, 1000 g/mol or less, 900 g/mol or less, 800 g/mol or less, 700 g/mol or less, 600 g/mol or less, or 500 g/mol or less. In embodiments, the PEGylated lipid has an average molecular weight of 400 g/mol or greater, 500 g/mol or greater, 600 g/mol or greater, 700 g/mol or greater, 800 g/mol or greater, 900 g/mol or greater, 1000 g/mol or greater, 1100 g/mol or greater, 1200 g/mol or greater, 1300 g/mol or greater, 1400 g/mol or greater, 1500 g/mol or greater, 1600 g/mol or grater, 1700 g/mol or greater, 1800 g/mol or greater, 1900 g/mol or greater, 2000 g/mol or greater, 2500 g/mol or greater, or 5000 g/mol or greater. In embodiments, the average molecular weight of the PEGylated lipid is 1000 g/mol to 2500 g/mol, 1500 g/mol to 2500 g/mol, or 1900 g/mol to 2100 g/mol.

The PEGylated lipid includes hydrophobic tail comprising one or more alkyl or alkenyl chains of C₅ to C₂₄. In embodiments, the hydrophobic tail includes one or more alkyl or alkenyl chains of C₅ or greater, C₁₀ or greater, C₁₅ or greater, or C₂₀ or greater. In embodiments, the hydrophobic tail includes one or more alkyl or alkenyl chains of C₂₄ or less, C₂₀ or less, C₁₅ or less, or C₁₀ or less. In embodiments, the hydrophobic tail includes one or more alkyl or alkenyl chains of C₅ to C₂₄, C₅ to C₂₀, C₅ to C₁₅, or C₅ to C₁₀. In embodiments, the hydrophobic tail includes one or more alkyl or alkenyl chains of C₁₀ to C₂₄, C₁₀ to C₂₀, or C₁₀ to C₁₅. In embodiments, the hydrophobic tail includes one or more alkyl or alkenyl chains of C₁₅ to C₂₄ or C₁₅ to C₂₀. In embodiments, the hydrophobic tail includes one or more alkyl or alkenyl chains of C₂₀ to C₂₄.

In embodiments, the PEGylated lipid is derived from an ionizable lipid or helper lipid such as those disclosed herein. In embodiments, the headgroup of the ionizable lipid or helper lipid may be modified to include a PEG chain. In embodiments where the PEGylated lipid is derived from an ionizable lipid or helper lipid, the resultant lipid may be neutral at physiological pH, positively charged at neutral pH, negatively charged at physiological pH, or possess a formal positive or negative charge. In embodiments, where the PEGylated lipid is derived from an ionizable or helper lipid, the ionizable or charged characteristics of the modified lipid may be the same or changed.

In embodiments, the PEGylated lipid is derived from a phospholipid. In embodiments, the PEGylated lipid is derived from a diglyceride (sometimes called a diacylglycerol).

In embodiments, the PEGylated lipid is a PEG-modified phosphatidylethanolamine, PEG-modified phosphatidic acid, a PEG-modified ceramide (e.g., PEG-CerC14 or PEG-CerC20), a PEG modified dialkylamine, a PEG-modified diacylglycerol, or a PEG-modified dialkylglycerols.

In embodiments the PEGylated lipid is PEGylated 1, 2-distearoyl-sn-glycero-3-phosphoethanolamine-poly (ethylene glycol) (DSPE-PEG), PEGylated 2-dipalmitoyl-sn-glycero-3-phosphoethanolamine-N-[(polyethylene glycol)-methoxy](DPPE-PEG); PEGylated 1,2-dioleoyl-sn-glycero-3-phosphoethanolamine-N-[amino (polyethylene glycol) (DOPE-PEG); PEGylated DMPE-PEG; PEGylated 1,2-dimyristoyl-sn-glycero-3-phosphoethanolamine-N-[(polyethylene glycol)-methoxy] (DMG-PEG); PEGylated DPG (DPG-PEG); PEGylated 1,2-distearoyl-rac-glycero-3-methylpolyoxyethylene (DSG-PEG); PEGylated 14:0 PE (14PE-PEG); PEGylated 16:0 PE (16PE-PEG); PEGylated 18:0 PE (18PE-PEG); PEGylated 18:1 PE (18-1PE-PEG); PEGylated C8 ceramide (C8-CER-PEG); PEGylated C16 ceramide (C16-CER-PEG); PEGylated stearic acid; or PEGylated pentacosadiynoic acid.

In embodiments, the conjugated PEGylated lipids may include the reaction product between a reactive handle (Rh) on a delivery construct and a PEGylated lipid of the following general structures:

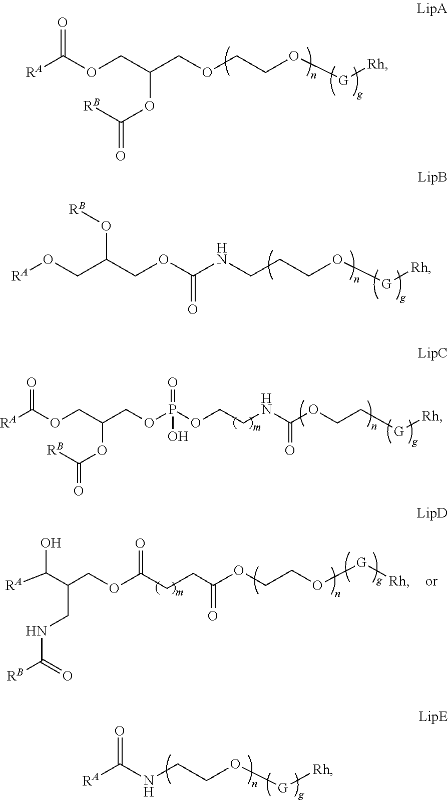

or an ionized form thereof wherein:
R^{A} and R^{B} (if present) are each independently an alkyl or alkenyl of C5 to C25, wherein one or more carbons of the alkyl or alkenyl are optionally replaced with a catenated heteroatom, optionally substituted with O to form a carbonyl, or both;
n is an integer between 1 and 50;
m (if present) is an integer between 0 and 10;
Rh is a reactive handle;
G is a spacer; and
g is 0 or 1.

In LipA, LipB, LipC, LipD, and LipE, R^{A} and R^{B} (if present) are each independently an alkyl or an alkenyl of C5 to C25. In embodiments, R^{A} and R^{B} are each independently an alkyl or alkenyl of C5 or greater, C8 or greater, C10 or greater, C12 or greater, C15 or greater, C18 or greater, C20 or greater, or C22 or greater. In embodiments, R^{A} and R^{B} are each independently an alkyl or alkenyl of C25 or less, C22 or less, C20 or less, C18 or less, C15 or less, C12 or less, C10 or less, or C8 or less. In embodiments, R^{A} and R^{B} are each independently an alkyl or alkenyl of C5 to C25, C5 to C22, C5 to C20, C5 to C18, C5 to C15, C5 to C12, C5 to C10, C5 to C8, C8 to C25, C8 to C22, C8 to C20, C8 to C18, C8 to C15, C8 to C12, C8 to C12, C8 to C10, C10 to C25, C10 to C22, C10 to C20, C10 to C18, C10 to C15, C10 to C12, C12 to C25, C12 to C22, C12 to C20, C12 to C18, C12 to C15, C15 to C25, C15 to C22, C15 to C20, C15 to C18, C18 to C25, C18 to C22, C18 to C20, C20 to C25, C20 to C22, or C22 to C25.

In embodiments, at least one of R_{A} and R^{B} are an alkyl of C13. In embodiments, R^{A} and R^{B} are an alkyl of C13. In embodiments, the lipid is of LipA wherein R^{A} and R^{B} are a C13 alkyl. In embodiments, the lipid is LipC wherein R^{A} and R^{B} are a C13 alkyl.

In embodiments, at least one of R^{A} and R^{B} are an alkyl of C13. In embodiments, R^{A} and R^{B} are an alkyl of C15. In embodiments, the lipid is LipA wherein R^{A} and R^{B} are a C15 alkyl. In embodiments, the lipid is LipC wherein R^{A} and R^{B} are a C15 alkyl.

In embodiments, at least one of R^{A} and R^{B} are an alkyl of C17. In embodiments, R^{A} and R^{B} are an alkyl of C17. In embodiments, the lipid is LipA wherein R^{A} and R^{B} are a C17 alkyl. In embodiments, the lipid is LipC wherein R^{A} and R^{B} are a C17 alkyl. In embodiments, the lipid of LipE wherein R^{A} is a C17 alkyl.

In embodiments, at least one of R^{A} and R^{B} are an alkyl of C7. In embodiments, R^{A} and R^{B} are an alkyl of C7.

In embodiments, at least one of R^{A} and R^{B} are an alkenyl of C17. In embodiments, R^{A} and RB are an alkenyl of C17. In embodiments, at least one of R^{A} and R^{B} are an alkenyl of C15. In embodiments, R^{A} and R^{B} are an alkenyl of C15. In embodiments, the lipid is LipC wherein R^{A} and R^{B} are a C17 alkenyl. In embodiments, the lipid is LipC wherein R^{A} and R^{B} are a C15 alkenyl.

In embodiments, at least one of R^{A} and R^{B} is an alkenyl of C17 and at least one of R^{A} and R^{B} is an alkyl of C7. In embodiments, R^{A} is an alkenyl of C17 and R^{B} is an alkyl of C7. In embodiments, the lipid is LipD wherein R^{A} is an alkenyl of C17 and R^{B} is an alkyl of C7.

In embodiments, at least one of R^{A} and R^{B} is an alkenyl of C15 and at least one of R^{A} and R^{B} is an alkyl of C15. In embodiments, R^{A} is an alkenyl of C15 and R^{B} is an alkyl of C15. In embodiments, the lipid is LipD wherein R^{A} is an alkenyl of C17 and R^{B} is an alkyl of C7.

In embodiments where R^{A} and/or R^{B} are alkenyl, the alkenyl may include one or more double bonds. The one or more double bonds may be located anywhere along the alkenyl. The first carbon of an alkenyl is the carbon that replaces R^{A} or R^{B} in the structure. In embodiments wherein R^{A} and/or R^{B} is an alkenyl of C17, the double bond may be between C8 and C9.

The alkyl or alkenyl group may include one or more catenated heteroatoms (e.g., O, S, or N). The alkyl or alkenyl group may include one or more carbonyls. The carbon of the carbonyl is catenated along the alkyl or alkenyl group. In embodiments, the alkyl or alkenyl group include one or more heteroatoms and one or more carbonyls. In embodiments, the alkyl or alkenyl includes one or more esters, amides, ureas, carbamates, or carbonates.

In LipA, LipB, LipC, LipD, and LipE, m may be 0, 1, 2, 3, 4, 5, 6, 7, 8, 9, or 10. In embodiments, m is 1. In embodiments, m is 2.

In LipA, LipB, LipC, LipD, and LipE, G is a spacer. The spacer may be an alkanediyl of C1 to C50. In embodiments, the spacer may be an alkanediyl of C1 or greater, C2 or greater, C3 or greater, C4 or greater, C5 or greater, C10 or greater, C15 or greater, C20 or greater, C30 or greater or C40 or greater. In embodiments, the spacer may an alkanediyl of C50 or less, C40 or less, C30 or less, C20 or less, C15 or less, C10 or less, C5 or less, C4 or less, C3, or less, or C2 or less. In embodiments, the spacer may be an alkanediyl of C1 to C50, C1 to C40, C1 to C30, C1 to C20, C1 to C15, C1 to C10, C1 to C5, C1 to C4, C1 to C3, C1 to C2, C2 to C50, C2 to C40, C2 to C30, C2 to C20, C2 to C15, C2 to C10, C2 to C10, C2 to C5, C2 to C4, C2 to C3, C3 to C50, C3 to C40, C3 to C30, C3 to C20, C3 to C15, C3 to C10, C3 to C5, C3 to C4, C4 to C50, C4 to C40, C4 to C30, C4 to C20, C4 to C15, C4 to C10, C4 to C5, C5 to C50, C5 to C40, C5 to C30, C5 to C20, C5 to C15, C5 to C10, C10 to C50, C10 to C40, C10 to C30, C10 to C20, C10 to C15, C15 to C50, C15 to C40, C15 to C30, C15 to C20, C20 to C50, C20 to C40, C20 to C30, C30 to C50, C30 to C40, or C40 to C50.

The spacer may include one or more catenated heteroatoms (e.g., O, S, or N). The spacer may include one or more carbonyls. The carbon of the carbonyl is catenated along the alkanediyl of the spear. In embodiments, the spacer includes one or more heteroatoms and one or more carbonyls. In embodiments, the spacer includes one or more esters, amides, ureas, carbamates, or carbonates.

In embodiments, the spacer is

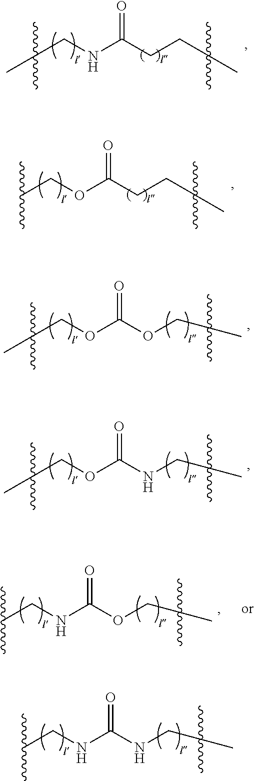

wherein l′ and l″ are each independently an integer from 0 to 10 (e.g., 1, 2, 3, 4, 5, 6, 7, 8, 9, or 10). In embodiments, l′ is 2 and l″ is 2. In embodiments, l′is 1 and l″ is 2.

g may be 1 or 0. In embodiments, g is 1. In embodiments g is 0. In embodiments where g is 0, the lipid does not include a spacer.

In LipA, LipB, LipC, LipD, and LipE, Rh is a reactive handle. The reactive handle may be any reactive handle as disclosed herein. The reactive handle is configured to react with a reactive handle on the delivery construct to form the lipid conjugate.

In LipA, LipB, LipC, LipD, and LipE n describes the number of PEG units. As such, n may be any number of PEG units as described herein.

In embodiments, the conjugated PEGylated lipids include the reaction product between a reactive handle on a delivery construct and a DSPE derived PEGylated lipid of Formula LipX

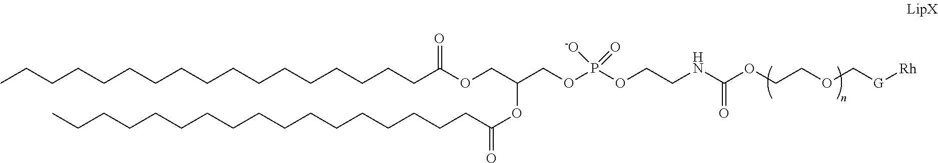

wherein n, G, and Rh are as defined herein. In embodiments, n is an integer from 10 to 100, 10 to 50, 10 to 20, or 30 to 50. In embodiments, n is 12. In embodiments, n is 44.

In embodiments, the lipid if Formula LipX is of Formula LipX (A)

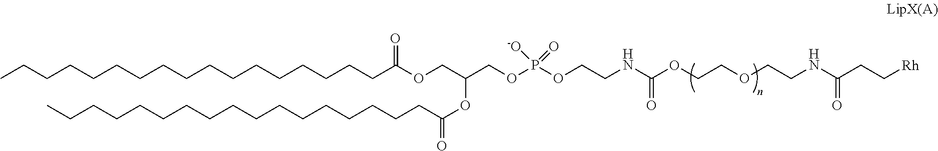

wherein n and Rh are as defined herein.

In embodiments, the lipid if Formula LipX(A) is of Formula LipX(A)(i)

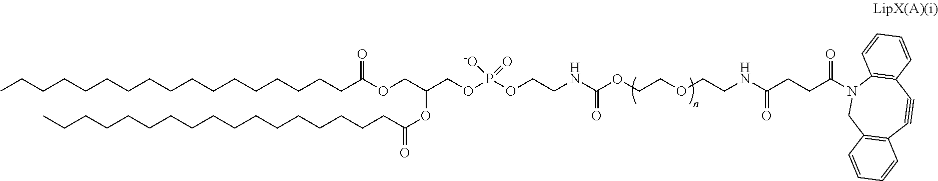

wherein n is as defined herein.

As described elsewhere herein, the reactive handle on the PEGylated lipids can be reacted with a cooperative reactive handle on a delivery construct to form the bonding group M of the linker. For example, the reactive handle of a PEGylated lipid of Formula LipA, LipB, LipC, LipD, LipE, LiX, LipX(A), or LipX(A)(i), may be reacted with the reactive handle of an EEV of Formula J1, J2, J3, or JJ1 to form a bonding group M.

In embodiments, the lipid conjugate comprises Formula J1c, J2c, J3c, or JJ1c (as described herein), wherein R¹⁰⁰ is

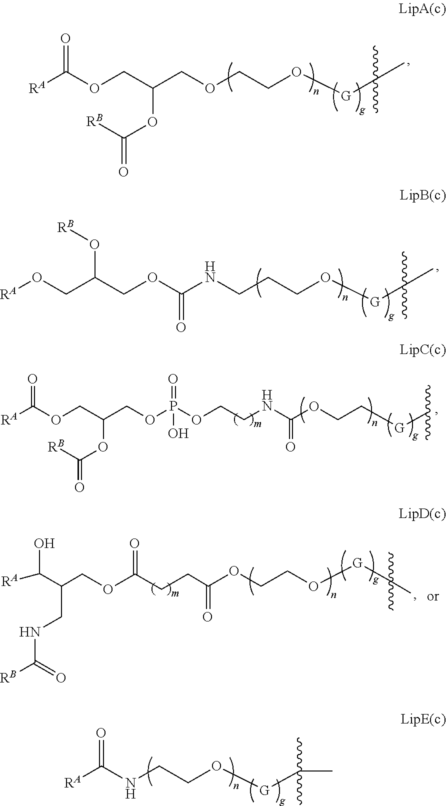

wherein R^{A}, R^{B}, n, g, m, and G of LipA(c), LipB(c), LipC(c), LipD(c), and LipE(c) are defined herein relative to LipA, LipB, LipC, LipD, and LipE.

In embodiments, the lipid conjugate comprises Formula LipX(c) or LipX(A)(c)

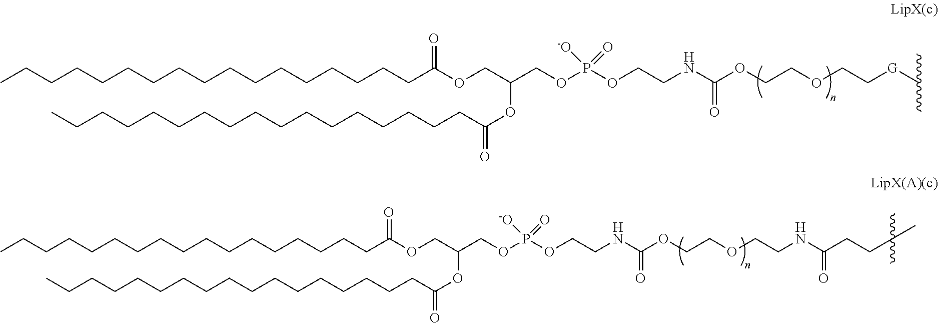

wherein G and n are defined herein.

Also provided are methods for synthesizing conjugated lipids. The method includes forming a mixutre that includes a PEGylated lipid, helper lipid, ionizable lipid, cationic lipid, or combinations thereof where the PEGylated lipid, helper lipid, ionizable lipid, cationic lipid includes a first reactive handle and a delivery construct that includes a second reactive handle. The method further includes allowing the conjugaiton reaction to proceed to form the conjugated lipid.

The first reactive handle and the second reactive handle should be compatible for a conjugation reaction. Example conjugation reactions include, but are not limited to, malemide conjugtion, N-hydroxysuccinimide (NHS) ester conjugation, click chemistry, and amide bond formation. In embodiments where a maleimide conjugation reaction is desired, one of the reactive handles includes a thiol or thiolate and the other reactive handle includes a maleimide group. In embodiments where an NHS ester conjugation reaction is desired, one of the reactive handles includes an amine and the other reactive handle includes an NHS ester group.

In embodiments where an amide bond formation conjugation reaction is desired, one reactive handle includes a carboxylic acid, or an activated carboxylic acid and the other reactive handle includes an amine. To form an activated carboxylic acid, the carboxylic acid may be reacted with an activating group such as various carbodiimides.

In embodiments where a click conjugation reaction is desired, one of the reactive handles includes an alkyne and the other reactive handle includes an azide. In embodiments, the click reaction is catalyst free. In embodiments, the click conjugation reaction is a strain-promoted click reaction where the alkyne is within a strained ring, such as, for example, a cyclooctyne.

The mixture may further include a solvent. Any solvent that confers solubility to the PEGylated lipid, helper lipid, ionizable lipid, cationic lipid, or combinations thereof, and the delivery construct may be used. In embodiments, the solvent is a mixture of acetonitrile and water. The mixture may further include catalysts, for example, including acids, bases, and/or metals.

The temperature of the reaction may be any temperature that does not induce decomposition of the reaction components or of the conjugated lipid product. In embodiments, the reaction temperature is between 4° C. and 60° C.

The stoichiometry of the PEGylated lipid to the delivery construct may vary.

The reaction may be monitored and/or characterized using a variety of methods known in the art including, but not limited to mass specrometry and size exclusion chromatography.

### Lipid-Based Particles Including a Lipid Conjugate

In embodiments, lipid-based particles are provided. The lipid-based particle may form a lipid nanoparticle (LNP). The lipid-based particle may form a liposome. In embodiments, the lipid-based particle includes a payload encapsulated within the LNP and/or liposome.

In embodiments, “decorated” lipid-based particles are provided. As used herein, the term “decorated lipid-based particles” refers to lipid-based particles that include one or more lipid conjugates. In embodiments, a liposome includes one or more lipid conjugates. In embodiments, an LNP includes one or more lipid conjugates. The lipid conjugates included in the lipid-based particle may be any of the lipid conjugates disclosed herein. In embodiments, the lipid conjugate may be included in the formulation used to make the decorated lipid-based particle. In embodiments, the lipid particle is made first and the lipid conjugate is subsequently formed to make the decorated lipid-based particle.

Without wishing to be bound by theory, it is thought that a delivery construct displayed on the exterior surface of a lipid-based particle may faciliates entry of the lipid-based particle into a cell. Additionally, it is thought that the delivery construct may facilaite endosomal escape of the components of the lipid-based particle.

### Lipid Nanoparticle (LNP)

In embodiments, the lipid-based particle is a lipid nanoparticle (LNP). In embodiments, the LNP includes a PEGylated lipid, a helper lipid, an ionizable, and a sterol. In embodiments, one or more of the PEGylated lipid, the helper lipid, or the ionizable lipid may be conjugated to a delivery construct form a lipid conjugate as described herein. In embodiments, the LNP comprises a lipid conjugate that includes a PEGylated lipid (also referred to as a PEGylated lipid conjugate).

The LNP may comprise non-conjugated versions of the lipid class of the lipid conjugate. For example, if the LNP comprises a lipid conjugate that includes a PEGylated lipid, the LNP may also comprise a PEGylated lipid that is not a lipid conjugate (a “non-conjugated PEGylated lipid”). In embodiments, the LNP comprises a lipid conjugate that includes a helper lipid and also comprises a helper lipid that is not a lipid conjugate (a “non-conjugated helper lipid”). In embodiments, the LNP comprises a lipid conjugate that includes an ionizable and also comprises an ionizable or cationic lipid that is not a lipid conjugate (a “non-conjugated ionizable lipid”). In embodiments, the lipid conjugates may be derived from the same lipid structure as the non-conjugated lipids. For example, if an LNP includes a lipid conjugate derived from PEGylated lipid X, the LNP may also include unconjugated PEGylated lipid X. In embodiments, the lipid conjugates are derived from a different lipid structure than the non-conjugated lipids. For example, if an LNP includes a lipid conjugate derived from PEGylated lipid X, the LNP may also include unconjugated PEGylated lipid Y.

In embodiments, the LNP does not comprise non-conjugated versions of the lipid conjugate. For example, the LNP can comprise a PEGylated lipid conjugate and not comprise a non-conjugated PEGylated lipid.

In embodiments, the LNP comprises (i) a lipid conjugate, which may be a PEGylated lipid conjugate, helper lipid conjugate, and/or an ionizable lipid conjugate; (ii) a PEGylated lipid, (iii) a helper lipid, (iv) an ionizable lipid; and (v) cholesterol or a derivative thereof.

In embodiments, the LNP comprises (i) a PEGylated lipid conjugate; (ii) a helper lipid, (iii) an ionizable lipid; and (iv) a sterol. In embodiments, the LNP further comprises a non-conjugated PEGylated lipid. In embodiments, the PEGylated lipid conjugate is derived from the PEGylated lipid. In embodiments, the PEGylated lipid conjugate is derived from a PEGylated lipid that is different from the non-conjugated PEGylated lipid.

The LNP may comprise sterol. In embodiments, the sterol comprises cholesterol or a derivative thereof. Examples of suitable cholesterol derivatives include, but are not limited to, DC-cholesterol, β-sitosterol, and BHEM-cholesterol. Additional suitable cholesterol derivatives include fucosterol, and campesterol.

The LNP may comprise any suitable helper lipid, ionizable lipid, PEGylated lipid, and sterol as disclosed herein. In embodiments, the LNPs include SM-102, D-Lin-MC3-DMA (also called MC3), or both as an ionizable lipid (FIG. 4). In embodiments, the LNPs include DSPC as a helper lipid (FIG. 4). In embodiments, the LNPs include DSPE-PEG as a PEGylated lipid.

In embodiments, the LNPs comprise a lipid conjugate; D-Lin-MC3-DMA or SM-102; DSPC; and cholesterol. In embodiments, the lipid conjugate is an EEV-PEGylated lipid conjugate. In embodiments, the PEGylated lipid conjugate is derived from DSPE-PEG. In embodiments, the LNPs further comprise a DSPE-PEG lipid.

The lipid conjugate, a PEGylated lipid, a helper lipid, an ionizable or cationic lipid, and sterol may be present in various amounts in the LNP.

The total amount of PEGylated lipids in an LNP may vary. The total amount of PEGylated lipids includes the amount of PEGylated lipid conjugate and the amount of non-conjugated PEGylated lipid. In embodiments, the total amount of PEGylated lipid in an LNP is 5 mol-% or less, 4 mol-% or less, 3.2 mol-% or less, 3.1 mol-% or less, 3 mol-% or less, 2.9 mol-% or less, 2.8 mol-% or less, 2.7 mol-% or less, 2.6 mol-% or less, 2.5 mol-% or less, 2.4 mol-% or less, 2.3 mol-% or less, 2.2 mol-% or less, 2.0 mol-% or less, 1.9 mol-% or less, 1.8 mol-% or less, 1.7 mol-% or less, 1.6 mol-% or less, 1.5 mol-% or less, 1.4 mol-% or less, 1.3 mol-% of less, 1.2 mol-% or less, 1.1 mol-% or less, 1.0 mol-% or less, 0.75 mol-% or less, 0.5 mol-% or less, 0.1 mol-% or less, or 0.05 mol-% or less. In embodiments, the total amount of PEGylated lipid in an LNP is 0.001 mol-% or greater, 0.05 mol-% or greater, 0.1 mol-% or greater, 0.25 mol-% or greater, 0.5 mol-% or greater, 0.75 mol-% or greater, 1.0 mol-% or greater 1.1 mol-% or greater, 1.2 mol-% or greater, 1.3 mol-% or greater, 1.4 mol-% or greater, 1.5 mol-% or greater, 1.6 mol-% or greater, 1.7 mol-% or greater, 1.8 mol-% or greater, 1.9 mol-% or greater, 2.0 mol-% or greater, 2.1 mol-% or greater, 2.2 mol-% or greater, 2.3 mol-% or greater, 2.4 mol-% or greater, 2.5 mol-% or greater, 2.6 mol-% or greater, 2.7 mol-% or greater, 2.8 mol-% or greater, 2.9 mol-% or greater, 3.0 mol-% or greater, 3.1 mol-% or greater, 3.2 mol-% or greater, or 4 mol-% or greater.

In embodiments, the lipid conjugate (e.g., PEGylated lipid conjugate) is present at 0.001 mol-% or greater, 0.0025 mol-% or greater, 0.005 mol-% or greater, 0.0075 mol-% or greater, 0.01 mol-% or greater, 0.02 mol-% or greater, 0.03 mol-% or greater, 0.04 mol-% or greater, 0.05 mol-% or greater, 0.06 mol-% or greater, 0.07 mol-% or greater, 0.08 mol-% or greater, 0.09 mol-% or greater, 0.1 mol-% or greater, 0.2 mol-% or greater, 0.3 mol-% or greater, 0.4 mol-% or greater, 0.5 mol-% or greater, 0.6 mol-% or greater, 0.7 mol-% or greater, 0.8 mol-% or greater, 0.9 mol-% or greater, 1.0 mol-% or greater, 1.25 mol-% or greater, 1.5 mol-% or greater, 1.75 mol-% or greater, 2 mol-% or greater, 2.25 mol-% or greater, 2.5 mol-% or greater, 2.75 mol-% or greater, 3.0 mol-% or greater, 3.25 mol-% or greater, 3.5 mol-% or greater, 3.75 mol-% or greater, 4.0 mol-% or greater, 4.25 mol-% or greater, 4.5 mol-% or greater, or 4.75 mol-% or greater. In embodiments, the lipid conjugate (e.g., PEGylated lipid conjugate) is present in the LNP at 5.0 mol-% or less, 4.75 mol-% or less, 4.5 mol-% or less, 4.25 mol-% or less, 4.0 mol-% or less, 3.75 mol-% or less, 3.5 mol-% or less, 3.25 mol-% or less, 3 mol-% or less, 2.75 mol-% or less, 2.5 mol-% or less, 2.25 mol-% or less, 2.0 mol-% or less, 1.75 mol-% or less, 1.5 mol-% or less, 1.25 mol-% or less, 1.0 mol-% or less, 0.9 mol-% or less, 0.8 mol-% or less, 0.7 mol-% or less, 0.6 mol-% or less, 0.5 mol-% or less, 0.4 mol-% or less, 0.3 mol-% or less, 0.2 mol-% or less, 0.1 mol-% or less,0.09 mol-% or less, 0.08 mol-% or less, 0.07 mol-% or less, 0.06 mol-% or less, 0.05 mol-% or less, 0.04 mol-% or less, 0.03 mol-% or less, 0.02 mol-% or less, 0.01 mol-% or less, 0.0075 mol-% or less, or 0.005 mol-% or less. In embodiments, the lipid conjugate (e.g., PEGylated lipid conjugate) is present at 0.0025 mol-% to 1.5 mol-% 0.01 mol-% to 1 mol-%, or 0.02 mol-% to 0.09 mol-%

In embodiments, the non-conjugated PEGylated lipid is present at 0.001 mol-% or greater, 0.0025 mol-% or greater, 0.005 mol-% or greater, 0.0075 mol-% or greater, 0.01 mol-% or greater, 0.02 mol-% or greater, 0.03 mol-% or greater, 0.04 mol-% or greater, 0.05 mol-% or greater, 0.06 mol-% or greater, 0.07 mol-% or greater, 0.08 mol-% or greater, 0.09 mol-% or greater, 0.1 mol-% or greater, 0.2 mol-% or greater, 0.3 mol-% or greater, 0.4 mol-% or greater, 0.5 mol-% or greater, 0.6 mol-% or greater, 0.7 mol-% or greater, 0.8 mol-% or greater, 0.9 mol-% or greater, 1.0 mol-% or greater, 1.25 mol-% or greater, 1.5 mol-% or greater, 1.75 mol-% or greater, 2 mol-% or greater, 2.25 mol-% or greater, 2.5 mol-% or greater, 2.75 mol-% or greater, 3 mol-% or greater, 3.25 mol-% or greater, 3.5 mol-% or greater, 3.75 mol-% or greater, 4 mol-% or greater, 4.25 mol-% or greater, 4.5 mol-% or greater, or 4.75 mol-% or greater. In embodiments, the non-conjugated PEGylated lipid is present in the LNP at 5 mol-% or less, 4.75 mol-% or less, 4.5 mol-% or less, 4.25 mol-% or less, 4 mol-% or less, 3.75 mol-% or less, 3.5 mol-% or less, 3.25 mol-% or less, 3 mol-% or less, 2.75 mol-% or less, 2.5 mol-% or less, 2.25 mol-% or less, 2.0 mol-% or less, 1.75 mol-% or less, 1.5 mol-% or less, 1.25 mol-% or less, 1.0 mol-% or less, 0.9 mol-% or less, 0.8 mol-% or less, 0.7 mol-% or less, 0.6 mol-% or less, 0.5 mol-% or less, 0.4 mol-% or less, 0.3 mol-% or less, 0.2 mol-% or less, 0.1 mol-% or less,0.09 mol-% or less, 0.08 mol-% or less, 0.07 mol-% or less, 0.06 mol-% or less, 0.05 mol-% or less, 0.04 mol-% or less, 0.03 mol-% or less, 0.02 mol-% or less, 0.01 mol-% or less, 0.0075 mol-% or less, or 0.005 mol-% or less. In embodiments, the non-conjugated PEGylated lipid is present at 0.0025 mol-% to 1.5 mol-% 0.01 mol-% to 1 mol-%, or 0.02 mol-% to 0.09 mol-%.

In embodiments, the helper lipid is present in the LNP at 5 mol-% or greater, 7.5 mol-% or greater, 10 mol-% or greater, 12.5 mol-% or greater, 15 mol-% or greater, 17.5 mol-% or greater, 20 mol-% or greater, 25 mol-% or greater, 30 mol-% or greater, 35 mol-% or greater, 40 mol-% or greater, or 45 mol-% or greater. In embodiments, the helper lipid is present in the LNP at 50 mol-% or less, 45 mol-% or less, 40 mol-% or less, 35 mol-% or less, 30 mol-% or less, 25 mol-% or less, 17.5 mol-% or less, 15 mol-% or less, 12.5 mol-% or less, 10 mol-% or less, or 7.5 mol-% or less. In embodiments, the helper lipid is present in the LNP at 5 mol-% to 15 mol-%, 5 mol-% to 12.5 mol-%, 5 mol-% to 10 mol-%, or 5 mol-% to 7.5 mol-%. In embodiments, the helper lipid is present in the LNP at 7.5 mol-% to 15 mol-%, 7.5 mol-% to 12.5 mol-%, or 7.5 mol-% to 10 mol-%. In embodiments, the helper lipid is present in the LNP at 10 mol-% to 15 mol-% or 10 mol-%. In embodiments, the helper lipid is present in the LNP at 12.5 mol-% to 15 mol-%.

In embodiments, cholesterol or derivative thereof, is present in the LNP at 5 mol-% or greater, 10 mol-% or greater, 15 mol-% or greater, 20 mol-% or greater, 30 mol-% or greater, 35 mol-% or greater, 40 mol-% or greater, or 50 mol-% or greater. In embodiments, cholesterol or derivative thereof, is present in the LNP at 60 mol-% or less, 50 mol-% or less, 40 mol-% or less, 35 mol-% or less, 30 mol-% or less, 25 mol-% or less, 20 mol-% or less, 15 mol-% or less, or 10 mol-% or less. In embodiments, cholesterol or derivative thereof, is present in the LNP at 30 mol-% to 60 mol-%, 30 mol-% to 50 mol-%, 30 mol-% to 40 mol-%, or 35 mol-% to 40 mol-%. In embodiments, cholesterol or derivative thereof, is present in the LNP at 40 mol-% to 60 mol-% or 40 mol-%. In embodiments, cholesterol or derivative thereof, is present in the LNP at 50 mol-% to 60 mol-%.

In embodiments, the ionizable lipid, is present in the LNP at 20 mol-% or greater, 30 mol-% or greater, 40 mol-% or greater, 45 mol-% or greater, 50 mol-% or greater, or 55 mol-% or greater. In embodiments, the ionizable lipid, is present in the LNP at 60 mol-% or less, 55 mol-% or less, 50 mol-% or less, 45 mol-% or less, 40 mol-% or less, or 20 mol-% or less. In embodiments, the ionizable lipid or cationic lipid, is present in the LNP at 30 mol-% to 60 mol-%, 30 mol-% to 50 mol-%, or 30 mol-% to 40 mol-%. In embodiments the ionizable lipid, is present in the LNP at 40 mol-% to 60 mol-% or 40 mol-%. In embodiments, the ionizable lipid is present in the LNP at 50 mol-% to 60 mol-%.

In embodiments, the LNP may include an ionizable lipid; helper lipid, sterol; lipid conjugate; and total amount of PEGylated lipids in the amounts of any one of LNP formulations LNP1-LNP75 in Table 5. In embodiments, the lipid conjugate of Table 5 is a PEGylated lipid conjugate.

TABLE 5
Various LNP formulations

| | | | Ion- | | |
|---|---|---|---|---|---|
| LNP | Total | lipid | izable | Helper | |
| formu- | PEGylated | conjugate | lipid | lipid | Sterol |
| lation | lipid mol-% | mol-% | mol-% | mol-% | mol-% |
| LNP1 | 0.001-5.0 | 0.001-5.0 | 20-60 | 5.0-50 | 5.0-60 |
| LNP2 | 0.001-5.0 | 0.001-3.0 | 30-60 | 5.0-15 | 20-60 |
| LNP3 | 0.001-5.0 | 0.0075-0.2 | 40-60 | 7.5-15 | 30-40 |
| LNP4 | 0.001-5.0 | 0.0075-0.08 | 45-55 | 7.5-12.5 | 35-40 |
| LNP5 | 0.001-5.0 | 0.01-0.06 | 45-55 | 7.5-12.5 | 35-40 |
| LNP6 | 0.25-5.0 | 0.001-5.0 | 20-60 | 5.0-50 | 5.0-60 |
| LNP7 | 0.25-5.0 | 0.001-3.0 | 30-60 | 5.0-15 | 20-60 |
| LNP8 | 0.25-5.0 | 0.0075-0.2 | 40-60 | 7.5-15 | 30-40 |
| LNP9 | 0.25-5.0 | 0.0075-0.08 | 45-55 | 7.5-12.5 | 35-40 |
| LNP10 | 0.25-5.0 | 0.01-0.06 | 45-55 | 7.5-12.5 | 35-40 |
| LNP11 | 1.0-4.0 | 0.001-4.0 | 20-60 | 5.0-50 | 5.0-60 |
| LNP12 | 1.0-4.0 | 0.001-3.0 | 30-60 | 5.0-15 | 20-60 |
| LNP13 | 1.0-4.0 | 0.0075-0.2 | 40-60 | 7.5-15 | 30-40 |
| LNP14 | 1.0-4.0 | 0.0075-0.08 | 45-55 | 7.5-12.5 | 35-40 |
| LNP15 | 1.0-4.0 | 0.01-0.06 | 45-55 | 7.5-12.5 | 35-40 |
| LNP16 | 1.0-3.5 | 0.001-3.5 | 20-60 | 5.0-50 | 5.0-60 |
| LNP17 | 1.0-3.5 | 0.001-3.0 | 30-60 | 5.0-15 | 20-60 |
| LNP18 | 1.0-3.5 | 0.0075-0.2 | 40-60 | 7.5-15 | 30-40 |
| LNP19 | 1.0-3.5 | 0.0075-0.08 | 45-55 | 7.5-12.5 | 35-40 |
| LNP20 | 1.0-3.5 | 0.01-0.06 | 45-55 | 7.5-12.5 | 35-40 |
| LNP21 | 1.0-3.0 | 0.001-3.0 | 20-60 | 5.0-50 | 5.0-60 |
| LNP22 | 1.0-3.0 | 0.001-3.0 | 30-60 | 5.0-15 | 20-60 |
| LNP23 | 1.0-3.0 | 0.0075-0.2 | 40-60 | 7.5-15 | 30-40 |
| LNP24 | 1.0-3.0 | 0.0075-0.08 | 45-55 | 7.5-12.5 | 35-40 |
| LNP25 | 1.0-3.0 | 0.01-0.06 | 45-55 | 7.5-12.5 | 35-40 |
| LNP26 | 1.0-2.0 | 0.001-2.0 | 20-60 | 5.0-50 | 5.0-60 |
| LNP27 | 1.0-2.0 | 0.001-2.0 | 30-60 | 5.0-15 | 20-60 |
| LNP28 | 1.0-2.0 | 0.0075-0.2 | 40-60 | 7.5-15 | 30-40 |
| LNP29 | 1.0-2.0 | 0.0075-0.08 | 45-55 | 7.5-12.5 | 35-40 |
| LNP30 | 1.0-2.0 | 0.01-0.06 | 45-55 | 7.5-12.5 | 35-40 |
| LNP31 | 1.2-1.8 | 0.001-1.8 | 20-60 | 5.0-50 | 5.0-60 |
| LNP32 | 1.2-1.8 | 0.001-1.8 | 30-60 | 5.0-15 | 20-60 |
| LNP33 | 1.2-1.8 | 0.0075-0.2 | 40-60 | 7.5-15 | 30-40 |
| LNP34 | 1.2-1.8 | 0.0075-0.08 | 45-55 | 7.5-12.5 | 35-40 |
| LNP35 | 1.2-1.8 | 0.01-0.06 | 45-55 | 7.5-12.5 | 35-40 |
| LNP36 | 1.3-1.7 | 0.001-1.7 | 20-60 | 5.0-50 | 5.0-60 |
| LNP37 | 1.3-1.7 | 0.001-1.7 | 30-60 | 5.0-15 | 20-60 |
| LNP38 | 1.3-1.7 | 0.0075-0.2 | 40-60 | 7.5-15 | 30-40 |
| LNP39 | 1.3-1.7 | 0.0075-0.08 | 45-55 | 7.5-12.5 | 35-40 |
| LNP40 | 1.3-1.7 | 0.01-0.06 | 45-55 | 7.5-12.5 | 35-40 |
| LNP41 | 1.4-1.6 | 0.001-1.6 | 20-60 | 5.0-50 | 5.0-60 |
| LNP42 | 1.4-1.6 | 0.001-1.6 | 30-60 | 5.0-15 | 20-60 |
| LNP43 | 1.4-1.6 | 0.0075-0.2 | 40-60 | 7.5-15 | 30-40 |
| LNP44 | 1.4-1.6 | 0.0075-0.08 | 45-55 | 7.5-12.5 | 35-40 |
| LNP45 | 1.4-1.6 | 0.01-0.06 | 45-55 | 7.5-12.5 | 35-40 |
| LNP46 | 1.5-3.2 | 0.001-3.2 | 20-60 | 5.0-50 | 5.0-60 |
| LNP47 | 1.5-3.2 | 0.001-3.0 | 30-60 | 5.0-15 | 20-60 |
| LNP48 | 1.5-3.2 | 0.0075-0.2 | 40-60 | 7.5-15 | 30-40 |
| LNP49 | 1.5-3.2 | 0.0075-0.08 | 45-55 | 7.5-12.5 | 35-40 |
| LNP50 | 1.5-3.2 | 0.01-0.06 | 45-55 | 7.5-12.5 | 35-40 |
| LNP51 | 1.8-3.2 | 0.001-3.2 | 20-60 | 5.0-50 | 5.0-60 |
| LNP52 | 1.8-3.2 | 0.001-3.0 | 30-60 | 5.0-15 | 20-60 |
| LNP53 | 1.8-3.2 | 0.0075-0.2 | 40-60 | 7.5-15 | 30-40 |
| LNP54 | 1.8-3.2 | 0.0075-0.08 | 45-55 | 7.5-12.5 | 35-40 |
| LNP55 | 1.8-3.2 | 0.01-0.06 | 45-55 | 7.5-12.5 | 35-40 |
| LNP56 | 0.001-0.5 | 0.001-0.5 | 20-60 | 5.0-50 | 5.0-60 |
| LNP57 | 0.001-0.5 | 0.001-0.5 | 30-60 | 5.0-15 | 20-60 |
| LNP58 | 0.001-0.5 | 0.0075-0.2 | 40-60 | 7.5-15 | 30-40 |
| LNP59 | 0.001-0.5 | 0.0075-0.08 | 45-55 | 7.5-12.5 | 35-40 |
| LNP60 | 0.001-0.5 | 0.01-0.06 | 45-55 | 7.5-12.5 | 35-40 |
| LNP61 | 2.8-3.2 | 0.001-3.2 | 20-60 | 5.0-50 | 5.0-60 |
| LNP62 | 2.8-3.2 | 0.001-3.0 | 30-60 | 5.0-15 | 20-60 |
| LNP63 | 2.8-3.2 | 0.0075-0.2 | 40-60 | 7.5-15 | 30-40 |
| LNP64 | 2.8-3.2 | 0.0075-0.08 | 45-55 | 7.5-12.5 | 35-40 |
| LNP65 | 2.8-3.2 | 0.01-0.06 | 45-55 | 7.5-12.5 | 35-40 |
| LNP66 | 0.01-0.5 | 0.001-0.5 | 20-60 | 5.0-50 | 5.0-60 |
| LNP67 | 0.01-0.5 | 0.001-0.5 | 30-60 | 5.0-15 | 20-60 |
| LNP68 | 0.01-0.5 | 0.0075-0.2 | 40-60 | 7.5-15 | 30-40 |
| LNP69 | 0.01-0.5 | 0.0075-0.08 | 45-55 | 7.5-12.5 | 35-40 |
| LNP70 | 0.01-0.5 | 0.01-0.06 | 45-55 | 7.5-12.5 | 35-40 |
| LNP71 | 0.02-0.06 | 0.001-0.06 | 20-60 | 5.0-50 | 5.0-60 |
| LNP72 | 0.02-0.06 | 0.002-0.06 | 30-60 | 5.0-15 | 20-60 |
| LNP73 | 0.02-0.06 | 0.0075-0.06 | 40-60 | 7.5-15 | 30-40 |
| LNP74 | 0.02-0.06 | 0.0075-0.06 | 45-55 | 7.5-12.5 | 35-40 |
| LNP75 | 0.02-0.06 | 0.02-0.05 | 45-55 | 7.5-12.5 | 35-40 |

The LNPs described herein may have any suitable size or any suitable average size. LNP size within a plurality of LNPs may vary. Methods of measuring the size of LNPs are known, for example, size exclusion chromatography. In embodiments, dynamic light scattering is used to measure the average hydrodynamic radius, referred to herein as the average size or average diameter, of a plurality of LNPs. For example, the average hydrodynamic radius can be measured according to the Particle Dimensional Analysis Test Method. In embodiments, the average hydrodynamic radius of a plurality of LNPs is 50 nm or greater, 75 nm or greater, 100 nm or greater, 125 nm or greater, 150 nm or greater, 175 nm or greater, 200 nm or greater, 225 nm or greater, 300 nm or greater, or 400 nm or greater according to the Particle Dimensional Analysis Test Method. In embodiments, the average hydrodynamic radius of a plurality of LNPs is 500 nm or less, 300 nm or less, 225 nm or less, 200 nm or less, 175 nm or less, 150 nm or less, 125 nm or less, 100 nm or less, or 75 nm or less according to the Particle Dimensional Analysis Test Method. In embodiments, the average hydrodynamic radius of a plurality of LNPs is 50 nm to 500 nm, 50 nm to 400 nm, 50 nm to 300 nm, 50 nm to 225 nm, 50 nm to 200 nm, 50 nm to 175 nm, 50 nm to 150 nm, 50 to 125 nm, 50 nm to 100 nm, or 50 to 75 nm according to the Particle Dimensional Analysis Test Method. In embodiments, the average hydrodynamic radius of a plurality of LNPs is 75 nm to 500 nm, 75 nm to 400 nm, 75 nm to 300 nm, 75 nm to 225 nm, 75 nm to 200 nm, 75 nm to 175 nm, 75 nm to 150 nm, 75 to 125 nm, or 75 nm to 100 nm according to the Particle Dimensional Analysis Test Method. In embodiments, the average hydrodynamic radius of a plurality of LNPs is 100 nm to 500 nm, 100 nm to 400 nm, 100 nm to 300 nm, 100 nm to 225 nm, 100 nm to 200 nm, 100 nm to 175 nm, 100 nm to 150 nm, or 100 to 125 nm according to the Particle Dimensional Analysis Test Method. In embodiments, the average hydrodynamic radius a plurality of LNPs is 125 nm to 500 nm, 125 nm to 400 nm, 125 nm to 300 nm, 125 nm to 225 nm, 125 nm to 200 nm, 125 nm to 175 nm, or 125 nm to 150 nm according to the Particle Dimensional Analysis Test Method. In embodiments, the average hydrodynamic radius of a plurality of LNPs is 150 nm to 500 nm, 150 nm to 400 nm, 150 nm to 300 nm, 150 nm to 225 nm, 150 nm to 200 nm, or 150 nm to 175 nm according to the Particle Dimensional Analysis Test Method. In embodiments, the average hydrodynamic radius a plurality of LNPs is 175 nm to 500 nm, 175 nm to 400 nm, 175 nm to 300 nm, 175 nm to 225 nm, or 175 to 200 nm according to the Particle Dimensional Analysis Test Method. In embodiments, the average hydrodynamic radius a plurality of LNPs is 200 nm to 500 nm, 200 nm to 400 nm, 200 nm to 300 nm, or 200 nm to 225 nm according to the Particle Dimensional Analysis Test Method. In embodiments, the average hydrodynamic radius of a plurality of LNPs is 300 nm to 500 nm or 300 nm to 400 nm according to the Particle Dimensional Analysis Test Method. In embodiments, the average hydrodynamic radius of a plurality of LNPs is 400 nm to 500 nm according to the Particle Dimensional Analysis Test Method. In embodiments, the average hydrodynamic radius of a plurality of LNPs is 50 nm to 200 nm or 50 nm to 100 nm according to the Particle Dimensional Analysis Test Method.

The variance in LNP size is termed the poly dispersity index (PDI). The smaller the PDI, the more uniform the LNP size distribution. PDI may be measured using dynamic light scattering, size exclusion chromatography, or other methods known in the art. For example, the average hydrodynamic radius can be measured according to the Particle Dimensional Analysis Test Method. In embodiments, a plurality of LNPs may have a PDI of 0.05 or greater, 0.1 or greater, 0.2 or greater, 0.3 or greater, or 0.4 or greater according to the Particle Dimensional Analysis Test Method. In embodiments, a plurality of LNPs may have a PDI of a PDI of 0.5 or less, 0.4 or less, 0.3 or less, 0.2 or less, or 0.1 or less according to the Particle Dimensional Analysis Test Method. In embodiments, a plurality of LNPs may have a PDI of 0.05 to 0.5, 0.05 to 0.4, 0.05 to 0.3, 0.05 to 0.2, or 0.05 to 0.1 according to the Particle Dimensional Analysis Test Method. In embodiments, a plurality of LNPs may have a PDI of 0.1 to 0.5, 0.1 to 0.4, 0.1 to 0.3, or 0.1 to 0.2 according to the Particle Dimensional Analysis Test Method. In embodiments, a plurality of LNPs may have a PDI of 0.2 to 0.5, 0.2 to 0.4, or 0.2 to 0.3 according to the Particle Dimensional Analysis Test Method. In embodiments, a plurality of LNPs may have a PDI of 0.3 to 0.5 or 0.3 to 0.4 according to the Particle Dimensional Analysis Test Method. In embodiments, a plurality of LNPs may have a PDI of 0.4 to 0.5 according to the Particle Dimensional Analysis Test Method. In embodiments, a plurality of LNPs may have a PDI of 0.05 to 0.2 or 0.05 to 0.1 according to the Particle Dimensional Analysis Test Method.

In embodiments, an LNP as described herein is loaded with therapeutic payload. The LNP encapsulates the therapeutic payload such that the therapeutic payload is within a core (interior) of the LNP. The therapeutic payload can be a biologic therapeutic, such as a peptide or oligonucleotide, or can be a small molecule therapeutic. In embodiments, the therapeutic payload comprises an oligonucleotide as described elsewhere herein. In embodiments, the therapeutic payload is RNA. In embodiments, the therapeutic payload is mRNA. The payload, or components thereof, may be conjugated or may not be conjugated to a delivery construct.

Encapsulation efficiency (EE or E.E.) is a measure of how much payload is encapsulated into an LNP or a plurality of LNPs. Encapsulation efficiency is given as the quotient of total payload attempted to load divided by the payload loaded into the LNP or a plurality of LNPs. To determine percent EE, the quotient is multiplied by 100. Any method or assay may be used to measure percent EE. An example of an assay that may be used is the QUANT-IT Ribo green RNA assay kit (available from Sigma; see the Encapsulation Efficiency Test Method). In embodiments, an LNP or a plurality of LNPs has a percent EE of 80% or greater, 85% or greater, 90% or greater 95% or greater according to the Encapsulation Efficiency Test Method. In embodiments, an LNP or a plurality of LNPs has a percent EE of 99% or less, 95% or less, 90% or less, or 80% or less according to the Encapsulation Efficiency Test Method. In embodiments, an LNP or a plurality of LNPs has a percent EE of 80% to 99%, 80% to 95%, 80% to 90%, or 80% to 85% according to the Encapsulation Efficiency Test Method. In embodiments, an LNP or a plurality of LNPs has a percent EE of 85% to 99%, 85% to 95%, or 85% to 90% according to the Encapsulation Efficiency Test Method. In embodiments, an LNP or a plurality of LNPs has a percent EE of 90% to 99% or 90% according to the Encapsulation Efficiency Test Method. In embodiments, an LNP has a percent EE of 95% to 99%. In embodiments, an LNP or a plurality of LNPs has a percent EE of 90% to 99% or 95% to 99% according to the Encapsulation Efficiency Test Method.

The amount of payload encapsulated into an LNP, liposome, plurality of LNPs, or plurality of liposomes may vary by application. Generally, the larger the payload, the fewer the payload molecules that will be encapsulated within a single LNP and/or liposome.

For an oligonucleotide payload, the N/P ratio is the ratio of amine groups in the ionizable or cationic lipid to the number of phosphate groups in the oligonucleotide (considered to be the length of the oligonucleotide). In embodiments, the N/P ratio is 1 or greater, 2 or greater, 3 or greater, 4 or greater, 5 or greater, 6 or greater, 7 or greater, 8 or greater, 9 or greater, 10 or greater, 12 or greater, 14 or greater, 16 or greater, or 18 or greater. In embodiments, the N/P ratio is 20 or less, 18 or less, 16 or less, 14 or less, 12 or less, 10 or less, 9 or less, 8 or less, 7 or less, 6 or less, 5 or less, or 4 or less. In embodiments, the N/P ratio is 1 to 20, 3 to 20, 3 to 7, 3 to 6, 3 to 5, or 3 to 4. In embodiments, the N/P ratio is 4 to 7, 4 to 6, or 4 to 5. In embodiments, the N/P ratio is S to 7 or 5 to 6. In embodiments, the N/P is 6 to 7. In embodiments, the N/P ratio is 4 to 6. In embodiments, the N/P ratio is 5.

Also described herein are methods for forming a lipid-based particle that includes a conjugated lipid. The lipid-based particle may be an LNP or a liposome. The lipid-based particle may also include a payload encapsulated within the particle. In embodiments, the lipid-based particle is a liposome, and the liposome encapsulates the payload. In embodiments, the lipid-based particleis an LNP, and the LNP encapsulated the payload.

In embodiments, a first method for forming an LNP includes creating a first mixutre that includes a conjugated lipid, a PEGylated lipid, an ionizable lipid or cationic lipid, a helper lipid, cholesterol or derivative thereof, and a solvent. The conjugated lipid, PEGylated lipid, ionizable lipid or cationic lipid, helper lipid, may be any conjugated lipid, PEGylated lipid, ionizable lipid or cationic lipid, helper lipid, and cholesterol or derivative thereof as described elsewhere herein. The components of the first mixture may be included in any ratio and/or mol-% to achieve the any desired mol-% as described elswhere herein.

The solvent may be any solvent that confers solubility to the conjugated lipid, a PEGylated lipid, an ionizable lipid or cationic lipid, cholesterol or derivative thereof, and/or helper lipid. In embodiments, the solvent is a polar protic solvent. Example solvents include, but are not limited to, methanol, ethanol, isopropanol, and combinations thereof.

The first method furhter includes creating a second mixture that includes the payload. The payload may be any payload as described elsewhere herein. The second mixture may be an acidic aquous mixutre. The aqueous acidic second mixutre may be at a pH of 2 to 6.5, 3 to 5, or 4 to 4. In embodimetns, the acid aqueous second mixure includes one or more salts. Example salts that may be include in the acidic aqueous second mixture include, but are not limited to, citrate salts, acetate salts, and phospahte salts. The salts may be present in the acidic aqueous second mixture at concentrations raging from 1 mM to 500 mM.

The first method futher includes mixing the first mixture with the second mixutre to create a third mixutre. In embodiments, the ratio of the volume of the second mixture (aquous) to the first mixture (organic) is one part, two parts, three parts, four parts, or five parts the first mixture to every one part of the first mixture.

In embodiments, mixing inlcudes vortexing, sonicating, pipette mixing, or combinations thereof. In embodiments, mixing includes using a microfluidics device.

The first method further includes allowing the third mixture to incubate following the mixing for a time period to produce the lipid-based particle. In embodiments, the time period is 1 min to 60 min, 10 min to 30 min, or 10 min to 15 min.

In embodiments, the first method further includes salt exchanging the LNP. Salt exchange can be achieved using any suitable method known in the art. In embodiments, salt exchange is achieved using dialysis and/or size exclusion chromatography using suitable parameters known in the art.

In embodiments, a second method for forming an LNP includes creating a first mixutre that includes, a PEGylated lipid having reactive handle (e.g., LipA, LipB, LipC, and LipD), an ionizable lipid, a helper lipid, a sterol, and a solvent. The PEGylated lipid having a reactive handle, ionizable lipid or cationic lipid, helper lipid, may be any PEGylated lipid having a reactive handle, ionizable lipid or cationic lipid, helper lipid, and sterol as described elsewhere herein. The components of the first mixture may be included in any ratio and/or mol-% to achieve the any desired mol-% as described elswhere herein. In embodimetns, the first mixutre also includes a PEGylated lipid that does not have a reactive handle. The solvent may be any solvent as describe relative to the first LNP formation method.

The second method includes creating a second mixutre that includes the payload. The payload may be any payload as described elsewhere herein. The second mixture may be an acidic aqueous mixture. The aqueous acidic second mixutre may be at a pH of 2 to 6.5, 3 to 5, or 4 to 4. In embodiments, the acid aqueous second mixure includes one or more salts. Example salts that may be include in the acidic aqueous second mixture include, but are not limited to, citrate salts, acetate salts, and phospahte salts. The salts may be present in the acidic aqueous second mixture at concentrations raging from 1 mM to 500 mM.

The second method futher includes mixing the first mixture with the second mixutre to create a third mixutre. In embodiments, the ratio of the volume of the second mixture (aquous) to the first mixture (organic) is one part, two parts, three parts, four parts, or five parts the first mixture to every one part of the first mixture.

In embodiments, mixing inlcudes vortexing, sonicating, pipette mixing, or combinations thereof. In embodiments, mixing includes using a microfluidics device.

The second method further includes allowing the third mixture to incubate following the mixing for a time period to produce the lipid-based particle. In embodiments, the time period is 1 min to 60 min, 10 min to 30 min, or 10 min to 15 min.

The second method further includes exposing the lipid-based particle to a delivery construct having a cooperative reactive handle to the reactive handle of the PEGylated lipid. The cooperative reactive handles may react to form a reaction product that covalently links the PEGylated lipid to the delivery construct thereby forming a lipid conjugate.

In embodiments, the amount delivery construct to the amount of PEGylated lipid having a reactive handle used to formulate the lipid-based particle is 10:1, 9:1, 8:1, 7:1, 6:1, 5:1, 4:1, 3:1, 2:1, 1:1, 0.75:1, 0.5:1, 0.25:1, 0.1:1, 0.075:1, 0.05:1, 0.025:1, 0.015:1, 0.01:1 or 0.005:1

In embodiments, the second method further includes exposing the lipid-based particle to a capping compound. The capping compound includes a reactive handle that is cooperative to the PEGylated lipid reactive handle. The capping compound may react with PEGylated lipid reactive handles that have not reacted with the delivery construct to from PEGylated lipid capping group conjugates.

In embodiments, an LNP formed using the second method that includes (a) lipid conjugates, PEGylated lipids having an unreacted reactive handle, and PEGylated lipid capping group conjugates; (b) lipid conjugates and PEGylated lipids having an unreacted reactive handle; lipid conjugates, and PEGylated lipid capping group conjugates; or (c) lipid conjugates only.

In embodiments, the second method further includes salt exchanging the LNP. Salt exchange can be achieved using any suitable method known in the art. In embodiments, salt exchange is achieved using dialysis and/or size exclusion chromatography using suitable parameters known in the art.

### Methods of Making

### Delivery Constructs and Lipid Conjugates

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The CPP disclosed herein can be prepared by solid phase peptide synthesis wherein the amino acid α-N-terminus is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, α,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group is particularly preferred for the synthesis of the disclosed compounds. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for asparticacid and glutamic acid, benzyl and t-butyl and for cysteine, triphenylmethyl (trityl).

In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxymethylphenoxymethyl-copoly (styrene-1% divinylbenzene) or 4-(2′,4′-dimethoxyphenyl-Fmoc-aminomethyl) phenoxyacetamidoethyl resin available from Applied Biosystems (Foster City, Calif.). The a-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis (2-oxo-3-oxazolidinyl) phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl) phenoxy-acetamidoethyl resin, the Fmoc group is cleaved with a secondary amine, preferably piperidine, prior to coupling with the α-C-terminal amino acid as described above. One method for coupling to the deprotected 4 (2′,4′-dimethoxyphenyl-Fmoc-aminomethyl) phenoxy-acetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the a-N-terminus in the amino acids of the growing peptide chain are protected with Fmoc. The removal of the Fmoc protecting group from the a-N-terminal side of the growing peptide is accomplished by treatment with a secondary amine, preferably piperidine. Each protected amino acid is then introduced in about 3-fold molar excess, and the coupling is preferably carried out in DMF. The coupling agent can be O-benzotriazol-1-yl-N,N,N′,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either successively or in a single operation. Removal of the polypeptide and deprotection can be accomplished in a single operation by treating the resin-bound polypeptide with a cleavage reagent comprising thianisole, water, ethanedithiol and trifluoroacetic acid. In cases wherein the α-C-terminal of the polypeptide is an alkylamide, the resin is cleaved by aminolysis with an alkylamine.

Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivitized polystyrene-divinylbenzene (for example, Amberlite XAD); silica gel adsorption chromatography, ion exchange chromatography on carboxymethylcellulose; partition chromatography, e.g. on Sephadex G-25, LH-20 or countercurrent distribution; high performance liquid chromatography (HPLC), especially reverse-phase HPLC on octyl-or octadecylsilyl-silica bonded phase column packing.

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Oligonucleotides provided herein can be conveniently and routinely made through the well-known technique of solid phase synthesis. Equipment for such synthesis is sold by several vendors including, for example, Applied Biosystems (Foster City, CA). Any other means for such synthesis known in the art may additionally or alternatively be employed. It is well known to use similar techniques to prepare oligonucleotides such as the phosphorothioates and alkylated derivatives. The invention is not limited by the method of oligonucleotide synthesis.

Polymers, such as PEG groups, can be attached to a cCPP, an EP, a linker, a lipid, an oligonucleotide, a protein under any suitable conditions. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group (e.g., an aldehyde, amino, ester, thiol, α-haloacetyl, maleimido or hydrazino group) on the component to which it is being conjugated. Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., a-iodo acetic acid, 60-bromoacetic acid, c-chloroacetic acid). If attached to a cCPP, EP, linker, lipid, protein, or oligonucleotide by reductive alkylation, the polymer selected should have a single reactive aldehyde so that the degree of polymerization is controlled. See, for example, Kinstler et al., Adv. Drug. Delivery Rev. (2002), 54:477-485; Roberts et al., Adv. Drug Delivery Rev. (2002), 54:459-476; and Zalipsky et al., Adv. Drug Delivery Rev. (1995), 16:157-182.

In order to directly covalently link the oligonucleotide, lipid, or linker to the CPP, appropriate amino acid residues of the CPP may be reacted with an organic derivatizing agent that is capable of reacting with a selected side chain or the N- or C-termini of an amino acids. Reactive groups on the peptide or conjugate moiety include, e.g., an aldehyde, amino, ester, thiol, α-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art.

Methods of making oligonucleotide and conjugating oligonucleotide to linear CPP are generally described in US Pub. No. 2018/0298383, which is herein incorporated by reference for all purposes. The methods may be applied to the cyclic CPPs disclosed herein.

Non-limiting examples of compounds that include a CPPs and a reactive group useful for conjugation to, for example, an oligonucleotide, are shown in Table 6. Example linker groups are also shown. Example reactive groups include tetrafluorophenyl ester (TFP), free carboxylic acid (COOH), and azide (N₃). In Table 6, n is an integer from 0 to 20; Pipa6 is AcRXRRBRRXRYQFLIRXRBRXRB wherein B is β-Alanine and X is aminohexanoic acid; Dap is 2,3-diaminopropionic acid; NLS is a nuclear localization sequence; βA is beta alanine; -ss- is a disulfide; PABC is poly(A) binding protein C-terminal domain; Cₓ where x is a number is an alkyl chain of length x; and BCN is bicyclo[6.1.0] nonyne.

TABLE 6
Compounds that include a CPPs and a reactive group

TFP-PEGₙ-K(CPP)
TFP-PEGₙ-K(CPP)-PEGₙ-Dap(palmitoyl)
TFP-PEGₙ-K(CPP)-PEGₙ-Dap(CPP)
TFP-Pip6a
CPP-PEGₙ-TFP
CPP-PEGₙ-K(CPP)-PEGₙ-TFP
CPP-PEGₙ-Lys(N₃)
CPP-K(CPP)-PEGₙ-K(N₃)
CPP-PEGₙ-K(PEGₙ-CPP)-PEGₙ-K(N₃)
CPP-PEGₙ-K(PEGₙ-CPP)-PEGₙ-K(N₃)
CPP-K(CPP)-K(CPP)-PEGₙ-K(N₃)
CPP-PEGₙ-K(PEGₙ-CPP)-K(PEGₙ-CPP)-PEGₙ-K(N₃)
CPP-PEGₙ-K(PEGₙ-CPP)-K(PEGₙ-CPP)-PEGₙ-K(N₃)
Ac-NLS-Lys(CPP)-PEGₙ-K(N₃)
K(N₃)-PEGₙ-NLS-ss-PEGₙ-CPP
BCN-NLS-ss-CPP
CPP-PEGₙ-Val-Cit-PABC-K(N₃)
CPP-PEGₙ-Cys-ss-Cys-K(N₃)
CPP-PEGₙ-Cys-ss-Cys-K(N₃)
CPP-PEGₙ-TFP
CPP-PEGₙ-Lys(N₃)
CPP-PEGₙ-Cys-prodisulfide-K(N₃)
CPP-PEGₙ-K(N₃)
CPP-K(CPP)-PEGₙ-K(N₃)
CPP-PEGₙ-K(CPP)-PEGₙ-TFP
CPP-C₆-TFP
CPP-PEGₙ-K(PEGₙ-CPP)PEGₙ-K(N₃)
Ac-T9-PEGn-Lys(CPP-PEGₙ)-K(N₃)
Ac-MSP-PEGₙ-K(CPP-PEGₙ)-K(N₃)
CPP-PEGₙ-TFP (ENTRD 802)
CPP-C₆-TFP (ENTRD 696)
CPP-PEGₙ-K(CPP)-PEGₙ-TFP (ENTRD-344)
CPP-PEGₙ-COOH
CPP-C₁₂-TFP (ENTD-695)
palmitoyl-PEGₙ-K(CPP)-PEGₙ-TFP (ENTD-343)
CPP-PEGₙ-K(N₃) (ENTRD-617)
Ac-T9-PEGₙ-K(CPP)-K(N₃) (ENTRD 673)
Ac-MSP-PEGₙ-K(CPP-PEGₙ)-K(N₃) (ENTRD 675)
Ac-NLS-K(CPP)-PEGₙ-K(N₃) (ENTRD 684)
K(N3)-PEGₙ-NLS-ss-PEGₙ-CPP (ETRD-681)
K(N3)-PEGₙ-NLS-K-βA-βA-CPP (ETRD-682)

In embodiments, the CPPs have free carboxylic acid groups that may be utilized for conjugation to an oligonucleotide. In embodiments, the EEVs have free carboxylic acid groups that may be utilized for conjugation to a lipid.

### Payload

In embodiments, the lipid-based particle includes an encapsulated payload. The payload may be an oligonucleotide, peptide, small molecule, or any combination thereof. In embodiments, the payload is conjugated to a delivery construct described herein. The delivery construct conjugated to the payload may be referred to as a payload conjugate. In embodiments, the lipid-based particle comprises one or more lipid conjugates and one or more payload conjugates. In embodiments, the lipid-based particle comprises one or more lipid conjugates and the payload is not conjugated to a delivery construct (i.e., an unconjugated payload).

### Oligonucleotides

In embodiments, the payload comprises an oligonucleotide. In embodiments, the payload comprises a payload conjugate where the delivery construct is conjugated to an oligonucleotide. The payload may include any suitable oligonucleotide. The oligonucleotide may include natural DNA bases, modified DNA bases, natural RNA bases, modified RNA bases, natural RNA sugars, modified RNA sugars, natural DNA sugars, modified DNA sugars, natural internucleoside linkages, modified internucleoside linkages, or any combinations thereof.

In embodiments, the oligonucleotide is RNA. RNA-based therapeutics hold great potential as an approach for the treatment or prevention of a variety of diseases. In general, RNA-based therapeutics make use of one of two approaches: (1) antisense RNA (RNAi), in which short oligonucleotides recognize and hybridize to complementary sequences in an endogenous RNA transcript to alter processing; or (2) message RNA (mRNA), in which an mRNAs encoding a peptide or protein of interest is introduced into the cytoplasm of a cell, wherein it can be expressed, for example, to replace a defective protein or present an antigen for vaccination.

To function in vivo, the RNA-based therapeutic must be translocated to the cytosol of the cell and protected from degradation by ubiquitous RNases. Lipid-based nanoparticle systems (LNP) are non-viral delivery systems that have been successfully used for the delivery of a variety of RNA-based therapeutics. LNPs translocate their cargo into cells via membrane-derived endocytic pathways. However, once endocytosed, the encapsulated cargo must then be released into the cytosol of the cell for translation and protein expression.

A major challenge that remains is the ability of the RNA to cross the endosomal membrane. Ineffective endosomal escape can result in increased dosages. Lipid-based particles described herein may provide for more efficient cytosolic transfer of payload, such as RNA payload.

mRNA

In embodiments, the payload comprises an mRNA. In embodiments, the payload comprises a payload conjugate where the delivery construct is conjugated to mRNA.

As used herein, the term “mRNA” refers to an RNA molecule that encodes a protein and includes pre-mRNA and mature mRNA. When introduced into a cell, the mRNA may be translated into a protein using the translation machinery of the cell. In embodiments, the mRNA is mature mRNA. In embodiments, the mature mRNA includes a 3′ poly-A tail and a 5′ cap and includes no introns.

In embodiments, the mRNA encodes a protein or a portion or a protein that can induce an immunological response. In embodiments, the mRNA may be a component of a vaccine. In embodiments, the mRNA may encode one or more gene editing machinery components such as discussed elsewhere herein.

### Antisense Compound (AC)

In embodiments, the payload comprises an antisense compound (AC). In embodiments, the payload comprises a payload conjugate where the delivery construct is conjugated to an AC.

The term “antisense compound” refers to an oligonucleotide sequence that is complementary, or at least partially complementary to a target nucleotide sequence. ACs include, but are not limited to, RNAi, microRNA, antagomirs, aptamers, ribozymes, immunostimulatory oligonucleotides, decoy oligonucleotides, supermir, miRNA mimics, miRNA inhibitors, U1 adapters, and any combination thereof.

In embodiments, the payload is an ASO oligonucleotide, including, but not limited to, a duplex capable of mediating RNA interference (RNAi). In embodiments, the payload is an RNAi molecule that includes an RNA sense strand and an RNA antisense strand (an RNA:RNA duplex); a DNA sense strand and an RNA antisense strand or an RNA sense strand and a DNA antisense strand (a DNA:RNA duplex) or a DNA sense strand and a DNA antisense strand (a DNA:DNA duplex).

In embodiments the payload is a small interfering RNA (siRNA). In embodiments, the siRNA is selected from a single strand siRNA compound, a hairpin siRNA compound, or a double strand siRNA compound.

In embodiments, the payload is a microRNA (miRNA). In embodiments, the payload is an antagomir. In embodiments, the payload is an aptamer. In embodiments, the payload is a ribozyme. In embodiments, the payload is a supermir. In embodiments, the payload is a miRNA mimic, a synthetic non-coding RNA that is capable of entering the RNAi pathway and regulating gene expression. In embodiments, the payload is a miRNA inhibitor. In embodiments, the payload is an immunostimulatory oligonucleotide. In embodiments, the payload is a decoy oligonucleotide. In embodiments, the payload is a U1 adaptor, a bifunctional oligonucleotide with a target domain complementary to a site in the terminal exon of a target gene and a ‘U1 domain’ that binds to the U1 smaller nuclear RNA component of the U1 snRNP.

In embodiments, the AC is the same length as the target nucleotide sequence. In embodiments, the AC is a different length than the target nucleotide sequence. In embodiments, the AC is longer than the target nucleotide sequence.

In embodiments, the AC is 5 or more, 10 or more, 15 or more, 20 or more, 25 or more, 30 or more, 35 or more, 40 or more, or 45 or more nucleotides in length. In embodiments, the AC is 50 or less, 45 or less, 40 or less, 35 or less, 30 or less, 25 or less, 20 or less, 15 or less, or 10 or less nucleotides in length. In embodiments, the AC is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In embodiments, the AC has 100% complementarity to a target nucleotide sequence. In embodiments, the AC does not have 100% complementarity to a target nucleotide sequence. As used herein, the term “percent complementarity” refers to the number of nucleobases of an AC that have nucleobase complementarity with a corresponding nucleobase of an oligomeric compound or nucleic acid (e.g., a target nucleotide sequence) divided by the total length (number of nucleobases) of the AC. One skilled in the art recognizes that the inclusion of mismatches is possible without eliminating the activity of the AC.

In embodiments, the AC has 80% or greater, 85% or greater, 90% or greater, 95% or greater, 96% or greater, 97% or greater, 98% or greater, or 99% or greater complementarity to a target nucleotide sequence. In embodiments, the AC has 100% or less, 99% or less, 98% or less, 97% or less, 96% or less 95% or less, 90% or less, 85% or less complementarity to a target nucleotide sequence. In embodiments, the AC has 80% to 100%, 80% to 99%, 80% to 98%, 80% to 97%, 80% to 96%, 80% to 95%, 80% to 90%, 80% to 85%, 85% to 100%, 85% to 99%, 85% to 98%, 85% to 97%, 85% to 96%, 85% to 95%, 85% to 90%, 90% to 100%, 90% to 99%, 90% to 98%, 90% to 97%, 90% to 96%, or 90% to 95%, 95% to 100%, 95% to 99%, 95% to 98%, 95% to 97%, 95% to 96%, 96% to 100%, 96% to 99%, 96% to 98%, or 96% to 97%, 97% to 100%, 97% to 99%, 97% to 98%, 98% to 100%, 98% to 99%, or 99% to 100% complementarity to a target nucleotide sequence.

In embodiments, incorporation of nucleotide affinity modifications allows for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between an AC and a target nucleotide sequence, such as by determining the thermal melting temperature (Tm). Tm or ΔTm can be calculated by techniques that are familiar to one of ordinary skill in the art. For example, techniques described in Freier et al. (Nucleic Acids Research, 1997, 25, 22:4429-4443) allow one of ordinary skill in the art to evaluate nucleotide modifications for their ability to increase the melting temperature of an RNA:DNA duplex.

The ACs described herein may contain one or more asymmetric centers and thus give rise to enantiomers, diastereomers, and other stereoisomeric configurations that may be defined, in terms of absolute stereochemistry, as (R) or (S); a or B; or as (D) or (L). Included in the antisense compounds provided herein are all such possible isomers, as well as their racemic and optically pure forms.

The efficacy of the ACs may be assessed by evaluating the antisense activity effected by their administration. As used herein, the term “antisense activity” refers to any detectable and/or measurable activity attributable to the hybridization of an antisense compound to its target nucleotide sequence. Such detection and/or measuring may be direct or indirect. In embodiments, antisense activity is assessed by detecting and or measuring the amount of the protein expressed from the transcript of interest. In embodiments, antisense activity is assessed by detecting and/or measuring the amount of the transcript of interest. In embodiments, antisense activity is assessed by detecting and/or measuring the amount of alternatively spliced RNA and/or the amount of protein isoforms translated from the target transcript.

### AC Structure

The AC includes an oligonucleotide and/or an oligonucleoside. Oligonucleotides and/or oligonucleotides are nucleotides or nucleosides linked through internucleoside linkages, sometimes called backbone linkages or simply backbone. Nucleosides include a pentose sugar (e.g., ribose or deoxyribose) and a nitrogenous base covalently attached to the sugar. The naturally occurring (or traditional basses) nucleobases found in DNA and/or RNA are adenine (A), guanine (G), thymine (T), cytosine (C), and uracil (U). The naturally occurring sugars (or traditional sugars) found in DNA and/or RNA are deoxyribose (DNA) and ribose (RNA). The naturally occurring nucleoside linkage (or traditional internucleoside linkage) is a phosphodiester bond. In embodiments, the ACs may have all natural sugars, bases, and internucleoside linkages.

Chemically modified nucleosides are routinely used for incorporation into antisense compounds to enhance one or more properties, such as nuclease resistance, pharmacokinetics, or affinity for a target RNA. In embodiments, the ACs may have one or more modified nucleosides. In embodiments, the ACs may have one or more modified sugars. In embodiments, the ACs may have one or more modified bases. In embodiments, the ACs may have one or more modified internucleoside linkages.

In general, a nucleobase is any group that contains one or more atom or groups of atoms capable of hydrogen bonding to a base of another nucleic acid. In addition to “unmodified” or “natural” nucleobases (A, G, T, C, and U) many modified nucleobases or nucleobase mimetics are known to those skilled in the art are amenable with the compounds described herein. Generally, a modified nucleobase refers to a nucleobase that is fairly similar in structure to the parent nucleobase, such as for example 7-deaza purine, 5-methyl cytosine, 2-thio-dT, and G-clamp. Generally, a nucleobase mimetic is a nucleobase that includes a structure that is more complicated than a modified nucleobase, such as for example a tricyclic phenoxazine nucleobase mimetic. Methods for preparation of the above noted modified nucleobases are well known to those skilled in the art.

In embodiments, the AC may include one or more nucleosides having a modified sugar moiety. In embodiments, the furanosyl sugar of a natural nucleoside may have a 2′ modification, modifications to make a constrained nucleoside, or other modifications. In embodiments, the furanosyl sugar ring of a natural nucleoside can be modified in a number of ways including, but not limited to, addition of a substituent group, bridging of two non-geminal ring atoms to form a bicyclic nucleic acid (BNA) or a locked nucleic acid; exchanging the oxygen of the furanosyl ring with C or N; and/or substitution of an atom or group. Modified sugars are well known and can be used to increase or decrease the affinity of the AC for its target nucleotide sequence. Modified sugars may also be used to increase AC resistance to nucleases. Sugars can also be replaced with sugar mimetic groups among others. In embodiments, one or more sugars of the nucleosides of the AC is replaced with a methylenemorpholine ring.

In embodiments, the AC includes one or more modified nucleosides that include a bridged nucleic acid (BNA), a locked nucleic acid (LNA), or both. Examples of BNAs and LNAs include, but are not limited to LNA (4′-(CH₂)-O-2′ bridge); 2′-thio-LNA (4′-(CH₂)-S-2′ bridge); 2′-amino-LNA (4′-(CH₂)-NR-2′ bridge); ENA (4′-(CH₂)₂-O-2′ bridge); 4′-(CH₂)₃-2′ bridged BNA; 4′-(CH₂CH(CH₃))-2′ bridged BNA; cEt (4′-(CH (CH₃)-O-2′ bridge); phosphorothioate-LNA; cMOE BNAs (4′-(CH(CH₂OCH₃)-O-2′ bridge); 240 -amino- and 2′-methylamino-LNA's; and alpha-L-LNAs. BNA monomers and oligonucleotides containing the same have been prepared and disclosed in the patent literature as well as in scientific literature.

### Internucleoside Linkages

Internucleoside linking groups link the nucleosides or otherwise modified nucleoside monomer units together thereby forming an oligonucleotide and/or an oligonucleotide containing AC. The ACs may include naturally occurring internucleoside linkages, unnatural internucleoside linkages, or both.

In naturally occurring DNA and RNA, the internucleoside linking group is a phosphodiester that covalently links adjacent nucleosides to one another to form a linear polymeric compound. In naturally occurring DNA and RNA, phosphodiester is linked to the 2′, 3′ or 5′ hydroxyl moiety of the sugar. Within oligonucleotides, the phosphate groups are commonly referred to as forming the backbone of the oligonucleotide. In naturally occurring DNA and RNA, the linkage or backbone of RNA and DNA, is a 3′ to 5′ phosphodiester linkage. In embodiments, the internucleoside linking groups of the ACs are phosphodiesters. In embodiments, the internucleoside linking groups of the ACs are 3′ to 5′ phosphodiester linkages.

The two main classes of unnatural internucleoside linking groups are defined by the presence or absence of a phosphorus atom. Representative phosphorus containing internucleoside linkages include, but are not limited to, phosphotriesters, methylphosphonates, phosphoramidates, phosphorodiamidates and phosphorothioates. Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H₂—O—); and N,N′-dimethylhydrazine (—CH₂—N(CH₃)—N(CH₃)—). ACs having phosphorus internucleoside linking groups are referred to as oligonucleotides. Antisense compounds having non-phosphorus internucleoside linking groups are referred to as oligonucleosides. Modified internucleoside linkages, compared to natural phosphodiester linkages, can be used to alter, typically increase, nuclease resistance of the antisense compound. Internucleoside linkages having a chiral atom can be prepared as racemic, chiral, or as a mixture. Representative chiral internucleoside linkages include, but are not limited to, alkylphosphonates and phosphorothioates. Methods of preparation of phosphorous-containing and non-phosphorous-containing linkages are well known to those skilled in the art.

In embodiments, two or more nucleosides having modified sugars and/or modified nucleobases may be joined using a phosphorodiamidate. In embodiments, two or more nucleosides having a methylenemorpholine ring may be connected through a phosphorodiamidate internucleoside linkage.

Antisense compounds that include nucleobases with a methylenemorpholine ring that are linked through phosphorodiamidate internucleoside linkage may be referred to as phosphorodiamidate morpholino oligomers (PMOs).

### Conjugate Groups

In embodiments, ACs are modified by covalent attachment of one or more conjugate groups. In general, conjugate groups modify one or more properties of the attached AC including but not limited to pharmacodynamic, pharmacokinetic, binding, absorption, cellular distribution, cellular uptake, charge, and clearance. Conjugate groups are routinely used in the chemical arts and are linked directly or via an optional linking moiety or linking group to a parent compound such as an AC. Conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, antbraquinone, adamantane, acridine, fluoresceins, rhodamines, coumarins and dyes. In embodiments, the conjugate group is a polyethylene glycol (PEG), and the PEG is conjugated to either the AC or the delivery construct.

### Gene-Editing Machinery (“GEM”)

In embodiments, the payload of a lipid-based particle that includes a lipid-conjugate includes one or more gene-editing machinery (GEM) components. In embodiments, the payload comprises a delivery construct conjugated to one or more GEM components, also referred to as a GEM conjugate.

As used herein, “gene-editing machinery” or “GEM” refers to one or more components of a gene editing system. A “gene editing system” is the combination of GEM components that can affect an edit in a target genome. Non-limiting examples of GEM components include targeting oligonucleotides, nucleases, nuclease inhibitors, and combinations thereof. Nucleic acids encoding protein GEM components, such as nucleases, are also considered GEM components for purposes of the present disclosure. Nucleic acids encoding GEM components may comprise an expression vector, plasmid, mRNA, or the like.

In embodiments, the one or more GEM components are components of a CRISPR-Cas gene-editing system. The following patent documents describe CRISPR gene-editing machinery: U.S. Pat. Nos. 8,697,359, 8,771,945, 8,795,965, 8,865,406, 8,871,445, 8,889,356, 8,895,308, 8,906,616, 8,932,814, 8,945,839, 8,993,233, 8,999,641, U.S. patent application Ser. No. 14/704,551, and U.S. patent application Ser. No. 13/842,859. Each of the aforementioned patent documents is incorporated by reference herein in its entirety.

### Nucleases

In embodiments, the GEM payload comprises a nuclease or a nuclease variant. In embodiments, the GEM payload is a GEM conjugate comprising a delivery construct conjugated to a nuclease or a nuclease variant, also called a nuclease conjugate.

The term “nuclease,” as used herein, refers to a protein that cleaves a phosphodiester bond connecting two adjacent nucleotide residues at a target site in a target nucleic acid. A “target site,” “recognition sequence,” or “nuclease target site” is the location that a nuclease nicks or breaks the target nucleic acid (also called the target substrate). Nucleases can affect single or double stranded breaks in a double stranded target nucleic acid. In embodiments, a nuclease comprises a “binding domain” that mediates the interaction of the protein with the target nucleic acid and/or the targeting oligonucleotide to which it may be complexed. In embodiments, a nuclease comprises a “cleavage domain” that catalyzes the cleavage of the phosphodiester bond within the nucleic acid backbone at the target site of the target substrate.

The nuclease may be a naturally occurring nuclease, an engineered nuclease, or a variant thereof. A naturally occurring nuclease is a nuclease found naturally in an organism. An engineered nuclease is a nuclease designed de novo. A nuclease variant is a nuclease derived from a naturally occurring nuclease or an engineered nuclease. A nuclease variant may be a nuclease that is truncated; fused to another protein such as another nuclease; include one or more mutations that increase binding affinity, decrease binding affinity, increase cleavage efficacy, decrease cleavage efficacy, remove cleavage ability (e.g., a dead nuclease); or any combination thereof. In embodiments, a nuclease variant may have at least 80%, at least 85%, at least 90%, at least 95%, at least 96%, at least 97%, at least 98%, at least 99%, or 100% sequence identity to the nuclease from which it was derived. An active fragment of a nuclease is a nuclease variant that includes a functional cleavage domain.

In embodiments, a nuclease binds and cleaves a nucleic acid molecule in a monomeric form. In embodiments, a nuclease protein dimerizes or multimerizes in order to cleave a target nucleic acid molecule. Binding domains and cleavage domains of naturally occurring nucleases, as well as modular binding domains and cleavage domains that can be fused to create nuclease binding specific target sites, are well known to those of skill in the art. For example, zinc fingers or transcriptional activator like elements can be used as binding domains to specifically bind a desired target site, and can be fused or conjugated to a cleavage domain, for example, the cleavage domain of FokI, to create nuclease cleaving the target site.

In embodiments, the nuclease or fragment thereof is an endonuclease. An endonuclease cleaves a phosphodiester bond between two internal adjacent nucleotides within a nucleic acid. In embodiments, the endonuclease cleaves a double-stranded nucleic acid target site symmetrically; that is, both strands are cleaved at the same position so that the ends comprise base-paired nucleotides, also referred to herein as blunt ends. In embodiments, the endonuclease cleaves a double-stranded nucleic acid target sites asymmetrically; that is, cleaving each strand of a double stranded nucleic acid at a different position so that the ends comprise unpaired nucleotides. Unpaired nucleotides at the end of a double-stranded DNA molecule are also referred to as “overhangs,” e.g., as “5′-overhang” or as “3′-overhang,” depending on whether the unpaired nucleotide(s) form(s) the 5′ or the 3′ end of the respective DNA strand. Double-stranded stranded DNA molecule ends ending with unpaired nucleotide(s) are also referred to as sticky ends, as they can “stick to” other double-stranded DNA molecule ends comprising complementary unpaired nucleotide(s).

In embodiments, a nuclease recognizes and binds to a single stranded target site. In embodiments, a nuclease recognizes and binds to a double-stranded target site, for example a double-stranded DNA target site.

In embodiments, the nuclease is an exonuclease. An exonuclease cleaves a phosphodiester bond between two adjacent nucleotides where one of the nucleotides is a terminal nucleotide.

In embodiments, a nuclease is a site-specific nuclease, binding and/or cleaving a specific phosphodiester bond within a specific nucleotide sequence, which is also referred to herein as the “recognition sequence,” the “nuclease target site,” or the “target site.” Examples of site-specific nucleases are DNA restriction nucleases. The target sites of DNA restriction nucleases, are well known to those of skill in the art. In embodiments, a restriction nuclease, such as EcoRI, HindIII, or BamHI, recognizes a palindromic, double-stranded DNA target site of 4 to 10 base pairs in length, and cuts each of the two DNA strands at a specific position within the target site.

In embodiments, a nuclease is a deoxyribonuclease (referred to DNase or DNA nuclease). A DNA nuclease catalyzes the hydrolytic cleavage of phosphodiester bonds in the DNA backbone. In embodiments, the DNA nuclease is deoxyribonuclease I, deoxyribonuclease II, or micrococcal nuclease. In embodiments, the DNA nuclease is an endonuclease. In embodiments, the DNA nuclease is a Type I nuclease; that is, a restriction enzyme that cleaves several tens of base pairs upstream or downstream of the recognition site. In embodiments, the DNA nuclease is a Type II nuclease; that is a restriction enzyme that cleaves within or proximate to the recognition site. In embodiments, the nuclease is a Type V nuclease.

In embodiments, a nuclease is a ribonuclease (referred to RNase or RNA nuclease). A RNA nuclease catalyzes the hydrolytic cleavage of phosphodiester bond between two adjacent nucleotides in RNA. In embodiments, the RNA nuclease is RNaseA, RNaseH, RNase III, RNase L, RNaseP, RNase PhyM, RNase T1, RNase T2, RNase U2, RNase V, RNaseE, PNPase, RNase PH, RNase R, RNase D, RNase T, oligoribonuclease, exoribonuclease I, exoribonuclease II, or RNaseG. In embodiments, the RNA nuclease is a Type VI nuclease.

In embodiments, the nuclease is a DNA and RNA nuclease. In embodiments, the DNA and RNA nuclease is a Type III nuclease.

In embodiments, the nuclease is a Type II, Type V-A, Type V-B, Type VC, Type V-U, Type VI-B nuclease.

In embodiments, the nuclease is part of a CRISPR-Cas system. In embodiments, the nuclease may be any Cas CRISPR protein or have any modification as described in the published PCT application No. PCT/US2016/038034 (WO2016205613A1) or U.S. Pat. No. 8,697,359 the entire contents of which are incorporated herein by reference.

In embodiments, the nuclease is from a CRISPR-Cas system where the nuclease can cleave a single stranded nucleotide sequence. In embodiments, the nuclease is from a CRISPR-Cas system where the nuclease can cleave a double stranded nucleotide sequence. In embodiments, the CRISPR-Cas system employs a nuclease that can cleave DNA. In embodiments, the CRISPR-Cas system employs a nuclease that can cleave RNA, mRNA, and/or pre-mRNA.

CRISPR-Cas systems fall into two classes. Class 1 systems use a complex of multiple Cas proteins to degrade foreign nucleic acids. In contrast, Class 2 systems use a single large Cas protein to degrade foreign nucleic acids. In embodiments, the nuclease is from a Class 1 CRISPR-Cas system. In embodiments, the Class 1 system is a Type I system. In embodiments, the Class 1, Type I system is a I-A, I-B, I-C, I-D, I-E, I-F, or I-G subtype. In embodiments, the nuclease of the Class 1, Type I system comprises Cas3, Cas8a, Cas5, Cas8b, Cas8c, Cas10d, Cse1, Cse2, Csy1, Csy2, Csy3, and/or GSU0054, an active variant thereof, or an active fragment thereof. In embodiments, the Class 1 system is a Type III system. In embodiments, the Class 1,Type III system is a III-A, III-B, III-C, III-D, III-E, or III-F subtype. In embodiments, the nuclease of the Class 1, Type III system comprises Cas10, Csm2, Cmr5, Cas10, Csx11, Csx10, an active variant thereof, or an active fragment thereof. In embodiments, the Class 1 system is a Type IV system. In embodiments, the Class 1, Type IV system is a IV-A, IV-B, or IV-C subtype. In embodiments, the nuclease from Class 1, Type IV system comprises Csf1, an active variant thereof, or an active fragment thereof.

In embodiments, the nuclease is from a Class 2 CRISPR-Cas system. In embodiments, the Class 2 system is a Type II system. In embodiments, the Class 2, Type II system is a II-A, II-B, or II-C subtype. In embodiments, the nuclease from a Class 2, Type II system comprises Cas9, Csn2, Cas4, an active variant thereof, or an active fragment thereof. In embodiments, the Class 2 system is a Type V system. In embodiments, the Class 2, Type V system is a V-A, V-B, V-C, V-D, V-E, V-F, V-G, V-H, V-I, V-K, or V-U subtype. In embodiments, the nuclease from a Class 2, Type V system comprise Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, an active variant thereof, or an active fragment thereof. In embodiments, the Class 2 system is a Type VI system. In embodiments, the Class 2, Type VI system is a VI-A, VI-B, VI-C, or VI-D subtype. In embodiments, the nuclease from Class 2, Type VI system comprises Cas13, Cas13a (C2c2), Cas13b, Cas13c, Cas13d, an active variant thereof, or an active fragment thereof.

In embodiments, the nuclease is a Cas9, Cas9 variant, Cas12a (Cpf1), Cas12b, Cas12c, Tnp-B like, Cas13a (C2c2), Cas13b, or Cas14 nuclease. In embodiments, the nuclease is a Cas9 nuclease or a Cpf1 nuclease.

In embodiments, the nuclease is a transcription, activator-like effector nuclease (TALEN); a meganuclease; or a zinc-finger nuclease.

In embodiments, the nuclease is a TALEN. The term “Transcriptional Activator-Like Element Nuclease,” (TALEN) as used herein, refers to an artificial nuclease comprising a transcriptional activator like effector DNA binding domain and a DNA cleavage domain, for example, a FokI domain. A number of modular assembly schemes for generating engineered TALEN constructs have been reported (see e.g., Zhang, Feng; et. al. (February 2011). “Efficient construction of sequence-specific TAL effectors for modulating mammalian transcription”. Nature Biotechnology 29 (2): 149-53; Geiβler, R.; Scholze, H.; Hahn, S.; Streubel, J.; Bonas, U.; Behrens, S. E.; Boch, J. (2011), Shiu, Shin-Han. ed. “Transcriptional Activators of Human Genes with Programmable DNA-Specificity”. PLOS ONE 6 (5): e19509; Cermak, T.; Doyle, E. L.; Christian, M.; Wang, L.; Zhang, Y.; Schmidt, C.; Bailer, J. A.; Somia, N. V. et al. (2011). “Efficient design and assembly of custom TALEN and other TAL effector-based constructs for DNA targeting”. Nucleic Acids Research; Morbitzer, R., Elsaesser, J., Hausner, J.; Lahaye, T. (2011). “Assembly of custom TALE-type DNA binding domains by modular cloning”. Nucleic Acids Research; Li, T.; Huang, S.; Zhao, X.; Wright, D. A.; Carpenter, S.; Spalding, M. H.; Weeks, D. P.; Yang, B. (2011). “Modularly assembled designer TAL effector nucleases for targeted gene knockout and gene replacement in eukaryotes”. Nucleic Acids Research; Weber, E.; Gruetzner, R., Werner, S.; Engler, C.; Marillonnet, S. (2011). Bendahmane, Mohammed. ed. “Assembly of Designer TAL Effectors by Golden Gate Cloning”. PLoS ONE 6 (5): e19722; the entire contents of each of which are incorporated herein by reference).

In embodiments, the nuclease is a zinc finger nuclease. The term “zinc finger nuclease,” as used herein, refers to a nuclease comprising a nucleic acid cleavage domain conjugated to a binding domain that comprises a zinc finger array. The term “zinc finger,” as used herein, refers to a small nucleic acid-binding protein structural motif characterized by a fold and the coordination of one or more zinc ions that stabilize the fold. Zinc fingers encompass a wide variety of differing protein structures (see, e.g., Klug A, Rhodes D (1987). “Zinc fingers: a novel protein fold for nucleic acid recognition”. Cold Spring Harb. Symp. Quant. Biol. 52:473-82, the entire contents of which are incorporated herein by reference). Zinc fingers can be designed to bind a specific sequence of nucleotides, and zinc finger arrays comprising fusions of a series of zinc fingers, can be designed to bind virtually any desired target sequence. Such zinc finger arrays can form a binding domain of a protein, for example, of a nuclease, e.g., if conjugated to a nucleic acid cleavage domain. Different type of zinc finger motifs are known to those of skill in the art, including, but not limited to, Cys2His2, Gag knuckle, Treble clef, Zinc ribbon, Zn2/Cys6, and TAZ2 domain-like motifs (see, e.g., Krishna S S, Majumdar I, Grishin N V (January 2003). “Structural classification of zinc fingers: survey and summary”. Nucleic Acids Res. 31 (2): 532-50). In embodiments, the zinc finger array comprises one or more different zinc finger motifs selected from Cys2His2, Gag knuckle, Treble clef, Zinc ribbon, Zn2/Cys6, and TAZ2 domain-like motifs.

In embodiments, a single zinc finger motif binds 3 or 4 nucleotides of a nucleic acid molecule. In embodiments, a zinc finger domain comprising 2 zinc finger motifs binds 6-8 nucleotides. In embodiments, a zinc finger domain comprising 3 zinc finger motifs binds 9-12 nucleotides. Any suitable protein engineering technique can be employed to alter the DNA-binding specificity of zinc fingers and/or design novel zinc finger fusions to bind virtually any desired target sequence from 3-30 nucleotides in length (see, e.g., Pabo C O, Peisach E, Grant R A (2001). “Design and selection of novel cys2His2 Zinc finger proteins”. Annual Review of Biochemistry 70:313-340; Jamieson A C, Miller J C, Pabo C O (2003). “Drug discovery with engineered zinc-finger proteins”. Nature Reviews Drug Discovery 2 (5): 361-368; and Liu Q, Segal D J, Ghiara J B, Barbas C F (May 1997). “Design of polydactyl zinc-finger proteins for unique addressing within complex genomes”. Proc. Natl. Acad. Sci. U.S.A. 94 (11); the entire contents of each of which are incorporated herein by reference). Fusions between engineered zinc finger arrays and protein domains that cleave a nucleic acid can be used to generate a “zinc finger nuclease.”

In embodiments, the nuclease is a modified form or variant of a Cas9, Cas12a (Cpf1), Cas12b, Cas12c, Tnp-B like, Cas13a (C2c2), Cas13b, or Cas14 nuclease. In embodiments, the nuclease is a modified form or variant of a TAL nuclease, a meganuclease, or a zinc-finger nuclease. In embodiments, the nuclease may have at least 80%, at least 85%, at least 90%, at least 95%, at least 96%, at least 97%, at least 98%, at least 99%, or 100% sequence identity to a naturally occurring Cas9, Cas12a (Cpf1), Cas12b, Cas12c, Inp-B like, Cas13a (C2c2), Cas13b, Cas14 nuclease, or a TALEN, meganuclease, or zinc-finger nuclease.

In embodiments, the nuclease has at least 90%, at least 95%, at least 96%, at least 97%, at least 98%, at least 99%, or 100% sequence identity to anyone of the Cas9 proteins in Table 7. In embodiments, the nuclease is a high-fidelity variant of any one of the nucleases in Table 7.

TABLE 7
Various Cas9 nucleases

| Cas9 nuclease | Organism |
|---|---|
| SpCas9 | S. pyogenes |
| SpCas9-HF | S. pyogenes |
| espCas9 | S. pyogenes |
| SaCas933 | S. aureus |
| SaCas9-HF | S. aureus |
| KKHSaCas9 | S. aureus |
| stCas934 | S. thermophilus |
| nmCas935 | N. meningitidis |
| fnCas936 | F. novicida |
| C. jejuni | cjCas937 |
| scCas938 | S. canis |
| SauriCas939 | S. auricularis |

In embodiments, the nucleus is Cpf1 a fragment thereof, or a variant thereof. In embodiments, the nuclease has at least 90%, at least 95%, at least 96%, at least 97%, at least 98%, at least 99% or 100% sequence identity to a Cpf1 enzyme from Acidaminococcus (species BV3L6, UniProt Accession No. U2UMQ6). In embodiments, the nuclease has at least 90%, at least 95%, at least 96%, at least 97%, at least 98%, at least 99% or 100% sequence identity to a Cpf1 enzyme from Lachnospiraceae (species ND2006, UniProt Accession No. A0A182DWE3).

In embodiments, the Cas9 variant binds to a protospacer adjacent motif (PAM) including, but not limited to, NGG, NNGRRT, NNGRRT, NNG, NNGG, NNNNGATT, NNNNRYAC, NNAGAAW, or TTTV where N is any nucleotide.

In embodiments, the nuclease is selected from Table 8 or is a nuclease that has at least 95%, at least 96%, at least 97%, at least 98%, at least 99% or 100% sequence identity to a nuclease from Table 8. In embodiments, the nuclease binds a PAM sequence of Table 8.

TABLE 8
Nucleases and PAM sequences

| PAM sequence | | |
|---|---|---|
| (5′ to 3′) | Nuclease | Isolated (I) of Derived (D) from |
| NGG | SpCas9 | (I) Streptococcus pyogenes |
| NGRRT or NGRRN | SaCas9 | (I) Staphylococcus aureus |
| NNNNGATT | NmeCas9 | (I) Neisseria meningitidis |
| NNNNRYAC | CjCas9 | (I) Campylobacter jejuni |
| NNAGAAW | StCas9 | (I) Streptococcus thermophiles |
| TTTV | LbCpf1 | (I) Lachnospiraceae bacterium |
| TTTV | AsCpf1 | (I) Acidaminococcus sp. |
| NGAG | SpCAs9-VQR | (D) Streptococcus pyogenes |
| NGCG | SpCas9-VRER | (D)D Streptococcus pyogenes |
| NGN | SpCas9-NG | (D) Streptococcus pyogenes |
| NG, GAA, GAT | SpCas9-xCas9 | (D)m Streptococcus pyogenes |
| NNG | SpCas9-Sc++ | (D) Streptococcus pyogenes |
| NGN | SpCas9-SpG | (D) Streptococcus pyogenes |
| N(G/A)N | SpCas9-SpRY | (D)Streptococcus pyogenes |
| NGG | FnCas9 | (I) Francisella novicida |
| (C/T)G | FnCas9-RHA | (D) Francisella novicida |
| NNN(G/A)(G/A)T | SaCas9-KKH | (D) Staphylococcus aureus |
| NNAGAA(A/T) | StlCas9 | (I) Streptococcus thermophilus |
| NNNNC(G/A)AA | GeoCas9 | (I) Geobacillus stearothermophilus |
| T(C/T)C(A/C/G) | AsCas12a-RR | (D) Acidaminococcus sp. |
| TAT(A/C/G) | AsCas12-RVR | (D) Acidaminococcus sp. |
| TTT(A/C/G) | FnCas12a | (I) Francisella novicida |
| TTN | Cas12j | phage |
| TTCN | Cas12e | |
| TTTN | Un1Cas12f1 | Uncultured archaeon |
| CCN | CnCas12f1 | |

In embodiments, the nuclease includes is linked to or fused with more heterologous functional domains. A heterologous functional domain is a domain that has a function and/or a structure that is not related to the nuclease. Heterologous domains may possess methylase activity, demethylate activity, transcription activation activity, transcription repression activity, transcription factor release activity, histone modification action, nuclease activity, single-strand RNA and/or DNA cleavage activity, double-stranded RNA and/or DNA cleavage activity, single strand cleavage activity, nucleic acid binding activity, or combinations thereof. Examples of heterologous functional domains that may be fused to or linked to a nuclease can be found in the published PCT application No. PCT/US2016/038034 (WO2016205613A1), which is hereby incorporated herein in its entirety. In embodiments, the nuclease is linked to or fused to a heterologous recruiting domain. The heterologous recruiting domain serves to recruit one or more heterologous domains that can possess any one of the activities described herein.

In embodiments, a nuclease of the present disclosure may include additional sequences, such as, for example, amino acids appended to the C-terminus or N-terminus of the nuclease, Such modifications can, for example, facilitate purification by trapping on columns, the use of antibodies, or facilitate recovery when expressed recombinantly in a microbe. Some additional sequences may be detectable tags. Detectable tags can be used to purify protein and/or visualize proteins. Example of detectable tags include, FLAG tags, poly-histidine tags (e.g. 6xHis (SEQ ID NO: 492)), SNAP tags, Halo tags, cMye tags, glutathione-S-transferase tags, maltose binding protein tags, avidin, enzymes, fluorescent proteins, luminescent proteins, chemiluminescent proteins, bioluminescent proteins, and phosphorescent proteins. In embodiments, the additional nucleotide sequences (e.g., purification tags and/or detectable tags) are cleaved from the nuclease using methods known in the art.

In embodiments, the nuclease is fused to a nuclear localization signal. Exemplary nuclear localization sequences include SV40: PKKKRKV (SEQ ID NO: 42); NLP: AVKRPAATKKAGQAKKKKLD (SEQ ID NO: 476); TUS: KLKIKRPVK (SEQ ID NO: 477); and EGL 13: MSRRRKANPTKLSENAKKLAKEVEN (SEQ ID NO: 478).

### Targeting Oligonucleotides

In embodiments, the GEM payload includes a targeting oligonucleotide. In embodiments, the GEM payload is a GEM conjugate comprising a delivery construct conjugated to a targeting oligonucleotide. In embodiments, the targeting oligonucleotide is not conjugated to a delivery construct.

A targeting oligonucleotide is an oligonucleotide that binds to at least one genomic location within a cell. Targeting oligonucleotides can include natural DNA bases, natural RNA bases, modified DNA bases, modified RNA bases, natural sugars, modified sugars, natural internucleoside linkages, modified internucleoside linkages, or any combination thereof. Various bases, sugars, internucleoside linkages, and modifications thereof are described herein.

In embodiments, the payload includes a guide RNA (gRNA). A gRNA targets a genomic location (e.g., a target sequence) in a prokaryotic or eukaryotic cell. In embodiments, the gRNA payload is conjugated to a delivery construct, also termed an gRNA conjugate. In embodiments, the gRNA payload is not conjugated to a delivery construct.

A gRNA is an oligonucleotide that directs the gene editing enzyme (e.g., a nuclease) to a genomic location by hybridizing to a target nucleic acid sequence that includes or is proximate to the location of the edit. A gRNA includes a nuclease binding portion and a target binding portion. The nuclease binding portion binds to the nucleus creating a ribonucleoprotein and the targeting binding portion hybridizes to a target nucleic acid sequence. A gRNA may include additional elements, such as an editing template, an RNA aptamer, a polyA tail, or any combination thereof. A guide nucleic acid may be designed to include combinations of guide nucleic acid elements to improve properties such as specificity, efficiency, and stability, as well as compatibility with a particular nuclease. A gRNA may include a single strand (i.e., sgRNA) or two or more strands that hybridize to form a gRNA having a nuclease binding portion and a target binding portion and/or any additional elements. For example, some natural gRNA systems include two strands, a crRNA and a tracrRNA. The erRNA includes targeting portion and a first nuclease binding portion. The tracrRNA includes a second nuclease binding portion. A portion of the crRNA and the tracrRNA hybridize to form the gRNA.

In embodiments, the gRNA includes a spacer sequence and a scaffold sequence. A spacer sequence is a nucleic acid sequence used to target a nuclease (e.g., a Cas nuclease) to a specific nucleotide sequence of interest such as the location of a desired genomic edit. At least a portion of the spacer sequence hybridizes to a target sequence. The spacer sequence is highly variable depending on the target nucleic acid sequence.

A spacer sequence can be of any length sufficient to direct a nuclease to the location of the desired edit. In embodiments, the spacer sequence is 10 to 50 nucleotides in length. In embodiments, the spacer sequence includes 10 or more, 15 or more, 20 or more, 25 or more, 30 or more, 35 or more, 40 or more, or 45 or more nucleotides. In embodiments, the spacer sequence includes 50 or less, 45 or less, 40 or less, 35 or less, 30 or less, 25 or less, 20 or less, or 15 or less nucleotides. In embodiments, the spacer is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 47, 48, 49, or 50 nucleotides in length. In embodiments, the spacer sequence has from about 40% to about 80% GC content such as from 40% to 60%, 40% to 50%, 50% to 60%, 60% to 80%, or 60% to 70% GC content.

In embodiments, a spacer may bind to and/or direct a nuclease to make an edit in a mammalian gene, such as a human gene. In embodiments, the spacer may bind to and/or direct a nuclease to make an edit in a gene comprising one or more mutations. In embodiments, the spacer may bind to and/or direct a nuclease to make an edit in an intron of a gene. In embodiments, the spacer may bind to and/or direct a nuclease to make an edit in an exon of a gene. In embodiments, the spacer may bind to and/or direct a nuclease to make an edit coding sequence. In embodiments, the spacer may bind to and/or direct a nuclease to make an edit exonic sequence. In embodiments, the spacer may bind to and/or direct and a nuclease to make an edit in the polyadenylation site (PS) of a gene. In embodiments, the spacer may bind to and/or direct a nuclease to make an edit in a sequence element of a PS. In embodiments, the spacer may bind to and/or direct a nuclease to make an edit in the polyadenylation signal (PAS), an intervening sequence (IS), a cleavage site (CS), a downstream element (DES), or a portion or combination thereof of a gene. In embodiments, a spacer may bind to and/or direct a nuclease to make an edit in a splicing element (SE) or a cis-splicing regulatory element (SRE).

In embodiments, the spacer targets a site that immediately precedes a 5′ protospacer adjacent motif (PAM). The PAM sequence may be selected based on the desired nuclease. For example, the PAM sequence may be any one of the PAM sequences shown in Table 8.

The scaffold sequence is the portion of the gRNA that has an affinity for and binds to the nuclease to form a ribonucleoprotein. The scaffold sequence may vary depending on the nuclease to which it binds.

The scaffold sequence may be of any length that allows for association with an appropriate nuclease. In embodiments, the scaffold sequence is from 10 to 200 nucleotides in length. In embodiments, the scaffold sequence includes 10 or more, 20 or more, 30 or more, 40 or more, 50 or more, 60 or more, 70 or more, 80 or more, 90 or more, 100 or more, 120 or more, 140 or more, 160 or more, or 180 or more nucleotides. In embodiments, the scaffold sequence includes 200 or less, 180 or less, 160 or less, 140 or less, 120 or less, 100 or less, 90 or less, 80 or less, 70 or less, 60 or less, 50 or less, 40 or less, 30 or less, or 20 or less nucleotides.

In embodiments, the GEM payload comprises a gRNA where the total number of nucleotides comprising the spacer and the scaffold is 20 to 250. In embodiments, the GEM payload comprises a gRNA where the total number of nucleotides comprising the spacer and the scaffold is more than 250 nucleotides. In embodiments, the GEM payload comprises a gRNA where the total number of nucleotides comprising the spacer and the scaffold includes 20 or more, 40 or more, 60 or more, 80 or more, 100 or more, 120 or more, 140 or more, 160 or more, 180 or more, or 200 or more nucleotides.

In embodiments, the GEM payload comprises a dead gRNA. Dead gRNA is gRNA that can bind to the target sequence of interest and the nuclease but renders the nuclease inactive. In embodiments, the dead gRNA is truncated relative to active gRNA. In embodiments, dead gRNA is targeted to an off-target site that active gRNA may affect. The dead gRNA targeted to the off-target site may prevent the active gRNA, along with the nuclease, from modifying the off-target site.

In embodiments, the gRNA of a GEM payload may include one or more modified nucleotides. Example modifications include 2′-O-methylated nucleotide (i.e., the addition of a methyl group to the 2′hydroxyl of a ribose or a nucleotide); a phosphorothioate modified nucleotide; a phosphorodithicate modified nucleotide; a 2′O-methyl 3′phosphorothiate modified nucleotides; 2′-O-methyl/tbiophosphonoacetate (thioPACE) modified nucleotides; and locked nucleic acid nucleotides (i.e., nucleotides having a methylene bridge between the 2′ and 4′ carbons of the ribose ring). In embodiments, it may be beneficial to incorporate a 2′-O-methylated modified nucleotide, a 2′O-methyl 3′phosphorothiate modified nucleotide, or a 2′-O-methyl thioPACE modified nucleotide at one or both of the terminal nucleotides in order to increase the stability and/or activity of the gRNA.

In embodiments, the payload comprises two or more gRNAs (e.g., 2, 3, 4, 5, 6, 7, 8, 9, 10, 11, 12, 13, 14, 15, 16, 17, 18, 19, or 20). In embodiments, the gRNAs recognize bind to or direct the nuclease to make an edit at the same location. In embodiments, the gRNAs recognize bind to or direct the nuclease to make an edit at different locations.

### Ribonucleoproteins (RNPs)

In embodiments, the GEM cargo includes a ribonucleoprotein (RNP). In embodiments, the GEM payload is a GEM conjugate comprising a delivery construct conjugated to a ribonucleoprotein (RNP), also called an RNP conjugate.

The term “ribonucleoprotein” refers to a non-covalent or covalent association between one or more targeting oligonucleotides and a nuclease. In embodiments, the targeting oligonucleotide is a gRNA. In embodiments, the nuclease is a Cas nuclease. In embodiments, the RNP is a CRISPR-Cas RNP; that is, the RNP comprises a gRNA and a Cas nuclease.

In embodiments, lipid-based particles may include a payload that comprises an RNP. In embodiments, the lipid-based particles may also include targeting oligonucleotides (e.g., a gRNA) and/or nucleases (e.g., Cas nuclease) that are not associated in an RNP but can form an RNP.

In embodiments where the GEM payload includes a GEM conjugate comprising delivery construct conjugated to an RNP, the delivery construct may be conjugated to the targeting oligonucleotide (e.g., gRNA), the nuclease (e.g., a Cas nucleus), or both. In embodiments, two or more delivery constructs are conjugated to the RNP. The delivery constructs may be the same or different.

### Nuclease Inhibitors

In embodiments, the GEM payload comprises a nuclease inhibitor. In embodiments, the GEM payload is a GEM conjugate comprising a delivery construct conjugated to a nuclease inhibitor, also called a nuclease inhibitor conjugate.

A limitation of gene editing is potential off-target editing. The delivery of a nuclease inhibitor will limit off-target editing. In embodiments, the nuclease inhibitor is a polypeptide, polynucleotide, or small molecule. Exemplary nuclease inhibitors are described in U.S. Publication No. 2020/087354, International Publication No. 2018/085288, U.S. Publication No. 2018/0382741, International Publication No. 2019/089761, International Publication No. 2020/068304, International Publication No. 2020/041384, and International Publication No. 2019/076651, each of which is incorporated by reference herein in its entirety.

### Nucleic Acids and Expression Vectors

In embodiments, the GEM payload comprises a nucleic acid encoding a nuclease (e.g., a Cas protein), a targeting oligonucleotide (e.g., gRNA), or both. In embodiments, the GEM payload is a GEM conjugate comprising delivery construct conjugated to a nucleic acid encoding a nuclease, a targeting oligonucleotide, or both, also called a GEM nucleic acid conjugate.

The nucleic acid encoding a nuclease, a targeting oligonucleotide, or both may be RNA or DNA. In embodiments, the nucleic acid is mRNA. Nucleic acids encoding GEM components may include any nucleobase modification, sugar modification, or internucleoside modification so long as the nucleic acid is transcribable and/or translatable. The nucleic acid may be single stranded, double stranded, or include one or more regions of both.

In embodiments, the payload includes a nucleic acid that encodes one or more targeting oligonucleotides (e.g., gRNAs). In embodiments the nucleic acid encodes 1, 2, 3, 4, 5, 6, 7, 8, 9, 10, 11, 12, 13, 14, 15, 16, 17, 18, 19, or 20 targeting nucleotides. In embodiments, the targeting nucleotides recognize the same target. In embodiments, the targeting nucleotides recognize different targets.

In embodiments, the payload includes a nucleic acid that encodes one or more nucleases. In embodiments, the payload includes a nucleic acid that encodes one or more nucleases and one or more targeting oligonucleotides (e.g., gRNAs).

The polynucleotides contemplated herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length being limited by the ease of preparation and use in the intended recombinant DNA protocol. Polynucleotides can be prepared, manipulated and/or expressed using any of a variety of well-established techniques known and available in the art.

In embodiments, a vector may also comprise a sequence encoding a signal peptide (e.g., for nuclear localization, nucleolar localization, mitochondrial localization), fused to the polynucleotide encoding the GEM component. For example, a vector may comprise a nuclear localization sequence (e.g., from SV40 or cMyc) fused to the polynucleotide encoding the GEM component. Exemplary nuclear localization sequences include SV40: PKKKRKV (SEQ ID NO: 42); NLP: AVKRPAATKKAGQAKKKKLD (SEQ ID NO: 476); TUS: KLKIKRPVK (SEQ ID NO: 477); and EGL-13: MSRRRKANPTKLSENAKKLAKEVEN (SEQ ID NO: 478).

In embodiments, the nucleic acid is a vector. The term “vector” is used herein to refer to a nucleic acid molecule capable of transferring or transporting another nucleic acid molecule into a cell. The transferred nucleic acid is generally linked to, e.g., inserted into, the vector nucleic acid molecule. A vector may include sequences that direct autonomous replication in a cell, or may include sequences sufficient to allow integration into host cell DNA.

In embodiments, the nucleic acid encoding a GEM component is included in an expression cassette. The term “expression cassette” as used herein refers to genetic sequences within a vector that direct a cell's machinery to make RNA and proteins. The nucleic acid cassette contains the nucleic acid encoding the GEM component (e.g., nuclease, nuclease variant, or targeting oligonucleotide). The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. In embodiments, the cassette has its 3′ and 5′ ends adapted for ready insertion into a vector, e.g., it has restriction endonuclease sites at each end. The cassette can be removed and inserted into a plasmid or viral vector as a single unit. Exemplary vectors include, without limitation, plasmids, phagemids, cosmids, transposons, artificial chromosomes such as yeast artificial chromosome (YAC), bacterial artificial chromosome (BAC), P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (available from Promega, Madison, WI) for expression in mammalian cells; pLenti4/V5-DEST™, pLenti6/V5-DEST™, and pLenti6.2/V5-GW/lacZ (available from Invitrogen, Carlsbad, CA) for lentivirus-mediated gene transfer and expression in mammalian cells.

In embodiments, the vector is a non-integrating vector, including but not limited to, an episomal vector or a vector that is maintained extrachromosomally. As used herein, the term “episomal” refers to a vector that is able to replicate without integration into host's chromosomal DNA and without gradual loss from a dividing host cell also meaning that the vector replicates extrachromosomally or episomally. The vector is engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In a particular aspect, the lymphotrophic herpes virus may be Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus. Typically, the host cell comprises the viral replication transactivator protein that activates the replication.

In embodiments, a polynucleotide is introduced into a target or host cell using a transposon vector system. In certain embodiments, the transposon vector system comprises a vector comprising transposable elements; a nucleic acid encoding one or more GEM components; and a transposase. In one embodiment, the transposon vector system is a single transposase vector system, see, e.g., WO 2008/027384. Exemplary transposases include, but are not limited to: piggyBac, Sleeping Beauty, Mos1, Tc1/mariner, Tol2, mini-Tol2, Tc3, MuA, Himar I, Frog Prince, and derivatives thereof. The piggyBac transposon and transposase are described, for example, in U.S. Pat. No. 6,962,810, which is incorporated herein by reference in its entirety. The Sleeping Beauty transposon and transposase are described, for example, in Izsvak et al., J. Mol. Biol. 302:93-102 (2000), which is incorporated herein by reference in its entirety. The Tol2 transposon which was first isolated from the medaka fish Oryzias latipes and belongs to the hAT family of transposons is described in Kawakami et al. (2000). Mini-Tol2 is a variant of Tol2 and is described in Balciunas et al. (2006). The Tol2 and Mini-Tol2 transposons facilitate integration of a transgene into the genome of an organism when co-acting with the Tol2 transposase. The Frog Prince transposon and transposase are described, for example, in Miskey et al., Nucleic Acids Res. 31:6873-6881 (2003).

The “control elements” or “regulatory sequences” present in an expression vector are those non-translated regions of the vector (e.g., origin of replication, selection cassettes, promoters, enhancers, translation initiation signals (Shine Dalgarno sequence or Kozak sequence) introns, a polyadenylation sequence, 5′ and 3′ untranslated regions) which interact with host cellular proteins to carry out transcription and translation. Such elements may vary in their strength and specificity. Depending on the vector system and host utilized, any number of suitable transcription and translation elements, including ubiquitous promoters and inducible promoters may be used. In embodiments, the polynucleotide of interest is operably linked to a control element or regulatory sequence. “Operably linked” refers to a juxtaposition wherein the components so described are in a relationship permitting them to function in their intended manner. For instance, a promoter is operably linked to a polynucleotide sequence if the promoter affects the transcription or expression of the polynucleotide sequence.

In embodiments, the nucleic acid encoding one or more GEM components is operably linked to a promoter sequence. The term “promoter” as used herein refers to a recognition site of a polynucleotide (DNA or RNA) to which an RNA polymerase binds. An RNA polymerase initiates and transcribes polynucleotides operably linked to the promoter. Illustrative ubiquitous promoters suitable for use in particular embodiments include, but are not limited to, a cytomegalovirus (CMV) immediate early promoter, a viral simian virus 40 (SV40) (e.g., early or late) promoter, a spleen focus forming virus (SFFV) promoter, a Moloney murine leukemia virus (MoMLV) LTR promoter, a Rous sarcoma virus (RSV) LTR, a herpes simplex virus (HSV) (thymidine kinase) promoter, HS, P7.5, and P11 promoters from vaccinia virus, an elongation factor 1-alpha (EF1α) promoter, early growth response 1 (EGR1) promoter, a ferritin H (FerH) promoter, a ferritin L (FerL) promoter, a Glyceraldehyde 3-phosphate dehydrogenase (GAPDH) promoter, a eukaryotic translation initiation factor 4A1 (EIF4A1) promoter, a heat shock 70 kDa protein 5 (HSPA5) promoter, a heat shock protein 90 kDa beta, member 1 (HSP90B1) promoter, a heat shock protein 70kDa (HSP70) promoter, a β-kinesin (β-KIN) promoter, the human ROSA 26 locus (Irions et al., Nature Biotechnology 25, 1477-1482 (2007)), a Ubiquitin C (UBC) promoter, a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, dl587rev primer-binding site substituted (MND) promoter (Challita et al., J Virol. 69(2):748-55 (1995)).

In embodiments, a vector comprising an expression cassette comprising nucleic acid sequence encoding a GEM is introduced into a host cell that is capable of expressing the encoded GEM component. Exemplary host cells include Chinese Hamster Ovary (CHO) cells, HEK 293 cells, BHK cells, murine NSO cells, or murine SP2/0 cells, and E. coli cells. The expressed protein is then purified from the culture system using any one of a variety of methods known in the art (e.g., Protein A columns, affinity chromatography, size-exclusion chromatography, and the like).

Numerous expression systems exist that are suitable for use in producing the GEM components described herein. Eukaryote-based systems in particular can be employed to produce nucleic acid sequences, or their cognate polypeptides, proteins and peptides. Many such systems are commercially and widely available.

### Analysis of Enzyme Activity

An advantage of the CRISPR system is the ability to target any sequence in a DNA sequence that contains a PAM motif on either strand of DNA for editing. Nuclease binding depends on the complementary base pairing of the guide RNA to the DNA target to produce a targeted double-strand or single-strand break in the DNA. This break is then repaired by the endogenous cellular repair machinery and can lead to local insertion and/or deletion events via the nonhomologous end-joining pathway or to precise sequence modification via homology-directed repair when a user-defined donor template is provided. However, not all guide RNAs are equally effective at directing the nuclease-mediated DNA modifications and the GEM disclosed herein may show different stability in vitro, ex vivo, or in vivo.

Any appropriate assay may be used to assess genome editing by GEM of the present disclosure. In embodiments, the assay includes, but is not limited to, a T7 endonuclease 1 (T7E1) mismatch detection assay, next-generation sequencing (NGS), tracking of indels by decomposition (TIDE) assay, Indel Detection by Amplicon Analysis (IDAA), and a DNA cleavage assay. In embodiments, nuclease activity is assayed in vitro. In embodiments nuclease activity is assayed ex vivo. In embodiments, the nuclease activity is assayed in vivo. In embodiments, the assay is a cell-based assay. In embodiments, the assay is a synthetic assay. In embodiments, the synthetic assay comprises one or more substrate DNA sequences known to be targets of the gRNA or RNP.

### GEM Conjugates

The present disclosure provides gene editing machinery (GEM) components conjugated to one or more delivery constructs, also called GEM conjugates. The GEM conjugates may, or may not, be employed as a GEM payload of a lipid-based particle. The GEM conjugates may include nuclease, nuclease variant, targeting oligonucleotide, or RNP such as those disclosed herein.

In embodiments, the GEM conjugate comprises one or more delivery constructs conjugated to a targeting oligonucleotide (e.g., a gRNA). The delivery constructs may be conjugated to the 5′ end, the 3′ end, or any place in between of a targeting oligonucleotide using any conjugation chemistry such as those disclosed herein.

In embodiments, the GEM conjugate comprises one or more delivery constructs conjugated to a ribonucleoprotein (RNP). In embodiments, the one or more delivery constructs may be conjugated to the nuclease of the RNP, the targeting oligonucleotide of the RNP, or both.

In embodiments, the GEM conjugate comprises one or more delivery constructs conjugated to a nuclease. In embodiments, the nuclease conjugate includes 1, 2, 3, 4, 5, 6, 7, 8, 9, or 10 delivery constructs. In embodiments where the payload of a lipid-based particle includes a plurality of nuclease conjugates, the payload may include nuclease conjugates having a differing number of delivery constructs conjugated to a single nuclease. For example, a payload may include a plurality of nuclease conjugates comprising a first portion of conjugates having a first number of delivery constructs, a second portion of conjugates having a second number of delivery constructs, and so on. The concentration of each portion may not be the same. The average number of delivery constructs per each nuclease of a plurality of nuclease conjugates can be determined using mass spectrometry. In embodiments, the average number of delivery constructs per nuclease of a plurality of nuclease conjugates is 1, 2, 3, 4, 5, 6, 7, 8, 9, or 10.

The one or more delivery constructs may be conjugated to any location on the nuclease using any conjugation chemistry, such as those described herein. In embodiments, delivery construct is conjugated to the N-terminus of the nuclease, the C-terminus of the nuclease, any position between the N-terminus and the C-terminus of the nuclease, or any combination thereof.

In embodiments, a delivery construct is conjugated to the side chain of one or more natural amino acid residues of the nuclease using any conjugation chemistry such as those disclosed herein. In embodiments, at least a portion of the side chain of a natural amino acid functions as a reactive handle that can react with a reactive handle on the delivery construct to form a reaction product such as those described herein. Examples of natural amino acids with side chains having reactive handles include lysine, cysteine, serine, threonine, aspartic acid, and glutamic acid. In embodiments, the nuclease sequence includes one or more amino acid residues having accessible reactive handles. In embodiments, a nuclease variant is used that includes one or more amino acid residues having accessible reactive handles placed at strategic locations on the nuclease (e.g., proximate to the N-terminus, the C-terminus, or both).

In embodiments, a delivery construct comprises a maleimide reactive handle (Rh^{H}) that can react with a thiol reactive handle (Rh^{B}) of a cysteine residue of the nuclease to form the reaction product between the thiol and maleimide (Rp^{C} where U⁴ is S). In embodiments, a delivery construct comprises an activated ester of a carboxylic acid (Rh^{D}) that can react with an amine reactive handle (Rh^{C}) of a lysine residue to form the reaction product between the activated ester and the amine (Rp^{A} where U⁰ is NH). In embodiments, the activated ester is a tetrafluophenyl ester. In embodiments, delivery construct comprises an NHS-ester (Rh^{G}) that can react with an amine reactive handle (Rh^{C}) of a lysine residue to form the reaction product between the NHS ester and the amine (Rp^{A} where U⁰ is NH). In embodiments, a delivery construct comprises a thiol reactive handle (Rh^{B}) that can react with a thiol reactive handle (Rh^{B}) of a cysteine residue to form a disulfide reaction product (Rp^{D}).

The number of delivery constructs per nuclease for a nuclease conjugate may be impacted by the method used to conjugate the delivery construct to the nuclease. For example, if maleimide chemistry is being used to conjugate a delivery construct to a nuclease that includes more than one accessible cysteine residue, more than one delivery construct may be conjugated to the nuclease.

In embodiments, the nuclease may include an unnatural amino acid that has a reactive handle. Examples of unnatural amino acids that have a reactive handles include, but are not limited to, azidohomoalanine; 2-homopropargylglycine; 3-homoallylglycine, 4-p-acetyl-phenylalanine; 5-p-azido-phenylalinie; N^{ε}-(cyclooct-2-yn-1-yloxy) carbonyl)L-lysine; N^{ε}-2-azideoethyloxycarbonyl-L-lysine; Nε-p-azidobenzyloxycarbonyl lysine; propargyl-L-lysine; trans-cyclooct-2-ene lysine; N^{ε}-p-azidobenzyloxycarbonyl lysine; propargyl-L-lysine; N^{ε}-(cyclooct-2-yn-1-yloxy)carbonyl)L-lysine; bicyclo[6.1.0] non-4-yn-9-ylmethanol lysine; trans-cyclooct-4-ene lysine; cyclooct-2-ynol tyrosine; (E)-2-(cyclooct-4-en-1-yloxyl)ethanol tyrosine; azidonorleucine; and N^{ε}-2-azideoethyloxycarbonyl-L-lysine. In embodiments, the nuclease includes one or more unnatural amino acids having an alkyne reactive handle that can react with an azide reactive handle on a delivery construct to form a click reaction product. In embodiments, the nuclease includes one or more unnatural amino acids having an azide reactive handle that can react with an alkyne reactive handle on a delivery construct to form a click reaction product.

In embodiments, a delivery sequence is directly incorporated into the sequence of a nuclease to form a nuclease conjugate. In such embodiments, no conjugation reaction takes place and the cCPP is a pseudo-cCPP. A “delivery sequence” is the sequence of a cCPP acting as a pseudo-cCPP. A pseudo-cCPP is a cCPP that is linear but is constrained in a cyclic configuration through incorporation into a looped region of a looped nuclease. Any cCPP or portion of a cCPP described herein may be linearized and used as a pseudo-cCPP.

The term “looped nuclease” refers to a nuclease with a secondary structure comprising one or more loops. Loops refer to regions of the protein other than alpha helices and beta-strands. Structurally, loops are generally located in regions where there is a change in direction in the secondary structure. In embodiments, the change in direction can be at least 120 degrees. In embodiments, the change of direction is determined across 200 amino acids or less. Loops that have only 4 or 5 amino acid residues which participate in internal hydrogen bonding can be referred to as “turns”. Protein loops include beta turns and omega loops. The most common types of loops and turns cause a change in direction of the polypeptide chain allowing it to fold back on itself to create a more compact structure. Looped regions in nucleases can be determined by means known in the art, such as queries of the Loops in Proteins database (See Michalesky and Preissner, Loops In Proteins (LIP)—a comprehensive loop database for homology modelling. Protein Engineering, Design, and Selection. (2003) 16:12;979-985), and the online protein fold recognition server Phyre 2 (Kelley et al., The Phyre2 Web Portal For Protein Modeling, Prediction And Analysis. Nat. Protoc 2015, 10 (6), 845-858). Looped regions in nucleases may be annotated within online databases, such as UniProt. For example, the secondary structure of Cas9 from Streptococcus pyogenes serotype Ml (Uniprot Accession Number Q99ZW2) is annotated within the Structure section of Uniprot.

In embodiments, one or more cCPPs or one or more portions of a cCPP can be fused into one or more loop regions of a nuclease. In embodiments, Cas9 from Streptococcus pyogenes serotype M1 comprises a cCPP sequence or portion thereof disclosed herein in one or more of Cas9's loop regions, which are described in Table 9. The amino acid ranges contained in Table 9 are numbered with respect to the following Cas9 sequence where beta strands are italicized and bolded and loops are double underlined.

(SEQ ID NO: 479)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGYDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMINEDK
NLPNEKVLPKHSLLYEYFTVYNELTKVKYVTEGMRKPAFLSGEQKKAIVD
LLFKTNRKVTVKQLKEDYFKKIECFDSVEISGVEDRENASLGTYHDLLKI
IKDKDFLDNEENEDILEDIVLTLTLFEDREMIEERLKTYAHLEDDKVMKQ
LKRRRYTGWGRLSRKLINGIRDKQSGKTILDELKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDERKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLEVEQHKHYLDEIIEQISEFSKRVILADANLDKYLSAYNKHRDK
PIREQAENIIHLETLTNLGAPAAFKYEDTTIDRKRYTSTKEVLDATLIHQ
SITGLYETRIDLSQLGGD

TABLE 9
Looped Regions of Cas9 from Streptococcus pyogenes serotype M1
Amino Acid Ranges of Cas9 that Adopt Turns

| | | |
|---|---|---|
| 23-25 | 552-555 | 1076-1078 |
| 103-105 | 568-573 | 1152-1155 |
| 117-119 | 673-675 | 1168-1170 |
| 196-198 | 687-689 | 1262-1264 |
| 253-257 | 751-753 | 1297-1299 |
| 300-305 | 771-774 | |
| 427-429 | 817-819 | |
| 450-452 | 844-846 | |
| 475-477 | 1053-1055 | |
| 532-534 | 1067-1069 | |
| 532-534 | 1067-1069 | |

In embodiments, a cCPP sequence or a portion thereof replaces one or more amino acids of the loop regions of Table 9; is inserted between amino acids of the loop regions in Table 9; is inserted before the first amino acid in any of the loop regions of Table 9; is inserted directly after the last amino acid residue in any of the loop regions of Table 9; or any combination thereof.

In embodiments, a CPP replaces one or more of, or is inserted between one or more of, amino acids 23-25 of Cas9, for example, amino acid 23, 24, 25, or combinations thereof. In embodiments, a CPP replaces one or more of, or is inserted between one or more of, amino acids 103-105 of Cas9, for example, amino acid 103, 104, 105, or combinations thereof. In embodiments, a CPP replaces one or more of, or is inserted between one or more of, amino acids 117-119 of Cas9, for example, amino acid 117, 118, 119, or combinations thereof. In embodiments, a CPP replaces one or more of, or is inserted between one or more of, amino acids 196-198 of Cas9, for example, amino acid 196, 197, 198, or combinations thereof. In embodiments, a CPP replaces one or more of, or is inserted between one or more of, amino acids 253-257 of Cas9, for example, amino acid 253, 254, 255, 256, 257, or combinations thereof. In embodiments, a CPP replaces one or more of, or is inserted between one or more of, amino acids 300-305 of Cas9, for example, amino acid 300, 301, 302, 303, 304, 305, or combinations thereof. In embodiments, a CPP replaces one or more of, or is inserted between one or more of, amino acids 427-429 of Cas9, for example, amino acid 427, 428, 429, or combinations thereof. In embodiments, a CPP replaces one or more of, or is inserted between one or more of, amino acids 450-452 of Cas9, for example, amino acid 450, 451, 452, or combinations thereof. In embodiments, a CPP replaces one or more of, or is inserted between one or more of, amino acids 475-477 of Cas9, for example, amino acid 475, 476, 477, or combinations thereof. In embodiments, a CPP replaces one or more of, or is inserted between one or more of, amino acids 532-534 of Cas9, for example, amino acid 532, 533, 534, or combinations thereof. In embodiments, a CPP replaces one or more of, or is inserted between one or more of, amino acids 552-555 of Cas9, for example, amino acid 552, 553, 554, 555, or combinations thereof. In embodiments, a CPP replaces one or more of, or is inserted between one or more of, amino acids 568-573 of Cas9, for example, amino acid 568, 569, 570, 571, 572, 573, or combinations thereof. In embodiments, a CPP replaces one or more of, or is inserted between one or more of, amino acids 673-675 of Cas9, for example, amino acid 673, 674, 675, or combinations thereof. In embodiments, a CPP replaces one or more of, or is inserted between one or more of, amino acids 687-689 of Cas9, for example, amino acid 687, 688, 689, or combinations thereof. In embodiments, a CPP replaces one or more of, or is inserted between one or more of, amino acids 751-753 of Cas9, for example, amino acid 751, 752, 753, or combinations thereof. In embodiments, a CPP replaces one or more of, or is inserted between one or more of, amino acids 771-774 of Cas9, for example, amino acid 771, 772, 773, 774, or combinations thereof. In embodiments, a CPP replaces one or more of, or is inserted between one or more of, amino acids 817-819 of Cas9, for example, amino acid 817, 818, 819, or combinations thereof. In embodiments, a CPP replaces one or more of, or is inserted between one or more of, amino acids 844-846 of Cas9, for example, amino acid 844, 845, 846, or combinations thereof. In embodiments, a CPP replaces one or more of, or is inserted between one or more of, amino acids 1053-1055 of Cas9, for example, amino acid 1053, 1054, 1055, or combinations thereof. In embodiments, a CPP replaces one or more of, or is inserted between one or more of, amino acids 1067-1069 of Cas9, for example, amino acid 1067, 1068, 1069, or combinations thereof. In embodiments, a CPP replaces one or more of, or is inserted between one or more of, amino acids 1076-1078 of Cas9, for example, amino acid 1076, 1077, 1078, or combinations thereof. In embodiments, a CPP replaces one or more of, or is inserted between one or more of, amino acids 1152-1155 of Cas9, for example, amino acid 1152, 1153, 1154, 1155, or combinations thereof. In embodiments, a CPP replaces one or more of, or is inserted between one or more of, amino acids 1168-1170 of Cas9, for example, amino acid 1168, 1169, 1170, or combinations thereof. In embodiments, a CPP replaces one or more of, or is inserted between one or more of, amino acids 1262-1264 of Cas9, for example, amino acid 1262, 1263, 1264, or combinations thereof. In embodiments, a CPP replaces one or more of, or is inserted between one or more of, amino acids 1297-1299 of Cas9, for example, amino acid 1297, 1298, 1299, or combinations thereof.

In embodiments, a CPP is inserted immediately after an amino acid within the range 23-25 of Cas9, for example, immediately after amino acid 23, 24, or 25. In embodiments, a CPP is inserted immediately after an amino acid within the range 103-105 of Cas9, for example, immediately after amino acid 103, 104, or 105. In embodiments, a CPP is inserted immediately after an amino acid within the range 117-119 of Cas9, for example, immediately after amino acid 117, 118, or 119. In embodiments, a CPP is inserted immediately after an amino acid within the range 196-198 of Cas9, for example, immediately after amino acid 196, 197, or 198. In embodiments, a CPP is inserted immediately after an amino acid within the range 253-257 of Cas9, for example, immediately after amino acid 253, 254, 255, 256, or 257. In embodiments, a CPP is inserted immediately after an amino acid within the range 300-305 of Cas9, for example, immediately after amino acid 300, 301, 302, 303, 304, or 305. In embodiments, a CPP is inserted immediately after an amino acid within the range 427-429 of Cas9, for example, immediately after amino acid 427, 428, or 429. In embodiments, a CPP is inserted immediately after an amino acid within the range 450-452 of Cas9, for example, immediately after amino acid 450, 451, or 452. In embodiments, a CPP is inserted immediately after an amino acid within the range 475-477 of Cas9, for example, immediately after amino acid 475, 476, or 477. In embodiments, a CPP is inserted immediately after an amino acid within the range 532-534 of Cas9, for example, immediately after amino acid 532, 533, or 534. In embodiments, a CPP is inserted immediately after an amino acid within the range 552-555 of Cas9, for example, immediately after amino acid 552, 553, or 554, 555. In embodiments, a CPP is inserted immediately after an amino acid within the range 568-573 of Cas9, for example, immediately after amino acid 568, 569, 570, 571, 572, or 573. In embodiments, a CPP is inserted immediately after an amino acid within the range 673-675 of Cas9, for example, immediately after amino acid 673, 674, or 675. In embodiments, a CPP is inserted immediately after an amino acid within the range 687-689 of Cas9, for example, immediately after amino acid 687, 688, or 689. In embodiments, a CPP is inserted immediately after an amino acid within the range 751-753 of Cas9, for example, immediately after amino acid 751, 752, or 753. In embodiments, a CPP is inserted immediately after an amino acid within the range 771-774 of Cas9, for example, immediately after amino acid 771, 772, 773, or 774. In embodiments, a CPP is inserted immediately after an amino acid within the range 817-819 of Cas9, for example, immediately after amino acid 817, 818, or 819. In embodiments, a CPP is inserted immediately after an amino acid within the range 844-846 of Cas9, for example, immediately after amino acid 844, 845, or 846. In embodiments, a CPP is inserted immediately after an amino acid within the range 1053-1055 of Cas9, for example, immediately after amino acid 1053, 1054, or 1055. In embodiments, a CPP is inserted immediately after an amino acid within the range 1067-1069 of Cas9, for example, immediately after amino acid 1067, 1068, or 1069. In embodiments, a CPP is inserted immediately after an amino acid within the range 1076-1078 of Cas9, for example, immediately after amino acid 1076, 1077, or 1078. In embodiments, a CPP is inserted immediately after an amino acid within the range 1152-1155 of Cas9, for example, immediately after amino acid 1152, 1153, 1154, or 1155. In embodiments, a CPP is inserted immediately after an amino acid within the range 1168-1170 of Cas9, for example, immediately after amino acid 1168, 1169, or 1170. In embodiments, a CPP is inserted immediately after an amino acid within the range 1262-1264 of Cas9, for example, immediately after amino acid 1262, 1263, or 1264. In embodiments, a CPP is inserted immediately after an amino acid within the range 1297-1299 of Cas9, for example, immediately after amino acid 1297, 1298, or 1299.

In embodiments, a CPP is inserted immediately before an amino acid within the range 23-25 of Cas9, for example, immediately before amino acid 23, 24, or 25. In embodiments, a CPP is inserted immediately before an amino acid within the range 103-105 of Cas9, for example, immediately before amino acid 103, 104, or 105. In embodiments, a CPP is inserted immediately before an amino acid within the range 117-119 of Cas9, for example, immediately before amino acid 117, 118, or 119. In embodiments, a CPP is inserted immediately before an amino acid within the range 196-198 of Cas9, for example, immediately before amino acid 196, 197, or 198. In embodiments, a CPP is inserted immediately before an amino acid within the range 253-257 of Cas9, for example, immediately before amino acid 253, 254, 255, 256, or 257. In embodiments, a CPP is inserted immediately before an amino acid within the range 300-305 of Cas9, for example, immediately before amino acid 300, 301, 302, 303, 304, or 305.In embodiments, a CPP is inserted immediately before an amino acid within the range 427-429 of Cas9, for example, immediately before amino acid 427, 428, or 429. In embodiments, a CPP is inserted immediately before an amino acid within the range 450-452 of Cas9, for example, immediately before amino acid 450, 451, or 452. In embodiments, a CPP is inserted immediately before an amino acid within the range 475-477 of Cas9, for example, immediately before amino acid 475, 476, or 477. In embodiments, a CPP is inserted immediately before an amino acid within the range 532-534 of Cas9, for example, immediately before amino acid 532, 533, or 534. In embodiments, a CPP is inserted immediately before an amino acid within the range 552-555 of Cas9, for example, immediately before amino acid 552, 553, or 554, 555. In embodiments, a CPP is inserted immediately before an amino acid within the range 568-573 of Cas9, for example, immediately before amino acid 568, 569, 570, 571, 572, or 573. In embodiments, a CPP is inserted immediately before an amino acid within the range 673-675 of Cas9, for example, immediately before amino acid 673, 674, or 675. In embodiments, a CPP is inserted immediately before an amino acid within the range 687-689 of Cas9, for example, immediately before amino acid 687, 688, or 689. In embodiments, a CPP is inserted immediately before an amino acid within the range 751-753 of Cas9, for example, immediately before amino acid 751, 752, or 753. In embodiments, a CPP is inserted immediately before an amino acid within the range 771-774 of Cas9, for example, immediately before amino acid 771, 772, 773, or 774. In embodiments, a CPP is inserted immediately before an amino acid within the range 817-819 of Cas9, for example, immediately before amino acid 817, 818, or 819. In embodiments, a CPP is inserted immediately before an amino acid within the range 844-846 of Cas9, for example, immediately before amino acid 844, 845, or 846. In embodiments, a CPP is inserted immediately before an amino acid within the range 1053-1055 of Cas9, for example, immediately before amino acid 1053, 1054, or 1055. In embodiments, a CPP is inserted immediately before an amino acid within the range 1067-1069 of Cas9, for example, immediately before amino acid 1067, 1068, or 1069. In embodiments, a CPP is inserted immediately before an amino acid within the range 1076-1078 of Cas9, for example, immediately before amino acid 1076, 1077, or 1078. In embodiments, a CPP is inserted immediately before an amino acid within the range 1152-1155 of Cas9, for example, immediately before amino acid 1152, 1153, 1154, or 1155. In embodiments, a CPP is inserted immediately before an amino acid within the range 1168-1170 of Cas9, for example, immediately before amino acid 1168, 1169, or 1170. In embodiments, a CPP is inserted immediately before an amino acid within the range 1262-1264 of Cas9, for example, immediately before amino acid 1262, 1263, or 1264. In embodiments, a CPP is inserted immediately before an amino acid within the range 1297-1299 of Cas9, for example, immediately before amino acid 1297, 1298, or 1299.

### Cellular Delivery of GEM Conjugates

GEM conjugates may be delivered to cells using a lipid-based particle or independently of a lipid-based particle.

In embodiments, a GEM conjugate can be delivered to a cell independently of a lipid-based particle. In embodiments, the GEM conjugate is delivered to a cell via free-uptake. As used herein, the terms “free-uptake” and “free uptake” refer to exposure of cells to a treatment (e.g., GEM conjugate) without the aid of a transfection agent. Transfection agents are molecules such as lipids, polymers, and the like that aid in the transfection of foreign substances into cells. Free uptake is a transfection method that does not make use of any physical means or chemical reagents to encourage transfection. Free uptake is accomplished by incubating the one or more GEM components such as a GEM conjugate and a cell for a period of time. Free uptake may occur at elevated temperature (e.g., 97° C.), at ambient temperature (e.g., 20° C.), or at any temperature in between, such as at body temperature (e.g., 37° C.).

Other non-lipid-particle based delivery methods that can be used to deliver a GEM conjugate to a cell include, but are not limited to electroporation; sonoporation; microinjection; DEAE-dextran-mediated transfer; biolistics; heat shock; antibody-targeted, bacterially derived, non-living nanocell-based delivery; and complexation with one or more transfection agents.

Some lipid-based transfection reagents may interact with the GEM conjugate to form complexes and/or may form lipid-based particles. Lipofection reagents such as TRANSFECTAM (Promega, Madison, WI); LIPOFECTIN and (ThermoFischer Scientific, Waltham, MA), and LIPOFECTAMINE and LIPOFECTAMINE CRISP MAX (ThermoFischer Scientific) are examples of lipid-based transfection reagents. Additionally, other cationic and neutral lipids transfection agents may be used (See e.g., Liu et al. (2003) Gene Therapy. 10:180-187; and Balazs et al. (2011) Journal of Drug Delivery. 2011:1-12.)

In embodiments, the GEM conjugates can be employed as payloads for lipid-based particles. In embodiments, the lipid-based particles comprise a lipid conjugate. In embodiments, the lipid-based particles do not comprise a lipid conjugate.

In embodiments, a lipid-based particle of the present disclosure comprising a lipid conjugate includes a GEM conjugate as a payload. In embodiments, a lipid-based particle that does not comprise a lipid conjugate includes a GEM conjugate as a payload.

Lipid-based particles including a GEM conjugate as a payload may be liposomes or lipid nanoparticles (LNPs). The LNPs may be formulated using any combination of lipids (e.g., PEGylated lipids, helper lipids, sterols, and ionizable lipids) such as those disclosed herein.

### Methods of Treatment

The present disclosure provides a method of treating disease in a patient in need thereof, that includes administering a lipid-based particle and/or a GEM conjugate disclosed herein. Example diseases include, but are not limited to, autoimmune diseases, inflammatory diseases, cardiovascular diseases, hepatic diseases, cancer, or combinations there.

In embodiments, the lipid-based particle containing an appropriate payload may be administered to decrease the likelihood of a subject contracting a disease such as in the form of a vaccine.

In various embodiments, treatment refers to partial or complete alleviation, amelioration, relief, inhibition, delaying onset, reducing severity and/or incidence of one or more symptoms in a patient.

The terms, “improve,” “increase,” “reduce,” “decrease,” and the like, as used herein, indicate values that are relative to a control. In embodiments, a suitable control is a baseline measurement, such as a measurement in the same individual prior to initiation of the treatment described herein, or a measurement in a control individual (or multiple control individuals) in the absence of the treatment described herein. A “control individual” is an individual afflicted with the same disease, who is about the same age and/or gender as the individual being treated (to ensure that the stages of the disease in the treated individual and the control individual(s) are comparable).

The individual (also referred to as “patient” or “subject”) being treated is an individual (fetus, infant, child, adolescent, or adult human) having a disease or having the potential to develop a disease. The individual may have a disease mediated by aberrant gene expression or aberrant gene splicing. In various embodiments, the individual having the disease may have wild type target protein expression or activity levels that are less than about 1-99% of normal protein expression or activity levels in an individual not afflicted with the disease. In embodiments, the range includes, but is not limited to less than about 80-99%, less than about 65-80%, less than about 50-65%, less than about 30-50%, less than about 25-30%, less than about 20-25%, less than about 15-20%, less than about 10-15%, less than about 5-10%, less than about 1-5% of normal thymidine phosphorylase expression or activity levels. In embodiments, the individual may have target protein expression or activity levels that are 1-500% higher than normal wild type target protein expression or activity levels. In embodiments, the range includes, but is not limited to, greater than about 1-10%, about 10-50%, about 50-100%, about 100-200%, about 200-300%, about 300-400%, about 400-500%, or about 500-1000%.

In embodiments, the individual is a patient who has been recently diagnosed with the disease. Typically, early treatment (treatment commencing as soon as possible after diagnosis) reduces the effects of the disease and to increase the benefits of treatment.

### Compositions and Methods of Administration

In embodiments, compositions are provided that include one or more of the lipid-based particles and/or GEM conjugates described herein. One or more components of the lipid-based particles and/or GEM conjugates described herein may be in the form of pharmaceutically acceptable salts thereof.

Pharmaceutically acceptable salts include salts of the disclosed compounds that are prepared with acids or bases, depending on the particular substituents found on the compounds. Under conditions where the compounds disclosed herein are sufficiently basic or acidic to form stable nontoxic acid or base salts, administration of the compounds as salts can be appropriate. Examples of pharmaceutically acceptable base addition salts include sodium, potassium, calcium, ammonium, or magnesium salt. Examples of physiologically acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral and parenteral routes of administration. As used herein, the term parenteral includes subcutaneous, intradermal, intravenous, intramuscular, intraperitoneal, intrasternal, and intrathecal administration, such as by injection. Administration of the disclosed compounds or compositions can be a single administration, or at continuous or distinct intervals as can be readily determined by a person skilled in the art.

The lipid-based particles and/or GEM conjugates disclosed berein, and compositions that include them, can also be administered utilizing liposome technology, slow-release capsules, implantable pumps, and biodegradable containers. These delivery methods can, advantageously, provide a uniform dosage over an extended period of time. The compounds can also be administered in their salt derivative forms or crystalline forms.

The lipid-based particles and/or GEM conjugates disclosed herein can be formulated according to known methods for preparing pharmaceutically acceptable compositions. Formulations are described in detail in a number of sources which are well known and readily available to those skilled in the art. For example, Remington's Pharmaceutical Science by E.W. Martin (1995) describes formulations that can be used in connection with the disclosed methods. In general, the lipid-based particles and/or GEM conjugates disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The form depends on the intended mode of administration and therapeutic application. The compositions can also include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously include between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient, and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

lipid-based particles and/or GEM conjugates disclosed herein, and compositions that include them, can be delivered to a cell either through direct contact with the cell or via a carrier means. Carrier means for delivering compounds and compositions to cells are known in the art and include, for example, attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. GEM conjugates can also be incorporated into polymers, examples of which include poly (D-L lactide-co-glycolide) polymer for intracranial tumors; poly[bis(p-carboxyphenoxy)propane:sebacic acid] in a 20:80 molar ratio (as used in GLIADEL); chondroitin, chitin; and chitosan.

Lipid-based particles and/or GEM conjugates and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders that include the active ingredient, which are adapted for the extemporaneous preparation of sterile injectable or infusible solutions or dispersions. The ultimate dosage form should be sterile, fluid and stable under the conditions of manufacture and storage. The liquid carrier or vehicle can be a solvent or liquid dispersion medium that includes, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it may be desirable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

For topical administration, lipid-based particles and/or GEM conjugates disclosed herein can be applied in as a liquid or solid. However, it will generally be desirable to administer them topically to the skin as compositions, in combination with a dermatologically acceptable carrier, which can be a solid or a liquid. Lipid-based particles and/or GEM conjugates disclosed herein can be applied topically to a patient's skin to reduce the size (and can include complete removal) of malignant or benign growths, or to treat an infection site. Lipid-based particles and/or GEM conjugates disclosed herein can be applied directly to the growth or infection site. In embodiments, the lipid-based particles and/or GEM conjugates are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to improve the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the lipid-based particle and payloads within the lipid-based particle and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that include a lipid-based particles and/or GEM conjugates disclosed herein in combination with a pharmaceutically acceptable carrier. In embodiments, the pharmaceutical composition is adapted for oral, topical or parenteral administration. The dose administered to a patient, particularly a human, should be sufficient to achieve a therapeutic response in the patient over a reasonable time frame, without lethal toxicity, and without causing more than an acceptable level of side effects or morbidity. One skilled in the art will recognize that dosage will depend upon a variety of factors including the condition (health) of the patient, the body weight of the patient, kind of concurrent treatment, if any, frequency of treatment, therapeutic ratio, as well as the severity and stage of the pathological condition.

Also disclosed are kits that include lipid-based particles and/or GEM conjugates disclosed herein in one or more containers. The disclosed kits can optionally include pharmaceutically acceptable carriers and/or diluents. In embodiments, a kit includes one or more other components, adjuncts, or adjuvants as described herein. In embodiments, a kit includes one or more anti-cancer agents, such as those agents described herein. In embodiments, a kit includes instructions or packaging materials that describe how to administer a compound or composition of the kit. Containers of the kit can be of any suitable material, e.g., glass, plastic, metal, etc., and of any suitable size, shape, or configuration. In embodiments, a compound and/or agent disclosed herein is provided in the kit as a solid, such as a tablet, pill, or powder form. In embodiments, a compound and/or agent disclosed herein is provided in the kit as a liquid or solution. In embodiments, the kit includes an ampoule or syringe containing a compound and/or agent disclosed herein in liquid or solution form. In embodiments, the kit is a vaccine kit.

### Certain Definitions

All publications, patents and patent applications mentioned in the specification are indicative of the level of skill of those skilled in the art to which this invention pertains. All publications, patents and patent applications are herein incorporated by reference to the same extent as if each individual publication or patent application was specifically and individually indicated to be incorporated by reference.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5.

Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively, to all values in the series, or the endpoints of the range.

“2-[2-[2-aminoethoxy]ethoxy]acetic acid” is also referred to as ABEA or miniPEG.

As used herein, “cell penetrating peptide” or “CPP” refers to a peptide that facilitates delivery of a cargo. In embodiments, the CPP is cyclic, and is represented as “cCPP”.

As used herein, the term “endosomal escape vehicle” (EEV) refers to a cCPP that is conjugated by a chemical linkage (i.e., a covalent bond or non-covalent interaction) to a linker and/or an exocyclic peptide (EP).

As used herein, the term “exocyclic peptide” (EP) refer to two or more amino acid residues linked by a peptide bond that can be conjugated to a cyclic cell penetrating peptide (cCPP) disclosed herein. The EP, when conjugated to a cyclic peptide disclosed herein, may alter the tissue distribution and/or retention of the compound. Typically, the EP comprises at least one positively charged amino acid residue, e.g., at least one lysine residue and/or at least one arginine residue. Non-limiting examples of EP are described herein. The EP can be a peptide that has been identified in the art as a “nuclear localization sequence” (NLS). Non-limiting examples of nuclear localization sequences include the nuclear localization sequence of the SV40 virus large T-antigen, the minimal functional unit of which is the seven amino acid sequence PKKKRKV (SEQ ID NO: 42), the nucleoplasmin bipartite NLS with the sequence NLSKRPAAIKKAGQAKKKK (SEQ ID NO: 169), the c-myc nuclear localization sequence having the amino acid sequence PAAKRVKLD (SEQ ID NO: 170) or RQRRNELKRSF (SEQ ID NO: 171) the sequence RMRKFKNKGKDTAELRRRRVEVSVELRKAKKDEQILKRRNV (SEQ ID NO: 480) of the IBB domain from importin-alpha, the sequences VSRKRPRP (SEQ ID NO: 174) and PPKKARED (SEQ ID NO: 175) of the myoma T protein, the sequence PQPKKKPL (SEQ ID NO: 176) of human p53, the sequence SALIKKKKKMAP (SEQ ID NO: 177) of mouse c-abl IV, the sequences DRLRR (SEQ ID NO: 178) and PKQKKRK (SEQ ID NO: 179) of the influenza virus NS1, the sequence RKLKKKIKKL (SEQ ID NO: 180) of the Hepatitis virus delta antigen and the sequence REKKKFLKRR (SEQ ID NO: 181) of the mouse Mxl protein, the sequence KRKGDEVDGVDEVAKKKSKK (SEQ ID NO: 182) of the human poly (ADP-ribose) polymerase and the sequence RKCLQAGMNLEARKTKK (SEQ ID NO: 183) of the steroid hormone receptors (human) glucocorticoid. International Publication No. 2001/038547 describes additional examples of NLSs and is incorporated by reference herein in its entirety.

As used herein, “linker” or “L” refers to a moiety that covalently bonds one or more moieties (e.g., an exocyclic peptide (EP) and/or a cargo) to the cyclic cell penetrating peptide (cCPP).

As used herein, a “delivery construct” refers to a construct that facilitates delivery of cargo to a cell. In embodiments, the delivery construct facilitates endosomal escape. In embodiments, the delivery construct comprises a cell penetrating peptide (CPP). In embodiments, the CPP comprises a cyclic cell penetrating peptide (cCPP). In embodiments, the delivery construct comprises a CPP conjugated to a linker. In embodiments the delivery construct comprises an endosomal escape vehicle (EEV). In embodiments, the EEV comprises a cCPP, a linker and an exocyclic peptide (EP). In embodiments, the delivery construct is conjugated to a lipid cargo and can be referred to herein as a “lipid delivery construct”. In embodiments, the delivery construct is conjugated to a payload of a lipid-based particle and can be referred to herein as a “payload delivery construct.”.

As used herein, “lipid conjugate” refers to a lipid that is conjugated to a delivery construct. In embodiments, the lipid is conjugated to the delivery construct via a linker.

As used herein, “GEM conjugate” refers to a component of a gene editing machinery (GEM) that is conjugated to a delivery construct. In embodiments, the GEM is conjugated to the delivery construct via a linker.

The terms “peptide,” “protein,” and “polypeptide” are used interchangeably to refer to a natural or synthetic molecule comprising two or more amino acids linked by the carboxyl group of one amino acid to the alpha amino group of another. Two or more amino acid residues can be linked by the carboxyl group of one amino acid to the alpha amino group. Two or more amino acids of the polypeptide can be joined by a peptide bond. The polypeptide can include a peptide backbone modification in which two or more amino acids are covalently attached by a bond other than a peptide bond. The polypeptide can include one or more non-natural amino acids, amino acid analogs, or other synthetic molecules that are capable of integrating into a polypeptide. The term polypeptide includes naturally occurring and artificially occurring amino acids. The term polypeptide includes peptides, for example, that include from about 2 to about 100 amino acid residues as well as proteins, that include more than about 100 amino acid residues, or more than about 1000 amino acid residues, including, but not limited to therapeutic proteins such as antibodies, enzymes, receptors, soluble proteins and the like.

As used herein, the term “contiguous” refers to two amino acids, which are connected by a covalent bond. For example, in the context of a representative cyclic cell penetrating peptide (cCPP) such as

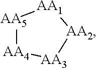

AA₁/AA₂, AA₂/AA₃, AA₃/AA₄, and AA₅/AA₁ exemplify pairs of contiguous amino acids.

A residue of a chemical species, as used herein, refers to a derivative of the chemical species that is present in a particular product. To form the product, at least one atom of the species is replaced by a bond to another moiety, such that the product contains a derivative, or residue, of the chemical species. For example, the cyclic cell penetrating peptides (cCPP) described herein have amino acids (e.g., arginine) incorporated therein through formation of one or more peptide bonds. The amino acids incorporated into the cCPP may be referred to residues, or simply as an amino acid. Thus, arginine or an arginine residue refers to

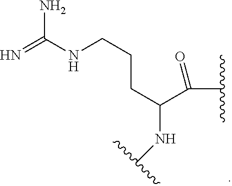 [SMILES: *NC(CCCNC(=N)N)C(*)=O]

The term “protonated form thereof” refers to a protonated form of an amino acid. For example, the guanidine group on the side chain of arginine may be protonated to form a guanidinium group. The structure of a protonated form of arginine is

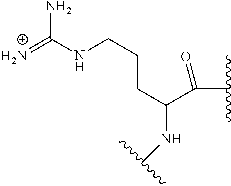 [SMILES: *NC(CCCNC(N)=[NH2+])C(*)=O]

As used herein, the term “chirality” refers to a molecule that has more than one stereoisomer that differs in the three-dimensional spatial arrangement of atoms, in which one stereoisomer is a non-superimposable mirror image of the other. Amino acids, except for glycine, have a chiral carbon atom adjacent to the carboxyl group. The term “enantiomer” refers to stereoisomers that are chiral. The chiral molecule can be an amino acid residue having a “D” and “L” enantiomer. Molecules without a chiral center, such as glycine, can be referred to as “achiral.”

As used herein, the term “hydrophobic” refers to a moiety that is not soluble in water or has minimal solubility in water. Generally, neutral moieties and/or non-polar moieties, or moieties that are predominately neutral and/or non-polar are hydrophobic. Hydrophobicity can be measured by one of the methods disclosed herein.

As used herein “aromatic” refers to an unsaturated cyclic molecule having 4n+2π electrons, wherein n is any integer. The term “non-aromatic” refers to any unsaturated cyclic molecule which does not fall within the definition of aromatic.

“Alkyl”, “alkyl chain” or “alkyl group” refer to a fully saturated, straight or branched hydrocarbon chain radical having from one to forty carbon atoms, and which is attached to the rest of the molecule by a single bond. Alkyls comprising any number of carbon atoms from 1 to 40 are included. An alkyl comprising up to 40 carbon atoms is a C₁-C₄₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C6 alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, f-butyl, n-pentyl, (-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene”, “alkylene chain” or “alkylene group” refers to a fully saturated, straight or branched divalent hydrocarbon chain radical, having from one to forty carbon atoms. Non-limiting examples of C₂-C₄₀ alkylene include ethylene, propylene, n-butylene, ethenylene, propenylene, n-butenylene, propynylene, n-butynylene, and the like. Unless stated otherwise specifically in the specification, an alkylene chain can be optionally substituted.

The term “alkanediyl” and “alkanediyl group” refer to a divalent group that is a diradical of an alkane. Alkanediyls may include straight-chain, branched, cyclic, and bicyclic alkyl groups, and combinations thereof, including both unsubstituted and substituted alkanediyl groups. (single radical of alkane is an alkyl)

“Alkenyl”, “alkenyl chain” or “alkenyl group” refers to a straight or branched hydrocarbon chain radical having from two to forty carbon atoms and having one or more carbon-carbon double bonds. Each alkenyl group is attached to the rest of the molecule by a single bond. Alkenyl groups comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene”, “alkenylene chain” or “alkenylene group” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

The terms “alkanediyl” and “alkanediyl group” refer to a divalent group that is a diradical of an alkene. Alkenediyls include includes groups that are linear, branched, cyclic, or any combination thereof. An alkanediyl has one or more double bonds. Each radical may be a radical of a double bond (e.g., ⋅CH═CH2—) or of a single bond (e.g., ⋅CHCH₂—).

“Alkoxy” or “alkoxy group” refers to the group —OR, where R is alkyl, alkenyl, alkynyl, cycloalkyl, or heterocyclyl as defined herein. Unless stated otherwise specifically in the specification, an alkoxy group can be optionally substituted.

“Acyl” or “acyl group” refers to groups —C(O)R, where R is hydrogen, alkyl, alkenyl, alkynyl, carbocyclyl, or heterocyclyl, as defined herein. Unless stated otherwise specifically in the specification, acyl can be optionally substituted.

“Alkylcarbamoyl” or “alkylcarbamoyl group” refers to the group —O—C(O)—NRₐR_{b}, where Rₐ and R_{b} are the same or different and are independently an alkyl, alkenyl, alkynyl, aryl, heteroaryl, as defined herein, or RₐR_{b} can be taken together to form a cycloalkyl group or heterocyclyl group, as defined herein. Unless stated otherwise specifically in the specification, an alkylcarbamoyl group can be optionally substituted.

“Alkylcarboxamidyl” or “alkylcarboxamidyl group” refers to the group —C(O)—NRₐR_{b}, where Rₐ and R_{b} are the same or different and are independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, an alkylcarboxamidyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Heteroaryl” refers to a 5- to 20-membered ring system radical comprising hydrogen atoms, one to thirteen carbon atoms, one to six heteroatoms selected from nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1/-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double-or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-beteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl. In addition, each of the foregoing substituents can also be optionally substituted with one or more of the above substituents.

The term “catenated” refers a carbon that is a part of a chain of linked carbons, such as an alkyl or alkenyl, or refers to a heteroatom (e.g., oxygen, sulfur, or nitrogen) that replaces at least one carbon of the chain. For example, ether groups contain one catenary oxygen atom with at least one carbon atom on each side of the catenary oxygen atom.

As used herein, the symbol

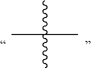

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

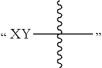

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

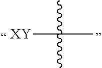

infers that when R³ is “XY”, the point of attachment bond is the same bond as the bond by which R³ is depicted as being bonded to CH₃.

As used herein, by a “subject” is an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The terms “inhibit”, “inhibiting” or “inhibition” refer to a decrease in an activity, expression, function or other biological parameter and can include, but does not require complete ablation of the activity, expression, function or other biological parameter. Inhibition can include, for example, at least about a 10% reduction in the activity, response, condition, or disease as compared to a control. In embodiments, expression, activity or function of a gene or protein is decreased by a statistically significant amount. In embodiments, activity or function is decreased by at least about 10%, about 20%, about 30%, about 40%, about 50%, and up to about 60%, about 70%, about 80%, about 90% or about 100%.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, the term “pharmaceutically acceptable carrier” refers to a carrier suitable for administration to a patient. A pharmaceutically acceptable carrier can be a sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

As used herein, the term “parenteral administration,” refers to administration through injection or infusion. Parenteral administration includes, but is not limited to, subcutaneous administration, intravenous administration, or intramuscular administration.

As used herein, the term “subcutaneous administration” refers to administration just below the skin. “Intravenous administration” means administration into a vein.

As used herein, the term “dose” refers to a specified quantity of a pharmaceutical agent provided in a single administration. In embodiments, a dose may be administered in two or more boluses, tablets, or injections. In embodiments, where subcutaneous administration is desired, the desired dose requires a volume not easily accommodated by a single injection. In embodiments, two or more injections may be used to achieve the desired dose. In embodiments, a dose may be administered in two or more injections to reduce injection site reaction in a patient.

As used herein, the term “dosage unit” refers to a form in which a pharmaceutical agent is provided. In embodiments, a dosage unit is a vial that includes lyophilized antisense oligonucleotide. In embodiments, a dosage unit is a vial that includes reconstituted antisense oligonucleotide.

The terms “modulate”, “modulating” and “modulation” refer to a perturbation of expression, function or activity when compared to the level of expression, function or activity prior to modulation. Modulation can include an increase (stimulation or induction) or a decrease (inhibition or reduction) in expression, function or activity. In embodiments, the compound disclosed herein includes a therapeutic moiety (TM) that decreases IRF-5 expression, function and/or activity. In embodiments, IRF-5 activity is modulated by modulating IRF-5 expression.

“Amino acid” refers to an organic compound that includes an amino group and a carboxylic acid group and has the general formula

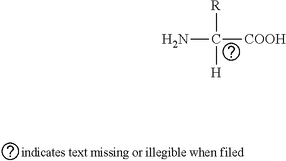

where R can be any organic group. An amino acid may be a naturally occurring amino acid or non-naturally occurring amino acid. An amino acid may be a proteogenic amino acid or a non-proteogenic amino acid. An amino acid can be an L-amino acid or a D-amino acid. The term “amino acid side chain” or “side chain” refers to the characterizing substituent (“R”) bound to the a-carbon of a natural or non-natural α-amino acid. An amino acid may be incorporated into a polypeptide via a peptide bond.

As used herein, the term “sequence identity” refers to the percentage of nucleic acids or amino acids between two oligonucleotide or polypeptide sequences, respectively, that are the same and in the same relative position. As such one sequence has a certain percentage of sequence identity compared to another sequence. For sequence comparison, typically one sequence acts as a reference sequence, to which test sequences are compared. Those of ordinary skill in the art will appreciate that two sequences are generally considered to be “substantially identical” if they contain identical residues in corresponding positions. In embodiments, the sequence identity between sequences may be determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., Trends Genet. (2000), 16: 276-277), in the version that exists as of the date of filing. The parameters used are gap open penalty of 10, gap extension penalty of 0.5, and the EBLOSUM62 (EMBOSS version of BLOSUM62) substitution matrix. The output of Needle labeled “longest identity” (obtained using the-nobrief option) is used as the percent identity and is calculated as follows: (Identical Residues×100)/(Length of Alignment−Total Number of Gaps in Alignment)

In embodiments, sequence identity may be determined using the Smith-Waterman algorithm, in the version that exists as of the date of filing.

As used herein, “sequence homology” refers to the percentage of amino acids between two polypeptide sequences that are homologous and in the same relative position. As such one polypeptide sequence has a certain percentage of sequence homology compared to another polypeptide sequence. As will be appreciated by those of ordinary skill in the art, two sequences are generally considered to be “substantially homologous” if they contain homologous residues in corresponding positions. Homologous residues may be identical residues. Alternatively, homologous residues may be non-identical residues with appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains, and substitution of one amino acid for another of the same type may often be considered a “homologous” substitution.

As is well known in this art, amino acid sequences may be compared using any of a variety of algorithms, including those available in commercial computer programs such as BLASTP, gapped BLAST, and PSI-BLAST, in existence as of the date of filing. Such programs are described in Altschul, et al., J. Mol. Biol., (1990),215(3): 403-410; Altschul, et al., Nucleic Acids Res. (1997), 25:3389-3402; Baxevanis et al., Bioinformatics A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology.

As used herein, the terms “antisense compound” and “AC” are used interchangeably to refer to a polymeric nucleic acid structure which is at least partially complementarity to a target nucleic acid molecule to which it (the AC) hybridizes. The AC may be a short (in embodiments, less than 50 bases) polynucleotide or polynucleotide homologue that includes a sequence complementary to a target sequence. In embodiments, the AC is a polynucleotide or polynucleotide homologue that includes a sequence complementary to a target sequence in a target pre-mRNA strand. The AC may be formed of natural nucleic acids, synthetic nucleic acids, nucleic acid homologues, or any combination thereof. In embodiments, the AC includes oligonucleosides. In embodiments, AC includes antisense oligonucleotides. In embodiments, the AC includes conjugate groups. Nonlimiting examples of ACs include, but are not limited to, primers, probes, antisense oligonucleotides, external guide sequence (EGS) oligonucleotides, siRNAs, oligonucleotides, oligonucleosides, oligonucleotide analogs, oligonucleotide mimetics, and chimeric combinations of these. As such, these compounds can be introduced in the form of single-stranded, double-stranded, circular, branched or hairpins and can contain structural elements such as internal or terminal bulges or loops. Oligomeric double-stranded compounds can be two strands hybridized to form double-stranded compounds or a single strand with sufficient self-complementarity to allow for hybridization and formation of a fully or partially double-stranded compound. In embodiments, an AC modulates (increases, decreases, or changes) expression of a target nucleic acid.

As used herein, the terms “targeting” or “targeted to” refer to the association of a an affecter molecule (e.g., an RNP, targeting oligonucleotide, nuclease, etc.) to a nucleic acid of interest.

As used herein, the terms “target nucleic acid sequence,” “target nucleotide sequence,” and “target sequence” refer to the nucleic acid sequence or the nucleotide sequence to which targeting oligonucleotide or antisense compound binds. Target nucleic acids include, but are not limited, to a portion of a target transcript; target RNA (including, but not limited to pre-mRNA and mRNA or portions thereof); a portion of target cDNA derived from such RNA; a portion of target non-translated RNA, such as miRNA; and/or a portion of genomic DNA. For example, in embodiments, a target nucleic acid can be a portion of genomic DNA. The term “portion” refers to a defined number of contiguous (i.e., linked) nucleotides of a nucleic acid.

As used herein, the term “transcript” or “gene transcript” refers an RNA molecule transcribed from DNA and includes, but is not limited to mRNA, pre-mRNA, and partially processed RNA.

The terms “target transcript” and “target RNA” refer to the pre-mRNA or mRNA transcript that is bound by the therapeutic moiety. The target transcript may include a target nucleotide sequence. In embodiments, the target transcript includes a splice site. In embodiments, the target RNA includes a polyadenylation site or a portion thereof.

The term “target gene” and “gene of interest” refer to the gene of which modulation of the expression and/or activity is desired or intended. The target gene may be transcribed into a target transcript that includes a target nucleotide sequence. The target transcript may be translated into a protein of interest.

The term “target protein” refers to the polypeptide or protein encoded by the target transcript (e.g., target mRNA).

As used herein, the term “expression,” “gene expression,” “expression of a gene,” or the like refers to all the functions and steps by which information encoded in a gene is converted into a functional gene product, such as a polypeptide or a non-coding RNA, in a cell. Examples of non-coding RNA include transfer RNA (IRNA) and ribosomal RNA. Gene expression of a polypeptide includes transcription of the gene to form a pre-mRNA, processing of the pre-mRNA to form a mature mRNA, translocating the mature mR.NA from the nucleus to the cytoplasm, translation of the mature mRNA into the polypeptide, and assembly of the encoded polypeptide. Expression includes partial expression. For example, expression of a gene may be referred to herein as generation of a gene transcript. Translation of a mature mRNA may be referred to herein as expression of the mature mRNA.

As used herein, “modulation of gene expression” or the like refers to modulation of one or more of the processes associated with gene expression. For example, modification of gene expression may include modification of one or more of gene transcription, RNA processing, RNA translocation from the nucleus to the cytoplasm, and translation of mRNA into a protein.

As used herein, the term “gene” refers to a nucleic acid sequence that encompasses a 5′ promoter region associated with the expression of the gene product, and any intron and exon regions and 3′ untranslated regions (“UTR”) associated with the expression of the gene product.

As used herein, the term “oligonucleotide” and “nucleic acid” are used interchangeable to refer to an oligomeric compound comprising a plurality of linked nucleotides or nucleosides. One or more nucleotides of an oligonucleotide can be modified. An oligonucleotide can comprise ribonucleic acid (RNA) or deoxyribonucleic acid (DNA). Oligonucleotides can be composed of natural and/or modified nucleobases, sugars and covalent internucleoside linkages, and can further include non-nucleic acid conjugates.

As used herein, the term “nucleoside” refers to a glycosylamine that includes a nucleobase and a sugar. Nucleosides include, but are not limited to, natural nucleosides, abasic nucleosides, modified nucleosides, and nucleosides having mimetic bases and/or sugar groups. A “natural nucleoside” or “unmodified nucleoside” is a nucleoside that includes a natural nucleobase and a natural sugar. Natural nucleosides include RNA and DNA nucleosides.

As used herein, the term “natural sugar” refers to a sugar of a nucleoside that is unmodified from its naturally occurring form in RNA (2′-OH) or DNA (2′-H).

As used herein, the term “nucleotide” refers to a nucleoside having a phosphate group covalently linked to the sugar. Nucleotides may be modified with any of a variety of substituents.

As used herein, the term “nucleobase” refers to the base portion of a nucleoside or nucleotide. A nucleobase may include any atom or group of atoms capable of hydrogen bonding to a base of another nucleic acid. A natural nucleobase is a nucleobase that is unmodified from its naturally occurring form in RNA or DNA.

As used herein, the term “heterocyclic base moiety” refers to a nucleobase that includes a heterocycle.

As used herein “internucleoside linkage” refers to a covalent linkage between adjacent nucleosides.

As used herein “natural internucleoside linkage” refers to a 3′ to 5′ phosphodiester linkage.

As used herein, the term “modified internucleoside linkage” refers to any linkage between nucleosides or nucleotides other than a naturally occurring internucleoside linkage.

As used herein the term “chimeric antisense compound” refers to an antisense compound, having at least one sugar, nucleobase and/or internucleoside linkage that is differentially modified as compared to the other sugars, nucleobases and internucleoside linkages within the same oligomeric compound. The remainder of the sugars, nucleobases and internucleoside linkages can be independently modified or unmodified. In general, a chimeric oligomeric compound will have modified nucleosides that can be in isolated positions or grouped together in regions that will define a particular motif. Any combination of modifications and or mimetic groups can include a chimeric oligomeric compound as described herein.

As used herein, the term “mixed-backbone antisense oligonucleotide” refers to an antisense oligonucleotide wherein at least one internucleoside linkage of the antisense oligonucleotide is different from at least one other internucleoside linkage of the antisense oligonucleotide.

As used herein, the term “nucleobase complementarity” refers to a nucleobase that is capable of base pairing with another nucleobase. For example, in DNA, adenine (A) is complementary to thymine (T). For example, in RNA, adenine (A) is complementary to uracil (U). In embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

As used herein, the term “non-complementary nucleobase” refers to a pair of nucleobases that do not form hydrogen bonds with one another or otherwise support hybridization.

As used herein, the term “complementary” refers to the capacity of an oligomeric compound to hybridize to another oligomeric compound or nucleic acid through nucleobase complementarity. In embodiments, an antisense compound and its target are complementary to each other when a sufficient number of corresponding positions in each molecule are occupied by nucleobases that can bond with each other to allow stable association between the antisense compound and the target. One skilled in the art recognizes that the inclusion of mismatches is possible without eliminating the ability of the oligomeric compounds to remain in association. Therefore, described herein are antisense compounds that may include up to about 20% nucleotides that are mismatched (i.e., are not nucleobase complementary to the corresponding nucleotides of the target). In embodiments, the antisense compounds contain no more than about 15%, for example, not more than about 10%, for example, not more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99% or 100% nucleobase complementary to a target nucleic acid.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymidine and uracil which pair through the formation of hydrogen bonds. The natural base guanine is nucleobase complementary to the natural bases cytosine and 5-methyl cytosine. Hybridization can occur under varying circumstances.

As used herein, the term “specifically hybridizes” refers to the ability of an oligomeric compound to hybridize to one nucleic acid site with greater affinity than it hybridizes to another nucleic acid site. In embodiments, an antisense oligonucleotide specifically hybridizes to more than one target site. In embodiments, an oligomeric compound specifically hybridizes with its target under stringent hybridization conditions.

“Stringent hybridization conditions” and “stringent hybridization wash conditions” in the context of nucleic acid hybridization are sequence dependent and are different under different environmental parameters. An extensive guide to the hybridization of nucleic acids is found in Tijssen, Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleotide sequences which have more than 100 complementary residues on a filter in a Southern or Northern blot is 50% formamide with 1 mg of heparin at 42° C., with the hybridization being carried out overnight. An example of highly stringent wash conditions is 0.15 M NaCl at 72° C. for about 15 minutes. An example of stringent wash conditions is a 0.2× SSC wash at 65° C. for 15 minutes (see, Sambrook and Russel, Molecular Cloning: A laboratory Manual, 3^{rd} ed., Cold Spring Harbor Laboratory Press, 2001 for a description of SSC buffer). Often, a high stringency wash is preceded by a low stringency wash to remove background probe signal. An example of a medium stringency wash for a duplex of, e.g., more than 100 nucleotides, is 1× SSC at 45° C. for 15 minutes. An example of a low stringency wash for a duplex of, e.g., more than 100 nucleotides, is 4-6× SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide.

As used herein, the term “2′-modified” or “2′-substituted” means a sugar that includes substituent at the 2′ position other than H or OH. 2′-modified monomers, include, but are not limited to, BNA's and monomers (e.g., nucleosides and nucleotides) with 2′-substituents, such as allyl, amino, azido, thio, O-allyl, O-C1-C10 alkyl, —OCF3, O—(CH2)2-O—CH3, 2′-O(CH2)2SCH3, O—(CH2)2—O—N(Rm)(Rn), or O—CH2—C(═O)—N(Rm)(Rn), where each Rm and Rn is, independently, H or substituted or unsubstituted C1-C10 alkyl.

As used herein, the term “MOE” refers to a 2′-O-methoxyethyl substituent.

As used herein, the term “high-affinity modified nucleotide” refers to a nucleotide having at least one modified nucleobase, internucleoside linkage or sugar moiety, such that the modification increases the affinity of an antisense compound that includes the modified nucleotide to a target nucleic acid. High-affinity modifications include, but are not limited to, BNAs, LNAs and 2′-MOE.

As used herein the term “mimetic” refers to groups that are substituted for a sugar, a nucleobase, and/or internucleoside linkage in an AC or targeting nucleotide. Generally, a mimetic is used in place of the sugar or sugar-internucleoside linkage combination, and the nucleobase is maintained for hybridization to a selected target. Representative examples of a sugar mimetic include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In embodiments, a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

As used herein, the term “bicyclic nucleoside” or “BNA” refers to a nucleoside wherein the furanose portion of the nucleoside includes a bridge connecting two atoms on the furanose ring, thereby forming a bicyclic ring system. BNAs include, but are not limited to, α-L-LNA, β-D-LNA, ENA, Oxyamino BNA (2′-O—N(CH3)—CH2-4′) and Aminooxy BNA (2′-N(CH3)—O—CH2-4′).

As used herein, the term “4′ to 2′ bicyclic nucleoside” refers to a BNA wherin the bridge connecting two atoms of the furanose ring bridges the 4′ carbon atom and the 2′ carbon atom of the furanose ring, thereby forming a bicyclic ring system.

As used herein, a “locked nucleic acid” or “LNA” refers to a nucleotide modified such that the 2′-hydroxyl group of the ribosyl sugar ring is linked to the 4′ carbon atom of the sugar ring via a methylene groups, thereby forming a 2′-C,4′-C-oxymethylene linkage. LNAs include, but are not limited to, α-L-LNA, and β-D-LNA.

As used herein, the term “cap structure” or “terminal cap moiety” refers to chemical modifications, which have been incorporated at either end of an AC.

The term “therapeutic polypeptide” refers to a naturally occurring or recombinantly produced macromolecule that includes two or more amino acids and has therapeutic, prophylactic or other biological activity.

Various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7.3, 4, 5, 5.3, and 6. This applies regardless of the breadth of the range.

In the description herein particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### EXAMPLES

The present disclosure is illustrated by the following examples. It is to be understood that the particular examples, materials, amounts, and procedures are to be interpreted broadly in accordance with the scope and spirit of the disclosure as set forth herein.

### General LNP Formulation Protocol

To formulate the LNPs of the Examples, stock solutions of each lipid, sterol, and cargo (e.g., mRNA) were prepared. Lipids (helper lipids, PEGylated lipids, ionizable lipids, conjugate lipids) and sterols (e.g., cholesterol) were dissolved in ethanol to concentrations indicated in each respective Example. The cargo was dissolved or diluted to form an aqueous stock solution. When the cargo was mRNA, the aqueous stock solution of mRNA included 50 mM sodium acetate. The pH of the stock solution of mRNA and sodium acetate was adjusted to 5 using acetic acid.

The stock solutions of lipids and cholesterol were combined, and ethanol was added to ensure the final formulation aqueous to ethanol ratio was 3:1 by volume. The lipid-cholesterol-ethanol solution was injected into the cargo solution (e.g., the acidic mRNA solution) to form the formulation having the specified component amounts for each formulation. The formulation was vortexed on high for 60 seconds, allowed to stand at room temperature for 15 minutes; the particle size determined according to the particle dimensional analysis test method; then dialyzed overnight. Following dialysis, the average particle size was determined a second time using according to the particle dimensional analysis test method and the encapsulation efficiency of the cargo was determined according to the encapsulation efficiency test method. Formulations were stored at 4° C. until further use/analysis.

### Test Methods

### Particle Dimensional Analysis

The average particle size and polydispersity index of the formulated LNPs were measure. All measurements were taken on a Zetasizer Ultra Red Label (available from Malvern and the Malvern Panalytical in Malvern, United Kingdom) using Zen0040 low-volume cuvettes (available from Malvern Panalytical) for measurements. Samples included 76 μL of twice-filtered (using 0.22 um nylon filters) polymerase chain reaction grade water and 4 μL of a given formulation. The “Size” measurement was taken on the Malvern software; the species of interest was selected as a liposome; and the dispersant was selected as water. The “Size” was measured a total of five times in series and the provided size and PDI was give as the computer generated average of these runs.

### Encapsulation Efficiency (EE)

For encapsulation efficiency (EE; displayed as a percent) of mRNA cargo, the QUANT-IT assay (available from Millipore Sigma in Burlington, MA) was used according to manufacture protocols. Standard curves for RNA detection was made and a separate standard curve for RNA concentration was prepared with the addition of 3 μL of Triton X-100 in each sample. LNP formulation samples were prepared by mixing 300 μL of 1× tris-EDTA (TE) buffer; 300 μL of 200-fold diluted RIBOGREEN Reagent (available from Millipore Sigma); and 3 uL of the formulation. One sample of this composition was analyzed and an analogous sample that included 3 μL Triton X-100 was also analyzed. All samples were measured on a flat-bottom black 96-well plate using a fluorescent plate reader measure excitation at 500 nm and emission at 525 nm. Data was analyzed by subtracting the 0 ng/ml intensity values from every other value obtained to get adjusted values. The EE % is found by the following formula EE %=(Intensity_triton−Intensity_formulated)/Intensity_Triton*100% where Intenisty_triton is the adjusted intensity of the formulation that was treated with Triton X-100 and Intensity_formulation is the adjusted intensity of the formulation sample that was NOT treated with Triton X-100. mRNA concentrations were determined by fitting the Intensity Triton values to the Triton X-100 RNA standard curve and back calculating concentrations of the formulation itself.

### Example 1. Comparison of LNPs Comprising DMG-PEG2K-Conjugated Lipids and LNPs not Comprising the DMG-PEG2K-Conjugated Lipids

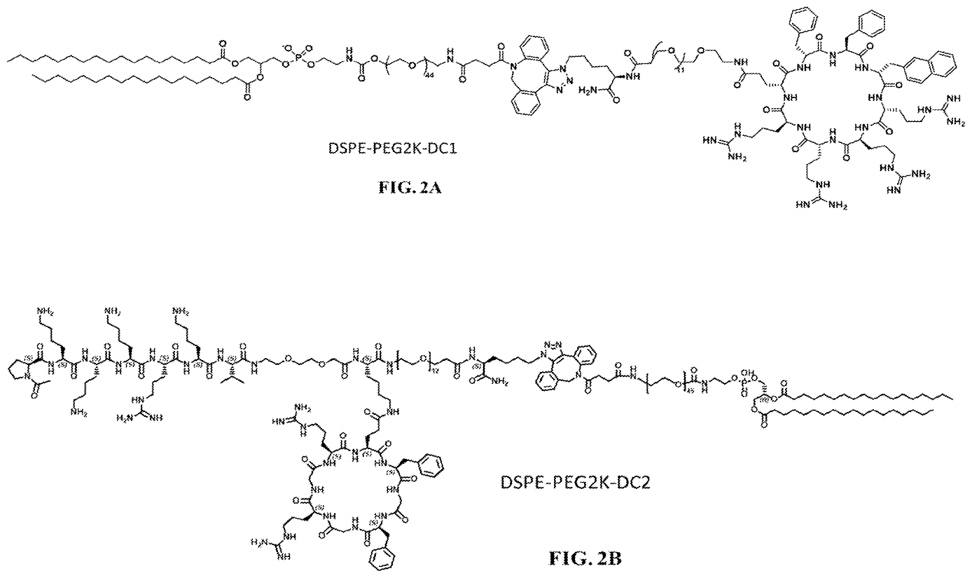

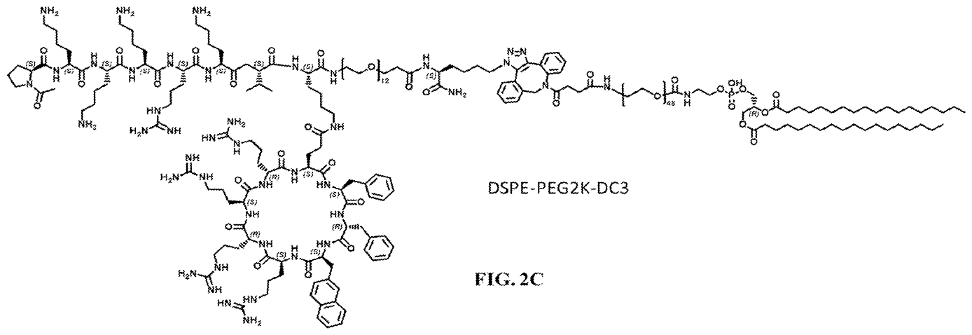

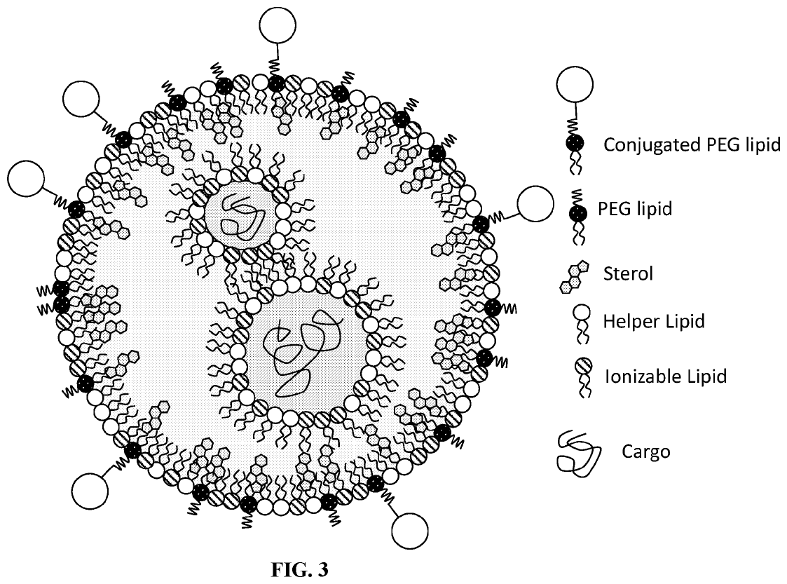

The properties of LNPs that included various amounts of a PEGylated conjugated lipid (DSPE-PEG2K-DC1; FIG. 2A) and equivalent LNPs that did not include the conjugated lipid were compared.

To synthesize the DSPE-PEG2K-DC1 lipid, 10 mg of DSPE-PEG-2K-DBCO (FIG. 4) was dissolved in 50/50 acetonitrile/water to form a solution having a concentration of 10 mg/mL DSPE-PEG2K-DBCO. To this solution, 6.59 mg of DC1 (1 equivalent by mass, but peptide is assumed to be 75% pure with solvent impurities) was added. The reaction was allowed to proceed at room temperature overnight and was monitored by HPLC (0-95% acetonitrile with 0.2% formic acid gradient in water with 0.2% formic acid). Once the reaction was deemed complete (>90% product relative to starting material), the solvents were removed by lyophilization and the crude lipid-DCI conjugate was used in formulations.

Seven LNP formulations were prepared. Formulations F1, F2, F3, F4, and F5 included DSPE-PEG2K-DC1. Formulation F6 was a control and did not include a lipid conjugate. Formulation F7 was also a control where the mRNA was pre-incubated with DCI for 30 minutes in the acidic aqueous solution and then the formulation proceeded identically to the General LNP Formulation Protocol. The cargo (therapeutic molecule) for each formulation was eGFP mRNA (mRNA length=996; mRNA molecular weight=329825 g/mol). Each formulation included an N:P ratio of 5.

Table 9 is a formulation table showing the composition of the stock solutions and the Formulation of F6, a four component LNP system (excluding the cargo).

TABLE 9
Formulation table of F6.

| | | Stock | Formulation | | | |
|---|---|---|---|---|---|---|
| | | Solutions | volume | | | |
| | MW | Concentration | of stock | Mass | | |
| Lipids | (g/mol) | mg/mL | (uL) | (mg) | Moles | Mol-% |
| SM-102 | 710.2 | 100 | 2.6808 | 0.2681 | 3.77E−07 | 50 |
| cholesterol | 386.65 | 20 | 5.4001 | 0.108 | 2.79322E−07 | 37 |
| DSPC | 790.14 | 15 | 3.9768 | 0.0597 | 7.55562E−08 | 10 |
| DMG-PEG200 | 2509.2 | 1 | 56.8293 | 0.0568 | 2.26E−08 | 3 |
| Ethanol | | | 14.4463 | | | |
| Total | | | 83.3333 | 0.4926 | 7.55E−07 | 100 |
| | | | Formulation | | | |
| | | Stock | | | | Final |
| | | Solutions | Volume | | Final | Volume of |
| | Concentration | Mass | of Stock | | Concentration | Formulation |
| mRNA | (mg/mL) | (mg) | (uL) | Moles | (mg/mL) | (mL) |
| eGFP | 1 | 0.025 | 250 | 7.57978E−11 | 0.1 | 0.25 |

Table 10 is a formulation table showing the composition of the stock solutions and the formulation of F1 and F2, five component LNP systems (excluding the cargo). The molar ratios and volumes used for the ethanolic lipid mixtures were adjusted to accommodate a five-component system as opposed to the four component LNP system of formulation F14 (Table 9). For the 5 component LNP systems (F1, F2, F3, F4, and F5) the molar amount of DCI used is equivalent to the moles of DSPE-PEG2K-DCI (labeled DBCO-DC1 in Table 9) used in a 0.5 mol-% or 0.1 mol-% formulation for the five-component system (excluding the cargo).

TABLE 10
Formulation table of formulations F1 and F2.

| | | Stock | Formulation | | | |
|---|---|---|---|---|---|---|
| | | Solutions | volume | | | |
| | MW | Concentration | of stock | Mass | | |
| Lipids | (g/mol) | mg/mL | (uL) | (mg) | Moles | mol-% |
| SM-102 | 710.2 | 100 | 2.6808 | 0.2681 | 3.77E−07 | 50.00 |
| cholesterol | 386.65 | 20 | 5.4001 | 0.108 | 2.79E−07 | 37.00 |
| DSPC | 790.14 | 15 | 3.9768 | 0.0597 | 7.56E−08 | 10.00 |
| DMG- | 2509.2 | 1 | 47.3578 | 0.0474 | 1.89E−08 | 2.50 |
| PEG2000 | | | | | | |
| DBCO-PEG2k- | 5076.21 | 10 | 1.9161 | 0.0192 | 3.78E−09 | 0.50 |
| DC1 | | | | | | |
| Ethanol | | | 22.0017 | | | |
| Total | | | 61.3316 | 0.5024 | 7.51E−07 | 100 |
| | | | Formulation | | | |
| | | Stock | | | | Final |
| | | Solutions | Volume | | Final | Volume of |
| | Concentration | Mass | of Stock | | Concentration | Formulation |
| mRNA | (mg/mL) | (mg) | (uL) | Moles | (mg/mL) | (mL) |
| eGFP | 1 | 0.025 | 250 | | 0.1 | 0.25 |

Table 11 shows the mol-% of each component (excluding cargo) in the LNPs of Formulations F1-F7. In all formulations, the total amount of PEGylated lipids (DMG-PEG200 and DBCO-DC1) was 3%.

TABLE 11
Components and amounts for various LNP formulations.

| | SM- | | | DMG- | DBCO- |
|---|---|---|---|---|---|
| | 102 | cholesterol | DSPC | PEG200 | DC1 |
| Formulation | mol-% | mol-% | mol-% | mol-% | mol-% |
| F1 | 50 | 37 | 10 | 2.5 | 0.5 |
| F1(a) | 50 | 37 | 10 | 2.5 | 0.5 |
| F2 | 50 | 37 | 10 | 2.9 | 0.1 |
| F3 | 50 | 37 | 10 | 2.5 | 0.5 |
| F4 | 50 | 37 | 10 | 2.9 | 0.1 |
| F5 | 50 | 37 | 10 | 3 | 0 |
| F6 | 50 | 37 | 10 | 3 | 0 |

The average size of the LNPs, polydispersity index (PDI), and encapsulation efficiency (EE %) were determined for the Formulations of Table 11 (Table 12). Formulations having the highest amount of the DCI-lipid conjugate had the highest LNP average size and highest PDI.

TABLE 12
Various properties of the LNP Formulations of Table 10

| | LNP size | | EE | mRNA | mRNA |
|---|---|---|---|---|---|
| Formulation | (nm) | PDI | % | (ug/mL) | (nM) |
| F1 | 214.7 | 0.2619 | 90.91 | 30.44 | 92.3 |
| F1(a) | 216.8 | 0.4046 | 89.67 | 33.56 | 101.75 |
| F2 | 123.3 | 0.1371 | 82.67 | 39.88 | 120.91 |
| F3 | 93.74 | 0.135 | 96.7 | 45.02 | 136.49 |
| F4 | 87.76 | 0.156 | 95.76 | 36.91 | 111.9 |
| F5 | 84.52 | 0.1432 | 94.54 | 41.67 | 126.34 |
| F6 | 110.5 | 0.1772 | 91 | 26.32 | 79.79 |

LNP cellular uptake for the LNP formulations in Table 10 was tested. HeLa cells (Human epithelial cells, ATCC) were seeded at 0.05×10⁶ cells per well in 1000 μL DMEM media (Dulbecco's Modified Eagle Medium, GIBCO) containing 10% FBS (GIBCO) and 5% penicillin streptomycin (GIBCO) in 24-well plate. The LNP formulations of Table 10 containing the eGFP mRNA were prepared with a total treatment dose of 250 ng and 1000 ng eGFP mRNA per well in triplicates. The cells were incubated for 24 h at 37° C. Cells were then trypsinized and resuspended in PBS and centrifuged at 200 g for 5 min. The supernatant was removed, and the pelleted cells were redispersed in 500 μL of PBS+2% FBS per well and transferred to FACS tubes and read on a flow cytometry analyzer (BD FACSCanto™ II).

The data presented are the mean fluorescent intensity of the signals from each cell treatment analyzed. For transfection efficiency, treated cells were gated for EGFP expression, and the data presented are the percentage of cells analyzed with higher EGFP expression than untreated cells.

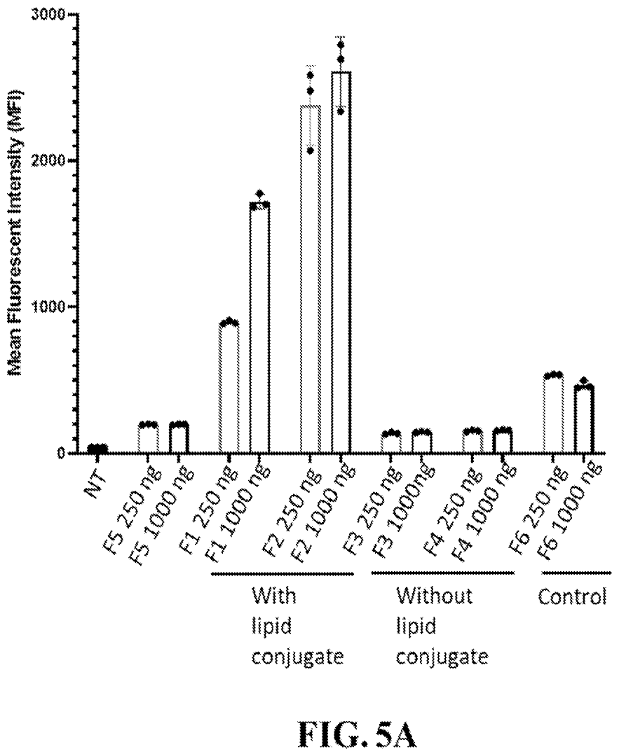

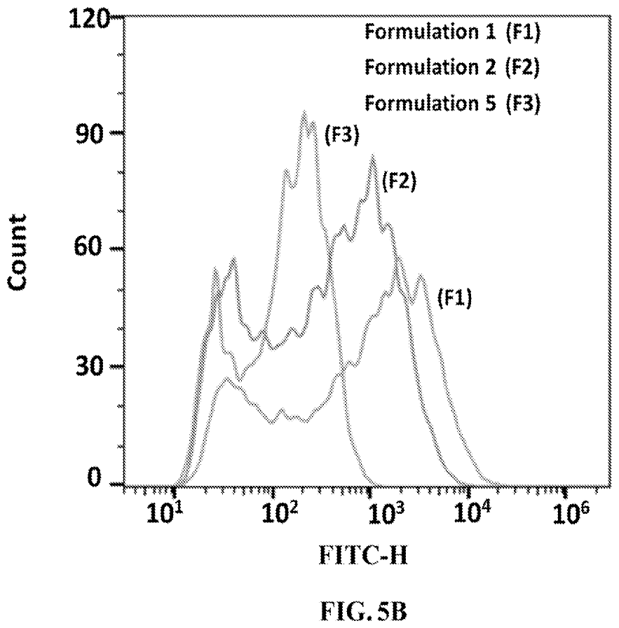

Cells treated with LNP formulations that include the lipid conjugate had a higher mean fluorescence intensity than cells treated with LNP formulations that did not include the lipid conjugate (FIG. 5A). The increase in mean fluorescence intensity for cells treated with LNP formulations that include a lipid conjugate was dose dependent.

FIG. 5B shows a shift in the FACS plot for the mean intensity of EGFP (x axis) for cell treated with LNP formulations that included lipid conjugates compared to cells treated with an LNP formulation that did not include a lipid conjugate. An increase in the meant intensity of EGFP indicated increased EGFP expression and therefore increased cellular uptake of LNPs that include lipid conjugates compared to LNPs that do not include lipid conjugates.

### Example 2. Evaluating the Properties of LNPs Comprising Various Amounts of Various Lipid Conjugates and the Ability of such LNPs to Infiltrate Cells

LNP formulations having various amounts of different PEGylated lipid conjugates were evaluated for their physical properties and their ability to enter cells. The PEGylated lipid conjugates DSPE-PEG2K-DC1 (FIG. 2A); DPSE-PEG2K-DC2 (FIG. 2B) and DSPE-PEG2K-DC3 (FIG. 2C) were synthesized using a method similar to Example 1.

Formulations of the present Example were analyzed using the dimensional analysis and encapsulation efficiency Tests Methods. In the experiments of this Example, to minimize delivery construct interference in the encapsulation efficiency Test Method, heparin (25 ug/ml) was used for assaying mRNA concentrations with LNPs formulated using DSPE-PEG2K-DC1. DC2 does not inhibit the fluorescence of RIBOGREEN in a substantive enough manner to indicate heparin rescue.

In each formulation of the present Example, the cargo (therapeutic molecule) was cGFP mRNA. HeLa cells were treated with the various formulations for 24 hours before using fluorescence activated cell sorting (FACS) flow cytometry to quantify eGFP expression in the treated cells. The higher the mean fluorescence intensity, the greater the expression of eGFP, ant therefore, the greater the transfection efficiency.

Four separate experiments were conducted to test the impact of identity and amount of the PEGylated lipid conjugate; the total amount of PEGylated lipid present in the LNP (PEGylated lipid+lipid conjugate); and the identity of the ionizable lipid in LNP formulation on the properties of the LNPs and the free uptake transfection efficiency of the LNPs.

In a first experiment, seven LNP formulations that included varying amounts (0, 0.01 mo-%, 0.025 mol-%, 0.05 mol-%, 0.1 mol-%, 0.25 mol-%, and 0.5 mol-%) of DSPE-PEG2K-DC1 and DPSE-PEG2K-DC2 were prepared according to the General LNP Formulation Protocol. In addition to the DSPE-PEG2K-DC1 lipid and DPSE-PEG2K-DC2 lipid, each formulation included 50 mol-% MC3; 38.5 mol-% cholesterol; and 10 mol-% DSPC. DMG-PEG2K was added to each formulation in an amount that made the total PEGylated lipid amount 3 mol-%. For example, in the Formulation where DSPE-PEG2K-DCI was present at 0.1 mol-%, DMG-PEG2K was present at 2.9 mol-%.

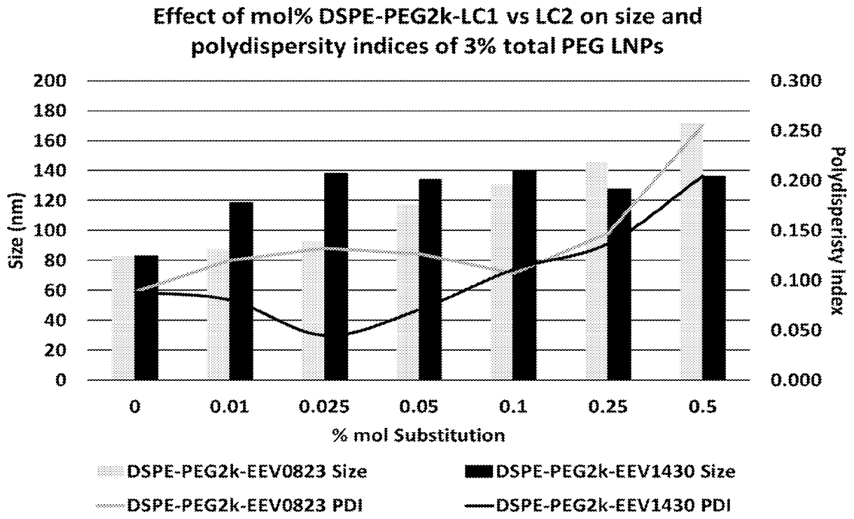

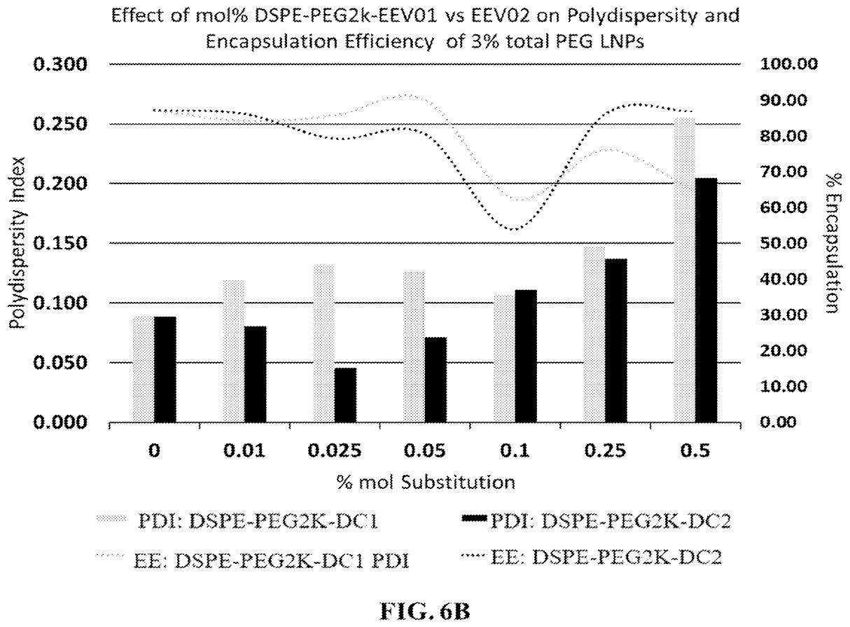

FIG. 6A and 6B show the physical properties of the formulations in the first experiment. Increased incorporation of lipid conjugates results in an increase in size and polydispersity of the LNP. Size and polydispersity of the LNP affects encapsulation efficiency. The rise in encapsulation efficiency after 0.1-mol-% incorporation of the lipid conjugate suggests reconfiguration of the LNP.

In a second experiment, thirteen LNP formulations having varying amounts of DSPE-PEG2K-DC1 and DPSE-PEG2K-DC2 were prepared according to the General LNP Formulation Protocol. Table 13A shows the components and amounts of the formulations used (Formulations 20-32). DMG-PEG2K was added to each formulation in an amount that made the total PEGylated lipid amount 3 mol-%. All formulations were formulated with eGFP mRNA at a N:P ratio of 6.

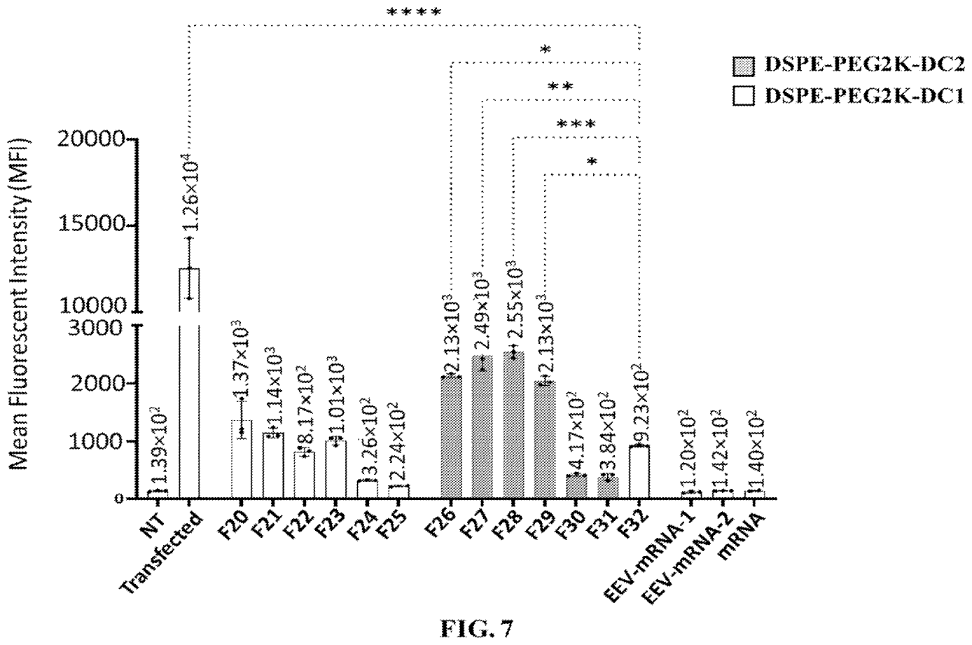

To investigate if the EEVs form a stoichiometric complex with mRNA, two aqueous formulations having an DC1:mRNA ratio and DC2:mRNA ratio of 5.92 (corresponding to an LNP formulation having 0.05 mol-% of a PEGylated lipid conjugate. Formulation EEV-mRNA-1 included 5 μg/mL EGFP mRNA and 0.18 μg/mL EEV01. Formulation EEV-mRNA-2 included 5 μg/mL EGFP mRNA and 0.260 μg/mL EEV01. A control mRNA formulation (mRNA in FIG. 7) included 10 μg/mL EGFP mRNA. Additionally, another mRNA only control was included where the mRNA was transfected (not free uptake).

TABLE 13A
Formulations F20-F32

| | | Total | | Mol-% | | | |
|---|---|---|---|---|---|---|---|
| | mol-% | mol-% | | PEGylated- | | | mol-% |
| Formulation | D-Lin- | PEGylated | Lipid | lipid | mol-% | mol-% | DMG- |
| No. | MC3-DMA | lipids | conjugate | conjugate | Cholesterol | DSPC | PEG2K |
| F20 | 50 | 3 | DSPE- | 0.01 | 37 | 10 | 2.99 |
| | | | PEG2k- | | | | |
| | | | EEV01 | | | | |
| F21 | 50 | 3 | DSPE- | 0.025 | 37 | 10 | 2.975 |
| | | | PEG2k- | | | | |
| | | | EEV01 | | | | |
| F22 | 50 | 3 | DSPE- | 0.05 | 37 | 10 | 2.95 |
| | | | PEG2k- | | | | |
| | | | EEV01 | | | | |
| F23 | 50 | 3 | DSPE- | 0.1 | 37 | 10 | 2.9 |
| | | | PEG2k- | | | | |
| | | | EEV01 | | | | |
| F24 | 50 | 3 | DSPE- | 0.25 | 37 | 10 | 2.75 |
| | | | PEG2k- | | | | |
| | | | EEV01 | | | | |
| F25 | 50 | 3 | DSPE- | 0.5 | 37 | 10 | 2.5 |
| | | | PEG2k- | | | | |
| | | | EEV01 | | | | |
| F26 | 50 | 3 | DSPE- | 0.01 | 37 | 10 | 2.99 |
| | | | PEG2k- | | | | |
| | | | EEV02 | | | | |
| F27 | 50 | 3 | DSPE- | 0.025 | 37 | 10 | 2.975 |
| | | | PEG2k- | | | | |
| | | | EEV02 | | | | |
| F28 | 50 | 3 | DSPE- | 0.05 | 37 | 10 | 2.95 |
| | | | PEG2k- | | | | |
| | | | EEV02 | | | | |
| F29 | 50 | 3 | DSPE- | 0.1 | 37 | 10 | 2.9 |
| | | | PEG2k- | | | | |
| | | | EEV02 | | | | |
| F30 | 50 | 3 | DSPE- | 0.25 | 37 | 10 | 2.75 |
| | | | PEG2k- | | | | |
| | | | EEV02 | | | | |
| F31 | 50 | 3 | DSPE- | 0.5 | 37 | 10 | 2.5 |
| | | | PEG2k- | | | | |
| | | | EEV02 | | | | |
| F32 | 50 | 3 | N/A | 0 | 37 | 10 | 3 |

Table 13B shows the physical properties of the LNPs of formulations F20-F32. Generally, the size of the LNPs and the polydispersity (PDI) increases as the mol-% of the lipid conjugate increases.

TABLE 13B
Physical properties of the LNPs of formulations F20-F32.

| | | Total | | |
|---|---|---|---|---|
| Formulation | | mRNA | % | mRNA |
| No. | PDI | (ug/ml) | E.E. | (nM) |
| F20 | 0.12 | 73.35 | 84.14 | 222.38 |
| F21 | 0.132 | 73.42 | 85.66 | 222.59 |
| F22 | 0.126 | 88.42 | 90.04 | 268.07 |
| F23 | 0.107 | 43.64 | 62.36 | 132.3 |
| F24 | 0.147 | 60.51 | 76.15 | 183.47 |
| F25 | 0.255 | 48.66 | 64.52 | 147.54 |
| F26 | 0.081 | 53.56 | 86.08 | 162.37 |
| F27 | 0.045 | 32.74 | 79.16 | 99.25 |
| F28 | 0.071 | 38 | 80.53 | 115.23 |
| F29 | 0.111 | 27.33 | 53.93 | 82.86 |
| F30 | 0.137 | 49.94 | 86.07 | 151.43 |
| F31 | 0.205 | 54.03 | 86.77 | 163.8 |
| F32 | 0.089 | 66.95 | 87.16 | 202.99 |

FIG. 7 shows FACS results after HeLa cells were incubated with 250 ng of each formulation for 24 hours. Cells dosed with LNPs comprising DSPE-PEG2K-DC2 lipids bad a higher fluorescence intensity than cells dosed with LNPs comprising DSPE-PEG2K-DC1 lipids. Cells dosed with LNPs comprising 0.01 mol-% (F26), 0.025 mol-% (F27), 0.05 mol-% (F28), and 0.1 mol-% (F29) DSPE-PEG2K-DC2 lipids had a significantly higher mean fluorescent intensity than control F32 (does not include DSPE-PEG2K-DC2 lipids). A sharp drop in fluorescence intensity was observed for all formulations having more than 0.1 mol-% DSPE-PEG2K-DC2 lipids or DSPE-PEG2K-DC1 lipids. The EEV-mRNA-1 and EEV-mRNA-2 formulations showed minimal fluorescence signal.

In a third experiment, LNP formulations having various ionizable lipids, various total amounts of PEGylated lipids, and varying amounts of DSPE-PEG2K-DC1 and DPSE-PEG2K-DC2 PEGylated conjugate lipids were prepared according to the General LNP Formulation

Protocol. LNPs were loaded with eGFP at an N:P ratio of 6. Table 14 shows the components and amounts of the formulations used in experiment 3.

TABLE 14
Formulations used in the third experiment

| | Ionizable | | | Total mol-% | | Lipid | DMG- |
|---|---|---|---|---|---|---|---|
| | Lipid (50 | Cholesterol | DSPC | PEGylated | Lipid | conj. | PEG2K |
| No. | mol-%) | mol-% | mol-% | Lipids | conj. | mol-% | mol-% |
| F40 | D-Lin- | 38.5 | 10 | 1.5 | N/A | 0 | 1.5 |
| | MC3-DMA | | | | | | |
| F41 | D-Lin- | 38.5 | 10 | 1.5 | DSPE- | 0.025 | 1.475 |
| | MC3-DMA | | | | PEG2k- | | |
| | | | | | EEV02 | | |
| F21 | D-Lin- | 37 | 10 | 3 | DSPE- | 0.025 | 2.975 |
| | MC3-DMA | | | | PEG2k- | | |
| | | | | | EEV01 | | |
| F27A | D-Lin- | 37 | 10 | 3 | DSPE- | 0.025 | 2.975 |
| | MC3-DMA | | | | PEG2k- | | |
| | | | | | EEV02 | | |
| F32A | D-Lin- | 37 | 10 | 3 | N/A | 0 | 3 |
| | MC3-DMA | | | | | | |
| F42 | SM-102 | 38.5 | 10 | 1.5 | N/A | 0 | 1.5 |
| F43 | SM-102 | 38.5 | 10 | 1.5 | DSPE- | 0.025 | 1.475 |
| | | | | | PEG2k- | | |
| | | | | | EEV02 | | |
| F44 | SM-102 | 38.5 | 10 | 1.5 | DSPE- | 0.025 | 1.475 |
| | | | | | PEG2k- | | |
| | | | | | EEV01 | | |
| F45 | SM-102 | 37 | 10 | 3 | N/A | 0.025 | 2.975 |
| F46 | SM-102 | 37 | 10 | 3 | DSPE- | 0.025 | 2.975 |
| | | | | | PEG2k- | | |
| | | | | | EEV02 | | |
| F47 | SM-102 | 37 | 10 | 3 | DSPE- | 0.025 | 2.975 |
| | | | | | PEG2k- | | |
| | | | | | EEV01 | | |

FIG. 8 shows FACS results after HeLa cells were incubated with 250 ng of each formulation for 24 hours. Cells dosed with LNPs comprising DSPE-PEG2K-DC2 lipids had a higher fluorescence intensity than cells dosed with LNPs comprising DSPE-PEG2K-DC1 lipids. Additionally, cells dosed with LNPs comprising the SM-102 ionizable lipid had a higher mean fluorescent intensity than LNPs comprising the D-Lin-MC3-DMA ionizable lipid (FIG. 8).

Cells dosed with formulations having a total PEGylated lipid amount of 1.5 mol-% had a higher mean fluorescent intensity than cell dosed with formulations having a total PEGylated lipid amount of 3 mol-% (FIG. 8). The PEG mediated depreciation of transfection competence is greater in formulations that include the SM-102 ionizable lipid as compared to formulations that include the D-Lin-MC3-DMA ionizable lipid. For example, in formulations with 3 mol-% total PEGylated lipids, the formulation including the SM-102 lipid showed lower transfection efficiency than the D-Lin-MC3-DMA containing LNP counterparts.

In a fourth experiment, LNP formulations having various ionizable lipids, various total amounts of PEGylated lipids, and either 0 mol-% or 0.025 mol-% of DSPE-PEG2K-DC1, DPSE-PEG2K-DC2 or DSPE-PEG2K-DC3 (FIG. 2C) lipids were evaluated for their ability to deliver eGFP mRNA (cargo) into cells via free uptake. Table 15A shows the components and amounts of the formulations used.

TABLE 15A
Formulations used in the fourth experiment

| | | Total | | mol % | mol-% | mol-% | | |
|---|---|---|---|---|---|---|---|---|
| | Ionizable | % | Lipid | lipid | DMG- | ionizable | mol-% | mol-% |
| No. | Lipid | PEG | conj. | conj. | PEG2K | lipid | Cholesterol | DSPC |
| F50 | D-Lin- | 1.5 | DSPE- | 0.025 | 1.475 | 50 | 38.5 | 10 |
| | MC3- | | PEG2k- | | | | | |
| | DMA | | EEV03 | | | | | |
| F41 | D-Lin- | 1.5 | DSPE- | 0.025 | 1.475 | 50 | 38.5 | 10 |
| | MC3- | | PEG2k- | | | | | |
| | DMA | | EEV02 | | | | | |
| F51A | D-Lin- | 1.5 | DSPE- | 0.025 | 1.475 | 50 | 38.5 | 10 |
| | MC3- | | PEG2k- | | | | | |
| | DMA | | EEV02 | | | | | |
| F40A | D-Lin- | 1.5 | N/A | 0 | 1.5 | 50 | 38.5 | 10 |
| | MC3- | | | | | | | |
| | DMA | | | | | | | |
| F95 | D-Lin- | 3 | DSPE- | 0.025 | 2.975 | 50 | 37 | 10 |
| | MC3- | | PEG2k- | | | | | |
| | DMA | | EEV01 | | | | | |
| F27A | D-Lin- | 3 | DSPE- | 0.025 | 2.975 | 50 | 37 | 10 |
| | MC3- | | PEG2k- | | | | | |
| | DMA | | EEV02 | | | | | |
| F32A | D-Lin- | 3 | N/A | 0 | 3 | 50 | 37 | 10 |
| | MC3- | | | | | | | |
| | DMA | | | | | | | |
| F52 | SM-102 | 1.5 | DSPE- | 0.025 | 1.475 | 50 | 38.5 | 10 |
| | | | PEG2k- | | | | | |
| | | | EEV03 | | | | | |
| F44 | SM-102 | 1.5 | DSPE- | 0.025 | 1.475 | 50 | 38.5 | 10 |
| | | | PEG2k- | | | | | |
| | | | EEV01 | | | | | |
| F43 | SM-102 | 1.5 | DSPE- | 0.025 | 1.475 | 50 | 38.5 | 10 |
| | | | PEG2k- | | | | | |
| | | | EEV02 | | | | | |
| F43A | SM-102 | 1.5 | DSPE- | 0.025 | 1.475 | 50 | 38.5 | 10 |
| | | | PEG2k- | | | | | |
| | | | EEV02 | | | | | |
| F42 | SM-102 | 1.5 | N/A | 0 | 1.5 | 50 | 38.5 | 10 |
| F47 | SM-102 | 3 | DSPE- | 0.025 | 2.975 | 50 | 37 | 10 |
| | | | PEG2k- | | | | | |
| | | | EEV01 | | | | | |
| F46 | SM-102 | 3 | DSPE- | 0.025 | 2.975 | 50 | 37 | 10 |
| | | | PEG2k- | | | | | |
| | | | EEV02 | | | | | |
| F45 | SM-102 | 3 | N/A | 0 | 3 | 50 | 37 | 10 |

Table 15B shows the physical properties of the LNPs used in the fourth experiment. Generally, the size of the LNPs and the polydispersity (PDI) increases as the mol-% of the lipid conjugate increases.

TABLE 15B
Physical properties of the LNPs of the third experiment.

| | | | Total | |
|---|---|---|---|---|
| | | | mRNA | % |
| No. | Size (nm) | PDI | (ug/ml) | E.E. |
| F50 | 136.2 | 0.079 | 55.73 | 95.58 |
| F41 | 147.2 | 0.087 | 34.69 | 88.95 |
| F51A | 146.7 | 0.074 | 29.12 | 90.95 |
| F40A | 105.3 | 0.122 | 395.97 | 96.71 |
| F95 | 112.2 | 0.132 | 43.7 | 79.21 |
| F27A | 110.5 | 0.103 | 40.8 | 87.6 |
| F32A | 91.96 | 0.186 | 438.56 | 95.14 |
| F52 | 139.5 | 0.175 | 18.09 | 71.43 |
| F44 | 124.6 | 0.129 | 32.79 | 58.61 |
| F43 | 127.9 | 0.112 | 29.6 | 88.72 |
| F43A | 130.6 | 0.122 | 52.62 | 99.06 |
| F42 | 79.07 | 0.044 | 24.39 | 83.23 |
| F47 | 81.45 | 0.18 | 61.21 | 68.24 |
| F46 | 101.4 | 0.193 | 19.09 | 67.13 |
| F45 | 58.76 | 0.13 | 78.49 | 98.2 |

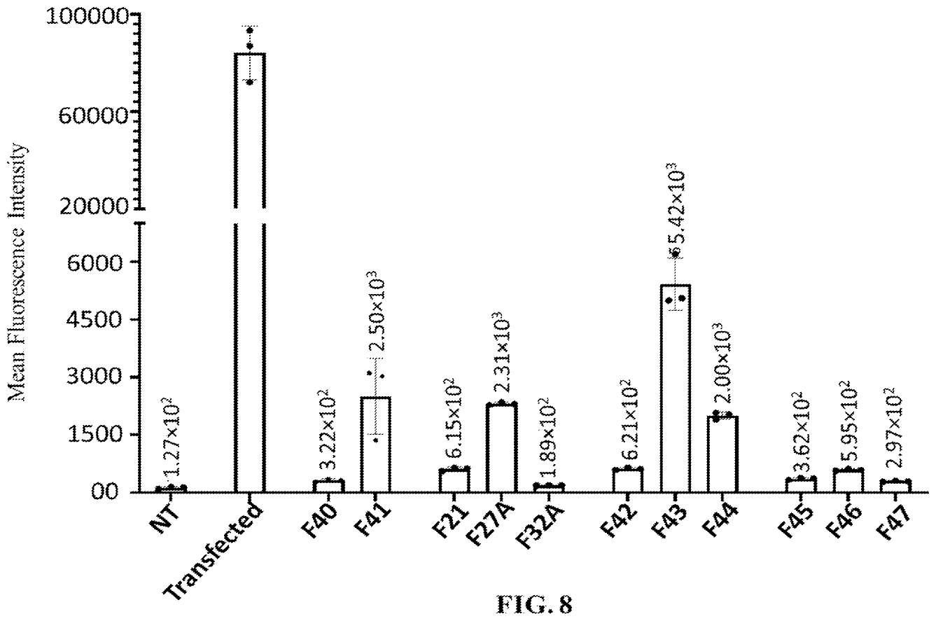

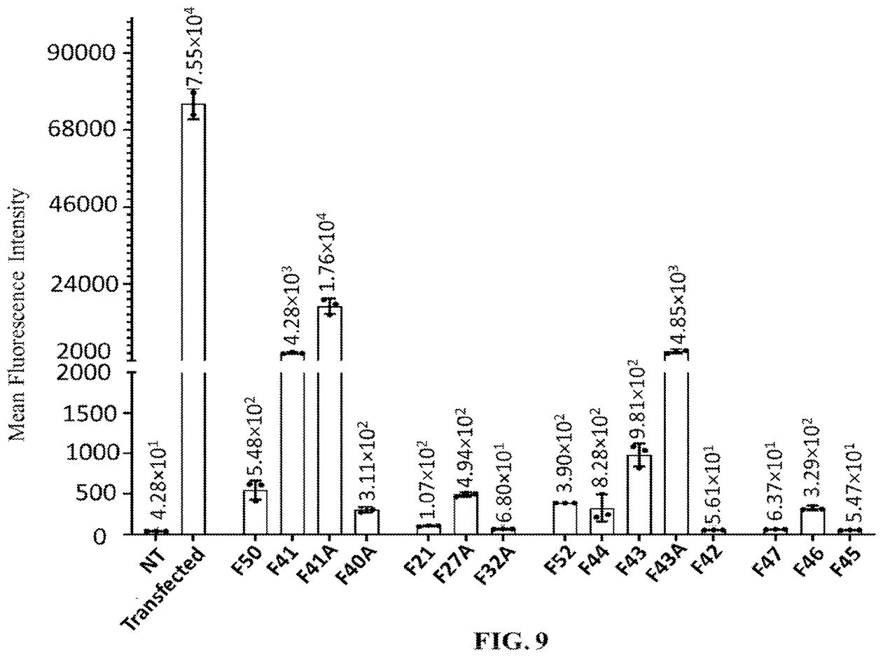

FIG. 9 shows FACS results after HeLa cells were incubated with 250 ng of each formulation for 24 hours. Cells dosed with LNPs comprising DSPE-PEG2K-DC2 lipids had a higher fluorescence intensity than cells dosed with LNPs comprising DSPE-PEG2K-DC1 lipids and DSPE-PEG2K-DC3 lipids. Additionally, cells dosed with LNPs having a total PEGylated lipid content of 1.5 mol-% had a higher fluorescence intensity than cells dosed with LNPs having a total PEGylated lipid content of 3 mol-%.

From all the experiments of Example 2, various findings are worth noting. Generally, LNPs having an average size of less than 100 nm and a polydispersity of less than 0.2 were the most efficacious. It is thought that increasing the mol-% of the lipid conjugate in the formulation results in a population of LNPs that are larger, more unstable, and more poly-disperse than formulations having a lower mol-% of the lipid conjugate. Increasing the total mol-% of PEGylated lipids included in the formulation results in LNPs having smaller average sizes and decreased transfection efficiency. The identity of the ionizable lipid impacts both the size of the LNPs and the transfection efficiency. SM-102 was a more efficacious ionizable lipid than D-Lin-MC3-DMA. The N:P Ratio as well as the size of the nucleic acid cargo has a significant impact on the size and the encapsulation efficiency of the LNP system.

### Example 3. Evaluating LNPs Comprising EEV02-DSPE-PEG2K Lipids for Delivery Gene Editing Machinery

The gene editing efficiency of LNP formulations that included DSPE-PEG2K-DC2-lipid conjugates was evaluated. LNPs were formulated with a gene editing system that included Cas9mRNA and gRNA designed to make an edit that turns off GFP expression in HEK293-uGFP cells. Successful gene editing is indicated by a population of cells that are not expressing GFP. Table 16A shows the components of the formulations of LNPs tested. The Cas9 mRNA to gRNA ratio was one to one (wt/wt).

TABLE 16A
LNP formulations for delivery of gene editing machinery

| | N:P | | | | | | | |
|---|---|---|---|---|---|---|---|---|
| | Ratio | | Total | mol-% | mol-% | | | |
| | (ng of | Lipid | PEGylated | lipid | DMG- | SM102 | mol-% | mol-% |
| No. | Cas9) | conjugate | lipid mol-% | conj. | PEG2K | mol-% | DSPC | cholesterol |
| F60 | 5 | N/A | 1.5 | 0 | 1.5 | 50 | 10 | 38.5 |
| | (500) | | | | | | | |
| F61 | 6 | N/A | 1.5 | 0 | 1.5 | 50 | 10 | 38.5 |
| | (250) | | | | | | | |
| F62 | 7 | N/A | 1.5 | 0 | 1.5 | 50 | 10 | 38.5 |
| | (125) | | | | | | | |
| F63 | 5 | DSPE- | 1.5 | 0.025 | 1.475 | 50 | 10 | 38.5 |
| | (500) | PEG2K- | | | | | | |
| | | EEV02 | | | | | | |
| F64 | 6 | DSPE- | 1.5 | 0.025 | 1.475 | 50 | 10 | 38.5 |
| | (250) | PEG2K- | | | | | | |
| | | EEV02 | | | | | | |
| F65 | 7 | DSPE- | 1.5 | 0.025 | 1.475 | 50 | 10 | 38.5 |
| | (125) | PEG2K- | | | | | | |
| | | EEV02 | | | | | | |

Table 16B shows the physical properties of the LNPs of Table 16A. The sizes of the LNPs formed from a formulation including lipid conjugates and those formed from a formulation that did not include lipid conjugates are comparable. The polydispersity of the LNPs containing lipid conjugates are generally higher than the LNPs not including lipid conjugates.

TABLE 16B
Physical properties of the LNPs of Table 15A

| No. | Size (nm) | PDI | % E.E. |
|---|---|---|---|
| F60 | 82.25 | 0.0961 | 75.6 |
| F61 | 84.44 | 0.0951 | 64.2 |
| F62 | 71.66 | 0.0951 | 57.3 |
| F63 | 98.92 | 0.1019 | 83.9 |
| F64 | 92.55 | 0.2104 | 74.2 |
| F65 | 83.45 | 0.0823 | 63.6 |

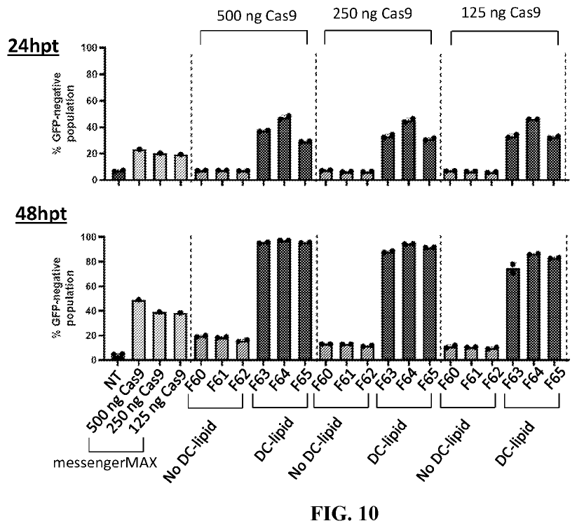

HEK293T-uGFP cells were incubated with LNPs of Table 16A or Cas9 mRNA-gRNA formulated using LIPOFECTAMINE MESSENGERMAX. LIPOFECTAMINE MESSENGERMAX is a commercially available (from ThermoFisher Scientific, Waltham, MA) lipid nanoparticle formulation. Twenty-four and forty-eight hours post incubation, cells were imaged and flow cytometry was used to quantify GFP knockout cells. FIG. 10 shows the percent of the cell population that was GFP negative (not expressing detectable GFP) at the 24 and 48 hour time points. Cells transfected by LNPs containing lipid conjugates showed a higher level of GFP knockout compared to cells transfected by LNPs not containing lipid conjugates the 24 and 48 hour time points, as well as different Cas9 doses (500, 250, 125 ng). The lipid conjugate containing LNP formulations had 7-fold higher gene editing efficiency compared to LNP formulations lacking the lipid conjugate. Additionally, the lipid conjugate containing LNP formulations had a 2-fold higher gene editing efficiency compared to the LIPOFECTAMINE MESSENGERMAX transfection agent. These results suggest that the lipid conjugate improves LNP delivery of the gene editing machinery to the cells. The N:P ratio seemed to have little impact on the gene editing efficiency in this experiment; however, the higher N:P ration seems to affect cell health. For example, it was observed that formulation 65 induced more cell stress than formulations 63 and 64. Cells treated with formulations lacking the-lipid conjugate showed no additional signs of stress.

A Cas9 dose-dependent GFP knockout study was conducted using LNP formulations in Table 17A having the physical properties shown in Table 17B.

TABLE 17A
LNP formulations 61A and 64A for delivery of gene editing machinery

| | | | | Total | mol-% | mol-% | | | |
|---|---|---|---|---|---|---|---|---|---|
| | N:P | Cargo | Lipid | PEGylated | lipid | DMG- | SM102 | mol-% | mol-% |
| No | Ratio | (wt/wt) | conj. | lipid wt-% | conj. | PEG2K | mol-% | DSPC | cholesterol |
| F61A | 6 | Cas9:Guide3 | N/A | 1.5 | 0 | 1.5 | 50 | 10 | 38.5 |
| | | (1:1) | | | | | | | |
| F64A | 6 | Cas9:Guide3 | DSPE- | 1.5 | 0.025 | 1.475 | 50 | 10 | 38.5 |
| | | (1:1) | PEG2k- | | | | | | |
| | | | EEV02 | | | | | | |

TABLE 17B
Physical properties of the LNPs of Table 16B

| No. | Size | PDI | % E.E. |
|---|---|---|---|
| F61A | 66.84 | 0.0428 | 76.5 |
| F64A | 81.06 | 0.0962 | 69.3 |

HEK293T-uGFP cells were incubated with varying amount of the LNPs of Table 17A. with total of I mL per well. The amount of LNP was varied so that cells were exposed to 250 ng/ml, 125 ng/ml, 100 ng/ml, 80 ng/ml, 60 ng/ml, 40 ng/ml, 20 ng/ml, 10 ng/ml, 5 ng/ml, or 1 ng/ml of the Cas9 mRNA gene editing machinery. Gene editing machinery formulated with the LIPOFECTAMINE MESSENGERMAX reagent was also tested.

FIG. 11 shows the percent of the cell population that was GFP negative (not expressing detectable GFP) compared to the amount of Cas9 used in the formulation. The lipid conjugate containing LNP formulations (labeled as DC-LNP in FIG. 11) showed a Cas9 dose-dependent gene editing in vitro. Additionally, the lipid conjugate containing LNP formulations had a higher gene editing efficiency than the LNP formulation not having the lipid conjugate (labeled as LNP in FIG. 11) and the LIPOECTAMINE MESSENGERMAX formulation. The lipid conjugate containing LNP formulations had 33-fold higher gene editing efficiency compared to LNP formulations lacking the lipid conjugate at a dose as low as 40 ng/mL Cas9. The lipid conjugate containing LNP formulations had 25-fold lower EC₅₀ than the LNP formulation lacking the lipid conjugate (EC50 EEV-LNP=40 ng/ml; EC50 LNP=1000 ng/mL according to Finn, et al., Cell Reports, 2018).

### Example 4. Evaluating the Impact of Ionic Strength on LNP Size

The size of LNPs formulated with and without lipid conjugates in buffers with different ionic strengths was elevated. LNPs without a lipid conjugate were formulated with 50 mol-% SM-102, 10 mol-% DSPC, 38.5 mol-% cholesterol, and 1.5 mol-% DMG-PEG2000. LNPs with a lipid conjugate were formulated with 50 mol-% SM-102, 10 mol-% DSPC, 38.5 mol-% cholesterol, 1.475 mol-% DMG-PEG2000, and DC2-DSPE-PEG2000 (lipid conjugate).

Formulations were made in phosphate buffered saline. Following LNP formation, then diluted into a second phosphate buffered saline buffer containing 2.7 mM KCl, 10 mM Na₂HPO₄, 1.8 mM KH₂PO₄, and various amounts of NaCl (25 mM to 1000 mM). The ionic strength of the second buffer was calculated using the following formula:

$$I = \frac{1}{2} \sum ( {c}_{i} \times {z}_{i}^{2} )$$

where I is ionic strength, cris molar concentration of an ion i, and zi is the charge number of ion i. Table 18 shows the ionic strength of the various second buffers that the LNPs were exposed to as well as the ionic strength of relevant biological fluids.

TABLE 18
Ionic strength of various buffers and biological fluids

| | Total Ionic Strength |
|---|---|
| NaCl (mM) | (mM) |
| 25 mM NaCl PBS | 59.5 |
| 50 mM NaCl PBS | 84.5 |
| 100 mM NaCl PBS | 134.5 |
| 150 mM NaCl PBS | 184.5 |
| 300 mM NaCl PBS | 334.5 |
| 500 mM NaCl PBS | 534.5 |
| 1000 mM NaCl PBS | 1034.5 |
| Gibco PBS | 164.8 |
| Teknova PBS | 152 |
| Blood | 150 |
| Cerebrospinal Fluid | 154 |
| Vitreous | 141 |

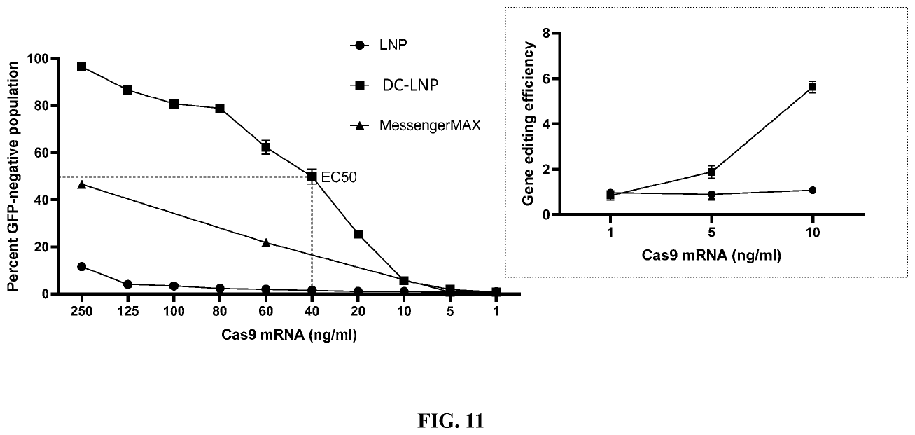

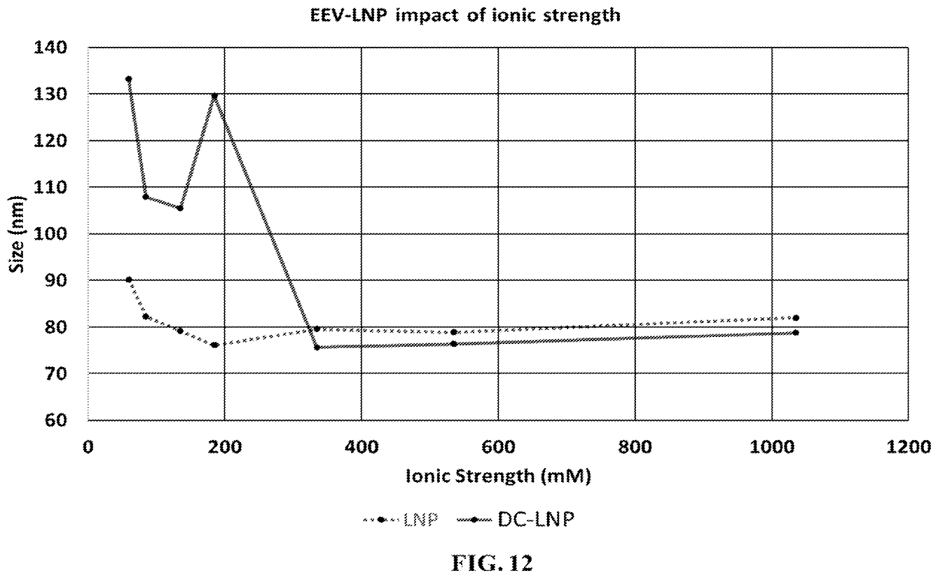

LNPs formulated without the lipid conjugate did not show any change in size over the range of ionic strengths tested (FIG. 12). LNPs formulated with the lipid conjugate showed a conformational change between 184.5 mM-334.5 mM ionic strength after which the LNPs containing lipid conjugates closely mirrored the LNPs not having lipid conjugates in terms of size. This inflection point could change with A) the EEV used B) mol-% of the EEV used and C) overall lipid composition. This transition might or might not be reversible and could have an impact on the in vitro and in vivo competence of the LNPs that include the lipid conjugates.

### Example 5. Assessing the Ability of GEM Conjugates to Induce Gene Editing

Various ribonuclear protein (RNP)—delivery construct (DC) were tested for their ability to induce gene editing in a HEK293/uGFP cell line.

HEK293/uGFP is a commercially available cell line that constitutively express unstable GFP having a half-life of about 2 hours, considerably shorter than the 26-hour half-life of wild-type GFP. The RNPs induce indel formation in the uGFP gene. Successful gene editing results in a decrease in uGFP signal as less full length uGFP is expressed. The RNPs included Cas9 (A) loaded with gRNA, the sequences of which are shown below. Cas9 (A) is a spCas9 derivative that includes a nuclear localization sequence (underlined in the sequence below) and hexahistidine tag (SEQ ID NO: 492) (italicized and bolded in the sequence below) on the N-terminus, as well as an added cysteine (bolded in the sequence below) and second NLS sequence on the C-terminus (underlined in the sequence below).

```
Cas9(A) sequence:
  (SEQ ID NO: 481)
MHHHHHHPKKKRKVDKKYSIGLDIGTNSVGWAVITDEYKVPSKKFKVLGNTDRHSIKK
NLIGALLFDSGETAEATRLKRTARRRYTRRKNRICYLQEIFSNEMAKVDDSFFHRLEESF
LVEEDKKHERHPIFGNIVDEVAYHEKYPTIYHLRKKLVDSTDKADLRLIYLALAHMIKFR
GHFLIEGDLNPDNSDVDKLFIQLVOTYNQLFEENPINASGVDAKAILSARLSKSRRLENLI
AQLPGEKKNGLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKORTF
DNGSIPHQIHLGELHAILRRQEDFYPFLKDNREKIEKILTFRIPYYVGPLARGNSRFAWMT
RKSEETITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEISGVED
RFNASLGTYHDLLKIIKDKDFLDNEENEDILEDIVLTLTLFEDREMIEERLKTYAHLFDDK
VMKQLKRRRYTGWGRLSRKLINGIRDKQSGKTILDFLKSDGFANRNFMQLIHDDSLTFK
EDIQKAQVSGQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVKKMK
NYWRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTALI
KKYPKLESEFVYGDYKVYDVRKMIAKSEQEIGKATAKYFFYSNIMNFFKTEITLANGEIR
KRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKN
PIDFLEAKGYKEVKKDLIIKLPKYSLFELENGRKRMLASAGELQKGNELALPSKYVNFLY
LASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETR
IDLSQLGGDCPKKKRKV
gRNA sequence:
  (SEQ ID NO: 482)
mG*mG*mU*rGrArArCrCrGrCrArUrCrGrArGrCrUrGrArGrUrUrUrUrArG
rArGrCrUrArGrArArArUrArGrCrArArGrUrUrArArArArUrArArGrGrCrUrArGrU
rCrCrGrUrUrArUrCrArArCrUrUrGrArArArArArGrUrGrGrCrArCrCrGrArGrUrC
rGrGrUrGrCmU*mU*mU* rU
(m denotes 2′-OMe modified bases; * denotes PS linkages; ris RNA)
```

DC4 (cyclo[Ff-Nal-RrRrQ]-PEG12-K (Maleimide)-NH2 (SEQ ID NO: 439)) was conjugated via maleimide chemistry to the Cas9 protein of the RNP. A single DC4(1DC-RNP, IDC4(A)-RNP, and 1DC4(B)-RNP) or two DCs (2DC(A)-RNP and 2DC4(B)-RNP) were conjugated to Cas9.

HEK293/uGFP cells were treated with various samples either via transfection with theLipofectamine CRISPRMAX transfection agent according to the manufactures protocol (ThermoFisher Scientific, Waltham, MA) or without a transfection reagent (free uptake). The cells were then incubated for 1 to 5 days followed by gene editing analysis using fluorescence-activated cell (FACS) sorting flow cytometry and next generation sequencing (NGS). Two experiments were conducted. In the first experiment, HEK293/uGFP cells were treated with various samples either via transfection or free uptake, incubated for 5 days, and analyzed using FACS, next generation sequencing (NGS), and a T7 Endonuclease I assay. The FACS results are shown in Table 19. Treatment of the cells via transfection with the RNP and DC4-RNP showed the highest level of GFP negative cells indicating successful gene editing. Both the untreated Table 20 where “modified” is the percent of reads where the uGFP gene was modified (e.g., gene edited) and “unmodified” is the percent of reads where the uGFP gene was not modified (e.g., not gene edited). Cells transfected with the RNP and cells transfected with the DC4-RNP showed an increase in the number of reads modified as compared to the control. Additionally, cells transfected with DC4-RNP showed a higher number of modified reads than cells transfect with just RNP (no DC4).

TABLE 19
FACS data of first gene editing experiment

| Sample | GFP − (%) | GFP + (%) |
|---|---|---|
| Untreated | 96.4 | 3.65 |
| Transfection of RNP | 33.7 | 66.3 |
| Transfection of DC4-RNP | 30.1 | 69.9 |
| Transfection of DC4-RNP with scrambled gRNA | 96.1 | 3.92 |
| RNP (free uptake) | 94.9 | 5.06 |
| DC4-RNP (free uptake) | 94.9 | 5.11 |

TABLE 20
NGS data of first gene editing experiment

| Sample | unmodified, % (reads) | modified, % (reads) |
|---|---|---|
| Untreated | 99.57 (291888) | 0.43 (1247) |
| Transfection of RNP | 76.7 (189800) | 23.3 (57643) |
| Transfection of DC4-RNP | 56.08 (122170) | 43.92 (95684) |

In the second experiment, HEK293/uGFP cells were treated with DC4(A)-RNP (DC4(A)-RNP), DC4(B)-RNP (DC4(B)-RNP), 2DC4(A)-RNP (2DC4(A)-RNP), or 2DC4(B)-RNP (2DC4(B)-RNP) via transfection with Lipofectamine CRISPRMAX or free uptake. Cells treated via transfection were incubated for 5 days prior to analysis. Various concentrations of the DC4-RNP conjugate (1 μM or 3 μM) and incubation times (1 day, 2 days, or 3 days) were tested.

DC4(A)-RNP and DC4(B)-RNP are two separate samples (A and B) of DC4-RNP. 2DC4(A)-RNP and 2DC4(B)-RNP are two separate samples (A and B) of 2DC4-RNP. 2DC4 (A)-RNP and 2DC4(B)-RNP have two DC4 groups covalently attached to the RNP via maleimide chemistry.

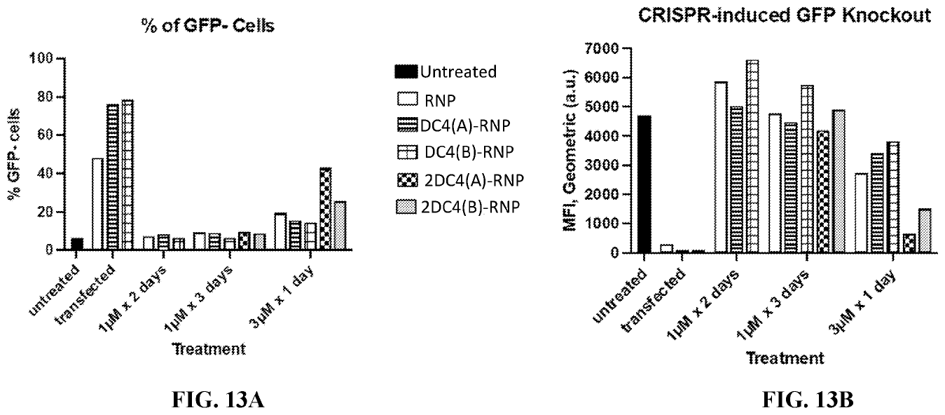

FIG. 13A and FIG. 13B show the results of the FACS and T7 Endonuclease I studies used to evaluate the ability of the DC4-RNP compounds to induce GFP-knockout. FACS analysis indicated that some of the DC4-RNP free-uptake conditions showed some GFP-knockdown, primarily the cells treated with 3 μM of the DC4-RNP conjugate and incubated for a single day. Those same samples showed some CRISPR-induced GFP knockout when analyzed via NGS and the T7 Endonuclease I assay. For example, in FIG. 13B, a decrease in mean fluorescence intensity (MFI) is indicative of CRISPR mediated GPF knockout.

### Example 6. Assessing LNPs Including a GEM Conjugate Cargo

LNPs having a delivery construct (DC)-RNP conjugate cargo (DC-RNP) were tested for their ability to induce gene editing in the gene editing in a HEK293/uGFP cell line of Example 5.

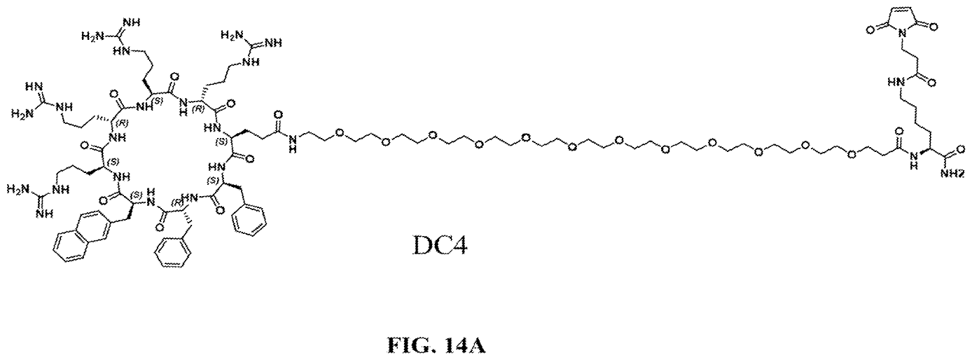

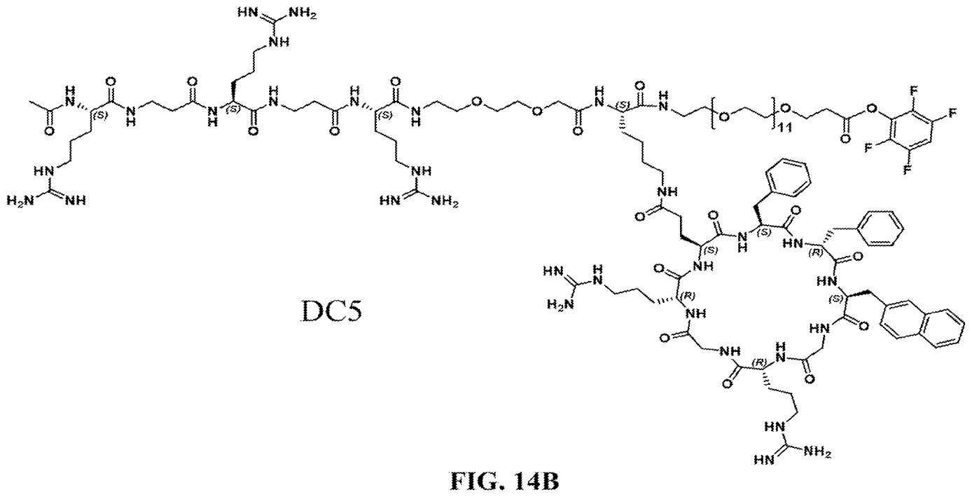

Three RNP-DC conjugates were prepared by conjugating DC4 (FIG. 14A), DC5 (FIG. 14B), and DC6 (FIG. 14C) to the Cas9(A) protein (see Example 5 for the sequence of Cas9(A)). EEV04 and EEV06 were conjugated to Cas9(A) using maleimide-thiol chemistry. DC5 was conjugated to Cas9(A) using amine-TFP activated ester chemistry. In brief, Cas9(A) in storage buffer (20 mm HEPES, 500 mM KCl, 2 mM MgCl2, 10% glycerol, ImM TCEP) was diluted with an equal volume of PBS, before adding 2-5 molar equivalents of an DC in DMSO. The reaction was allowed to proceed at 4° C. overnight, and conjugation was confirmed via MALDI-TOF (Bruker AutoFlex, Billerica, MA) using sinapinic acid matrix (dissolved in 1:1 acetotnitrile:H2O +0.1% trifluoroacetic acid). MALDI-TOF analysis indicated that each DC-Cas9(A) conjugate included 1, 2, or more DCs. Ultracentrifugal filters (Amicon, 50 kDa MWCO) were used to remove unconjugated DC and buffer exchange into HEPES storage buffer. The Cas9(A) concentration was calculated from the A280 absorbance (Nanodrop One, ThermoFisher, Waltham, MA). Samples were also checked for size and aggregation via SDS-PAGE (Bio-rad Mini-Protean TGX precast 4-20% gel, 30 min at 200V) Final DC-Cas9(A) conjugates were stored at −80° C. and thawed on ice for experimental use.

Enzymatic activity of Cas9(A) and the Cas9-DC conjugates was assessed through an in tube cleavage assay. Cas9(A) or Cas9(A)-DC was pre-complexed with a gRNA at a 1.1:1 molar ratio for 10 min at 25° C. DNA substrate for the complementary gRNA region was added and the mixture was incubated for 20 min at 37° C. to enable cleavage of the DNA substrate. The reaction was then incubated with proteinase K for 15 min at 25° C. to degrade Cas9. Samples were run on a 1.2% agarose gel and the resulting product bands were used to determine the extent of Cas9 activity. Cas9(A) and Cas9(A)-DC conjugates showed enzymatic activity.

DC-RNP conjugates were formulated with LNPs via two methods to give formulations F200-F700 (Table 21A and Table 21B). The average number of DCs conjugated to each DC-Cas9 (A) conjugate molecule is shown for each formulation.

In the first method (formulations F200 and F300), Cas9(A) and the DC-Cas9(A) conjugate, were combined with an equimolar amount of sgRNA (sgGFP3) and kept at room temperature for 10-30 min for RNP complexation. Lipids were added in molar percentages indicated to ensure the weight ratio between the sgRNA and total lipid was maintained at 1:20. The RNP was rapidly diluted with 20 mM sodium acetate buffer and then microfluidically mixed with an ethanolic solution of lipids with a flowrate of 1 ml/min and an aqueous to ethanolic ratio of 9:1. The resulting lipid-RNP nanoassembly was diluted out and buffer exchanged in PBS pH =7.2 using prior to characterization.

In the second method (formulations F400 to F700), the RNP and the LNPs were formed separately then the LNPs were complexed with various RNPs at a 1:1, 2:1, and 1:2 RNP:LNP (v/v) ratios. LNPs were made separate from the RNP by diluting the ethanolic lipids constituents in molar percentages indicated below with 20 mM acetate buffer in a volumetric ratio of 1:3 and a total flow rate of 20 ml/min. The lipid nanoassembly was diluted further and buffer exchanged in 20 mM sodium acetate buffer prior to storage & characterization. RNPs were formed by mixing 10 μM Cas9(A) or Cas9(A)-DC conjugates in a 1:1 molar ratio (5:1 wt ratio) with 10 μM guide RNA (sgGFP3) and incubated at room temperature for 10 min. Next, the NPs were added at a 1:1, 2:1, or 1:2 volume ratio to the 5 μM RNP mixture and complexed for 5 minutes before diluting with PBS to 1 μM final concentration of RNP. LNP only controls were made in the same manner, substituting PBS and H2O for Cas9(A) and gRNA, respectively. For the cell experiments, 50 μl of each of formulations F400 to F700 were added to cells.

TABLE 21A
LNP formulations using method I

| | | Average | | | | DMG- |
|---|---|---|---|---|---|---|
| | | DCs per | SM-102 | Cholesterol | DSPC | PEG |
| Formulation | Cas9(A) | conjugate | wt-% | wt-% | wt-% | wt-% |
| F200 | Cas9(A) | N/A | 50 | 38.5 | 10 | 1.5 |
| F300 | Cas9(A)- | 1 | 50 | 38.5 | 10 | 1.5 |
| | EEV04 | | | | | |

TABLE 21B
LNP formulations using method 3

| | | Average | | | | | |
|---|---|---|---|---|---|---|---|
| | Cas9 | DCs per | LNP:RNP | DODMA | Cholesterol | DSPC | DOPE |
| Formulation | conjugate | conjugate | ratio | wt-% | wt-% | wt-% | wt-% |
| F400 | Cas9(A) | N/A | 1:1 | 20 | 40 | 10 | 30 |
| F402 | Cas9(A) | N/A | 2:1 | 20 | 40 | 10 | 30 |
| F404 | Cas9(A) | N/A | 1:2 | 20 | 40 | 10 | 30 |
| F450 | none | N/A | 1:2 | 20 | 40 | 10 | 30 |
| F450 | none | N/A | 2:1 | 20 | 40 | 10 | 30 |
| F500 | Cas9(A)- | 1 | 1:2 | 20 | 40 | 10 | 30 |
| | EEV04 | | | | | | |
| F502 | Cas9(A)- | 1 | 2:1 | 20 | 40 | 10 | 30 |
| | EEV04 | | | | | | |
| F504 | Cas9(A)- | 1 | 1:1 | 20 | 40 | 10 | 30 |
| | EEV04 | | | | | | |
| F506 | Cas9(A)- | 2 | 1:1 | 20 | 40 | 10 | 30 |
| | EEV04 | | | | | | |
| F600 | Cas9(A)- | 1 | 1:1 | 20 | 40 | 10 | 30 |
| | EEV05 | | | | | | |
| F602 | Cas9(A)- | 2 | 1:1 | 20 | 40 | 10 | 30 |
| | EEV05 | | | | | | |
| F700 | Cas9(A)- | 1 | 1:2 | 20 | 40 | 10 | 30 |
| | EEV06 | | | | | | |
| F702 | Cas9(A)- | 1 | 2:1 | 20 | 40 | 10 | 30 |
| | EEV06 | | | | | | |
| F704 | Cas9(A)- | 2 | 1:1 | 20 | 40 | 10 | 30 |
| | EEV06 | | | | | | |
| F706 | Cas9(A)- | 1 | 1:1 | 20 | 40 | 10 | 30 |
| | EEV06 | | | | | | |

HEK293 cells expressing unstable GFP (HEK239-uGFP) were purchased from GenTarget Inc. (San Diego, CA) and cultured at 37° C. with 5% CO₂ in Dulbecco's Modified Eagle Medium (DMEM) with 10% fetal bovine serum (FBS) and 1% penicillin-streptomycin (P/S). The day before the experiment, cells were seeded into 24-well plates at 80,000 cells/well in 500 μl complete media and allowed to adhere overnight.

The following day, cells were treated with the LNP formulations or RNPs (DC conjugate or not unconjugated) not encapsulated within an LNP. RNPs not encapsulated within and LNP were transfected into the cells using Lipofectamine CRISPRMAX (ThermoFischer, Waltham, MA). LNP formulations were at or diluted to 2.6 ng/ml sgRNA in PBS (based on Ribogreen values, estimated equivalent to 1300 ng/ml Cas9) and dosed at 260 ng sgRNA/well in triplicate. CRISPRMAX transfection controls of RNPs not encapsulated within LNPs were made according to the manufacturer's protocol for 24-well plates. In brief, 1250 ng Cas9(A) or Cas9(A)-DC conjugate was mixed with 250 ng gRNA and 2.5 μl Plus Reagent in 25 μl OptiMEM. 1.5 μl CRISPRMAX reagent was diluted into 25 μl OptiMEM, added to the RNP complex, and incubated for 10 min before 50 μl (˜1250 ng RNP) was added to each well. Two negative control conditions were included: cells with no treatment (NT) and untreated cells (UT) dosed with LNP buffer but no LNPs. CRIPSRMAX transfected RNPs (prepared as described above) were included as positive controls. However, to evaluate the effect of the LNP buffers on RNP activity, Cas9(A) and Cas9(A)-DC stocks were diluted into 20 mM sodium acetate pH 4.3 or 1× PBS pH 7.2 and used in the CRISPRMAX complexes. Cells were incubated for 2.5 days to allow for GFP knockout before endpoint assessment by flow cytometry.

Two and half days after treatment, the cell samples were prepared for flow cytometry. In brief, cells were detached using 250 μl 0.25% trypsin EDTA per well at 37° C. for 5-10 min, quenched with 750 μl complete media, and pelleted by spinning for 5 min at 200 g (Sorvall Legend XIR Centrifguge, Thermo Fisher Scientific, Waltham, MA). Residual media was aspirated and the pellets were rinsed once with PBS and respun, before being resuspended in 150 μl cold PBS (with Ca⁺², Mg⁺²), and filtered through 40 μM Flowmi tips into a black 96-well plate. Samples were analyzed using the BD FACSCanto II flow cytometer (Becton Dickinson Biosciences, Franklin lakes, NJ) and subsequent data was processed with FlowJo software (Ashland, OR) and GraphPad Prism.

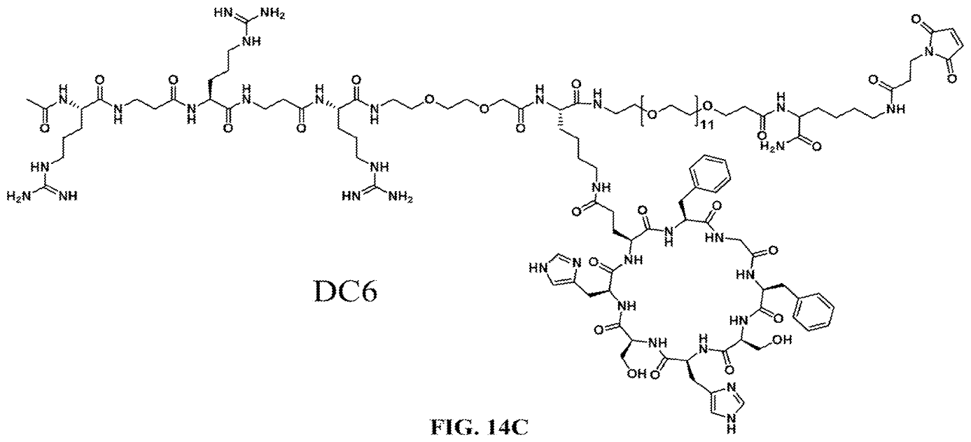

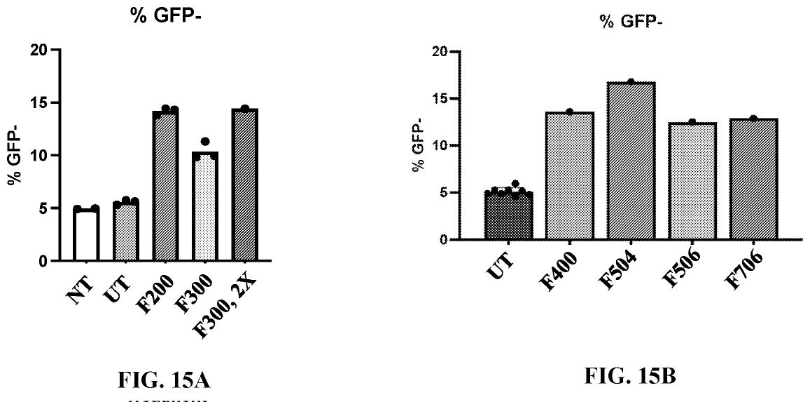

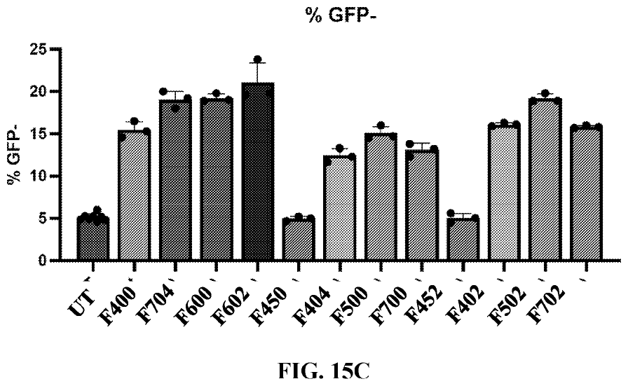

FIGS. 15A-15C show the results of the flow cytometry data. GFP negative cells indicate successful gene editing. Cells treated with formulations F200 and F300, as well as a double dose of F300 (F300, 2X), showed increased gene editing compared to the not treated (NT) and untreated (UT) cells (FIG. 15A). Cells treated with formulations F400-F404 and F500-F706 showed increased gene editing compared to the untreated (UT) cells and cells treated with control LNPs that did not include an RNP (formulations F450 and F452; FIG. 15B-15C). These experiments demonstrated that DC-RNP conjugates encapsulated in LNPs are able to affect gene editing.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1.-99. (canceled)

100. A lipid-based particle comprising:
a PEGylated lipid conjugate comprising:
(i) a lipid delivery construct comprising a cyclic cell penetrating peptide (cCPP) comprising: 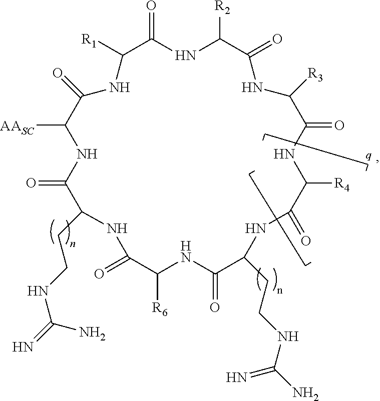
or a protonated form or salt thereof,
wherein:
each m is, independently, an integer from 0-3;
R₁, R₂, and R₃ are, independently, H or a side chain comprising an aryl group; and
R₄ and R₆ are, independently, H or an amino acid side chain; and
(ii) a PEGylated lipid comprising: 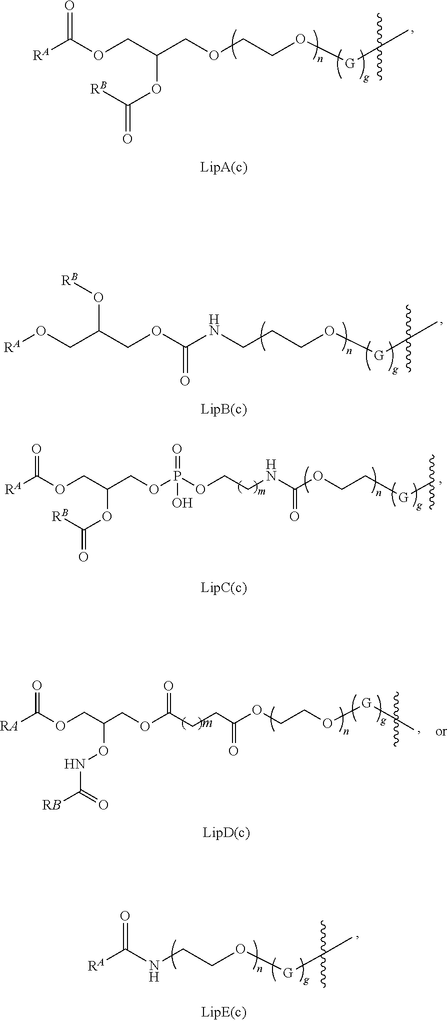
wherein:
R^{A} and R^{B} are each independently an alkyl or alkenyl of C5 to C25, wherein one or more carbons of the alkyl or alkenyl are optionally replaced with a catenated heteroatom, optionally substituted with O to form a carbonyl, or both;
n is an integer from 1 to 50;
m is an integer from 0 to 10;
g is 0 or 1; and
G is 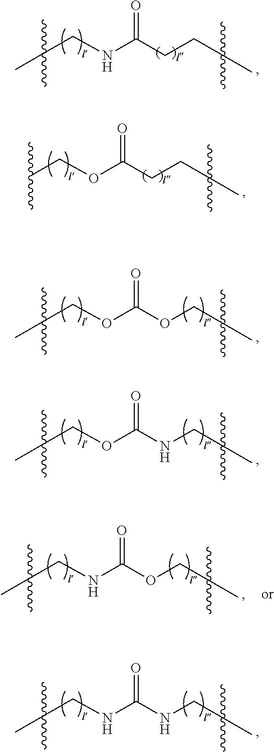
wherein l′ and 1″ are each independently an integer from 0 to 10.

101. The lipid-based particle of claim 100, wherein R^{A}, R^{B}, or both are:
(a) an alkyl or alkenyl of C8 to C22;
(b) an alkyl or alkenyl of C10 to C20;
(c) an alkyl or alkenyl of C15 to C20; or
(d) an alkyl or alkenyl of C17.

102. The lipid-based particle of claim 100, wherein m is 1, 2, or 3.

103. The lipid-based particle of claim 100, wherein:
(a) n is an integer from 10 to 50;
(b) n is an integer from 10 to 20;
(c) n is an integer from 30 to 50;
(d) n is an integer from 40 to 50;
(e) n is 12; or
(f) n is 44.

104. The lipid-based particle of claim 100, wherein g is 1.

105. The lipid-based particle of claim 100, wherein:
(a) l′ and l″ are 2; or
(b) l′is 1 and 1″is 2.

106. The lipid-based particle of claim 100, wherein the PEGylated lipid comprises: 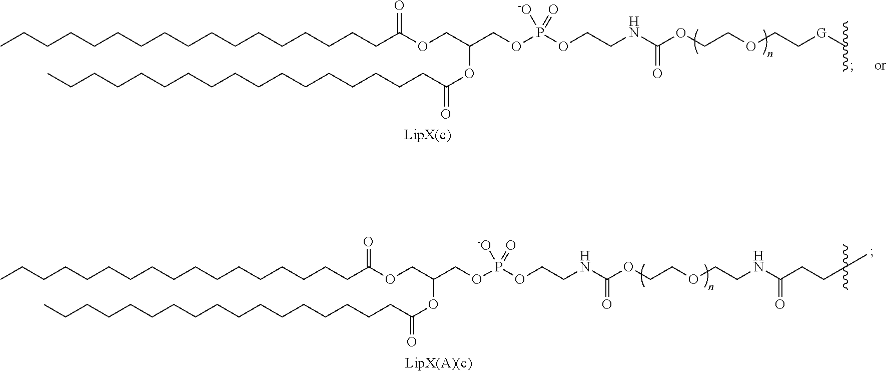
wherein n is an integer from 1 to 50.

107. The lipid-based particle of claim 100, further comprising 5 mol-% or less of a non-conjugated PEGylated lipid; and wherein the non-conjugated PEGylated lipid comprises DMG-PEG2K.

108. The lipid-based particle of claim 107, wherein a total amount of the non-conjugated PEGylated lipid and the PEGylated lipid conjugate is
(a) 5.0 mol-% or less;
(b) 3.0 mol-% or less;
(c) 2.0 mol-% or less; or
(d) 1.5 mol-% or less.

109. The lipid-based particle of claim 100, wherein the lipid-based particle is a liposome.

110. The lipid-based particle of claim 100, wherein the lipid-based particle is a lipid nanoparticle (LNP).

111. The lipid-based particle of claim 110, wherein the LNP comprises:
(a) (i) 0.0075 mol-% to 0.5 mol-% of the PEGylated lipid conjugate;
(ii) 30 mol-% to 60 mol-% of an ionizable lipid;
(iii) 5.0 mol-% to 25 mol-% of a helper lipid; and
(iv) 20 mol-% to 60 mol-% of a sterol; or
(b) (i) 0.0075 mol-% to 0.2 mol-% of the PEGylated lipid conjugate;
(ii) 30 mol-% to 60 mol-% of an ionizable lipid;
(iii) 5.0 mol-% to 15 mol-% of a helper lipid; and
(iv) 20 mol-% to 60 mol-% of a sterol; or
(c) (i) 0.0075 mol-% to 0.08 mol-% of the PEGylated lipid conjugate;
(ii) 40 mol-% to 60 mol-% of an ionizable lipid;
(iii) 7.5 mol-% to 15 mol-% of a helper lipid; and
(iv) 30 mol-% to 40 mol-% of a sterol; or
(d) (i) 0.01 mol-% to 0.06 mol-% of the PEGylated lipid conjugate;
(ii) 45 mol-% to 55 mol-% of an ionizable lipid;
(iii) 7.5 mol-% to 12.5 mol-% of a helper lipid; and
(iv) 35 mol-% to 40 mol-% of a sterol.

112. The lipid-based particle of claim 111, wherein:
(a) the ionizable lipid comprises SM-102, MC3, or lipid 5;
(b) the helper lipid comprises distearoylphosphatidylcholine (DSPC) or dioleoyl-phosphatidylethanolamine (DOPE); and/or
(c) the PEGylated lipid of the PEGylated lipid conjugate comprises DMG-PEG2K.

113. The lipid-based particle of claim 100, further comprising a payload comprising an oligonucleotide, peptide, small molecule, or any combination thereof.

114. The lipid based particle of claim 113, wherein the payload comprises:
(a) an oligonucleotide;
(b) ribonucleic acid (RNA);
(c) an antisense RNA (RNAi) or a message RNA (mRNA); or
(d) two or more guide RNAs (gRNAs).

115. The lipid-based particle of claim 114, wherein the payload comprises a ribonucleoprotein (RNP) comprising gRNA and a nuclease; or the payload comprises gRNA and a nucleic acid encoding a nuclease.

116. The lipid-based particle of claim 100, wherein two of R₁, R₂, and R₃ are a side chain of phenylalanine.

117. The lipid-based particle of claim 100, wherein two of R₁, R₂, R₃, and R₄ are H.

118. The lipid-based particle of claim 100, wherein R₄ and R₆ are H.

119. The lipid-based particle of claim 100, wherein the lipid delivery construct comprises: 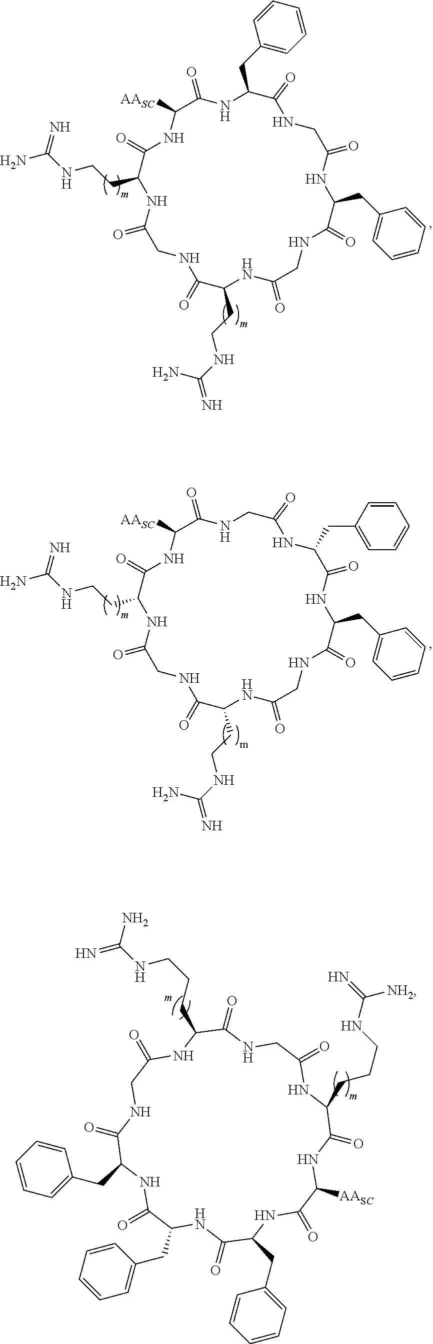 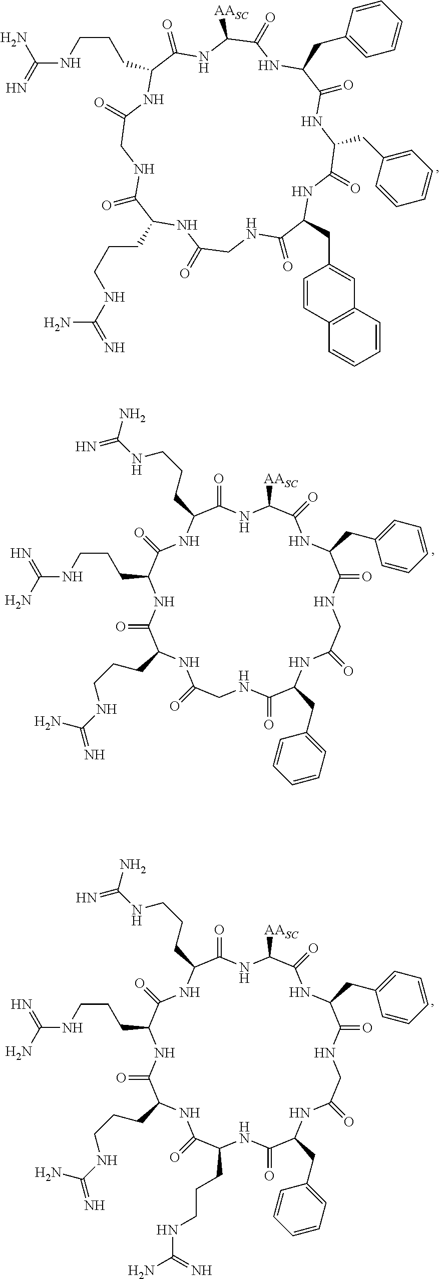
or a protonated form thereof; wherein each m independently comprises an integer from 0-3 and AAsc comprises a side chain of a glutamic acid residue.

120. The lipid-based particle of claim 100, wherein the lipid delivery construct is selected from: FfΦRrRrQ, FfΦCit-r-Cit-rQ, FfΦGrGrQ, FfFGRGRQ, FGFGRGRQ, GfFGrGrQ, FGFGRRRQ, or FGFRRRRQ.

121. The lipid-based particle of claim 100, wherein the delivery construct comprises an endosomal escape vehicle (EEV) comprising cCPP, an exocyclic peptide (EP) and a linker, wherein:
(a) the linker comprises: 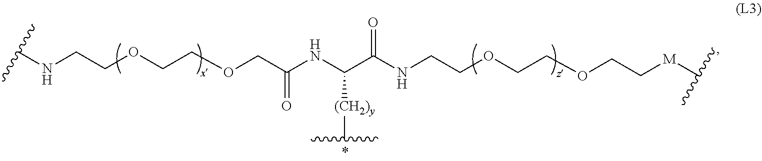
wherein:
x′ is an integer from 1-23;
y is an integer from 1-5;
z′ is an integer from 1-23;
* is the point of attachment to the AA_{SC}, wherein AA_{SC} is a side chain of an amino acid residue of the cCPP; and
M is a bonding group; and
(b) the exocyclic peptide (EP) comprises:
from 4 to 8 amino acid residues;
2, 3, or 4 lysine residues;
at least 2 amino acid residues with a hydrophobic side chain selected from valine, proline, alanine, leucine, isoleucine, and methionine; and
and M is a bonding group comprising: 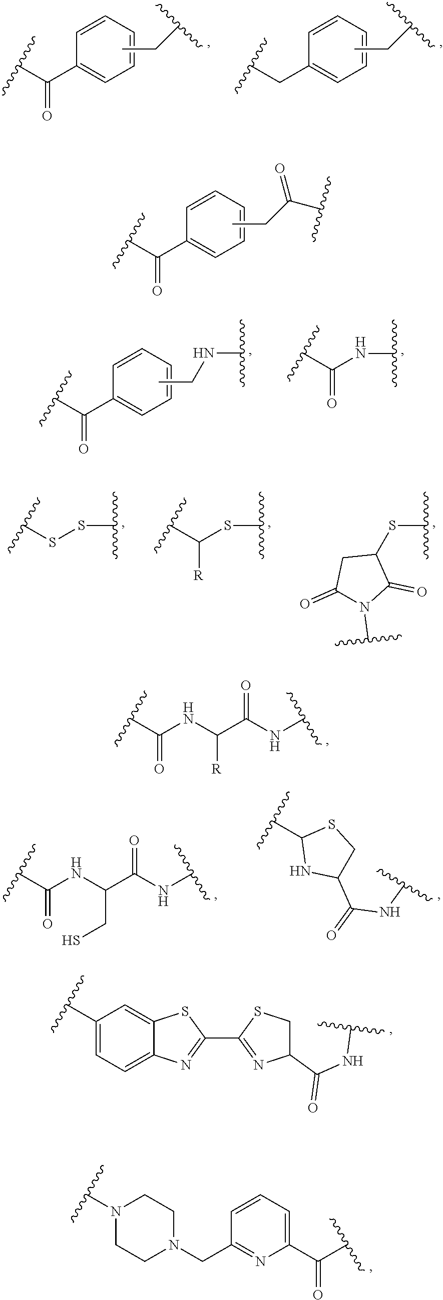 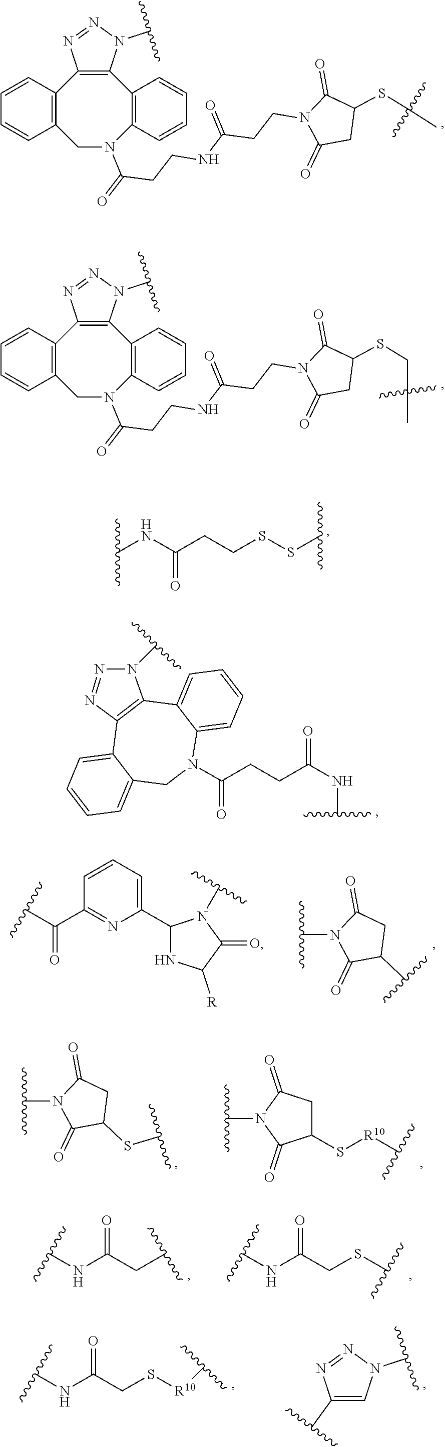 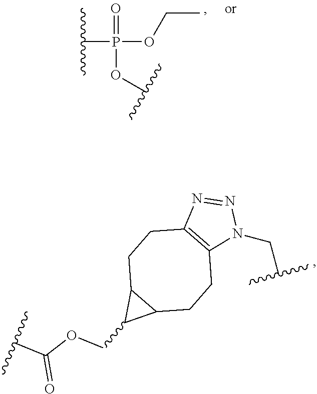
wherein R is alkyl, alkenyl, alkynyl, carbocyclyl, or heterocyclyl; and R¹⁰ is alkylene, cycloalkyl, or 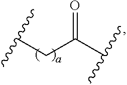
and wherein a is 0 to 10.

122. The lipid-based particle of claim 121, wherein the exocyclic peptide (EP) is selected from:
(a) KK, KR, RR, HH, HK, HR, RH, KKK, KGK, KBK, KBR, KRK, KRR, RKK, RRR, KKH, KHK, HKK, HRR, HRH, HHR, HBH, HHH, HHHH, KHKK, KKHK, KKKH, KHKH, HKHK, KKKK, KKRK, KRKK, KRRK, RKKR, RRRR, KGKK, KKGK, HBHBH, HBKBH, RRRRR, KKKKK, KKKRK, RKKKK, KRKKK, KKRKK, KKKKR, KBKBK, RKKKKG, KRKKKG, KKRKKG, KKKKRG, RKKKKB, KRKKKB, KKRKKB, KKKKRB, KKKRKV, RRRRRR, HHHHHH, RHRHRH, HRHRHR, KRKRKR, RKRKRK, RBRBRB, KBKBKB, PKKKRKV, PGKKRKV, PKGKRKV, PKKGRKV, PKKKGKV, PKKKRGV or PKKKRKG, wherein B is β-alanine;
(b) KK, KR, RR, KKK, KGK, KBK, KBR, KRK, KRR, RKK, RRR, KKKK, KKRK, KRKK, KRRK, RKKR, RRRR, KGKK, KKGK, KKKKK, KKKRK, KBKBK, KKKRKV, PKKKRKV, PGKKRKV, PKGKRKV, PKKGRKV, PKKKGKV, PKKKRGV, or PKKKRKG, wherein B is β-alanine; or
(c) PKKKRKV, RR, RRR, RHR, RBR, RBRBR, RBHBR, or HBRBH, wherein B is β-alanine.

123. The lipid-based particle of claim 121, wherein the EEV is selected from:
Ac-PKKKRKV-K(cyclo[Ff-Nal-RrRrQ])-PEG₁₂-K(N₃)-NH₂;
(cyclo[Ff-Nal-RrRrQ)-PEG12-K(N₃);
Ac-PKKKRKV-K(cyclo[Ff-Nal-GrGrQ])-PEG₁₂-K(N3)-NH₂;
Ac-PKKKRKV-PEG₂-K(cyclo[Ff-Nal-GrGrQ])-PEG2-K(N₃)-NH₂;
Ac-PKKKRKV-PEG₂-K(cyclo[GfFGrGrQ])-PEG₂-K(N₃)-NH₂;
Ac-PKKKRKV-PEG₂-K(cyclo[FfFGRGRQ])-PEG₂-K(N₃)-NH₂;
Ac-PKKKRKV-PEG₂-K(cyclo[FGFGRGRQ])-PEG₁₂-K(N3)-NH₂;
or
Ac-PKKKRKV-PEG₂-K(cyclo[FGFRRRRQ])-PEG₁₂-K(N₃)-NH₂.

124. The lipid-based particle of claim 123, wherein the EEV comprises Ac-PKKKRKV-PEG₂-K(cyclo[FGFGRGRQ])-PEG₁₂-K(N₃)-NH₂.

125. A lipid conjugate comprising:
(a) a PEGylated lipid conjugate comprising a PEGylated lipid conjugated to a lipid delivery construct, the lipid delivery construct comprising a cyclic cell penetrating peptide (cCPP) comprising: 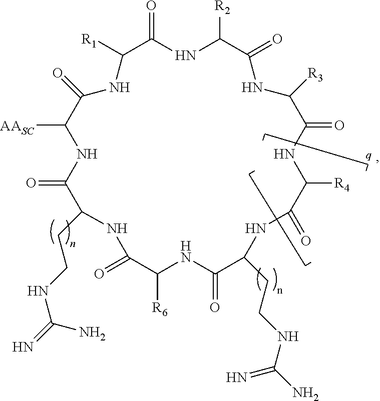
or a protonated form or salt thereof,
wherein:
each m is independently an integer from 0-3;
R₁, R₂, and R₃ are, independently, H or a side chain comprising an aryl group; and
R₄ and R₆ are, independently, H or an amino acid side chain; and
(b) the PEGylated lipid comprising: 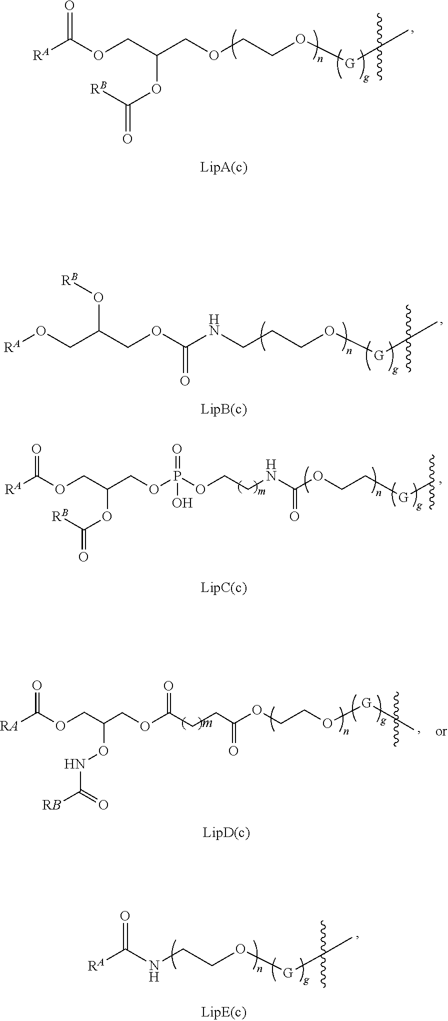
wherein:
R^{A} and R^{B} are each independently an alkyl or alkenyl of C5 to C25, wherein one or more carbons of the alkyl or alkenyl are optionally replaced with a catenated heteroatom, optionally substituted with O to form a carbonyl, or both;
n is an integer from 1 and 50;
m is an integer from 0 and 10;
g is 0 or 1; and
G is 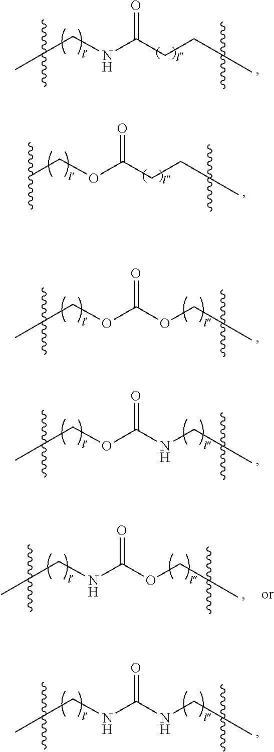
wherein l′ and l″ are each independently an integer from 0 to 10.

126. A method of making a lipid-based particle comprising:
(a) creating a first mixture that includes a conjugated lipid, a non-conjugated PEGylated lipid, an ionizable lipid, a helper lipid, a cholesterol or derivative thereof, and a solvent; wherien:
(i) the conjugated lipid comprises a PEGylated lipid conjugate comprising a PEGylated lipid conjugated to a lipid delivery construct, the lipid delivery construct comprising a cyclic cell penetrating peptide (cCPP) comprising: 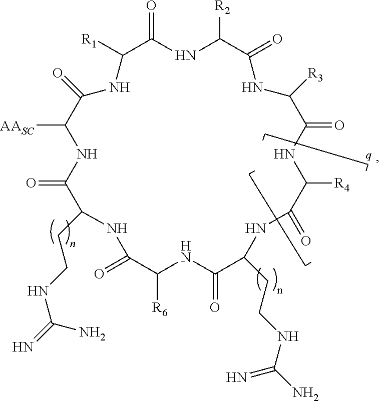
or a protonated form or salt thereof,
wherein:
each m is independently an integer from 0-3;
R₁, R₂, and R₃ are, independently, H or a side chain comprising an aryl group; and
R₄ and R₆ are, independently, H or an amino acid side chain; and
(ii) the PEGylated lipid comprises: 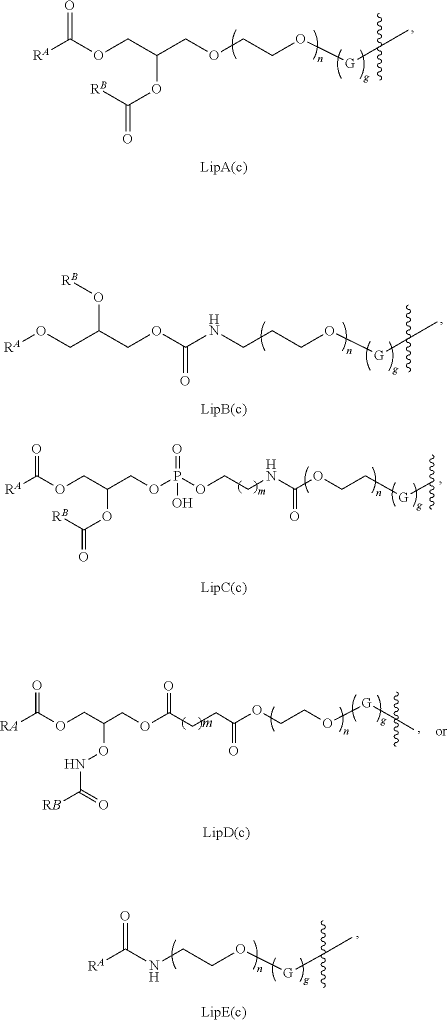
wherein:
R^{A} and R^{B} are each independently an alkyl or alkenyl of C5 to C25, wherein one or more carbons of the alkyl or alkenyl are optionally replaced with a catenated heteroatom, optionally substituted with O to form a carbonyl, or both;
n is an integer between 1 and 50;
m is an integer between 0 and 10;
g is 0 or 1; and
G is 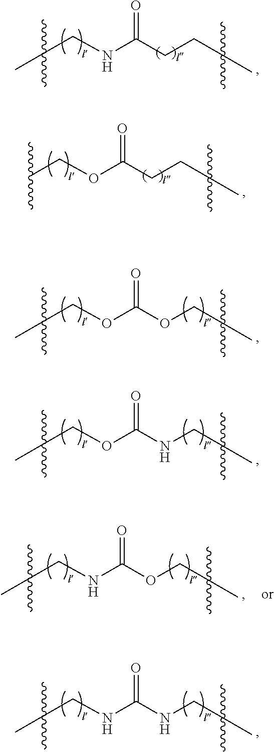
wherein l′ and l″ are each independently an integer from 0 to 10; and
(b) creating a second mixture that includes a payload;
(c) mixing the first mixture with the second mixture to create a third mixture; and
wherein mixing inlcudes vortexing, sonicating, pipette mixing, or combinations thereof; or using a microfluidics device; and
(d) allowing the third mixture to incubate following mixing for a time period to produce the lipid-based particle.

127. The method of claim 126, wherein the time period to produce the lipid-based particle comprises 1 min to 60 min; 10 min to 30 min; or 10 min to 15 min.

128. The method of claim 126, wherein creating the first mixture comprises mixing components comprising:
(a) (i) 0.0075 mol-% to 0.5 mol-% of the PEGylated lipid conjugate;
(ii) 30 mol-% to 60 mol-% of the ionizable lipid;
(iii) 5.0 mol-% to 25 mol-% of the helper lipid;
(iv) 20 mol-% to 60 mol-% of the cholesterol; and
(v) 5 mol-% or less of the non-conjugated PEGylated lipid; or
(b) (i) 0.0075 mol-% to 0.2 mol-% of the PEGylated lipid conjugate;
(ii) 30 mol-% to 60 mol-% of the ionizable lipid;
(iii) 5.0 mol-% to 15 mol-% of the helper lipid;
(iv) 20 mol-% to 60 mol-% of the cholesterol; and
(v) 3 mol-% or less of the non-conjugated PEGylated lipid; or
(c) (i) 0.0075 mol-% to 0.08 mol-% of the PEGylated lipid conjugate;
(ii) 40 mol-% to 60 mol-% of the ionizable lipid;
(iii) 7.5 mol-% to 15 mol-% of the helper lipid;
(iv) 30 mol-% to 40 mol-% of the cholesterol; and
(v) 2 mol-% or less of the non-conjugated PEGylated lipid; or
(d) (i) 0.01 mol-% to 0.06 mol-% of the PEGylated lipid conjugate;
(ii) 45 mol-% to 55 mol-% of the ionizable lipid;
(iii) 7.5 mol-% to 12.5 mol-% of the helper lipid;
(iv) 35 mol-% to 40 mol-% of the cholesterol; and
(v) 1.5 mol-% or less of the non-conjugated PEGylated lipid.

129. The method of claim 126, wherein:
(a) the ionizable lipid comprises SM-102, MC3, or lipid 5;
(b) the helper lipid comprises distearoylphosphatidylcholine (DSPC) or dioleoyl-phosphatidylethanolamine (DOPE);
(c) the PEGylated lipid of the PEGylated lipid conjugate comprises DMG-PEG2K; and/or
(d) the non-conjugated PEGylated lipid comprises DMG-PEG2K.

130. A pharmaceutical composition comprising the lipid-based particle of claim 100 and a pharmaceutically acceptable carrier.

131. A method of administering the pharmaceutical composition of claim 130 to a patient comprising parenteral administration.

132. The method of claim 131, wherein the patient is a human.

133. The method of claim 132, wherein the patient is suffering from a disease selected from an autoimmune disease, inflammatory disease, cardiovascular disease, hepatic disease, or cancer.
